SEQUENCE LISTING (55 sequences)

SEQ ID NO: 1 - protein, 32 aa, HOMO SAPIENS
  VARIANT 24: Xaa = 6-aminohexanoic acid
GDWIDSILAFSRSLHSLLVDKKCXRRRRRRRR

SEQ ID NO: 2 - protein, 25 aa, HOMO SAPIENS
  VARIANT 17: Xaa = 6-aminohexanoic acid
SILAFSRSLHSLLVDGXRRRRRRRR

SEQ ID NO: 3 - protein, 24 aa, HOMO SAPIENS
  VARIANT 16: Xaa = 6-aminohexanoic acid
ILAFSRSLHSLLVDGXRRRRRRRR

SEQ ID NO: 4 - protein, 23 aa, HOMO SAPIENS
  VARIANT 15: Xaa = 6-aminohexanoic acid
LAFSRSLHSLLVDGXRRRRRRRR

SEQ ID NO: 5 - protein, 22 aa, HOMO SAPIENS
  VARIANT 14: Xaa = 6-aminohexanoic acid
AFSRSLHSLLVDGXRRRRRRRR

SEQ ID NO: 6 - protein, 21 aa, HOMO SAPIENS
  VARIANT 13: Xaa = 6-aminohexanoic acid
FSRSLHSLLVDGXRRRRRRRR

SEQ ID NO: 7 - protein, 20 aa, HOMO SAPIENS
  VARIANT 12: Xaa = 6-aminohexanoic acid
SRSLHSLLVDGXRRRRRRRR

SEQ ID NO: 8 - protein, 20 aa, HOMO SAPIENS
  VARIANT 12: Xaa = 6-aminohexanoic acid
FSRSLHSLLVGXRRRRRRRR

SEQ ID NO: 9 - protein, 19 aa, HOMO SAPIENS
  VARIANT 11: Xaa = 6-aminohexanoic acid
FSRSLHSLLGXRRRRRRRR

SEQ ID NO: 10 - protein, 18 aa, HOMO SAPIENS
  VARIANT 10: Xaa = 6-aminohexanoic acid
FSRSLHSLGXRRRRRRRR

SEQ ID NO: 11 - protein, 17 aa, HOMO SAPIENS
  VARIANT 9: Xaa = 6-aminohexanoic acid
FSRSLHSGXRRRRRRRR

SEQ ID NO: 12 - protein, 19 aa, HOMO SAPIENS
  VARIANT 11: Xaa = 6-aminohexanoic acid
ASRSLHSLAGXRRRRRRRR

SEQ ID NO: 13 - protein, 19 aa, HOMO SAPIENS
  VARIANT 11: Xaa = 6-aminohexanoic acid
FSRSLHSLLGXRRRRRRRR

SEQ ID NO: 14 - protein, 19 aa, HOMO SAPIENS
  VARIANT 11: Xaa = 6-aminohexanoic acid
LLSHLSRSFGXRRRRRRRR

SEQ ID NO: 15 - protein, 19 aa, HOMO SAPIENS
  VARIANT 11: Xaa = 6-aminohexanoic acid
LLSHLSRSFGXRRRRRRRR

SEQ ID NO: 16 - protein, 19 aa, HOMO SAPIENS
  VARIANT 11: Xaa = 6-aminohexanoic acid
FGDWIDSILGXRRRRRRRR

SEQ ID NO: 17 - protein, 19 aa, HOMO SAPIENS
  VARIANT 11: Xaa = 6-aminohexanoic acid
FAALSALVLGXRRRRRRRR

SEQ ID NO: 18 - protein, 19 aa, HOMO SAPIENS
  VARIANT 11: Xaa = 6-aminohexanoic acid
FYLKLEDLVGXRRRRRRRR

SEQ ID NO: 19 - protein, 24 aa, HOMO SAPIENS
SIKDFSLNLQSLNLDGRRRRRRRR

SEQ ID NO: 20 - protein, 24 aa, HOMO SAPIENS
SIVEFSSNLQNMNIDGRRRRRRRR

SEQ ID NO: 21 - protein, 12 aa, HOMO SAPIENS
RRRFRRRLRRLL

SEQ ID NO: 22 - protein, 12 aa, HOMO SAPIENS
RRRFRRRLRRLL

SEQ ID NO: 23 - protein, 19 aa, HOMO SAPIENS
  VARIANT 11: Xaa = 6-aminohexanoic acid
FSRSLHSLLGXRRRRRRRR

SEQ ID NO: 24 - protein, 19 aa, HOMO SAPIENS
  VARIANT 11: Xaa = 6-aminohexanoic acid
FSRSLHSLLGXRRRRRRRR

SEQ ID NO: 25 - protein, 19 aa, HOMO SAPIENS
  VARIANT 11: Xaa = 6-aminohexanoic acid
FARSLHSLLGXRRRRRRRR

SEQ ID NO: 26 - protein, 19 aa, HOMO SAPIENS
  VARIANT 11: Xaa = 6-aminohexanoic acid
FSASLHSLLGXRRRRRRRR

SEQ ID NO: 27 - protein, 19 aa, HOMO SAPIENS
  VARIANT 11: Xaa = 6-aminohexanoic acid
FSRALHSLLGXRRRRRRRR

SEQ ID NO: 28 - protein, 19 aa, HOMO SAPIENS
  VARIANT 11: Xaa = 6-aminohexanoic acid
FSRSLASLLGXRRRRRRRR

SEQ ID NO: 29 - protein, 19 aa, HOMO SAPIENS
  VARIANT 11: Xaa = 6-aminohexanoic acid
FSRSLHALLGXRRRRRRRR

SEQ ID NO: 30 - protein, 18 aa, HOMO SAPIENS
  VARIANT 10: Xaa = 6-aminohexanoic acid
FRSLHSLLGXRRRRRRRR

SEQ ID NO: 31 - protein, 18 aa, HOMO SAPIENS
  VARIANT 10: Xaa = 6-aminohexanoic acid
FSSLHSLLGXRRRRRRRR

SEQ ID NO: 32 - protein, 18 aa, HOMO SAPIENS
  VARIANT 10: Xaa = 6-aminohexanoic acid
FSRLHSLLGXRRRRRRRR

SEQ ID NO: 33 - protein, 18 aa, HOMO SAPIENS
  VARIANT 10: Xaa = 6-aminohexanoic acid
FSRSLSLLGXRRRRRRRR

SEQ ID NO: 34 - protein, 18 aa, HOMO SAPIENS
  VARIANT 10: Xaa = 6-aminohexanoic acid
FSRSLHLLGXRRRRRRRR

SEQ ID NO: 35 - protein, 17 aa, HOMO SAPIENS
  VARIANT 9: Xaa = 6-aminohexanoic acid
FSLHSLLGXRRRRRRRR

SEQ ID NO: 36 - protein, 18 aa, HOMO SAPIENS
  VARIANT 10: Xaa = 6-aminohexanoic acid
FRSLHSLLGXRRRRRRRR

SEQ ID NO: 37 - protein, 18 aa, HOMO SAPIENS
  VARIANT 2, 10: Xaa = 6-aminohexanoic acid
FXSLHSLLGXRRRRRRRR

SEQ ID NO: 38 - protein, 19 aa, HOMO SAPIENS
  VARIANT 11: Xaa = 6-aminohexanoic acid
FSRSLHSLLGXCGNKRTAC

SEQ ID NO: 39 - protein, 45 aa, HOMO SAPIENS
  VARIANT 11: Xaa = 6-aminohexanoic acid
FSRSLHSLLGXAKVKDEPQRRSARLSAKPAPPKPEPKPKKAPAKK

SEQ ID NO: 40 - protein, 14 aa, HOMO SAPIENS
  VARIANT 6: Xaa = 6-aminohexanoic acid
FLLLGXRRRRRRRR

SEQ ID NO: 41 - protein, 14 aa, HOMO SAPIENS
  VARIANT 6: Xaa = 6-aminohexanoic acid
FLLLGXRRRRRRRR

SEQ ID NO: 42 - protein, 27 aa, HOMO SAPIENS
FSRSLHSLLCCRQIKIWFQNRRMKWKK

SEQ ID NO: 43 - protein, 27 aa, HOMO SAPIENS
FSRSLHSLLCCRQVKVWFQNRRMKWKK

SEQ ID NO: 44 - protein, 18 aa, HOMO SAPIENS
  VARIANT 10: Xaa = 6-aminohexanoic acid
FSDLWKLLGXRRRRRRRR

SEQ ID NO: 45 - protein, 29 aa, HOMO SAPIENS
NFQHALQEVLQALKQVQARCCRRRRRRRR

SEQ ID NO: 46 - protein, 19 aa, HOMO SAPIENS
  VARIANT 11: Xaa = 6-aminohexanoic acid
FSRSLHSLLGXRRRRRRRR

SEQ ID NO: 47 - protein, 19 aa, HOMO SAPIENS
  VARIANT 11: Xaa = 6-aminohexanoic acid
FSRSLHSLLGXRRRRRRRR

SEQ ID NO: 48 - protein, 27 aa, HOMO SAPIENS
FSRSLHSLLCCRQIKIWFQNRRMKWKK

SEQ ID NO: 49 - protein, 27 aa, HOMO SAPIENS
FSRSLHSLLCCRQVKVWFQNRRMKWKK

SEQ ID NO: 50 - protein, 32 aa, HOMO SAPIENS
FSRSLHSLLCCAGYLLGKINLKALAALAKKIL

SEQ ID NO: 51 - protein, 32 aa, HOMO SAPIENS
FSRSLHSLLCCAGYLLGKVNLKALAALAKKVL

SEQ ID NO: 52 - protein, 32 aa, HOMO SAPIENS; identical to SEQ ID NO: 50

SEQ ID NO: 53 - protein, 32 aa, HOMO SAPIENS; identical to SEQ ID NO: 51

SEQ ID NO: 54 - protein, 37 aa, HOMO SAPIENS
FSRSLHSLLCCGWTLNSAGYLLGKINKALAALAKKIL

SEQ ID NO: 55 - protein, 20 aa, HOMO SAPIENS
EDIIRNIARHLAQVGDSMDR

(19) United States
(12) Patent Application Publication
(10) Pub. No.: US 2009/0118135 A1
(43) Pub. Date: May 7, 2009

(54) METHODS AND COMPOUNDS FOR REGULATING APOPTOSIS

(75) Inventors: John C. Reed, Rancho Santa Fe, CA (US); Kenneth Yip, San Diego, CA (US); Eduard Sergienko, San Diego, CA (US); Ying Su, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: The Burnham Institute, La Jolla, CA (US)

(21) Appl. No.: 12/131,427

(22) Filed: Jun. 2, 2008

Related U.S. Application Data
(60) Provisional application No. 60/942,924, filed on Jun. 8, 2007.
(60) Provisional application No. 61/038,031, filed on Mar. 19, 2008.

(51) Int. Cl. C40B 30/04; G01N 33/68; G01N 33/50; C07K 1/113

(52) U.S. Cl. 506/9; 436/86; 435/7.92; 530/402; 530/409

(57) ABSTRACT

An assay for determining compounds that inhibit activity of a BCl-2 protein, or affect conversion of Bcl-2 from an antiapoptotic to a proapoptotic form are described. In addition, compounds that modulate the function of anti-apoptotic proteins such as Bcl-2 and related Bcl-2 family members are identified.

### RELATED APPLICATIONS

This application claims priority under 35 U.S.C. §119(e) to U.S. Provisional Application No. 60/942,924, filed Jun. 8, 2007, and U.S. Provisional Application No. 61/038,031, filed Mar. 19, 2008, the entire contents of which are incorporated herein by reference.

### STATEMENT REGARDING FEDERALLY SPONSORED R&D

This invention was made in part with United States government support under grant number 2 R01 GM060554, awarded by the National Institutes of Health. The U.S. Government has certain rights in this invention.

### BACKGROUND OF THE INVENTION

1. Field of the Invention

The present invention relates to screening assays for identifying inhibitors of Bcl-2 proteins, and compounds which convert Bcl-2 proteins from inhibitors of apoptosis to promoters of apoptosis (“converters”) Because such inhibitors promote apoptosis of proliferative cells, compounds identified as competitive inhibitors will also promote apoptosis. The competitive inhibitors and converters include peptides, peptide analogs and small molecules.

2. Description of the Related Art

Apoptosis, also known as programmed cell death, is a physiological process through which the body disposes of unneeded or undesirable native cells. The process of apoptosis is used during development to remove cells from areas where they are no longer required, such as the space between digits. Apoptosis is also important in the body's response to disease. Cells that are infected with some viruses can be stimulated to undergo apoptosis, thus preventing further replication of the virus in the host organism.

Impaired apoptosis due to blockade of the cell death-signaling pathways is involved in tumor initiation and progression, since apoptosis normally eliminates cells with increased malignant potential such as those with damaged DNA or aberrant cell cycling (White, 1996 Genes Dev 10:1-15). The majority of solid tumors are protected by at least one of the two cell death antagonists, Bcl-2 or BCl-X_{L}. Members of the Bcl-2-family are known to modulate apoptosis in different cell types in response to various stimuli. Some members of the family act to inhibit apoptosis, such as Bcl-2 and Bcl-X_{L}, while others, such as Bax, Bak, Bid, and Bad, promote apoptosis. The ratio at which these proteins are expressed can decide whether a cell undergoes apoptosis or not. For instance, if the Bcl-2 level is higher than the Bax level, apoptosis is suppressed. If the opposite is true, apoptosis is promoted. Bcl-2 overexpression contributes to cancer cell progression by preventing normal cell turnover caused by physiological cell death mechanisms, and has been observed in a majority of cancers (Reed, 1997 Sem Hematol 34:9-19; Buolamwini, 1999 Curr Opin Chem Biol 3:500-509). The expression levels of Bcl-2 proteins often correlate with resistance to a wide spectrum of chemotherapeutic drugs and γ-radiation therapy. Paradoxically, high levels of Bcl-2 also associate with favorable clinical outcomes for patients with some types of cancers.

Biological approaches targeted at reducing Bcl-2 levels using antisense oligonucleotides have been shown to enhance tumor cell chemosensitivity. Antisense oligonucleotides targeted to Bcl-2 in combination with chemotherapy are currently in phase II/III clinical trials for the treatment of patients with lymphoma and malignant melanoma, and further trials with patients with lung, prostate, renal, or breast carcinoma are ongoing or planned (Reed, 1997 Sem Hematol 34:9-19; Piche et al. 1998 Cancer Res 2134-2140; Webb et al. 1997 Lancet 349:1137-1141; Jansen et al. 1998 Nat Med 4:232-234; Waters et al. 2000 J Clin Oncol 18:1812-1823). Recently, cell-permeable Bcl-2 binding peptides and chemical inhibitors that target Bcl-2 have been developed, and some of them have been shown to induce apoptosis in vitro and in vivo (Finnegan et al. 2001 Br J Cancer 85:115-121; Enyedy et al. 2001 J Med Chem 44:4313-4324; Tzung et al. 2001 Nat Cell Biol 3:183-191; Degterev et al. 2001 Nat Cell Bio 3:173-182; Walensky et al. 2004 Science 305: 1466-1470; Oltersdorf et al. 2005 Nature 435: 677-681).).

One well-established apoptotic pathway involves mitochondria (Green and Reed, 1998 Science 281:1309-1312; Green and Kroemer, 1998 Trends Cell Biol 8:267-271). Cytochrome c is exclusively present in mitochondria and is released from mitochondria in response to a variety of apoptotic stimuli. Many Bcl-2-family proteins reside on the mitochondrial outer membrane. Bcl-2 prevents mitochondrial disruption and the release of cytochrome c from mitochondria, while Bax and Bak create pores in mitochondrial membranes and induce cytochrome c release. Recent evidence has indicated, however, that Bcl-2 under certain conditions can function as a pro-apoptotic molecule (Finnegan et al. 2001 Br J Cancer 85:115-121; Fujita et al 1998 Biochem Biophys Res Commun 246:484-488; Fadeel et al 1999 Leukemia 13:719-728; Grandgirard et al 1998 EMBO J. 17:1268-1278; Cheng et al. 1997 Science 278:1966-1968; Del Bello et al. 2001 Oncogene 20:4591-4595). Bcl-2 can be cleaved by caspase-3 and thus be converted to a pro-apoptotic protein similar to Bax (Cheng et al. 1997 Science 278:1966-1968). Conversely, Bax has also been shown to inhibit neuronal cell death when infected with Sindbis virus (Lewis et al 1999 Nat Med 5:832-835). These observations suggest that members of the Bcl-2-family have reversible roles in the regulation of apoptosis and have the potential to function either as a pro-apoptotic or anti-apoptotic molecule.

Bcl-2 proteins include Bcl-2, Bcl-X_{L}, Mcl-1, Bfl-1 (A1), Bcl-W and Bcl-B. Members of the Bcl-2-family of proteins are highly related in one or more specific regions, commonly referred to as Bcl-2 homology (BH) domains. BH domains contribute at multiple levels to the function of these proteins in cell death and survival. The BH3 domain, an amphipathic α-helical domain, was first delineated as a stretch of 16 amino acids in Bak that is required for this protein to heterodimerize with anti-apoptotic members of the Bcl-2-family and to promote cell death. All proteins in the Bcl-2-family contain a BH3 domain, and this domain can have a death-promoting activity that is functionally important. The BH3 domain acts as a potent “death domain” and there is a family of pro-apoptotic proteins that contain BH3 domains which dimerize via those BH3 domains with Bcl-2, Bcl-X_{L} and other anti-apoptotic members of the Bcl-2 family. Structural studies revealed the presence of a hydrophobic pocket on the surface of Bcl-X_{L} and Bcl-2 that binds the BH3 peptide. Interestingly, the anti-apoptotic proteins Bcl-X_{L} and Bcl-2 also possess BH3 domains, but in these anti-apoptotic proteins, the BH3 domain is buried in the core of the protein and not exposed for dimerization (Kelekar and Thompson 1998 Trends Cell Biol 8:324). NMR structural analysis of the Bcl-X_{L}/Bak BH3 peptide complex showed that the Bak BH3 domain binds to the hydrophobic cleft formed in part by the BH1, BH2 and BH3 domains of Bcl-X_{L} (Sattler 1997 Science 275:983; Degterev 2001 Nature Cell Biol 3:173-182). BH3-domain-mediated homodimerizations and heterodimerizations have a key role in regulating apoptotic functions of the Bcl-2-family (Diaz et al. 1997 J Biol Chem 272:11350; Degterev 2001 Nature Cell Biol 3:173-182).

The orphan receptor Nur77 (also known as TR3 or nerve growth factor-induced clone B NGFI-B, GenBank Accession No.: L13740, SEQ ID NO: 55) (Chang and Kokontis 1988 Biochem Biophys Res Commun 155:971; Hazel et al. 1988 PNAS USA 85:8444) functions as a nuclear transcription factor in the regulation of target gene expression (Zhang and Pfahl 1993 Trends Endocrinol Metab 4:156-162; Tsai and O'Malley 1994 Annu Rev Biochem 63:451; Kastner et al. 1995 Cell 83:859; Mangelsdorf and Evans 1995 Cell 83:841). Nur77 was originally isolated as an immediate-early gene rapidly expressed in response to serum or phorbol ester stimulation of quiescent fibroblasts (Hazel et al. 1988 PNAS USA 85:8444; Ryseck, et al. 1989 EMBO J. 8:3327; Nakai et al 1990 Mol Endocrinol 4:1438; Herschman 1991 Annul Rev Riochem 60:281). Other diverse signals, such as membrane depolarization and nerve growth factor, also increase Nur77 expression (Yoon and Lau 1993 J Biol Chem 268:9148). Nur77 is also involved in the regulation of apoptosis in different cell types (Woronicz et al 1994 Nature 367:277; Liu et al. 1994 Nature 367:281; Weih et al PNAS USA 93:5533; Chang et al, 1997 EMBO J. 16:1865; Li et al. 1998 Mol Cell Biol 18:4719; Uemura and Chang 1998 Endocrinology 129:2329; Young et al. 1994 Oncol Res 6:203). It is rapidly induced during apoptosis of immature thymocytes and T-cell hybridomas (Woronicz et al 1994 Nature 367:277; Liu et al 1994 Nature 367:281), in lung cancer cells treated with the synthetic retinoid 6-[3-(1-adamantyl)-4-hydroxyphenyl]-2-naphthalene carboxylic acid (AHPN) (Li et al 1998 Mol Cell Biol 18:4719) (also called CD437), and in prostate cancer cells treated with different apoptosis inducers (Uemura and Chang 1998 Endocrinology 129:2329; Young et al 1994 Oncol Res 6:203). Inhibition of Nur77 activity by overexpression of dominant-negative Nur77 or its antisense RNA inhibits apoptosis, whereas constitutive expression of Nur77 results in massive apoptosis (Weih et al PNAS USA 93:5533; Chang et al, 1997 EMBO J 16:1865).

Further studies of Nur77 have yielded a better understanding of its mechanism of action in apoptosis (Li et al. 2000 Science 289:1159). First, several apoptosis inducing agents which also induced Nur77 expression in human prostate cancer cells were identified. These included the AHPN analog 6-[3-(1-adamantyl)-4-hydroxyphenyl]-3-chloro-2-naphthalenecarboxylic acid (MM11453), the retinoid (Z)-4-[2-bromo-3-(5,6,7,8-tetrahydro-3,5,5,8,8-pentamethyl-2-naphthalenyl)propenoyl]benzoic acid (MM11384), the phorbol ester 12-O-tetradecanoyl phorbol-13-acetate (TPA), the calcium ionophore A23187, and the etoposide VP-16. Second, it was found that the transactivation activity of Nur77 was not required for its role in inducing apoptosis, as demonstrated by an experiment that showed that apoptosis-inducing agents blocked the expression of a Nur77 target reporter gene. This was further supported by the finding that a Nur77 mutant deprived of its DNA binding domain (DBD) was still competent for inducing apoptosis. Third, Nur77 was found to relocalize to the outer surface of the mitochondria in response to some apoptotic stimuli, and mitochondrial association of Nur77 is essential for its apoptotic effects.

Nur77, visualized in vivo by tagging with Green Fluorescent Protein (GFP), was shown to relocalize from the nucleus to the mitochondria in response to apoptosis-inducing agents. Fractionation studies showed that Nur77 was associating with the mitochondria-enriched heavy membrane fraction, and proteolysis accessibility studies on purified mitochondria confirmed that Nur77 was associating with the outer surface of the mitochondria, where Bcl-2-family members are also found, Fourth, Nur77 was shown to be involved in the regulation of cytochrome c release from the mitochondria. Inhibition of Nur77 activity by expression of Nur77 antisense RNA blocked the release of cytochrome c and mitochondrial membrane depolarization in cells stimulated with TPA and MM11453. Furthermore, incubating purified mitochondria with recombinant Nur77 protein resulted in cytochrome c release.

Li et al. (2000 Science 289:1159) further explored the function of Nur77 through mutation of the protein. A Nur77 mutant which had the DNA-binding domain (amino acid residues 168-467) removed (Nur77/ΔDBD) no longer localized in the nucleus in non-stimulated cells, but instead was consistently found in mitochondria. This localization phenotype was accompanied by a constant release of cytochrome c from the mitochondria. Three other deletion mutants were also generated and assayed: an amino-terminal deletion of 152 amino acids referred to as Nur77/Δ1, a 26 amino acid carboxy-terminal deletion referred to as Nur77/Δ2, and a 120 amino acid carboxy-terminal deletion referred to as Nur77/Δ3. The Nur77/Δ1 protein did not relocalize to the mitochondria in response to TPA, but maintained a nuclear localization. Nur77/Δ1 had a dominant-negative effect, preventing the relocalization of full-length Nur77 to the mitochondria and inhibiting apoptosis. Mitochondrial targeting was still observed in Nur77/Δ2 protein expressing cells, but not in Nur77/A3 protein cells in response to TPA treatment. These results indicated that carboxy-terminal and amino-terminal sequences are crucial for mitochondrial targeting of Nur77 and its regulation.

Experiments designed to alter the localization of Nur77/ΔDBD by fusing it to various cellular localization signals showed that Nur77 must have access to the mitochondria to induce its pro-apoptotic effect. When Nur77/ΔDBD was fused to a nuclear localization sequence, a plasma membrane targeting sequence, or an ER-targeting sequence, Nur77/ΔDBD was not targeted to the mitochondria and no induction of cytochrome c release was observed.

### SUMMARY OF THE INVENTION

The present invention provides a method of screening for compounds capable of converting a Bcl-B protein from an antiapoptotic form to a proapoptotic form, comprising providing a Bcl-B protein; providing a fluorescently labeled compound known to bind to and convert the Bcl-B protein to a proapoptotic form; contacting the Bcl-B protein and the binding compound in the presence or absence of a test compound or library of test compounds; and determining the fluorescence of the Bcl-B protein, wherein a decrease in fluorescence indicates that the test compound inhibits binding of the binding compound to the Bcl-B protein. In one embodiment, the test compound is a natural product or natural product derivative. In yet another embodiment, the fluorescent label is Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine or Texas Red. The compound known to bind to and convert the Bcl-B protein to an apoptotic form may be a peptide, peptide analog or small molecule. In one aspect of this embodiment, the peptide is TR3-9-r8 peptide. The method may further comprise at least one secondary screen to confirm that the test compound converts the Bcl-B protein from an antiapoptotic form to a proapoptotic form. In one embodiment, the secondary screen is an apoptosis assay. In one embodiment, the fluorescence is measured by fluorescence polarization. In other embodiments, the fluorescence is measured by time-resolved fluorescence resonance energy transfer (TR-FRET), solid phase amplification (SPA) or an ELISA-like assay. In another embodiment, the screening method is in high throughput format. In other embodiments, the decrease in fluorescence is at least 20%, at least 30%, at least 40% or at least 50%.

The present invention also provides a method of converting a Bcl-B protein from an antiapoptotic to a proapoptotic form, comprising contacting the Bcl-B protein with a small molecule. In one embodiment, the small molecule is selected from the group of molecules shown in Tables 5, 6, 7 and 8.

The present invention also provides a method of screening for compounds capable of inhibiting a Bcl-B protein, comprising: providing a Bcl-B protein; providing a fluorescently labeled compound known to bind to said Bcl-B protein; and contacting the Bcl-B protein and the fluorescently labeled binding compound in the presence or absence of a test compound or library of test compounds; and determining fluorescence of the Bcl-B protein, wherein a decrease in fluorescence indicates that the test compound inhibits binding of the fluorescently labeled binding compound to the Bcl-B protein. In one embodiment, the test compound is a natural product or natural product derivative. In another embodiment, the fluorescent label is Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine or Texas Red. The compound known to bind to the Bcl-B protein may be a peptide, peptide analog or small molecule. In one aspect of this embodiment, the peptide is TR3-9-r8 peptide. The method may further comprise at least one secondary screen to confirm that the test compound binds to the Bcl-B protein. In one embodiment, the secondary screen is an apoptosis assay. In one embodiment, the fluorescence is measured by fluorescence polarization. In other embodiments, the fluorescence is measured by time-resolved fluorescence resonance energy transfer (TR-FRET), solid phase amplification (SPA) or an ELISA-like assay. In another embodiment, the screening method is in high throughput format. In other embodiments, the decrease in fluorescence is at least 20%, at least 30%, at least 40% or at least 50%.

The present invention also provides a method of inhibiting a Bcl-B protein, comprising contacting the protein with a small molecule.

In one embodiment, the small molecule is selected from the group of molecules shown in Tables 5, 6, 7 and 8 or an analog thereof.

In one embodiment, the molecule has the following structure:

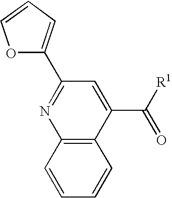

In which R¹ is —NH═Naryl, —NHaryl, —O[(CH₂)ₚNR¹⁰R¹¹], —O[(CH₂)ₚC(O)NR¹⁰R¹¹], or —O[(CH₂)ₚNR¹⁰R¹¹], each optionally substituted with one or more substituents, each independently halo, cyano, hydroxy, C₁₋₆ alkyl, C₁₋₆ alkoxy, phenyl, or and NR¹⁰R¹¹;

p is 1, 2, or 3; and

R¹⁰ and R¹¹ are each separately hydrogen, C₁₋₆ alkyl, aryl C₁₋₆ alkyl; or R¹⁴ and R¹⁵ are taken together with the nitrogen to which they are attached to form indolinyl, pyrrolidinyl, piperidinyl, piperazinyl, or morpholinyl.

In another embodiment, the molecule has the following structure:

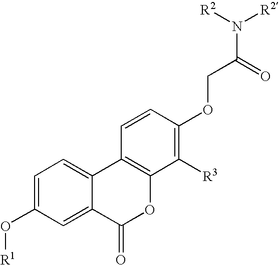

In which R¹ is hydrogen, aryl, heteroaryl, heterocyclyl, and C₁₋₆ alkyl optionally substituted with up to five fluoro;

R² and R^{2′} are each separately hydrogen or C₁₋₆ alkyl, —(CH₂)_{q}C₃₋₇cycloalkyl, aryl, heteroaryl, and heterocyclyl, each optionally substituted with one or more substituents each independently selected from halo, cyano, hydroxy, —(CH₂)_{q}C₃₋₇cycloalkyl, C₁₋₆ alkyl optionally substituted with up to 5 fluoro, and C₁₋₆ alkoxy optionally substituted with up to 5 fluoro; or R² and R^{2′} are taken together with the nitrogen to which they are attached to form a heterocyclyl;

R³ is hydrogen or selected from the group consisting of C₁₋₆ alkyl, —(CH₂)_{q}C₃₋₇cycloalkyl, and aryl each optionally substituted with one or more substituents each independently halo, cyano, and hydroxy; and

Q is 0, 1, 2, or 3.

In another embodiment, the molecule has the following structure:

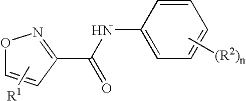

In which R¹ is hydrogen or selected from C₁₋₆ alkyl, and aryl; or R¹ is a fused C₃₋₇cycloalkyl;

R² is —SC₁₋₆alkyl, C₁₋₆alkoxy, C₁₋₆alkyl, —C(O)OC₁₋₆alkyl, and —C(O)NHC₁₋₆alkyl; and

n is 1, 2, 3, 4, or 5.

Another embodiment of the invention is a method of optimizing a target compound. This method includes providing a Bcl-B protein; providing a fluorescently labeled compound known to bind to said Bcl-B protein; contacting said Bcl-B protein and said fluorescently labeled binding compound in the presence or absence of a test compound or library of test compounds; determining fluorescence of said Bcl-B protein, wherein a decrease in fluorescence indicates that said test compound inhibits binding of said fluorescently labeled binding compound to said Bcl-B protein; reacting said test compound with a library of chemical fragments in the presence of Bcl-B protein to determine one or more chemical fragments that bind to a site adjacent said test compound; and linking said chemical fragment to said test compound If the chemical fragment binds adjacent said test compound.

### BRIEF DESCRIPTION OF THE DRAWINGS

FIG. 1A is a schematic diagram showing the differences in fluorescence polarization between an unbound fluorescently-labeled Bcl-B converter peptide (FITC-TR3-r8) and FITC-TR3-r8 bound to GST-labeled Bcl-B.

FIG. 1B is a schematic diagram illustrating the differences in fluorescence polarization of FITC-TR3-r8 (TR3-r8) in the presence of an unlabeled TR3-r8 peptide versus in the absence of the unlabeled peptide. When there is no competition, high polarization is observed due to more binding sites of Bcl-B being occupied by the fluorescently labeled TR3-r8. In the presence of unlabeled TR3-r8, fewer binding sites are occupied by the labeled converter peptide due to competition by the unlabeled peptide, resulting in decreased polarization.

FIGS. 2A-2B are graphs showing that FITC-TR3-9-r8 binds to three of the five anti-apoptotic Bcl-2 family proteins, and that this binding is dependent on the protein concentration. Binding to GST-Bcl-2 and GST-Bcl-B, but not GST-Bcl-XL, is shown in FIG. 2A. Binding to GST-Bfl-1, but not GST-Bcl-W, is shown in FIG. 2B.

FIG. 3 is a graph showing the effect of buffer components on the Bcl-B fluorescence polarization assay signal window. Fluorescence polarization of 20 nM FITC-TR3-9-r8 alone or in the presence of 200 nM Bcl-B was measured 10 min after sample preparation. Vertical bars correspond to the difference of fluorescence polarization values of samples with and without Bcl-B; error bars represent a sum of standard deviations of triplicates for each value. Buffers were tested with (right bars) and without (left bars) 1 mM TCEP and 0.005% Tween 20.

FIGS. 4A-4D are graphs showing the fluorescence signal stability in PBS (FIGS. 4A-B) and β-glycerophosphate (HβG) (FIGS. 4C-D) buffers with (FIGS. 4B and 4D) or without (FIGS. 4A and 4C) 1 mM TCEP and 0.005% Tween 20. Samples (20 nM FITC-TR3-9-r8 and 20 nM FITC-TR3-9-r8/200 nM Bcl-B) were prepared in polypropylene (PP) plates, then transferred to polystyrene (PS) plates and measured every 5 min. The same samples were incubated in PP plates for 2 h, then transferred into PS plates immediately before measurement (the last bar in each panel). Error bars represent standard deviation of triplicate samples.

FIG. 5 is a graph showing Bcl-B fluorescence signal stability in the buffers from the extended panel (Table 2). Samples (20 nM FITC-TR3-9-r8 and 20 nM FITC-TR3-9-r8/200 nM Bcl-B) were prepared in polypropylene (PP) plates, then transferred to polystyrene (PS) plates and measured at different time points. ΔmP is the difference in fluorescence polarization values of samples with and without Bcl-B; error bars represent the sum of standard deviations of triplicates for each of the two values.

FIGS. 6A-6B are graphs showing the binding of FITC-TR3-9-r8 to Bcl-B in HβG (FIG. 6A) and PBS (FIG. 6B) buffers added with 1 mM TCEP, 0.005% Tween 20. Bcl-B was diluted to different concentrations in HβG/Tween 20/TCEP buffer and added to 20 nM FITC-TR3-9-r8 in LJL HE 96 B plates. Fluorescence polarization was measured at the indicated time points (FIG. 6A). A similar experiment was also performed in PBS/Tween 20/TCEP (FIG. 6B). The information under the graphs contain the results of non-linear regression to hyperbolic equation with an offset, where V=nPₘₐₓ, Vₒ=mPₘᵢₙ, and K=apparent dissociation constant.

FIG. 7 is a graph showing the fluorescent signal stabilization at different concentrations of Bcl-B in HβG/Tween 20/TCEP buffer. Bcl-B was diluted to different concentrations in HβG/Tween 20/TCEP buffer and added to 20 nM FITC-TR3-9-r8 in LJL HE 96 B plates. Fluorescence polarization was measured after the indicated time.

FIG. 8 is a graph showing the results of a high throughput fluorescence polarization assay using Bcl-B in combination with FITC-Tr3 alone, or Bcl-B in the presence of FITC-Tr3-9-R8 and unlabeled T3r-9-R8. Decreased fluorescence signal is observed in the presence of Tr3 which acts as a competitive inhibitor and prevents FITC-Tr3 from binding at the binding site. mP_{high} is the mean fluorescence polarization signal (ex/em: 4851530) of negative controls in the corresponding plate. mP_{low} is the mean fluorescence polarization signal (ex/em: 485/530) of positive controls in the corresponding plate. 3SD means three standard deviations of the corresponding control. Given the mean (μ) and standard deviation (s) of both the positive (p) and negative (n) controls (μₚ,sₚ,μₙ,sₙ, respectively),

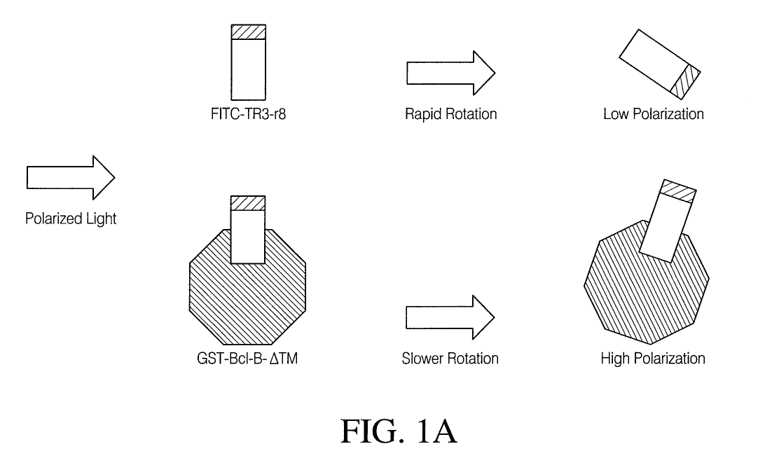

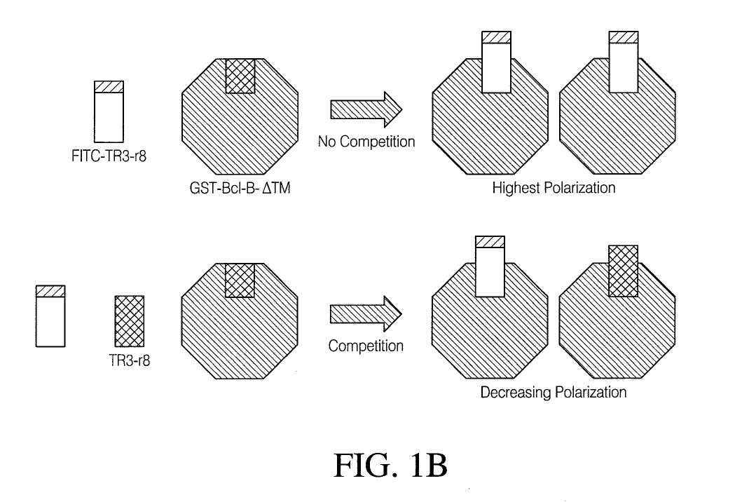

$$Zfactor = 1 - \frac{3 \times ( {σ}_{p} + {σ}_{n} )}{\vert {μ}_{p} - {μ}_{n} \vert}$$

If Z-factor-1.0, then screen/assay is statistically “perfect.”

If Z-factor=0.5-1.0° then screen/assay is statistically “excellent.”

If Z-factor=0-0.5, then screen/assay is statistically “marginal.”

If Z-factor=less than 0-0.5, then screen/assay is statistically “useless.”

### (Zhang J H, Chung T D, Oldenburg K R, “A Simple Statistical Parameter for Use in Evaluation and Validation of High Throughput Screening Assays.” J Biomol Screen. 1999; 4(2):67-73.)

FIG. 9 is a graph showing the % competition versus fluorescence intensity in a high throughput fluorescence polarization assay in which 50,000 compounds from a Chembridge library were used. Out of the 50,000 compounds screened, 427 (0.85%) exhibited at least 50% competition, and 332 (0.66%) had an F-ratio of 1.25 or lower. The F-ratio is fluorescence intensity normalized to the average fluorescence intensity value of the plate negative controls. The Z-factor was 0.75.

FIG. 10 is a graph showing the fluorescence polarization observed upon binding of FITC-Bid BH3 peptide to various concentrations of Bcl-2 family fusion proteins. Various concentrations of GST or GST-Bcl-2 family fusion proteins were incubated with 5 nM FITC-Bid BH3 peptide in PBS, pH 7.4. Fluorescence polarization (in milli-Polars) was measured after 10 min.

FIGS. 11A-G are graphs showing the results of competition assays using a fluorescence polarization assay with the green tea compound epigallecatechin (EGCG) and various Bcl-2 family proteins. 100 nM of GST-Bcl-2 fusion proteins were incubated with various concentrations of EGCG or control compound ECG (“C”) for 2 min in PBS in 50 μl. 5 nM FITC-Bid BH3 peptide was added to bring the final volume to 100 μl and the final DMSO concentration to 1%. Fluorescence polarization was measured after 20 min.

FIG. 12 is a graph showing representative screening results using Bfl-1 fluorescence polarization analysis of a library of compounds. This is a graphical representation of the data presented in Table 6. Y-axis=fluorescence polarization in milli-Polars; x-axis=well number (1-960 (A1 to H12). Wells A1 to H1 are the negative control (BH3 peptide without GST-Bfl-1 protein), and wells A12 to H12 are the positive control (no compounds). A Bfl-1 inhibitory compound is found in well B9 (candidate hit).

FIG. 13A shows graphs comparing the fluorescent signal strength in PBS versus HEPES-β-3-glycerophosphate buffer. FITC-TR3-9-r8 (20 nM) was incubated with various concentrations of GST-Bcl-B, and fluorescence polarization was measured. FIG. 13A contrasts the results obtained in PBS (left panel) vs. HEPES, pH7.5 with β-glycerophosphate (right panel), showing that binding is stable for several hours in HEPES-β-glycerophosphate buffer, but not PBS.

FIG. 13B is a graph showing a competitive displacement assay using increasing concentrations of unlabeled TR3 peptide to compete with a fixed concentration of FITC-TR3-9-r8 peptide for binding to Bcl-B in HEPES-β-glycerophosphate buffer. Fluorescence polarization was measured every 20 min over 2 h. Unlabeled TR3 is an effective competitive inhibitor of the FITC-labeled peptide.

FIG. 13C is a graph showing the stability of the fluorescence polarization signal over time. The mPₘₐₓ (squares), mPₘᵢₙ (triangles), and apparent K_{d} (circles) for the FPA were measured at various times from the same plate to assess the stability of the assay when conducted using β-glycerophosphate buffer. Data represent mean±SD for n=3.

FIGS. 14A-14B are graphs showing competition assays to determine whether the TR3 peptide binds to the same site on Bcl-2 where BH3 peptides bind. Fixed concentrations of FITC-TR3-9-r8 peptide and Bcl-2 protein were incubated in the presence or absence of unlabeled TR3 peptide, mutant TR3 peptide, BH3 peptide, or compound ABT-737, a known inhibitor of Bcl-2 proteins. As expected, unlabeled TR3 peptide competed with FITC-TR3-9-r8 for binding to Bcl-2, whereas the mutant TR3 peptide was less active (FIG. 14A). In contrast, BH3 peptide and ABT-737 failed to block FITC-TR3-9-r8 peptide binding to Bcl-2 (FIG. 14B).

FIGS. 15A-H are graphs showing the effect of different buffers on Bcl-B/FITC-TR3 binding and signal stability. FIG. 15A: BES, pH 7.0; FIG. 15B: PIPES, pH 7.0; FIG. 15C: MOPS, pH 7.0; FIG. 15D; TES, pH 7.0; FIG. 15E, Imidazole, pH 7.0; FIG. 15F, Bis-Tris, pH 7.0; FIG. 15G, HEPES, pH 7.6; FIG. 15H, pH 7.0. Each buffer contained 25 mM buffer concentration, 1 mM TCEP and 0.005% Tween 20, 20 mM FITC-Tr3-r8. Curves were analyzed using 4-parameter sigmoidal equation (mP=mP_WINDOW*[Bcl-B]/(K+[Bcl-B])+mP_OFFSET).

FIG. 16 is a graph showing Displacement of FITC-TR3-r8 from a complex with Bcl-B with TR3-r8. Bcl-B (15 nM) in 25 mM PIPES, pH 7.0, containing 1 mM TCEP, 0.005% Tween 20 and 20 nM FITC-TR3 was added with different concentrations of TR3-r8. Fluorescence polarization was measured after 15 min incubation. Nonlinear regression analysis was performed using 4-parameter sigmoidal equation (mP=Assay_WINDOW*KD̂H/(KD̂H+[TR3-r8]̂H)+mP_MIN).

FIG. 17 shows the order of component addition for Bcl-B FPA. squares: Bcl-B preincubated with FITC-Tr3-R8 for 1 h and then added with serially diluted Tr3-R8. triangles: Bcl-B preincubated with serially diluted Tr3-R8 for 1 h and then added with FITC-Tr3-R8. Final composition of all samples: 25 nM Bcl-B, 20 nM FITC-Tr3-R8 and Tr3-R8 present at 0 to 500 nM in assay buffer (25 mM HEPES, 20 mM β-glycerophosphate, pH 7.5, 1 mM TCEP, 0.005% Tween 20). Fluorescence polarization was measured 5 min after the last addition.

FIG. 18 shows the stability of the Bcl-B/FITC-Tr3-R8 mixture in different storage conditions. 25 nM Bcl-B and 20 nM FITC-Tr3-R8 were added together in assay buffer. The mixture was dispensed into wells containing 10% DMSO or Tr3-R8 in 10% DMSO to give 1% DMSO and 5 uM peptide, respectively. Fluorescence polarization in the wells was measured right after mixing (group 1) and 2 h after storage in polystyrene (PS) assay plate at room temperature (group 2). Part of the assay mixture was kept for 2 h in polypropylene (PP) container at +4° C. (group 3) or at room temperature (group 4) and was dispensed to the plate right before the measurement. first bar at each time point—mP in the presence of 1% DMSO, second bar at each time point mP values in the presence of 5 uM Tr3-R8, third bar at each time point—assay window. Error bars represent standard deviations of the data.

FIGS. 19A-19C are line graphs showing that TR3-r8 binds to Bcl-B. FIG. 19A shows that FITC-TR3-r8 binds to Bcl-B. Increasing concentrations of GST-Bcl-B were incubated with 20 nM FITC-TR3-r8 in a 25 mM HEPES-KOH, 20 mM β-glycerophosphate, 0.005% Tween-20, pH 7.5 buffer for 10 min, after which fluorescence polarization was measured. The apparent K_{d} was 20 nM. FIG. 19B shows that TR3-r8 can displace FITC-TR3-r8 from Bcl-B, and that FITC-TR3-r8 likely binds to a different site than Bim-BH3, Bax-BH3 (BH3 peptides that inhibit Bcl-B), and EGCG (a compound that inhibits Bcl-B and some other Bcl-2 family members). GST-Bcl-B (20 nM) was incubated with FITC-TR3-r8 (20 nM) and either TR3-r8, Bim-BH3, Bax-BH3, EGCG, or GST in a 25 mM HEPES-KOH, 20 mM β-glycerophosphate, 0.005% Tween-20, pH 7.5 buffer for 10 min, after which fluorescence polarization was measured. In FIG. 19C shows that TR3-r8 can displace FITC-TR3-r8 from Bcl-2, and that FITC-TR3-r8 likely binds to a different site than Bak-BH3 9a BH3 peptide that binds Bcl-2) and ABT-737 (a compound that inhibits Bcl-2 and some other Bcl-2 family members). GST-Bcl-2 (200 nM) was incubated with FITC-TR3-r8 (20 nM) and either TR3-r8, Bak-BH3, ABT-737, or GST in a PBS, 0.005% Tween-20, pH 7.5 buffer for 10 min, after which fluorescence polarization was measured. All data shown represent mean±standard deviation (n=3).

FIGS. 20A-B are graphs showing the results of a Bcl-B/TR3-r8 Fluorescence Polarization Compound Library Screen. In FIG. 20A GST-Bcl-B (20 nM) was incubated with either FITC-TR3-r8 (20 nM; squares) or with FITC-TR3-r8 (20 nM) and TR3-r8 (5 μM; diamonds) for 10 min, after which fluorescence polarization was measured. The data represent fluorescence polarization (mP) measured in individual wells, constituting 192 replicates for each condition. In FIG. 20B GST-Bcl-B (20 nM) was incubated with either FITC-TR3-r8 (20 nM) alone (negative control; cross-hatched peak), FITC-TR3-r8 (20 nM) and 5 μM TR3-r8 (positive control; lighter peak), or FITC-TR3-r8 (20 nM) and 3.75 mg/L ChemBridge DIVERSet compounds (darker peak).

FIG. 21 shows a workflow chart for characterization of Bcl-B-binding compounds. Compounds from the ChemBridge DIVERSet library were screened using the FITC-TR3-r8/GST-Bcl-B fluorescence polarization assay (FPA), and primary hits were confirmed twice at the screening concentration (3.75 mg/L). Dose-response curves were then generated (fluorescent compounds were eliminated), followed by counter-screening using a biologically unrelated FPA (GST-Hsp70/FITC-ATP). After further analyses of the FITC-TR3-r8/GST-Bcl-B dose-response curves, separate stocks of compounds were used for dose-response confirmation. Finally, 1D-NMR was used to confirm specific compound binding to GST-Bcl-B, and not to GST.

FIGS. 22A-22F are line graphs showing a ¹H-NMR spectra confirmation of compound binding to Bcl-B. Compounds 5804000, 5954623, and 2011727 were diluted in PBS/D₂O buffer to 1000 μM, then incubated with either GST (FIGS. 22A, C, E) or GST-Bcl-B (FIGS. 22B, D, F) at 0, 10 or 20 μM concentrations. FIGS. 22A-B show compound 5804000; FIGS. 22C-D show compound 5954623; and FIGS. 22E-F show the NMR spectra for the negative control compound 2011727. The most prominent peak shift for each compound is shown.

FIGS. 23A-23D show the characterization of compound competition with FITC-TR3-r8 for Bcl-B binding by FPA. FIG. 23A shows structures of confirmed-compound hits. In FIGS. 23B GST-Bcl-B (20 nM) was incubated with FITC-TR3-r8 (20 nM) and either unlabeled TR3-r8 or the indicated compounds in a 25 mM HEPES-KOH, 20 mM β-glycerophosphate, 0.005% Tween-20, pH 7.5 buffer for 10 min, after which fluorescence polarization was measured. 5729206 is a negative control compound. In FIG. 23C GST-Bcl-B (16.4 nM) was incubated with FITC-Puma-BH3 (5 nM) and various concentrations of peptides, compounds, or GST in PBS pH 7.5, 0.005% Tween-20 for 10 min, after which fluorescence polarization was measured. In FIG. 23D GST-Bcl-B (8.8 nM) was incubated with FITC-Bim-BH3 (5 nM) and different concentrations of peptides, compounds, or GST in PBS pH 7.5, 0.005% Tween-20 for 10 min, after which fluorescence polarization was measured. All data shown represent mean±standard deviation (n=3).

FIGS. 24A-24C show that compound 5804000 Exhibits Bcl-B-Dependent Biological Activity. FIG. 24A is a photograph of a blot wherein HeLa Tet-On (HTO1) and HeLa Tet-On-Bcl-B (HTO2) cells were treated with or without doxycycline (dox) for 24 h to induce their tetracycline-responsive promoters. Protein lysates were collected, and immunoblotting was used to analyze Bcl-B and Hsc70 (for loading control) expression. In FIG. 24B the cells were treated with or without doxycycline for 24 h, and then with staurosporine at the indicated concentrations for 24 h. Cell viability was measured using ATPlite. In FIG. 24C HeLa Tet-On-Bcl-B (HTO2) cells were treated with or without doxacycline as above. Compounds were then added at the indicated concentrations, and cell viability was measured 24 h later using ATPlite. The data shown represent mean±standard deviation (n=3).

FIGS. 25A-H show a furylquinoline scaffold structure (FIG. 25A), and active furylquinoline-based inhibitors of Bcl-B (FIGS. 25A-H) as determined using the Bcl-B/TR3 fluorescence polarization assay described herein. The structure shown in FIG. 25G had much lower activity in the FPA than the other structures. All of these inhibitors had an IC50 of >100 μM against Bfl-1/Bid, demonstrating the specificity of the assay for Bcl-B. The MLS numbers shown in each figure stands for Molecular Library Screening Centers Network at the National Institutes of Health.

FIGS. 26A-H show a benzo[c]chrome-6-one scaffold structure (FIG. 26A), and active benzo[c]chrome-6-one-based inhibitors of Bcl-B (FIGS. 26B-H) as determined using the Bcl-B/TR3 fluorescence polarization assay described herein. The structures shown in FIGS. 25D, E and F had much lower activity than the other structures. All of these inhibitors had an IC50 of >50 μM against Bfl-1/Bid, demonstrating the specificity of the assay for Bcl-B.

FIGS. 27A-I show a isoxazol-benzamide scaffold structure (FIG. 27A), and active isoxazol-benzamide-based inhibitors of Bcl-B as determined using the Bcl-B/TR3 fluorescence polarization assay described herein. The structures shown in FIGS. 27C and D had much lower activity than the other structures. All of these inhibitors had an IC50 of >100 μM against Bfl-1/Bid, demonstrating the specificity of the assay for Bcl-B.

### DETAILED DESCRIPTION OF THE PREFERRED EMBODIMENTS

The present invention provides methods for identifying compounds that bind to members of the Bcl-2 family of proteins, and convert these proteins from inhibitors of apoptosis to promoters of apoptosis (antiapoptotic to proapoptotic state). These compounds are referred to herein as “converters”, and peptides capable of such conversion are referred to as “converter peptides.”

In another embodiment, compounds are identified which bind inhibit the activity of members of the Bcl-2 family of proteins. These compounds are referred to herein as “inhibitors”, and peptides capable of such inhibition are referred to as “inhibitor peptides.” In addition, the term “pro-apoptotic modulator” as used herein is meant to include converters, inhibitors, and any other compound that promotes apoptosis.

In one embodiment, the method involves binding a fluorescently labeled compound known to convert a Bcl-2 protein to a pro-apoptotic form to a Bcl-2 protein in the presence or absence of a test compound or library of test compounds; and determining the fluorescence of the Bcl-2 protein, wherein a decrease in fluorescence indicates that the test compound inhibits binding of the compound to the Bcl-2 protein.

In another embodiment, the method involves binding a fluorescently labeled compound known to bind to a Bcl-2 protein in the presence or absence of a test compound or library of test compounds; and determining the fluorescence of the Bcl-2 protein, wherein a decrease in fluorescence indicates that the test compound inhibits binding of the compound to the Bcl-2 protein. In other embodiments, these methods relate to a fluorescence polarization assay (FPA) using a fluorescently-labeled, known converter or inhibitor of a member of the Bcl-2 family of proteins. Other fluorescence-based assays well known in the art may also be used, including time-resolved fluorescence resonance energy transfer (TR-FRET), solid phase amplification (SPA) and ELISA-like assays.

In the FPA, a known agent that binds to a Bcl-2 protein, or converts a Bcl-2 protein into a pro-apoptotic state (e.g., peptide TR3-9-r8; FSRSLHSLLGXrrrrrrrr—SEQ ID NO:9) is fluorescently labeled and incubated with a Bcl-2 protein in the presence or absence of a test compound, or library of compounds, followed by determination of the resulting level of Bcl-2 protein fluorescence polarization. If the polarization of the Bcl-2 protein in the presence of the test compound is significantly less than in the absence of the test compound, then the test compound inhibits binding of the fluorescently labeled compound. Such compounds may act as inhibitors of the Bcl-2 protein, or may be capable of converting the Bcl-2 protein from an antiapoptotic to a proapoptotic form. Peptides which convert a Bcl-2 protein into a pro-apoptotic state are referred to herein as “converter peptides.” Once converters and inhibitors are identified, they are subjected to one or more secondary screens as described below to determine their ability to convert a Bcl-2 protein from an inhibitor to a promoter of apoptosis (converters), or to determine their ability to promote apoptosis by inhibiting a Bcl-2 protein. Although the use of TR3-9-r8 is described herein, the use of other peptides that bind to the same binding site on Bcl-2 proteins is also within the scope of the embodiments described herein.

By “significantly less”, it is meant that the amount of fluorescence or fluorescence polarization observed in the presence of the test compound is about 99% less, 95% less, 90% less, 85% less, 80% less, 75% less, 70% less, 65% less, 60% less, 55% less, 50% less, 45% less, 40% less, 35% less, 30% less, 25% less or 20% less than the fluorescence or fluorescence polarization observed in the absence of the test compound. In one embodiment, the amount of fluorescence or fluorescence polarization observed in the presence of the test compound is about 50% of the fluorescence or fluorescence polarization observed in the presence of the test compound. Once such competitive inhibitors are identified, their ability to promote apoptosis in transformed cell lines is confirmed using cellular apoptosis assays well known in the art, such as those described herein. Other pro-apoptotic modulators of Bcl-2 are shown in Table 1 below.

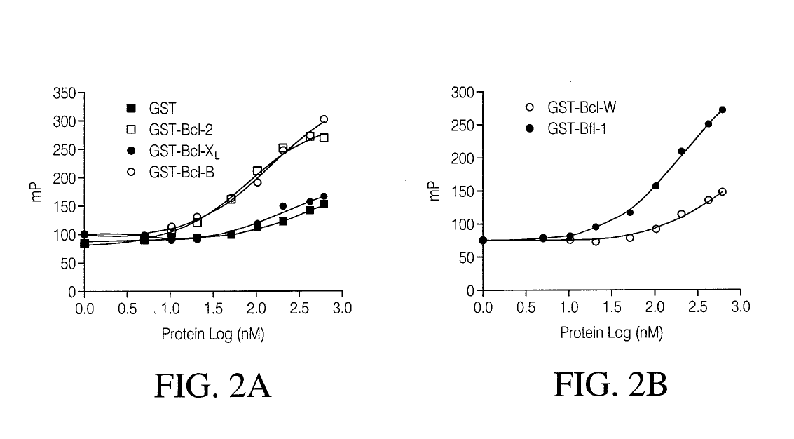

As illustrated in FIG. 2A, binding of a Bcl-2 converter (e.g., FITC-TR3-r8) to a Bcl-2 protein (e.g., GST-Bcl-B) resulted in slower rotation of polarized light and increased polarization compared to unbound FITC-TR3-r8. As illustrated in FIG. 2B, TR3-9-r8 which does not contain a fluorescent label is a competitive inhibitor of FITC-TR3-9-r8, resulting in decreased polarization. When a test compound, or library of compounds, is screened using this assay in a 96-well format or 384-well (or greater) high throughput format, compounds that have minimal effect on the interaction of a Bcl-2 protein exhibit high levels of polarization because very little of the fluorescently-labeled inhibitor is displaced from the Bcl-2 protein. In contrast, competitive inhibitors result in reduced polarization due to competitive displacement of the fluorescently-labeled inhibitor from the Bcl-2 protein, and replacement by the non-labeled competitive inhibitor.

Although this assay is exemplified herein using certain Bcl-2 proteins and inhibitors, it will be appreciated that any member of the Bcl-2 family of proteins (e.g., Bcl-2, Bcl-X_{L}, Mcl-1, Bfl-1 (A1), Bcl-W and Bcl-B), and any fluorescently labeled inhibitor known to bind to these proteins, can be used within the assay described herein. Although fluorescein isothiocyanate (FITC)-labeled peptide inhibitors are exemplified herein, other fluorescent labels may also be used, including Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

The pro-apoptotic modulators of Bcl-2 activity discovered using the FPA assays described herein can be linked to cell-penetrating peptide sequences such as penetratin, transportan or tat either directly or through intervening sequences. In addition, they can be linked to peptides that target cancer cells directly or through intervening sequences.

For example, a pro-apoptotic modulator of Bc-2 can be linked through GX to a cyclic disulfide loop peptide, Lyp-1, that binds specifically to breast cancer cells. Alternatively, the pro-apoptotic modulator of Bcl-2 can be linked through GX to F3, a 31-residue peptide that binds specifically to breast cancer cells. Additional examples of cell-permeability enhancers that can be conjugated to the pro-apoptotic modulator of Bcl-2 are provided below. The peptides can also have the inverso-configuration.

A compound of the invention may have the following length prior to being conjugated to a cell-permeability enhancer: 4-597 amino acids, preferably 4-400 amino acids, preferably 4-300 amino acids, preferably 4-200 amino acids, preferably 4-100 amino acids, preferably 4-50 amino acids, preferably 4-40 amino acids, preferably 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, or 4 amino acids. In one embodiment, the compound has fewer than 30, 25, 20, 15 or 10 amino acids.

Exemplary pro-apoptotic modulators of Bcl-2 are listed below in Table 1. As shown in Table 1, the peptide labels have changed over time. Table 1 lists the previous names of each peptide, and the current name of the same peptide.

TABLE 1
Exemplary pro-apoptotic modulators of Bc1-2

| | |
|---|---|
| Pro-apoptotic modulator of Bc1-2 | SEQ |

| | | | |
|---|---|---|---|
| Previous | | | ID |
| Name | Current Name | Sequence | NO: |

| | | | |
|---|---|---|---|
| TR3 peptide; | NuBCP-20-r8 | acetyl---GDWIDSILAFSRSLHSLLVDKKC-X-rrrrrrrr | 1 |
| Nur77 peptide | | | |
| Nur77-15 | NuBCP-15 | acetyl--------SILAFSRSLHSLLVDGXrrrrrrrr-amide | 2 |
| Nur77-14 | NuBCP-14 | acetyl---------ILAFSRSLHSLLVDGXrrrrrrrr-amide | 3 |
| Nur77-13 | NuBCP-13 | acetyl----------LAFSRSLRSLLVDGXrrrrrrrr-amide | 4 |
| Nur77-12 | NuBCP-12 | acetyl-----------AFSRSLHSLLVDGXrrrrrrrr-amide | 5 |
| Nur77-11 | NuBCP-11 | acetyl------------FSRSLHSLLVDGXrrrrrrrr-amide | 6 |
| Nur77-10 | NuBCP-10 | acetyl-------------SRSLESLLVDGXrrrrrrrr-amide | 7 |
| Nur77-N10 | NuBGP-N10 | acetyl------------FSRSLHSLLVGXrrrrrrrr-amide | 8 |
| TR3/1; | NuBCP-9-r8, | acetyl------------FSRSLHSLLGXrrrrrrrr-amide | 9 |
| Nur77-9 | Nur77/1 | | |
| Nur77-8 | NUBCP-8 | acetyl------------FSRSLHSLGXrrrrrrrr-amide | 10 |
| Nur77-7 | NuBCP-7 | acetyl------------FSRSLHSGXrrrrrrrr-amide | 11 |
| Nur77-9/AA | NuBCP-9/AA | acetyl------------ASRSLHSLAGXrrrrrrrr-amide | 12 |
| TR3/1(D) | D-NuBCP-9-r8 | acetyl------------fsrslhsllGXrrrrrrrr-amide | 13 |
| TR3/1 | Nur77/1 | acetyl------------LLSHLSRSFGXrrrrrrrr-amide | 14 |
| (inverso) | (inverso) | | |
| TR3/1 (retro- | Nur77/1 | acetyl------------llshlsrsfGXrrrrrrrr-amide | 15 |
| inverso) | (retro- | | |
| | inverso) | | |
| TR3/2 | Nur77/2 | acetyl------------FGDWTDSILGXrrrrrrrr-amide | 16 |
| TR3/3 | Nur77/3 | acetyl------------FAALSALVLGXrrrrrrrr-amide | 17 |
| TR3/4 | Nur77/4 | acetyl------------FYLKLEDLVGXrrrrrrrr-amide | 18 |
| NOR1 peptide | Nor1 peptide | acetyl--------SIKDFSLNLQSLNLDG rrrrrrrr-amide | 19 |
| Nurr1 | NOT peptide | acetyl---SIVEFSSNLQNMNIDG rrrrrrrr-amide | 20 |
| TR3/1 | Nur77/1 | acetyl----rrrFrrrLrrLL-amide | 21 |
| (embedded) | (embedded) | | |
| TR3/1: | Nur77/1 | acetyl----rrrfrrrlrrll-amide | 22 |
| (D/embedded) | (D/embedded) | | |
| | | acetyl-------fSrSlHsLlGxrrrrrrrr-amide | 23 |
| | | acetyl-------fSRslHsllGXrrrrrrrr-amide | 24 |
| | | acetyl-------FARSLHSLLGXrrrrrrrr-amide | 25 |
| | | acetyl-------FSASLHSLLGXrrrrrrrr-amide | 26 |
| | | acetyl-------FSRALHSLLGXrrrrrrrr-amide | 27 |
| | | acetyl-------FSRSLASLLGXrrrrrrrr-amide | 28 |
| | | acetyl-------FSRSLRALLGXrrrrrrrr-amide | 29 |
| | | acetyl-------F_RSLHSLLGXrrrrrrrr-amide | 30 |
| | | acetyl-------FS_SLHSLLGXrrrrrrrr-amide | 31 |
| | | acetyl-------FSR_LHSLLGXrrrrrrrr-amide | 32 |
| | | acetyl-------FSRSL_SLLGXrrrrrrrr-amide | 33 |
| | | acetyl-------FSRSLH_LLGXrrrrrrrr-amide | 34 |
| | | acetyl-------F__SLHSLLGXrrrrrrrr-amide | 35 |
| | | acetyl-------f_rslhsllGXrrrrrrrr-amide | 36 |
| | | acetyl-------FXSLHSLLGXrrrrrrrr-amide | 37 |
| | | acetyl-------FSRSLHSLLGX(CGNKRTAC)-amide | 38 |
| | | acetyl---FSRSLHSLLGXAKVKDEPQRRSARLSAKPAPPKPEPSPICKAPAKK-amide | 39 |
| | Nur77/short | acetyl---F___L__LLGXrrrrrrrr-amide | 40 |
| | Nur77/short D | acetyl---f___l__llGXrrrrrrrr-amide | 41 |
| | Nur77/1 Ant | acetyl---FSRSLHSLLC-CRQIKIWFQNRRMKWKK-amide | 42 |
| | Nur77/Ant (D) | acetyl---fsrslhsllc-crqvkvwfqnrrmkwkk-amide | 43 |
| | p53 peptide | acetyl---FSD_LWKLL-GXrrrrrrrr-amide | 44 |
| | Nur77 | acetyl-NFQHALQEVLQALKQVQAR-C—C-rrrrrrrr-amide | 45 |
| | (embedded2) | | |
| | Nur77/1 (D/L) | acetyl---fSrSlHsLl-GXrrrrrrrr-amide | 46 |
| | Nur77/1 | acetyl---fSRslHSll-GXrrrrrrrr-amide | 47 |
| | (DD/LL) | | |
| Nur77/1 | NuBCP-9- | acetyl---FSRSLHSLL-(C—C)RQIKIWFQNRRMKWKK-amide | 48 |
| Penetratin | Penetratin | | |
| | Nur77/1 | acetyl---fsrslhsll-(c—c)rqvkvwfqnrrmkwkk-amide | 49 |
| | (D) Penetratin | | |
| | (D) | | |
| | Nur77/1 | acetyl---FSRSLHSLL-(C—C)AGYLLGKINLKALAALAKKIL-amide | 50 |
| | Transportan10 | | |
| | Nur77/1(D) | acetyl---fsrslhsll-(c—c)agyllgkvnlkalaalakkvl-amide | 51 |
| | Transportan10 | | |
| | (D) | | |
| | Nur77/1 (L/D) | acetyl---FsRsLhSlL-(c—C)aGyLlGkInLkAlAaLaKkIl-amide | 52 |
| | Transportan10 | | |
| | (L/D): | | |
| | Nur77/1 (LLDD) | acetyl---FSrsLHslL-(C—c)aGYllGKvnLKalAalaKkvl-amide | 53 |
| | Transportan10 | | |
| | (LLDD) | | |
| | NuBCP-9- | acetyl---FSRSLHSLL-CCGWTLNSAGYLLGKINKALAALAKKIL-amide | 54 |
| | Transportan | | |

Single letter code is used for L-amino acids (capitalized), while D-amino acids are lower case. Substituted and added amino acids are in bold. r is aminoacid Arginine. X is 6-aminohexanoic acid. The C-X bond is formed from the reaction of a C-terminal Cys thiol group with a chloracetylated aminohexanoyl group. C—C bond is formed by the oxidation of two cysteine amino acids to form a disulfide bond. Brackets ( ) indicate that the two cysteines oxidize to form a disulfide loop.

In one embodiment, the analog shares at least about 50%, at least about 60%, at least about 70%, at least about 80%, at least about 90%, sequence identity with the peptides listed above.

The cell permeability of a conjugate can be verified by directly or indirectly labeling the conjugate with a detectable label which can be visualized inside a cell with the aid of microscopy. For example, a fluorescein derivative of the conjugate can be made by methods well known to those skilled in the art for conjugating fluorescein molecules to peptides. The fluorescein conjugate is incubated with the relevant target cells in vitro. The cells are harvested and fixed, then stained with Streptavidin-fluorescein and observed in the dark under confocal microscopy. Internalization of the exogenous molecule to which the carrier is conjugated is apparent by fluorescence.

For peptide conjugates, the peptide portion can be a recombinant peptide, a natural peptide, or a synthetic peptide. The peptide can also be chemically synthesized, using, for example, solid phase synthesis methods.

### I. DEFINITIONS AND GENERAL PARAMETERS

The following definitions are set forth to illustrate and define the meaning and scope of the various terms used to describe the invention herein.

As used herein, “pharmaceutically or therapeutically acceptable carrier” refers to a carrier medium which does not interfere with the effectiveness of the biological activity of the active ingredients and which is minimally toxic to the host or patient.

As used herein, “stereoisomer” refers to a chemical compound having the same molecular weight, chemical composition, and constitution as another, but with the atoms grouped differently. That is, certain identical chemical moieties are at different orientations in space and, therefore, when pure, have the ability to rotate the plane of polarized light. However, some pure stereoisomers can have an optical rotation that is so slight that it is undetectable with present instrumentation. The compounds described herein can have one or more asymmetrical carbon atoms and therefore include various stereoisomers. All stereoisomers are included within the scope of the present invention,

As used herein, “therapeutically- or pharmaceutically-effective amount” as applied to the disclosed compositions refers to the amount of composition sufficient to induce a desired biological result. That result can be alleviation of the signs, symptoms, or causes of a disease, or any other desired alteration of a biological system. For example, the result can involve a decrease and/or reversal of cancerous cell growth.

As used herein, “homology” or “identity” or “similarity” refers to sequence similarity between two peptides or between two nucleic acid molecules. Homology can be determined by comparing a position in each sequence which can be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. An “unrelated” or “non-homologous” sequence shares less than about 40% identity, though preferably less than about 25% identity, with one of the sequences described herein.

As used herein, the term “inhibitor” is interchangeably used to denote “antagonist”. Both these terms define compositions which have the capability of decreasing certain enzyme activity or competing with the activity or function of a substrate of said enzyme.

As used herein “peptide” indicates a sequence of amino acids linked by peptide bonds.

The term “peptidomimetic” means that a peptide according to the invention is modified in such a way that it includes at least one non-coded residue or non-peptidic bond.

Such modifications include, e.g., alkylation and, more specifically, methylation of one or more residues, insertion of or replacement of natural amino acid by non-natural amino acids, and replacement of an amide bond with other covalent bond. A peptidomimetic can optionally comprise at least one bond which is an amide-replacement bond such as urea bond, carbamate bond, sulfonamide bond, hydrazine bond, or any other covalent bond. The design of appropriate “peptidomimetic” can be computer assisted.

The term “spacer” denotes a chemical moiety whose purpose is to link, covalently, a cell-permeability moiety and a peptide or peptidomimetic. The spacer can be used to allow distance between the cell-permeability moiety and the peptide, or it is a chemical bond of any type. Linker denotes a direct chemical bond or a spacer.

The term “core” refers to the peptidic segment or moiety of the pro-apoptotic modulator of Bcl-2 which comprises peptide or peptidomimetic and is optionally attached to a cell-permeability enhancer.

The term “permeability” refers to the ability of an agent or substance to penetrate, pervade, or diffuse through a barrier, membrane, or a skin layer. “Cell permeability” or a “cell-penetration” moiety refers to any molecule known in the art which is able to facilitate or enhance penetration of molecules through membranes. Non-limiting examples include: hydrophobic moieties such as lipids, fatty acids, steroids and bulky aromatic or aliphatic compounds; moieties which can have cell-membrane receptors or carriers, such as steroids, vitamins and sugars, natural and non-natural amino acids and transporter peptides. Examples for lipid moieties which can be used are: Lipofectamine; Transfectace; Transfectam; Cytofectin; DMRIE; DLRIE; GAP-DLRIE; DOTAP; DOPE; DMEAP; DODMP; DOPC; DDAB; DOSPA; EDLPC; EDMPC; DPH; TMADPH; CTAB; lysyl-PE; DC-Cho; -alanyl cholesterol; DCGS; DPPES; PCPE; DMAP; DMPE; DOGS; DOHME; DPEPC; Pluronic; Tween; BRIJ; plasmalogen; phosphatidylethanolamine; phosphatidyleholine; glycerol-3-ethylphosphatidylcholine; dimethyl ammonium propane; trimethyl ammonium propane; diethylammonium propane; triethylammonium propane; dimethyldioctadecylammonium bromide; a sphingolipid; sphingomyelin; a lysolipid; a glycolipid; a sulfatide; a glycosphingolipid; cholesterol; cholesterol ester; cholesterol salt; oil; N-succinyldioleoylphosphatidylethanolamine; 1,2-dioleoyl-sn-glycerol; 1,3-dipalmitoyl-2 succinylglycerol; 1,2-dipalmitoyl-sn-3-succinylglycerol; 1-hexadecyl-2-palmitoylglycerophosphatidylethanolamine; palmitoythomocysteine; N,N′-Bis(do-decyaminocarbonylmethylene)-N,N′-bis((-N,N,N-trimethylammoniumethyl-aminocarbonyl-methylene)ethylencdiamine tetraiodide; N,N′-Bis(exadecylaminocarbonylmethylene)-N,N′,N″-tris((-N,N,N-trimethylammonium-ethylaminocarbonylmethylenediethylenetriaminehexaiodide; N,N′-Bis(dodecylaminocarbonylmethylene)-N,N″-bis((-N,N,N-trimethylammoniumethylamino-carbonylmethylene)cy-clohexylene-1,4-diaminetetra-iodide; 1,7,7-tetra-((N,N,N,N-tetramethylammonium methylamino-carbonylmethylene)-3-hexadecylaminocarbonyl methylene-1,3,7-triaazaheptane heptaiodide; N,N,N′,N′-tetra((-N,N,N-trimethylammonium-ethylaminocarbonylmethylene)-N′-(1,2-dioleoylglycero-3-phosphoethanolaminocarbonyl methylene)diethylenetriamine tetraiodide; dioleoylphosphatidyl ethanolamine; a fatty acid; a lysolipid; phosphatidylcholine; phosphatidylethanolamine; phosphatidylserine; phosphatidylglycerol; phosphatidylinositol; a sphingolipid; a glycolipid; a glucolipid; a sulfatide; a glycosphingolipid; phosphatidic acid; palmitic acid; stearic acid; arachidonic acid; oleic acid; a lipid bearing a polymer; a lipid bearing a sulfonated saccharide; cholesterol; tocopherol hemisuccinate; a lipid with an ether-linked fatty acid; a lipid with an ester-linked fatty acid; a polymerized lipid; diacetyl phosphate; stearylamine; cardiolipin; a phospholipid with a fatty acid of 6-8 carbons in length; a phospholipid with asymmetric acyl chains; 6-(5cholesten-3b-yloxy)-1-thio-b-D-galactopyranoside; digalactosyldiglyceride; 6-(5-cholesten-3b-yloxy)hexyl-6-amino-6-deoxy-1-thio-b-D-galactopyranoside; 6-(5-cholesten-3b-yloxy)hexyl-6-amino-6-deoxyl-1-thio-a-D-mannopyranoside; 12-(((7′-diethylamino-coumarin-3-yl)carbonyl)methylamino)-octadecanoic acid; N-[12-(((7′-diethylaminocoumarin-3-yl)carbonyl)methyl-amino) octadecanoyl]; -2-aminopalmitic acid; cholesteryl(4′-trimethyl-ammonio)butanoate; N-succinyldioleoyl-phosphatidylethanolamine; 1,2-dioleoyl-sn-glycerol; 1,2-dipalmitoyl-sn-3-succinyl-glycerol; 1,3-dipalmitoyl-2-succinylglycerol; 1-hexadecyl-2-palmitoylglycero-phosphoethanolamine; palmitoylhomocysteine; cyclic 9-amino-acid peptide as described in Laakkonen et al. 2002 Nature Med 8:751-755; a peptide described in Porkka et al. 2002 PNAS USA 99:7444-7449; and polymers of L- or D-arginine as described in Mitchell et al. 2000 J Peptide Res 56:318-325.

As used herein, “cancer” and “cancerous” refer to any malignant proliferation of cells in a mammal.

As used herein, “neurodegenerative disease” is a condition which affects brain function and is a result of deterioration of neurons. The neurodegenerative diseases are divided into two groups: a) conditions causing problems with movements, and conditions affecting memory and conditions related to dementia. Neurodegenerative diseases include, for example, Huntington's disease, spinocerebellar ataxias, Machado-Joseph disease, Spinal and Bulbar muscular atrophy (SBMA or Kennedy's disease), Dentatorubral Pallidoluysian Atrophy (DRPLA), Fragile X syndrome, Fragile XE mental retardation, Friedreich ataxia, myotonic dystrophy, Spinocerebellar ataxias (types 8, 10 and 12), spinal muscular atrophy (Werdnig-Hoffman disease, Kugelberg-Welander disease), Alzheimer's disease, amyotrophic lateral sclerosis, Parkinson's disease, Pick's disease, and spongiform encephalopathies. Additional neurodegenerative diseases include, for example, age-related memory impairment, agyrophilic grain dementia, Parkinsonism-dementia complex of Guam, auto-immune conditions (e.g., Guillain-Barre syndrome, Lupus), Biswanger's disease, brain and spinal tumors (including neurofibromatosis), cerebral amyloid angiopathies, cerebral palsy, chronic fatigue syndrome, corticobasal degeneration, conditions due to developmental dysfunction of the CNS parenchyma, conditions due to developmental dysfunction of the cerebrovasculature, dementia—multi infarct, dementia—subcortical, dementia with Lewy bodies, dementia of human immunodeficiency virus (HIV), dementia lacking distinct histology, Dementia Pugilistica, diseases of the eye, ear and vestibular systems involving neurodegeneration (including macular degeneration and glaucoma), Down's syndrome, dyskinesias (Paroxysmal), dystonias, essential tremor, Fahr's syndrome, fronto-temporal dementia and Parkinsonism linked to chromosome 17 (FTDP-17), frontotemporal lobar degeneration, frontal lobe dementia, hepatic encephalopathy, hereditary spastic paraplegia, hydrocephalus, pseudotumor cerebri and other conditions involving CSF dysfunction, Gaucher's disease, Hallervorden-Spatz disease, Korsakoff s syndrome, mild cognitive impairment, monomeric amyotrophy, motor neuron diseases, multiple system atrophy, multiple sclerosis and other demyelinating conditions (e.g., leukodystrophies), myalgic encephalomyelitis, myoclonus, neurodegeneration induced by chemicals, drugs and toxins, neurological manifestations of AIDS including AIDS dementia, neurological/cognitive manifestations and consequences of bacterial and/or viral infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyugeal muscular dystrophy, neurological manifestations of Polio myelitis including non-paralytic polio and post-polio-syndrome, primary lateral sclerosis, prion diseases including Creutzfeldt-Jakob disease (including variant form), kuru, fatal familial insomnia, Gerstmann-Straussler-Scheinker disease and other transmissible spongiform encephalopathies, prion protein cerebral amyloid angiopathy, postencephalitic Parkinsonism, progressive muscular atrophy, progressive bulbar palsy, progressive subcortical gliosis, progressive supranuclear palsy, restless leg syndrome, Rett syndrome, Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sachs disease, Tourette's syndrome, vascular dementia, Wilson disease, Alexander disease, Alper's disease, ataxia telangiectasia, Canavan disease, Cockayne syndrome, Krabbe disease, multiple system atrophy, Pelizaeus-Merzbacher Disease, primary lateral sclerosis, Refsum's disease, Sandhoff disease, Schilder's disease, Steele-Richardson-Olszewski disease, tabes dorsalis.

When two compounds are administered in combination or used in combination therapy according to the invention the term “combination” in this context means that the drugs are given contemporaneously, either simultaneously or sequentially. This term is exchangeable with the term “coadministration” which in the context of this invention is defined to mean the administration of more than one therapeutic in the course of a coordinated treatment to achieve an improved clinical outcome. Such coadministration can also be coextensive, that is, occurring during overlapping periods of time.

In addition to peptides consisting only of naturally-occurring amino acids, peptidomimetics or peptide analogs are also considered. Peptide analogs are commonly used in the pharmaceutical industry as non-peptide drugs with properties analogous to those of the template peptide. These types of non-peptide compounds are termed “peptide mimetics” or “peptidomimetics” (see, e.g., Luthman et al. 1996 A Textbook of Drug Design and Development, 14:386-406, 2nd Ed., Harwood Academic Publishers; Grante 1994 Angew Chem Int Ed Engl 33:1699-1720; Fauchere 1986 Adv Drug Res 15:29; Evans et al. 1987 J Med Chem 30:229). Peptide mimetics that are structurally similar to therapeutically useful peptides can be used to produce an equivalent or enhanced therapeutic or prophylactic effect. Generally, peptidomimetics are structurally similar to a paradigm polypeptide (i.e., a polypeptide that has a biological or pharmacological activity), such as naturally-occurring receptor-binding polypeptide, but have one or more peptide linkages optionally replaced by a linkage selected from the group consisting of: —CH₂ NH—, —CH₂S—, —CH₂—CH₂—, CH═CH— (cis and trans), —COCH₂—, —CH(OH)CH₂—, and —CH₂ SO—, by methods known in the art and farther described in the following references: Spatola, 1983, In: Chemistry and Biochemistry of Amino Acids, Peptides, and Proteins, B. Weinstein, eds., Marcel Dekker, New York, p. 267; Hudson et al 1979 Int J Pept Prot Res 14:177-185 (1979) (—CH₂NH—, CH₂ CH₂—); Spatola et al. 1986 Life Sci 38:1243-1249 (—CH₂—S); Hann 1982 J Chem Soc Perkins Trans I, 307-314 (—CH—CH—, cis and trans); Almquist et al. 1980 J Med Chem 23:1392-1398 (—COCH₂—); Jennings-White et al. 1982 Tetrahedron Lett 23:2533 (—COCH₂—); Szelke, et al European Appln. EP 45665 (1982) (—CH(OH)CH₂—); Holladay et al. 1983 Tetrahedron Lett 24:4401-4404 (—C(OH)CH₂—); and Hruby, 1982 Life Sci 31:189-199 (—CH₂—S—); each of which is incorporated herein by reference. A particularly preferred non-peptide linkage is —CH₂NH—. Such peptide mimetics can have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) (e.g., immunoglobulin superfamily molecules) to which the peptidomimetic binds to produce the therapeutic effect. Derivatization (e.g., labeling) of peptidomimetics should not substantially interfere with the desired biological or pharmacological activity of the peptidomimetic. Generally, peptidomimetics of receptor-binding peptides bind to the receptor with high affinity and possess detectable biological activity (i.e., are agonistic or antagonistic to one or more receptor-mediated phenotypic changes).

Systematic substitution of one or more amino acids of a consensus sequence with a D-amino acid of the same type (e.g., D-lysine in place of L-lysine) can be used to generate more stable peptides. In addition, constrained peptides comprising a consensus sequence or a substantially identical consensus sequence variation can be generated by methods known in the art (Rizo, et al. 1992 Annu Rev Biochem 61:387, incorporated herein by reference); for example, by adding internal cysteine residues capable of forming intramolecular disulfide bridges which cyclize the peptide.

Synthetic or non-naturally occurring amino acids refer to amino acids which do not naturally occur in vivo but which, nevertheless, can be incorporated into the peptide structures described herein. Preferred synthetic amino acids are the D-α-amino acids of naturally occurring L-α-amino acid as well as non-naturally occurring D- and L-α-amino acids represented by the formula H₂NCHR⁵COOH where R⁵ is 1) a lower alkyl group, 2) a cycloalkyl group of from 3 to 7 carbon atoms, 3) a heterocycle of from 3 to 7 carbon atoms and 1 to 2 heteroatoms selected from the group consisting of oxygen, sulfur, and nitrogen, 4) an aromatic residue of from 6 to 10 carbon atoms optionally having from 1 to 3 substituents on the aromatic nucleus selected from the group consisting of hydroxyl, lower alkoxy, amino, and carboxyl, 5)-alkylene-Y where alkylene is an alkylene group of from 1 to 7 carbon atoms and Y is selected from the group consisting of (a) hydroxy, (b) amino, (c) cycloalkyl and cycloalkenyl of from 3 to 7 carbon atoms, (d) aryl of from 6 to 10 carbon atoms optionally having from 1 to 3 substituents on the aromatic nucleus selected from the group consisting of hydroxyl, lower alkoxy, amino and carboxyl, (e) heterocyclic of from 3 to 7 carbon atoms and 1 to 2 heteroatoms selected from the group consisting of oxygen, sulfur, and nitrogen, (f) —C(O)R where R² is selected from the group consisting of hydrogen, hydroxy, lower alkyl, lower alkoxy, and —NR³R⁴ where R¹³ and R¹⁴ are independently selected from the group consisting of hydrogen and lower alkyl, (g) —S(O)ₙR⁶ where n is an integer from 1 to 2 and R⁶ is lower alkyl and with the proviso that R⁵ does not define a side chain of a naturally occurring amino acid.

Other preferred synthetic amino acids include amino acids wherein the amino group is separated from the carboxyl group by more than one carbon atom such as beta (β)-alanine, gamma (γ)-aminobutyric acid, and the like.

Particularly preferred synthetic amino acids include, by way of example, the D-amino acids of naturally occurring L-amino acids, L-(1-naphthyl)-alanine, L-(2-naphthyl)-alanine, L-cyclohexylalanine, L-2-aminoisobutyric acid, the sulfoxide and sulfone derivatives of methionine (i.e., HOOC—(H₂NCH)CH₂ CH₂—S(O)ₙR⁶) where n and R⁶ are as defined above as well as the lower alkoxy derivative of methionine (i.e., HOOC—(H₂NCH)CH₂ CH₂—OR⁶ where R⁶ is as defined above).

### II. Overview

Compounds identified using the FPA described herein that bind to Bcl-2-family members and alter their function in apoptosis are also provided. These compounds include “lead” peptide compounds and “derivative” compounds constructed so as to have the same or similar molecular structure or shape as the lead compounds but that differ from the lead compounds either with respect to susceptibility to hydrolysis or proteolysis and/or with respect to other biological properties, such as increased affinity for the receptor.

### III. Preparation of Peptides and Peptide Mimetics

Peptides can be synthesized using any method known in the art, including peptidomimetic methodologies. These methods include solid phase as well as solution phase synthesis methods. The conjugation of the peptidic and permeability moieties can be performed using any methods known in the art, either by solid phase or solution phase chemistry. Non-limiting examples for these methods are described herein. Some of the preferred compounds disclosed herein can conveniently be prepared using solution phase synthesis methods. Other methods known in the art to prepare compounds like those described herein, can be used and are within the scope of the present invention.

The amino acids used are those which are available commercially or are available by routine synthetic methods. Certain residues can require special methods for incorporation into the peptide, and either sequential, divergent or convergent synthetic approaches to the peptide sequence are useful in this invention. Natural coded amino acids and their derivatives are represented by three-letter codes according to IUPAC conventions.

When there is no indication, the L isomer was used. The D isomers are indicated by lower case font.

Conservative substitutions of amino acids as known to those skilled in the art are within the scope of the present invention. Conservative amino acid substitutions includes replacement of one amino acid with another having the same type of functional group or side chain e.g., aliphatic, aromatic, positively charged, negatively charged. These substitutions can enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

| | |
|---|---|
| Aromatic | Phenylalanine |
| | Tryptophan |
| | Tyrosine |
| Ionizable: Acidic | Aspartic acid |
| | Glutamic Acid |
| Ionizable: Basic | Arginine |
| | Histidine |
| | Lysine |
| Nonionizable Polar | Asparagine |
| | Glutamine |
| | Serine |
| | Cysteine |
| | Threonine |
| Nonpolar (Hydrophobic) | Alanine |
| | Glycine |
| | Isoleucine |
| | Leucine |
| | Methionine |
| | Proline |
| | Valine |
| Sulfur Containing | Cysteine |
| | Methionine |

The following is a list of non limiting examples of non-coded amino acids: Abu refers to 2-aminobutyric acid, Ahx6 refers to aminohexanoic acid, Ape5 refers to aminopentanoic acid, ArgO1 refers to argininol, βAla refers to β-Alanine, Bpa refers to 4-Benzoylphenylalanine, Bip refers to Beta (4-biphenyl)-alanine, Dab refers to diaminobutyric acid, Dap refers to Diaminopropionic acid, Dim refers to Dimethoxyphenylalanine, Dpr refers to Diaminopropionic acid, Hol refers to homoleucine, HPhe refers to Homophenylalanine, GABA refers to gamma aminobutyric acid, GlyNH₂ refers to Aminoglycine, Nle refers to Norleucine, Nva refers to Norvaline, Orn refers to Ornithine, PheCarboxy refers to para carboxy Phenylalanine, PheC1 refers to para chloro Phenylalanine, PheF refers to para fluoro Phenylalanine, PheMe refers to pare methyl Phenylalanine, PheNH2 refers to pare amino Phenylalanine, PheNO2 refers to para nitro Phenylalanine, Phg refers to Phenylglycine, Thi refers to Thienylalanine.

In conventional solution phase peptide synthesis, the peptide chain can be prepared by a series of coupling reactions in which the constituent amino acids are added to the growing peptide chain in the desired sequence. The use of various N-protecting groups, e.g., the carbobenzyloxy group or the t-butyloxycarbonyl group, various coupling reagents (e.g., dicyclohexylcarbodiimide or carbonyldiimidazole, various active esters, e.g., esters of N-hydroxyphthalimide or N-hydroxy-succinimide, and the various cleavage reagents, e.g., trifluoroacetic acid (TEA), HCl in dioxane, boron tris-(trifluoracetate) and cyanogen bromide, and reaction in solution with isolation and purification of intermediates is well-known classical peptide methodology. The preferred peptide synthesis method follows conventional Merrifield solid-phase procedures (see Merrifield, 1963 J Amer Chem Soc 85:2149-54; and 1965 Science 50:178-85). Additional information about the solid phase synthesis procedure can be had by reference to the treatise by Steward and Young (Solid Phase Peptide Synthesis, W. H. Freeman & Co., San Francisco, 1969, and the review chapter by Merrifield in Advances in Enzymology 32:221-296, F. F. Nold, Ed., Interscience Publishers, New York, 1969; and Erickson and Merrifield, The Proteins 2:255 et seq. (ea. Neurath and Hill), Academic Press, New York, 1976. The synthesis of peptides by solution methods is described in Neurath et al., eds. (The Proteins, Vol. II, 3d Ed., Academic Press, NY (1976)).

Crude peptides can be purified using preparative high performance liquid chromatography. The amino terminus can be blocked according, for example, to the methods described by Yang et al. 1990 FEBS Lett 272:61-64.

Peptide synthesis includes both manual and automated techniques employing commercially available peptide synthesizers. The peptides described herein can be prepared by chemical synthesis and biological activity can be tested using the methods disclosed herein.

The peptides described herein can be synthesized in a manner such that one or more of the bonds linking amino acid residues are non-peptide bonds. These non-peptide bonds can be formed by chemical reactions well known to those skilled in the art. In yet another embodiment of the invention, peptides comprising the sequences described above can be synthesized with additional chemical groups present at their amino and/or carboxy termini, such that, for example, the stability, bio-availability, and/or inhibitory activity of the peptides is enhanced. For example, hydrophobic groups such as carbobenzoxyl, dansyl, or t-butyloxycarbonyl groups, can be added to the peptides' amino terminus. Likewise, an acetyl group or a 9-fluorenylmethoxy-carbonyl group can be placed at the peptides' amino terminus. Additionally, the hydrophobic group, t-butyloxycarbonyl, or an amido group, can be added to the peptides' carboxy terminus.

In addition, the peptides can be engineered to contain additional functional groups to promote cell uptake. For example, carbohydrate moieties such as glucose or xylose can be attached to the peptide, such as by attachment to the hydroxyl function of a serine or threonine amino acid of the peptide.

Further, the peptides of the invention can be synthesized such that their stearic configuration is altered. For example, the D-isomer of one or more of the amino acid residues of the peptide can be used, rather than the usual L-isomer. Still further, at least one of the amino acid residues of the peptide can be substituted by one of the well known non-naturally occurring amino acid residues. Alterations such as these can serve to increase the stability, bioavailability and/or inhibitory action of the peptides.

### A. Solid Phase Synthesis

The peptides disclosed herein can be prepared by classical methods known in the art, for example, by using standard solid phase techniques. The standard methods include exclusive solid phase synthesis, partial solid phase synthesis methods, fragment condensation, classical solution synthesis, and even recombinant DNA technology (see, e.g., Merrifield 1963 J Am Chem Soc 85:2149). On solid phase, the synthesis is typically commenced from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. One such chloromethylated resin is sold under the trade name BIO-BEADS SX-1™ by Bio Rad Laboratories (Richmond, Calif.) and the preparation of the hydroxymethyl resin is described by Bodonszky et al. 1966 Chem Ind (London) 38:1597. The benzhydrylamine (BHA) resin has been described by Pietta and Marshall 1970 Chem Comm 650, and is commercially available from Beckman Instruments, Inc. (Palo Alto, Calif.) in the hydrochloride form.

Thus, the compounds disclosed herein can be prepared by coupling an alpha-amino protected amino acid to the chloromethylated resin with the aid of, for example, a cesium bicarbonate catalyst, according to the method described by Gisin, 1973 Helv Chi/m Acta 56:1467. After the initial coupling, the alpha-amino protecting group is removed by a choice of reagents including trifluoroacetic acid (TFA) or hydrochloric acid (HCl) solutions in organic solvents at room temperature.

The alpha-amino protecting groups are those known to be useful in the art of stepwise synthesis of peptides. Included are acyl type protecting groups (e.g., formyl, trifluoroacetyl, acetyl), aromatic urethane type protecting groups (e.g., benzyloxycarboyl (Cbz) and substituted Cbz), aliphatic urethane protecting groups (e.g., t-butyloxycarbonyl (Boc), isopropyloxycarbonyl, cyclohexyloxycarbonyl) and alkyl type protecting groups (e.g., benzyl, triphenylmethyl). Boc and Fmoc are preferred protecting groups. The side-chain protecting group remains intact during coupling and is not split off during the deprotection of the amino-terminus protecting group or during coupling. The side-chain protecting group must be removable upon the completion of the synthesis of the final peptide and under reaction conditions that will not alter the target peptide.

The side-chain protecting groups for Tyr include tetrahydropyranyl, tert-butyl, trityl, benzyl, Cbz, Z—Br—Cbz, and 2,5-dichlorobenzyl. The side-chain protecting groups for Asp include benzyl, 2,6-dichlorobenzyl, methyl, ethyl, and cyclohexyl. The side-chain protecting groups for Thr and Ser include acetyl, benzoyl, trityl, tetrahydropyranyl, benzyl, 2,6-dichlorobenzyl, and Cbz. The side-chain protecting group for Thr and Ser is benzyl. The side-chain protecting groups for Arg include nitro, Tosyl (Tos), Cbz, adamantyloxycarbonyl mesitoylsulfonyl (Mts), or Boc. The side-chain protecting groups for Lys include Cbz, 2-chlorobenzyloxycarbonyl (2Cl-Cbz), 2-bromobenzyloxycarbonyl (2-BrCbz), Tos, or Boc.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride (CH₂Cl₂), dimethyl formamide (DMF) mixtures.

After the desired amino acid sequence has been completed, the desired peptide is decoupled from the resin support by treatment with a reagent such as trifluoroacetic acid or hydrogen fluoride (HF), which not only cleaves the peptide from the resin, but also cleaves all remaining side chain protecting groups. When the chloromethylated resin is used, hydrogen fluoride treatment results in the formation of the free peptide acids. When the benzhydrylamine resin is used, hydrogen fluoride treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

These solid phase peptide synthesis procedures are well known in the art and further described by Stewart and Young, Solid Phase Peptide Syntheses (2nd Ed., Pierce Chemical Company, 1984).

### B. Synthetic Amino Acids

These procedures can also be used to synthesize peptides in which amino acids other than the 20 naturally occurring, genetically encoded amino acids are substituted at one, two, or more positions of any of the compounds described herein. For instance, naphthylalanine can be substituted for tryptophan, facilitating synthesis. Other synthetic amino acids that can be substituted into the peptides include L-hydroxypropyl, L-3,4-dihydroxy-phenylalanyl, amino acids such as L-α-hydroxylysyl and D-a-methylalanyl, L-a-methylalanyl, β amino acids, and isoquinolyl. D amino acids and non-naturally occurring synthetic amino acids can also be incorporated into the peptides.

One can replace the naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. In particular, proline analogs in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

One can also readily modify the peptides by phosphorylation (see, e.g., Bannwarth et al 1996 Bioorg Med Chem Letters 6:2141-2146), and other methods for making peptide derivatives of the compounds disclosed herein are described in Hruby et al. 1990 Biochem J 268:249-262. Thus, the peptide compounds can also serve as a basis to prepare peptide mimetics with similar biological activity.

### C. Terminal Modifications

Those of skill in the art recognize that a variety of techniques are available for constructing peptide mimetics with the same or similar desired biological activity as the corresponding peptide compound but with more favorable activity than the peptide with respect to solubility, stability, and susceptibility to hydrolysis and proteolysis (see, e.g., Morgan et al 1989 Ann Rep Med Chem 24:243-252). The following describes methods for preparing peptide mimetics modified at the N-terminal amino group, the C-terminal carboxyl group, and/or changing one or more of the amido linkages in the peptide to a non-amido linkage. It being understood that two or more such modifications can be coupled in one peptide mimetic structure (e.g., modification at the C-terminal carboxyl group and inclusion of a —CH₂-carbamate linkage between two amino acids in the peptide).

1. N-Terminal Modifications

The peptides typically are synthesized as the free acid but, as noted above, could be readily prepared as the amide or ester. One can also modify the amino and/or carboxy terminus of the peptide compounds to produce other compounds. Amino terminus modifications include methylation (i.e., —NHCH₃ or —NH(CH₃)₂), acetylation, adding a benzyloxycarbonyl group, or blocking the amino terminus with any blocking group containing a carboxylate functionality defined by RCOO—, where R is selected from the group consisting of naphthyl, acridinyl, steroidyl, and similar groups. Carboxy terminus modifications include replacing the free acid with a carboxamide group or forming a cyclic lactam at the carboxy terminus to introduce structural constraints.

Amino terminus modifications are as recited above and include alkylating, acetylating, adding a carbobenzoyl group, forming a succinimide group, etc. (see, e.g., Murray et al. 1995 Burger's Medicinal Chemistry and Drug Discovery, 5th Ed., Vol. 1, Wolf, ed., John Wiley and Sons, Inc.). Specifically, the N-terminal amino group can then be reacted as follows:

a) to form an amide group of the formula RC(O)NH— where R is as defined above by reaction with an acid halide (e.g., RC(O)Cl) or symmetric anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide for N-alkyl amide group of the formula RC(O)NR—; or

b) to form a succinimide group by reaction with succinic anhydride. As before, an approximately equimolar amount or an excess of succinic anhydride (e.g., about 5 equivalents) can be employed and the amino group is converted to the succinimide by methods well known in the art including the use of an excess (e.g., ten equivalents) of a tertiary amine such as diisopropylethylamine in a suitable inert solvent (e.g., dichloromethane) (see, for example, Wollenberg et al., U.S. Pat. No. 4,612,132 which is incorporated herein by reference in its entirety). It is understood that the succinic group can be substituted with, for example, C₂-C₆ alkyl or —SR substituents which are prepared in a conventional manner to provide for substituted succinimide at the N-terminus of the peptide. Such alkyl substituents are prepared by reaction of a lower olefin (C₂-C) with maleic anhydride in the manner described by Wollenberg et al., supra and —SR substituents are prepared by reaction of RSH with maleic anhydride where R is as defined above; or

c) to form a benzyloxycarbonyl-NH— or a substituted benzyloxycarbonyl-NH— group by reaction with approximately an equivalent amount or an excess of CBZ-Cl (i.e., benzyloxycarbonyl chloride) or a substituted CBZ-Cl in a suitable inert diluent (e.g., dichloromethane) preferably containing a tertiary amine to scavenge the acid generated during the reaction; or

d) to form a sulfonamide group by reaction with an equivalent amount or an excess (e.g., 5 equivalents) of R—S(O)₂Cl in a suitable inert diluent (dichloromethane) to convert the terminal amine into a sulfonamide where R is as defined above. Preferably, the inert diluent contains excess tertiary amine (e.g., ten equivalents) such as diisopropylethylamine, to scavenge the acid generated during reaction. Reaction conditions are otherwise conventional (e.g., room temperature for 30 minutes); or

e) to form a carbamate group by reaction with an equivalent amount or an excess (e.g., 5 equivalents) of R—OC(O)Cl or R—OC(O)OC₆H₄-p-NO₂ in a suitable inert diluent (e.g., dichloromethane) to convert the terminal amine into a carbamate where R is as defined above. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes); or

f) to form a urea group by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. Preferably, the inert diluent contains an excess (e.g., about 10 equivalents) of a tertiary amine, such as diisopropylethylamine. Reaction conditions are otherwise conventional (e.g., room temperature for about 30 minutes).

2. C-Terminal Modifications

In preparing peptide mimetics wherein the C-terminal carboxyl group is replaced by an ester (i.e., —C(O)OR where R is as defined above), the resins used to prepare the peptide acids are employed, and the side chain protected peptide is cleaved with base and the appropriate alcohol, e.g., methanol. Side chain protecting groups are then removed in the usual fashion by treatment with hydrogen fluoride to obtain the desired ester.

In preparing peptide mimetics wherein the C-terminal carboxyl group is replaced by the amide —C(O)NR³R⁴, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain protected peptide from the support yields the free peptide amide and reaction with an alkylamine or a dialkylamine yields a side chain protected alkylamide or dialkylamide (i.e., the C-terminus is —C(O)NRR¹ where R and R¹ are as defined above). Side chain protection is then removed in the usual fashion by treatment with hydrogen fluoride to give the free amides, alkylamides, or dialkylamides.

In another alternative embodiment, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

One can also cyclize the peptides herein, or incorporate a desamino or descarboxy residue at the termini of the peptide, so that there is no terminal amino or carboxyl group, to decrease susceptibility to proteases or to restrict the conformation of the peptide. C-terminal functional groups of the compounds include amide, amide lower alkyl, amide di(lower alkyl), lower alkoxy, hydroxy, and carboxy, and the lower ester derivatives thereof, and the pharmaceutically acceptable salts thereof.

In addition to the foregoing N-terminal and C-terminal modifications, the peptide compounds, including peptidomimetics, can advantageously be modified with or covalently coupled to one or more of a variety of hydrophilic polymers. It has been found that when the peptide compounds are derivatized with a hydrophilic polymer, their solubility and circulation half-lives are increased and their immunogenicity is masked. Quite surprisingly, the foregoing can be accomplished with little, if any, diminishment in their binding activity. Nonproteinaceous polymers suitable for use include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, more preferably from about 2,000 to about 40,000 daltons and, even more preferably, from about 5,000 to about 20,000 daltons. In preferred embodiments, such hydrophilic polymers have an average molecular weights of about 5,000 daltons, 10,000 daltons and 20,000 daltons.

The peptide compounds can be derivatized with or coupled to such polymers using, but not limited to, any of the methods set forth in Zallipsky, 1995 Bioconjugate Chem 6:150-165 and Monfardini et al. 1995 Bioconjugate Chem 6:62-69, all of which are incorporated by reference in their entirety herein.

In a presently preferred embodiment, the peptide compounds are derivatized with polyethylene glycol (PEG). PEG is a linear, water-soluble polymer of ethylene oxide repeating units with two terminal hydroxyl groups. PEGs are classified by their molecular weights which typically range from about 500 daltons to about 40,000 daltons. In a presently preferred embodiment, the PEGs employed have molecular weights ranging from 5,000 daltons to about 20,000 daltons. PEGs coupled to the peptide compounds can be either branched or unbranched. (see, e.g., Monfardini et al. 1995 Bioconjugate Chem 6:62-69). PEGs are commercially available from Shearwater Polymers, Inc. (Huntsville, Ala.), Sigma Chemical Co. and other companies. Such PEGs include, but are not limited to, monomethoxypolyethylene glycol (MePEG-OH), monomethoxypolyethylene glycol-succinate (MePEG-S), monomethoxypolyethylene glycol-succinimidyl succinate (MePEG-S—NHS), monomethoxypolyethylene glycol-amine (MePEG-NH₂), monomethoxypolyethylene glycol-tresylate (MePEG-TRES), and monomethoxypolyethylene glycol-imidazolyl-carbonyl (MePEG-IM).

Briefly, in one exemplar embodiment, the hydrophilic polymer which is employed, e.g., PEG, is preferably capped at one end by an unreactive group such as a methoxy or ethoxy group. Thereafter, the polymer is activated at the other end by reaction with a suitable activating agent, such as cyanuric halides (e.g., cyanuric chloride, bromide or fluoride), diimadozle, an anhydride reagent (e.g., a dihalosuccinic anhydride, such as dibromosuccinic anhydride), acyl azide, p-diazoiumbenzyl ether, 3-(p-diazoniumphenoxy)-2-hydroxypropylether) and the like. The activated polymer is then reacted with a peptide compound disclosed or taught herein to produce a peptide compound derivatized with a polymer. Alternatively, a functional group in the peptide compounds can be activated for reaction with the polymer, or the two groups can be joined in a concerted coupling reaction using known coupling methods. It will be readily appreciated that the peptide compounds can be derivatized with PEG using a myriad of reaction schemes known to and used by those of skill in the art.

In addition to derivatizing the peptide compounds with a hydrophilic polymer (e.g., PEG), it has been discovered that other small peptides, e.g., other peptides or ligands that bind to a receptor, can also be derivatized with such hydrophilic polymers with little, if any, loss in biological activity (e.g., binding activity, agonist activity, antagonist activity, etc.). It has been found that when these small peptides are derivatized with a hydrophilic polymer, their solubility and circulation half-lives are increased and their immunogenicity is decreased. Again, quite surprisingly, the foregoing can be accomplished with little, if any, loss in biological activity. In fact, in preferred embodiments, the derivatized peptides have an activity that is 0.1 to 0.01-fold that of the unmodified peptides. In more preferred embodiments, the derivatized peptides have an activity that is 0.1 to 1-fold that of the unmodified peptides. In even more preferred embodiments, the derivatized peptides have an activity that is greater than the unmodified peptides.

Peptides suitable for use in this embodiment generally include those peptides identified using the FPA described herein, i.e., ligands that competitively inhibit binding of known inhibitors of the Bcl-2 receptor family. Such peptides typically comprise about 150 amino acid residues or less and, more preferably, about 100 amino acid residues or less (e.g., about 10-12 kDa), even more preferably about 10 amino acids or less. Hydrophilic polymers suitable for use herein include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, more preferably from about 2,000 to about 40,000 daltons and, even more preferably, from about 5,000 to about 20,000 daltons. In preferred embodiments, such hydrophilic polymers have average molecular weights of about 5,000 daltons, 10,000 daltons and 20,000 daltons. The peptide compounds can be derivatized with using the methods described above and in the cited references.

### D. Backbone Modifications

Other methods for making peptide derivatives of the compounds described herein are described in Hruby et al. 1990 Biochem J 268:249-262, incorporated herein by reference. Thus, the peptide compounds also serve as structural models for non-peptidic compounds with similar biological activity. Those of skill in the art recognize that a variety of techniques are available for constructing compounds with the same or similar desired biological activity as the lead peptide compound but with more favorable activity than the lead with respect to solubility, stability, and susceptibility to hydrolysis and proteolysis (see Morgan et al. 1989 Ann Rep Med Chem 24:243-252, incorporated herein by reference). These techniques include replacing the peptide backbone with a backbone composed of phosphonates, amidates, carbamates, sulfonamides, secondary amines, and N-methylamino acids.

Peptide mimetics wherein one or more of the peptidyl linkages [—C(O)NH—] have been replaced by such linkages as a —CH₂-carbamate linkage, a phosphonate linkage, a —CH₂-sulfonamide linkage, a urea linkage, a secondary amine (—CH₂ NH—) linkage, and an alkylated peptidyl linkage [—C(O)NR⁶— where R⁶ is lower alkyl] are prepared during conventional peptide synthesis by merely substituting a suitably protected amino acid analogue for the amino acid reagent at the appropriate point during synthesis.

Suitable reagents include, for example, amino acid analogues wherein the carboxyl group of the amino acid has been replaced with a moiety suitable for forming one of the above linkages. For example, if one desires to replace a —C(O)NR—linkage in the peptide with a—CH₂-carbamate linkage (—CH₂OC(O)NR—), then the carboxyl (—COOH) group of a suitably protected amino acid is first reduced to the —CH₂ OH group which is then converted by conventional methods to a —OC(O)Cl functionality or a para-nitrocarbonate —OC(O)O—C₆H₄-p-NO₂ functionality. Reaction of either of such functional groups with the free amine or an alkylated amine on the N-terminus of the partially fabricated peptide found on the solid support leads to the formation of a—CH₂OC(O)NR—linkage. For a more detailed description of the formation of such —CH₂-carbamate linkages, see Cho et al., 1993, Science 261:1303-1305.

Similarly, replacement of an amido linkage in the peptide with a phosphonate linkage can be achieved in the manner set forth in U.S. patent application Ser. Nos. 07/943,805, 08/081,577 and 08/119,700, the disclosures of which are incorporated herein by reference in their entirety.

Replacement of an amido linkage in the peptide with a —CH₂-sulfonamide linkage can be achieved by reducing the carboxyl (—COOH) group of a suitably protected amino acid to the —CH₂ OH group and the hydroxyl group is then converted to a suitable leaving group such as a tosyl group by conventional methods. Reaction of the tosylated derivative with, for example, thioacetic acid followed by hydrolysis and oxidative chlorination will provide for the —CH₂—S(O)₂ Cl functional group which replaces the carboxyl group of the otherwise suitably protected amino acid. Use of this suitably protected amino acid analogue in peptide synthesis provides for inclusion of an —CH₂S(O)₂ NR— linkage which replaces the amido linkage in the peptide thereby providing a peptide mimetic. For a more complete description on the conversion of the carboxyl group of the amino acid to a —CH₂S(O)₂ Cl group, see, for example, Weinstein, Chemistry & Biochemistry of Amino Acids, Peptides and Proteins, Vol. 7, pp. 267-357, Marcel Dekker, Inc., New York (1983) which is incorporated herein by reference.

Replacement of an amido linkage in the peptide with a urea linkage can be achieved in the manner set forth in U.S. patent application Ser. No. 08/147,805, which is incorporated herein by reference.

Secondary amine linkages wherein a CH₂NH linkage replaces the amido linkage in the peptide can be prepared by employing, for example, a suitably protected dipeptide analogue wherein the carbonyl bond of the amido linkage has been reduced to a CH₂ group by conventional methods. For example, in the case of diglycine, reduction of the amide to the amine will yield after deprotection H₂NCH₂ CH₂ NHCH₂ COOH which is then used in N-protected form in the next coupling reaction. The preparation of such analogues by reduction of the carbonyl group of the amido linkage in the dipeptide is well known in the art (see, e.g., Remington, 1994 Meth Mol Bio 35:241-247).

The suitably protected amino acid analogue is employed in the conventional peptide synthesis in the same manner as would the corresponding amino acid. For example, typically about 3 equivalents of the protected amino acid analogue are employed in this reaction. An inert organic diluent such as methylene chloride or DMF is employed and, when an acid is generated as a reaction by-product, the reaction solvent will typically contain an excess amount of a tertiary amine to scavenge the acid generated during the reaction. One particularly preferred tertiary amine is diisopropylethylamine which is typically employed in about 10 fold excess. The reaction results in incorporation into the peptide mimetic of an amino acid analogue having a non-peptidyl linkage. Such substitution can be repeated as desired such that from zero to all of the amido bonds in the peptide have been replaced by non-amido bonds.

One can also cyclize the peptides described herein, or incorporate a desamino or descarboxy residue at the termini of the peptide, so that there is no terminal amino or carboxyl group, to decrease susceptibility to proteases or to restrict the conformation of the peptide. C-terminal functional groups of the compounds include amide, amide lower alkyl, amide di(lower alkyl), lower alkoxy, hydroxy, and carboxy, and the lower ester derivatives thereof, and the pharmaceutically acceptable salts thereof.

### E. Disulfide Bond Formation

The compounds described herein can exist in a cyclized form with an intramolecular disulfide bond between the thiol groups of the cysteines. Alternatively, an intermolecular disulfide bond between the thiol groups of the cysteines can be produced to yield a dimeric (or higher oligomeric) compound. One or more of the cysteine residues can also be substituted with a homocysteine.

Other embodiments of this invention provide for analogs of these disulfide derivatives in which one of the sulfurs has been replaced by a CH₂ group or other isostere for sulfur. These analogs can be made via an intramolecular or intermolecular displacement, using methods known in the art.

Alternatively, the amino-terminus of the peptide can be capped with an alpha-substituted acetic acid, wherein the alpha substituent is a leaving group, such as an α-haloacetic acid, for example, α-chloroacetic acid, α-bromoacetic acid, or α-iodoacetic acid. The compounds can be cyclized or dimerized via displacement of the leaving group by the sulfur of the cysteine or homocysteine residue. See, e.g., Andreu et al 1994 Meth Mol Bio 35:91-169; Barker et al 1992 J Med Chem 35:2040-2048; and Or et al. 1991 J Org Chem 56:3146-3149, each of which is incorporated herein by reference.

Alternatively, the peptides can be prepared utilizing recombinant DNA technology, which comprises combining a nucleic acid encoding the peptide thereof in a suitable vector, inserting the resulting vector into a suitable host cell, recovering the peptide produced by the resulting host cell, and purifying the polypeptide recovered. The techniques of recombinant DNA technology are known to those of ordinary skill in the art. General methods for the cloning and expression of recombinant molecules are described in Maniatis (Molecular Cloning, Cold Spring Harbor Laboratories, 1982), and in Sambrook (Molecular Cloning, Cold Spring Harbor Laboratories, Second Ed., 1989), and in Ausubel (Current Protocols in Molecular Biology, Wiley and Sons, 1987), which are incorporated by reference.

The peptides can be labeled, for further use as biomedical reagents or clinical diagnostic reagents. For example, a peptide of the invention can be conjugated with a fluorescent reagent, such as a fluorescein isothiocyanate (FITC), tetramethylrhodamine isothiocyanate (TRITC), or other fluorescent. The fluorescent reagent can be coupled to the peptide through the peptide N-terminus or free amine side chains by any one of the following chemistries, where R is the fluorescent reagent.

Alternatively, the peptide can be radiolabeled by peptide radiolabeling techniques well-known to those skilled in the art.

### IV Methods for Screening Peptides, Analogs, and Small Molecules that Modulate Bcl-2-Family Member Protein Activity

The FPAs described herein are designed to identify compounds that competitively inhibit binding of known converters of Bcl-2 and other anti-apoptotic members of the Bcl-2-family of proteins. These competitive inhibitors modify the ability of these Bcl-2 proteins to regulate apoptosis by mimicking or blocking the actions of the endogenous modulator Nur77. This regulation can be by mimicking the inhibitor, by inducing an equivalent conformation change, by enhancing the inhibitor effect or by inhibiting the inhibitor effect.

The compounds which can be screened include, but are not limited to peptides, fragments thereof, and other organic compounds (e.g., peptidomimetics) that competitively inhibit binding of a known modulator of the Bcl-2-family member. The inhibitors discovered by the FPA mimic the activity triggered by the natural regulatory ligand, enhance the activity triggered by the natural regulatory ligand or inhibit the activity triggered by the natural ligand; as well as peptides, antibodies or fragments thereof, and other organic compounds that mimic the binding domain of the Bcl-2-family member and bind to and “neutralize” natural ligand.

Such compounds include, but are not limited to, peptides such as, for example, soluble peptides, including but not limited to members of random peptide libraries; (see, e.g., Lam et al. 1991 Nature 354:82-84; Houghten et al. 1991 Nature 354:84-86), and combinatorial chemistry-derived molecular libraries made of D- and/or L-configuration amino acids, phosphopeptides (including, but not limited to, members of random or partially degenerate, directed phosphopeptide libraries; see, e.g., Songyang et al. 1993 Cell 72:767-778), antibodies including, but not limited to, polyclonal, monoclonal, humanized, anti-idiotypic, chimeric or single chain antibodies, and FAb, F(ab′)₂ and FAb expression library fragments, and epitope-binding fragments thereof, and small organic or inorganic molecules.

Computer modeling and searching technologies permit identification of compounds, or the improvement of already identified compounds that can modulate Bcl-2-family member activity. Having identified such a compound or composition, the active sites or regions are identified. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures can be measured with a complexed ligand, natural or artificial, which can increase the accuracy of the active site structure determined.

If an incomplete or insufficiently accurate structure is determined, the methods of computer based numerical modeling can be used to complete the structure or improve its accuracy. Any recognized modeling method can be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential Bcl-2-family member modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of Bcl-2 and related proteins will be apparent to those of skill in the art.

Examples of molecular modeling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen et al. 1988 Acta Pharm Fennica 97:159-166; McKinaly and Rossmann 1989 Annu Rev Pharmacol Toxicol 29:111-122; Perry and Davies, OSAR: Quantitative Structure-Activity Relationships in Drug Design, pp. 189-193, Alan R. Liss, Inc. (1989); Lewis and Dean 1989 Proc R Soc Lond 236:125-140 and 141-162; and, with respect to a model receptor for nucleic acid components, Askew et al. 1989 J Am Chem Soc 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario).

One could also screen libraries of known compounds, including natural products or synthetic chemicals, and biologically active materials, including proteins, for compounds which exhibit binding properties similar to those of FITC-Tr3-r8.

Once identified, these compounds can be subjected to assays such as those described in the examples to identify whether the compounds increase apoptosis or decrease apoptosis in cells.

Compounds identified via assays such as those described herein can be useful, for example, in inducing or inhibiting apoptosis.

### V. In Vitro Screening Assays for Compounds that Bind to Bcl-2-Family Member Proteins

In vitro systems can be designed to identify compounds capable of interacting with (e.g., binding to) Bcl-2-family members. Compounds identified can be useful, for example, in modulating the activity of wild type and/or mutant Bcl related proteins; can be useful in elaborating the biological function of the Bcl related proteins; can be utilized in screens for identifying compounds that disrupt normal Bcl-2-family member interactions; or can in themselves disrupt such interactions.

The FPA used to identify compounds that bind to the Bcl-2-family member involves preparing a first reaction mixture comprising a Bcl-2 protein, fluorescently labeled inhibitor and test compound, and a second mixture comprising the same Bcl-2 protein and fluorescently labeled inhibitor under conditions and for a time sufficient to allow the components to interact and bind, thus forming a complex which can be removed and/or detected in the reaction mixture.

In one embodiment, a FPA is used to identify converters of anti-apoptotic Bcl-2 proteins as follows: a) Bcl-2 or related anti-apoptotic Bcl-2 family member is produced and purified; b) Bcl-2 or a related Bcl-2 family member is incubated with a known inhibitor peptide (e.g., TR3-9-r8) labeled with a fluorescent tag, in the presence or absence of compounds being tested; c) after incubation under suitable conditions, the amount of labeled converter peptide bound to Bcl-2 or a related anti-apoptotic Bcl-2 family member is measured by assessing the quantity of polarized UV light; and e) the amount of labeled converter peptide bound in the presence of various test compounds is compared with the amount of labeled converter peptide bound in the absence of test compounds, and the ability of each test compound to compete for Bcl-2 or related Bcl-2 family member binding sites is determined.

In practice, microtiter plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, can be directly labeled or indirectly labeled with a labeled anti-Ig antibody).

Alternatively, a reaction can be conducted in a liquid phase, the reaction products separated from unreacted components, and complexes detected, e.g., using an immobilized antibody specific for the Bcl related protein, polypeptide, peptide or fusion protein or the test compound to anchor any complexes formed in solution, and a labeled antibody specific for the other component of the possible complex to detect anchored complexes.

Alternatively, cell-based assays can be used to identify compounds that interact with Bcl-2-family members or compounds that enhance or inhibit the interaction of Bcl-2 or related Bcl-2 family members with inhibitor peptide. To this end, cell lines that express Bcl related proteins, or cell lines (e.g., COS cells, CHO cells, fibroblasts, etc.) that have been genetically engineered to express Bcl-2 related proteins (e.g., by transfection or transduction of DNA) can be used.

### VI. Structure-Based Drug Design

To aid in the characterization and optimization of compounds that can alter the activity of Bcl-2-family proteins, structure-based drug design has become a useful tool. Solution nuclear magnetic resonance (NMR) techniques can be used to map the interactions between the BH3 domain of the Bcl-2-family protein and chemical compounds that target these anti-apoptotic proteins. NMR chemical shift perturbation is an efficient tool for rapid mapping of interaction interfaces on proteins. Structure-activity relationships (SAR) can be obtained by using nuclear magnetic resonance (NMR), using the method known as “SAR by NMR” (Shuker et al 1996 Science 274:1531; Lugovskoy et al. 2002 J Am Chem Soc 124:1234). SAR by NMR can be used to identify, optimize and link together small organic molecules that bind to proximal subsites of a protein to produce high-affinity ligands.

In using NMR to structurally characterize protein-protein and ligand-protein interactions, isotope labeling can result in increased sensitivity and resolution, and in reduced complexity of the NMR spectra. The three most commonly used stable isotopes for macromolecular NMR are ¹³C, ¹⁵N and ²H. Isotope labeling has enabled the efficient use of heteronuclear multi-dimensional NMR experiments, providing alternative approaches to the spectral assignment process and additional structural constraints from spin-spin coupling. Uniform isotope labeling of the protein enables the assignment process through sequential assignment with multidimensional triple-resonance experiments and supports the collection of conformational constraints in de novo protein structure determinations (Kay et al 1990 J Magn Reson 89:496; Kay et al 1997 Curr Opin Struct Biol 7:722). These assignments can be used to map the interactions of a ligand by following chemical-shift changes upon ligand binding. In addition, intermolecular NOE (nuclear Overhauser effect) derived inter-molecular distances can be obtained to structurally characterize protein-ligand complexes.

In addition to uniform labeling, selective labeling of individual amino acids or labeling of only certain types of amino acids in proteins can result in a dramatic simplification of the spectrum and, in certain cases, enable the study of significantly larger macromolecules. For example, the methyl groups of certain amino acids can be specifically labeled with ¹³C and ¹H in an otherwise fully ²H-labeled protein. This results in well resolved heteronuclear [¹³C,¹H]-correlation spectra, which enables straightforward ligand-binding studies either by chemical shift mapping or by protein methyl-ligand inter-molecular NOEs, thus providing key information for structure-based drug design in proteins as large as 170 kDa (Pellecehia et al. 2002 Nature Rev Drug Discovery 1:211). 2D [¹³C, ¹H]-HMQC (heteronuclear multiple quantum coherence) and ¹³C-edited [¹H,¹H]-NOESY NMR experiments on a ligand-receptor complex can be used to detect binding, determine the dissociation constant for the complex, and provide a low-resolution model based on the available three-dimensional structure of the target, thus revealing the relative position of the ligand with respect to labeled side-chains. Thus, NMR can be used to identify molecules that induce apoptosis. Compounds can be screened for binding to labeled Bcl-B, for example. Such labels include 15N and 13C. The interaction between the compound and Bcl-B, and therefore its ability to induce apoptosis, are determined via NMR. Accordingly, one embodiment of the invention is a method of optimizing compounds discovered by the methods described herein through NMR analysis. A target compound that is found to affect the binding between Bcl-B and a compound known to bind to and convert the Bcl-B protein to a proapoptotic form is provided. That target compound is then reacted with a library of chemical fragments in the presence of BCL-B in order to determine chemical fragments that bind a site adjacent to the target compound. Chemical fragments discovered to bind a site adjacent to the binding site of the target compound are covalently linked to the target compound to provide an optimized target compound.

### VII. Pharmacology

In one embodiment, methods for treating cancer by inducing apoptosis of cancer cells in an afflicted individual are provided. Accordingly, one or more inducers of apoptosis is administered to a patient in need of such treatment. A therapeutically effective amount of the drug can be administered as a composition in combination with a pharmaceutical vehicle. In other embodiments of the invention the apoptosis modulator targets a death antagonist associated with virally infected cells or self-reacting lymphocytes to comprise a treatment for viral infection or autoimmune disease.

For a review of apoptosis in the pathogenesis of disease, see Thompson, 1995 Science 267:1456-1462.

In particular, pro-apoptotic modulators of Bcl-2 or related Bcl-2 family members can be used to treat any condition characterized by the accumulation of cells which are regulated by Bcl-2 or related Bcl-2 family members. By “regulated by Bcl-2” with respect to the condition of a cell is meant that the balance between cell proliferation and apoptotic cell death is controlled, at least in part, by Bcl-2 or related Bcl-2 family members. For the most part, the cells express or overexpress Bcl-2 or related Bcl-2 family members. Enhancement of Bcl-2 or related Bcl-2 family members expression has been demonstrated to increase the resistance of cells to almost any apoptotic signal (Hockenbery et al. 1990 Nature 348:334; Nunez et al. 1990 Immunol 144:3602; Vaux et al. 1988 Nature 335:440; Hockenbery et al. 1993 Cell 75:241; Ohmori et al. 1993 Res Commun 192:30; Lotem et al. 1993 Cell Growth Differ 4:41; Miyashita et al. 1993 Blood 81:115). Principally, the proliferative disorders associated with the inhibition of cell apoptosis include cancer, autoimmune disorders and viral infections. Overexpression of Bcl-2 or related Bcl-2 family members specifically prevents cells from initiating apoptosis in response to a number of stimuli (Hockenbery et al. 1990 Nature 348:334; Nunez et al. 1990 J Immunol 144:3602; Vaux et al. 1988 Nature 335:440; Hockenbery et al. 1993 Cell 75:241). The induction of genes that inhibit Bcl-2 or related Bcl-2 family members can induce apoptosis in a wide variety of tumor types, suggesting that many tumors continually rely on Bcl-2 or related gene products to prevent cell death. Expression of Bcl-2 or related Bcl-2 family members has been associated with a poor prognosis in at least prostatic cancer, colon cancer and neuroblastoma (McDonnell et al. 1992 Cancer Res 52:6940; Hague et al. 1994 Oncogene 9:3367; Castle et al. 1993 Am J Pathol 143:1543). Bcl-2 or the related gene has been found to confer resistance to cell death in response to several chemotherapeutic agents (Ohmon et al. 1993 Res Commun 192:30; Lotem et al. 1993 Cell Growth Differ 4:41; Miyashita et al. 1993 Blood 81:115).

Physiologic cell death is important for the removal of potentially autoreactive lymphocytes during development and for the removal of excess cells after the completion of an immune response. Failure to remove these cells can result in autoimmune disease. A lupus-like autoimmune disease has been reported in transgenic mice constitutively overexpressing Bcl-2 or related Bcl-2 family members in their B cells (Strasser et al. 1991 PNAS USA 88:8661). Linkage analysis has established an association between the Bcl-2 locus and autoimmune diabetes in non-obese diabetic mice (Garchon et al. 1994 Eur J Immunol 24:380). The compositions described herein which comprise inhibitors of Bcl-2 function can be used to induce apoptosis of self-reactive lymphocytes. By “self-reactive” is meant a lymphocyte which participates in an immune response against antigens of host cells or host tissues.

Compositions comprising pro-apoptotic modulators of Bcl-2 or related Bcl-2 family members can be used in the treatment of viral infection, to induce apoptosis of virally infected cells. Viruses have developed mechanisms to circumvent the normal regulation of apoptosis in virus-infected cells, and these mechanisms have implicated Bcl-2 or related Bcl-2 family members. For example, the E1B 19-kDa protein is instrumental in the establishment of effective adenoviral infection. The apoptosis-blocking ability of E1B can be replaced in adenoviruses by Bcl-2 (Boyd et al. 1994 Cell 79:341). Genes of certain other viruses have been shown to have sequence and functional homology to Bcl-2 (Neilan et al. 1993 J Virol 67:4391; Henderson et al. 1993 PNAS USA 90:8479). The viral gene LMP-1 specifically upregulates Bcl-2 providing a survival advantage over latently infected cells (Henderson et al. 1991 Cell 65:1107). Sindbis infection is dependent on the host cell's expression of Bcl-2 (Levine et al. 1993 Nature 361:739).

Apart from other considerations, the fact that the novel active ingredients of the compositions described herein are peptides, peptide analogs or peptidomimetics, dictates that the formulation be suitable for delivery of these type of compounds. Clearly, peptides are less suitable for oral administration due to susceptibility to digestion by gastric acids or intestinal enzymes. The preferred routes of administration of peptides are intra-articular, intravenous, intramuscular, subcutaneous, intradermal, or intrathecal. A more preferred route is by direct injection at or near the site of disorder or disease. However, some of the compounds disclosed herein were proved to be highly resistance to metabolic degradation in addition to having the ability to cross cell membrane. These properties make them potentially suitable for oral administration. Pharmaceutical compositions as described herein can be manufactured by processes well known in the art, e.g., by means of conventional mixing, dissolving, granulating, grinding, pulverizing, dragee-making, levigating, emulsifying, encapsulating, entrapping or lyophilizing processes.

Toxicity and therapeutic efficacy of the pro-apoptotic modulators of Bcl-2 described herein can be determined by standard pharmaceutical procedures in cell cultures or experimental animals, e.g., by determining the IC₅₀ (the concentration which provides 50% inhibition) and the LD₅₀ (lethal dose causing death in 50% of the tested animals) for a subject compound. The data obtained from these cell culture assays and animal studies can be used in formulating a range of dosage for use in humans. The dosage can vary depending upon the dosage form employed and the route of administration utilized. The exact formulation, route of administration and dosage can be chosen by the individual physician in view of the patient's condition (e.g., Fingl et al. 1975, in “The Pharmacological Basis of Therapeutics”, Ch. 1 Al; and Remington's Pharmaceutical Sciences, by Joseph P. Remington, Mack Pub. Co. 1985).

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

The targeted cell can be solitary and isolated from other like cells (such as a single cell in culture or a metastatic or disseminated neoplastic cell in viva), or the targeted cell can be a member of a collection of cells (e.g., within a tumor). Preferably, the cell is a neoplastic cell (e.g., a type of cell exhibiting uncontrolled proliferation, such as cancerous or transformed cells). Neoplastic cells can be isolated (e.g., a single cell in culture or a metastatic or disseminated neoplastic cell in vivo) or present in an agglomeration, either homogeneously or, in heterogeneous combination with other cell types (neoplastic or otherwise) in a tumor or other collection of cells. Where the cell is within a tumor, some embodiments described herein provide a method of retarding the growth of the tumor by administering pro-apoptotic modulator of Bcl-2 to the tumor and subsequently administering a cytotoxic agent to the tumor.

By virtue of the cytopathic effect on individual cells, the inventive method can reduce or substantially eliminate the number of cells added to the tumor mass over time. Preferably, the inventive method effects a reduction in the number of cells within a tumor, and, most preferably, the method leads to the partial or complete destruction of the tumor (e.g., via killing a portion or substantially all of the cells within the tumor).

Where the targeted cell is associated with a neoplastic disorder within a patient (e.g., a human), some embodiments of the invention provide a method of treating the patient by first administering a pro-apoptotic modulator of Bcl-2 or related Bcl-2 family members to the patient (“pretreatment”) and subsequently administering a cytotoxic agent to the patient. This approach is effective in treating mammals bearing intact or disseminated cancer. For example, where the cells are disseminated cells (e.g., metastatic neoplasia), the cytopathic effects of the inventive method can reduce or substantially eliminate the potential for further spread of neoplastic cells throughout the patient, thereby also reducing or minimizing the probability that such cells will proliferate to form novel tumors within the patient. Furthermore, by retarding the growth of tumors including neoplastic cells, the inventive method reduces the likelihood that cells from such tumors will eventually metastasize or disseminate. Of course, when the inventive method achieves actual reduction in tumor size (and especially elimination of the tumor), the method attenuates the pathogenic effects of such tumors within the patient. Another application is in high-dose chemotherapy requiring bone marrow transplant or reconstruction (e.g., to treat leukemic disorders) to reduce the likelihood that neoplastic cells will persist or successfully regrow.

In many instances, the pretreatment of cells or tumors with pro-apoptotic modulator of Bcl-2 or related Bcl-2 family members before treatment with the cytotoxic agent effects an additive and often synergistic degree of cell death. In this context, if the effect of two compounds administered together in vitro (at a given concentration) is greater than the sum of the effects of each compound administered individually (at the same concentration), then the two compounds are considered to act synergistically. Such synergy is often achieved with cytotoxic agents able to act against cells in the Go-Go phase of the cell cycle.

Any period of pretreatment can be employed. For example, in therapeutic applications, such pretreatment can be for as little as about a day to as long as about 5 days or more; the pretreatment period can be between about 2 and about 4 days (e.g., about 3 days). Following pretreatment, a cytotoxic agent is administered. However, in other embodiments, a glucocorticoid (e.g., cortisol, dexamethasone, hydrocortisone, methylprednisolone, prednisolone, prednisone, etc.), diphenhydramine, rantidine, antiemetic-ondasteron, or ganistron can be adjunctively administered, and such agents can be administered with the pro-apoptotic modulator of Bcl-2 or related Bcl-2 family members. The cytotoxic agent can be administered either alone or in combination with continued administration of the pro-apoptotic modulator of Bcl-2 or related Bcl-2 family members following pretreatment. While, according to certain embodiments, treatment ceases upon administration of the cytotoxic agent, it can be administered continuously for a period of time (e.g., periodically over several days) as desired.

Any cytotoxic agent can be employed in the context of the invention, and as mentioned, many cytotoxic agents suitable for chemotherapy are known in the art. Such an agent can be, for example, any compound mediating cell death by any mechanism including, but not limited; to, inhibition of metabolism or DNA synthesis, interference with cytoskeletal organization, destabilization or chemical modification of DNA, apoptosis, etc. For example, the cytotoxic agent can be an antimetabolite (e.g., 5-fiourouricil (5-FU), methotrexate (MTX), fiudarabine, etc.), an anti-microtubule agent (e.g., vincristine, vinblastine, taxanes (such as paclitaxel and docetaxel), etc.), an alkylating agent (e.g., cyclophasphamide, melphalan, bischloroethylnitrosurea (BCNU), etc.), platinum agents (e.g., cisplatin (also termed cDDP), carboplatin, oxaliplatin, JM-216, CI-973, etc.), anthracyclines (e.g., doxorubicin, daunorubicin, etc.), antibiotic agents (e.g., mitomycin-C), topoisomerase inhibitors (e.g., etoposide, camptothecins, etc.), or other cytotoxic agents (e.g., dexamethasone). The choice of cytotoxic agent depends upon the application of the inventive method. For research, any potential cytotoxic agent (even a novel cytotoxic agent) can be employed to study the effect of the toxin on cells or tumors pretreated with vitamin D (or a derivative). For therapeutic applications, the selection of a suitable cytotoxic agent will often depend upon parameters unique to a patient; however, selecting a regimen of cytotoxins for a given chemotherapeutic protocol is within the skill of the art.

For in vivo application, the appropriate dose of a given cytotoxic agent depends on the agent and its formulation, and it is well within the ordinary skill of the art to optimize dosage and formulation for a given patient. Thus, for example, such agents can be formulated for administration via oral, subcutaneous, parenteral, submucosal, intravenous, or other suitable routes using standard methods of formulation. For example, carboplatin can be administered at daily dosages calculated to achieve an AUC (“area under the curve”) of from about 4 to about 15 (such as from about 5 to about 12), or even from about 6 to about 10. Typically, AUC is calculated using the Calvert formula, based on the glomerular filtration rate of creatinine (e.g., assessed by analyzing a plasma sample) (see, e.g., Martino et al. 1999 Anticancer Res 19:5587-91). Paclitaxel can be employed at concentrations ranging from about 50 mg/ml to about 100 mg/ml (e.g., about 80 mg/ml). Where dexamethasone is employed, it can be used in patients at doses ranging between about 1 mg to about 10 mg (e.g., from about 2 mg to about 8 mg), and more particularly from about 4 mg to about 6 mg, particularly where the patient is human. The dosage of the tyrosine kinase inhibitor is from 1 g/kg to 1 g/kg of body weight per day. According to one embodiment, the dosage of the tyrosine kinase inhibitor is from 0.01 mg/kg to 100 mg/kg of body weight per day. The optimal dosage of the tyrosine kinase inhibitor will vary, depending on factors such as type; and extent of progression of the cancer, the overall health status of the patient, the potency of the tyrosine kinase inhibitor, and route of administration. Optimization of the tyrosine kinase dosage is within ordinary skill in the art.

The pharmaceutical compositions disclosed herein can be most preferably used for prevention and treatment of malignancies selected from the group of hormone-refractory-prostate cancer; prostate cancer (Zin et al 2001 Clin Cancer Res 7:2475-9); breast cancer (Perez-Tenorio and Stal 2002 Brit J Cancer 86:540-45, Salh et al. 2002 Int J Cancer 98:148-54); ovarian cancer (Liu et al. 1998 Cancer Res 15:2973-7); colon cancer (Semba at al. 2002 Clin Cancer Res 8:1957-63); melanoma and skin cancer (Walderman, Wecker and Diechmann 2002 Melanoma Res 12:45-50); lung cancer (Zin et al. 2001 Clin Cancer Res 7:2475-9); and hepatocarcinoma (Fang et al. 2001 Eur J Biochem 268:45 13-9).

Additional specific types of cancers that can be treated using this invention include acute myelogenous leukemia, bladder, cervical, cholangiocarcinoma, chronic myelogenous leukemia, colorectal, gastric sarcoma, glioma, leukemia, lymphoma, multiple myeloma, osteosarcoma, pancreatic, stomach, or tumors at localized sites including inoperable tumors or in tumors where localized treatment of tumors would be beneficial, and solid tumors.

According to one preferred embodiment, the pro-apoptotic modulators of Bcl-2 can be administered in circumstances where the underlying cancer resists treatment with other chemotherapeutics or irradiation, due to the action of Bcl-2 blocking apoptosis.

Another embodiment of the invention provides a method of treating prostate cancer within a patient by administrating pro-apoptotic modulator of Bcl-2 or related Bcl-2 family members, and possibly a glucocorticoid, to the patient. Any pro-apoptotic modulator of Bcl-2 and glucocorticoid can be employed in accordance with this aspect of the invention, many of which are discussed elsewhere herein and others are generally known in the art. Moreover, pro-apoptotic modulator of Bcl-2 or related Bcl-2 family members and the glucocorticoid are delivered to the patient by any appropriate method, some of which are set forth herein. Thus, they can be formulated into suitable preparations and delivered subcutaneously, intravenously, orally, etc., as appropriate. Also, for example, the glucocorticoid is administered to the patient concurrently, prior to, or after the administration of the pro-apoptotic modulator of Bcl-2 or related Bcl-2 family members. One effective dosing schedule is to deliver between about 5 μg and about 25 g/kg, pro-apoptotic modulator of Bcl-2 or related Bcl-2 family members daily on alternative days (e.g., between 2 and 4 days a week, such as Mon-Wed-Fri or Tues-Thus-Sat, etc.), and also between about 1 mg/kg and 20 mg/kg dexamethasone to a human patient also on alternative days. In such a regimen, the alternative days on which pro-apoptotic modulator of Bcl-2 or related Bcl-2 family members and on which the glucocorticoid are administered can be different, although preferably they are administered on the same days. Even more preferably, the glucocorticoid is administered once, by itself, prior to concurrent treatment. Of course, the treatment can continue for any desirable length of time, and it can be repeated, as appropriate to achieve the desired end results. Such results can include the attenuation of the progression of the prostate cancer, shrinkage of such tumors, or, desirably, remission of all symptoms. However, any degree of effect is considered a successful application of this method. A convenient method of assessing the efficacy of the method is to note the change in the concentration of prostate-specific antigen (PSA) within a patient. Typically, such a response is gauged by measuring the PSA levels over a period of time of about 6 weeks.

Desirably, the method results in at least about a 50% decrease in PSA levels after 6 weeks of application, and more desirably at least about 80% reduction in PSA. Of course, the most desirable outcome is for the PSA levels to decrease to about normal levels.

Another embodiment of the invention provides a method of treating breast cancer within a patient by administrating the non-naturally occurring pro-apoptotic modulator of Bcl-2 or related Bcl-2 family members alone or in combination with any other treatment regimen for breast cancer. Treatments for breast cancer are well known in the art and continue to be developed. Treatments include but are not limited to surgery, including axillary dissection, sentinel lymph node biopsy, reconstructive surgery, surgery to relieve symptoms of advanced cancer, lumpectomy (also called breast conservation therapy), partial (segmental) mastectomy, simple or total mastectomy, modified radical mastectomy, and radical mastectomy; hormone therapy using a drug such as tamoxifen, which blocks the effects of estrogen; aromatase inhibitors, which stop the body from making estrogen; immunotherapy, e.g., using Herceptin™ (trastozumab), an anti-HER2 humanized monoclonal antibody developed to block the HER2 receptor; bone marrow transplantation; peripheral blood stem cell therapy; bisphosphonates; additional chemotherapy agents; radiation therapy; acupressure; and acupuncture. Particularly preferred chemotherapy agents for use in combination with the non-naturally-occurring compounds or peptides of the present invention include doxorubicin, paclitaxel, fluorouracil, cyclophosphamide, and tamoxifen. Any combination of therapies may be used in conjunction with the present invention.

In some embodiments, the pro-apoptotic modulators of Bcl-2 or related Bcl-2 family members can be used in the form of a pharmaceutically acceptable salt.

Suitable acids which are capable of forming salts include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like.

Suitable bases capable of forming salts include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

Pharmaceutically acceptable vehicles for delivery of the pro-apoptotic modulators of Bcl-2 or related Bcl-2 family members include physiologically tolerable or acceptable diluents, excipients, solvents, or adjuvants, for parenteral injection, for intranasal or sublingual delivery, for oral administration, for rectal or topical administration or the like. The compositions are preferably sterile and nonpyrogenic. Examples of suitable carriers include but are not limited to water, saline, dextrose, mannitol, lactose, or other sugars, lecithin, albumin, sodium glutamate cysteine hydrochloride, ethanol, polyols (propyleneglycol, ethylene, polyethyleneglycol, glycerol, and the like), vegetable oils (such as olive oil), injectable organic esters such as ethyl oleate, ethoxylated isostearyl alcohols, polyoxyethylene sorbitol and sorbitan esters, microcrystalline cellulose, aluminum methahydroxide, bentonite, agar-agar and tragacanth, or mixtures of these substances, and the like.

The pharmaceutical compositions can also contain minor amounts of nontoxic auxiliary substances such as wetting agents, emulsifying agents, pH buffering agents, antibacterial and antifungal agents (such as parabens, chlorobutanol, phenol, sorbic acid, and the like). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions.

Compositions containing the pro-apoptotic modulators of Bcl-2 or related Bcl-2 family members can be administered by any convenient route which will result in delivery of the conjugate to cells expressing the intracellular target. Modes of administration include, for example, orally, rectally, parenterally (intravenously, intramuscularly, intraarterially, or subcutaneously), intracistemally, intravaginally, intraperitoneally, locally (powders, ointments or drops), or as a buccal or nasal spray or aerosol.

The pharmaceutical compositions are most effectively administered parenterally, preferably intravenously or subcutaneously. For intravenous administration, they can be dissolved in any appropriate intravenous delivery vehicle containing physiologically compatible substances, such as sodium chloride, glycine, and the like, having a buffered pH compatible with physiologic conditions. Such intravenous delivery vehicles are known to those skilled in the art. In a preferred embodiment, the vehicle is a sterile saline solution. If the peptides are sufficiently small, other preferred routes of administration are intranasal, sublingual, and the like. Intravenous or subcutaneous administration can comprise, for example, injection or infusion.

The effective amount and method of administration of the pro-apoptotic modulators of Bcl-2 or related Bcl-2 family members will vary based upon the sex, age, weight and disease stage of the patient, whether the administration is therapeutic or prophylactic, and other factors apparent to those skilled in the art. Based upon the in vitro studies described herein, a suitable dosage is a dosage which will attain a tissue concentration of from about 1 to about 100 μM, more preferably from about 10 to about 75 μM. It is contemplated that lower or higher concentrations would also be effective. The tissue concentration can be derived from peptide conjugate blood levels. Such a dosage can comprise, for example, from about 0.1 to about 100 mg/kg.

Those skilled in the art will derive appropriate dosages and schedules of administration to suit the specific circumstances and needs of the patient. Doses are contemplated on the order of from about 1 to about 500, preferably from about 10 to about 100, most preferably from about 30 to about 80, mg/kg of body weight. The pro-apoptotic modulator of Bcl-2 or related Bcl-2 family members can be administered by injection daily, over a course of therapy lasting two to three weeks, for example. Alternatively, the agent can be administered by continuous infusion, such as via an implanted subcutaneous pump, as is well-known in cancer therapy.

The pro-apoptotic modulators of Bcl-2 or related Bcl-2 family members according described herein can be labeled with a fluorescent, radiographic or other visually detectable label and utilized in in vitro studies to identify cells expressing an intracellular target, or to identify the location of the target inside of such cells. For example, a pro-apoptotic modulator of Bcl-2 or related Bcl-2 family members can be synthesized with an attached biotin molecule and incubated with cells suspected of expressing the target. The cells are then incubated with streptavidin-fluorescein. Cells expressing the intracellular target will bind the biotin conjugate, and the streptavidin-fluorescein complex. The result is a pattern of fluorescence inside the cell. In particular, a pro-apoptotic modulator of Bcl-2 or related Bcl-2 family members which binds the Bcl-2 protein or related Bcl-2 family members can be utilized to identify tumor cells which express Bcl-2 or related Bcl-2 family members. Assessment of Bcl-2 expression has prognostic value, as tumors expressing high levels of Bcl-2 or related Bcl-2 family members are likely to be chemoresistant and/or radiation resistant.

Selected compounds described herein are peptide-based substrate mimetic pro-apoptotic modulators of Bcl-2 that are stable in plasma for 6-24 hours, slowly metabolized by hepatic cells and are membrane permeable. The pro-apoptotic modulators of Bcl-2 induce apoptosis in cancer cells in the same concentrations that cell death is induced, while no cytotoxic death is observed at these concentrations by cell cycle analysis.

In addition, additional indications that can be treated using the pharmaceutical compositions described herein include any condition involving undesirable or uncontrolled cell proliferation Such indications include restenosis, benign tumors, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Specific types of restenotic lesions that can be treated include coronary, carotid, and cerebral lesions. Specific types of benign tumors that can be treated include hemangiomas, acoustic neuromas, neurofibroma, trachomas and pyogenic granulomas.

Treatment of cell proliferation due to insults to body tissue during surgery can be possible for a variety of surgical procedures, including joint surgery, bowel surgery, and keloid scarring. Diseases that produce fibrotic tissue include emphysema. Repetitive motion disorders that can be treated include carpal tunnel syndrome. An example of cell proliferative disorders that can be treated is a bone tumor.

Abnormal angiogenesis that can be treated include those abnormal angiogenesis accompanying rheumatoid arthritis, psoriasis, diabetic retinopathy, and other ocular angiogenic diseases such as retinopathy of prematurity (detrimental fibroplastic), macular degeneration, corneal graft rejection, neuromuscular glaucoma and Ouster Webber syndrome.

The proliferative responses associated with organ transplantation that can be treated include those proliferative responses contributing to potential organ rejections or associated complications. Specifically, these proliferative responses can occur during transplantation of the heart, lung, liver, kidney, and other body organs or organ systems.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art.

Accordingly, it is intended to embrace all such modifications and variations that fall within the spirit and broad scope of the appended claims.

### Materials and Methods.

Peptide synthesis. Peptides were synthesized on MBHA resin using Fmoc synthesis and DIC/HOBt coupling with an Advanced Chem Tech 350 and 396 multiple peptide synthesizer. All peptides except FITC-peptides were acetylated on their N-termini and all were amidated on their C-termini. Standard deprotection conditions were used for all peptides except those with Pbf-protected D-arginine octamers which were treated for 6 hr. Peptides were purified by HPLC on C18 columns and confirmed by MALDI mass analysis. Disulfide linked peptides were prepared as described (Giriat, I. & Muir, T. W. 2003 J Am Chem Soc 125:7180-1). Peptides with C-terminal cysteines were covalently linked to chloroacetylated N-aminocaproic acid in a displacement reaction.

Apoptosis assays—For nuclear morphological change analysis, cells were trypsinized, washed with PBS, fixed with 3.7% paraformaldehyde, and stained with DAPI (4,6-diamidino-2-phenylindole) (50 μg/ml) to visualize the nuclei by UV-microscopy. The percentages of apoptotic cells were determined by counting 300 GFP-positive cells, scoring cells having nuclear fragmentation and/or chromatin condensation.

Circular dichroism (CD) spectroscopy. Stock solutions of 3 mM peptide in 30% acetonitrile/water were added to 0.5 mL of 2 μM purified GST-proteins in PBS, pH 7.6. CD spectra were obtained in a 0.2 cm pathlength cell at 20° C. using an AVIV 62 DS spectropolarimeter for a wavelength range from 200 to 260 nm with a step size of 1 nm averaged for 5 sec. Three spectra were corrected for background and averaged for each sample. The Kd was determined using nonlinear regression analysis for a one-site-binding model (χ2>0.98). Stoichiometry was determined from a Zhou plot (Jones, G. et al. 2002 Tet. Let

### Example 1

### Fluorescence Polarization Assay

The following examples describe one embodiment of a FPA used to identify compounds that target Bcl-2-family members and regulate their apoptotic functions.

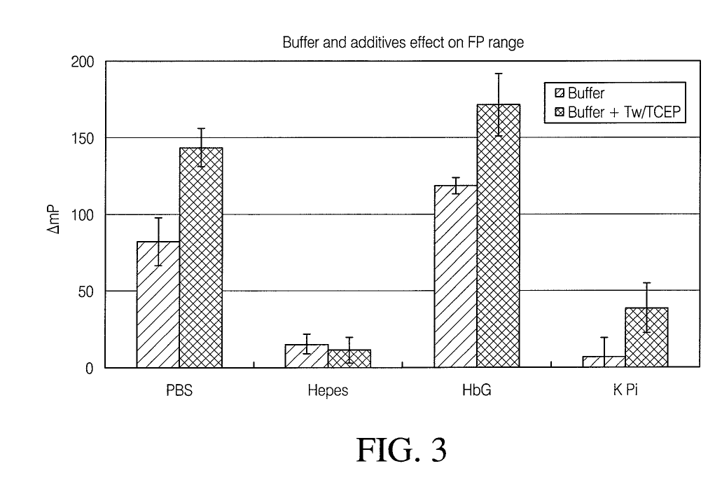

Fluorescence polarization (FP) analysis was used to determine whether FITC-TR3-9-r8 interacted directly with Bcl-2 proteins according to Zhai et al. (Biochem J. 376:229-236, 2003). Briefly, a serial concentration of GST-Bcl-2, GST-Bcl-X_{L}, GST-Bcl-B, GST-Bcl-W and GST-Bfl-1 was incubated with 5 nM FITC-conjugated TR3-9-r8 in PBS in a 96-well plate Greiner Fluotrac 600 or bio-one). Fluorescence polarization was measured after 10 min using an Analyst TM AD Assay Detection System (LJL Biosystem, Sunnyvale, Calif.)) with excitation wavelength set at 485 nm and dynamic polarizer for emission at 530 nm in PBS, pH 7.4. The results (FIGS. 3A-B) show that this peptide binds to all of these Bcl-2 family members in a concentration-dependent manner. Optimization of this assay for high throughput screening is described below.

### Example 2

### Optimization of Fluorescence Polarization Assay for High Throughput Screening

The FPA may also be used for high-throughput screening (HTS) of compounds that bind to Bcl-B, or to any of the Bcl-2 family of proteins. The HTS FPA uses reduced volumes for compatibility with 384-well plate formats, resulting in stabilization of the assay signal for easy assay automation. In the optimization studies described below, GST-Bcl-B ΔTM (MW 46 kDa) and FITC-Tr3-r8 (9-mer) were utilized.

For easy reformatting of the assay into 384-well plates, the volume was decreased to 20 uL. LJL HE 96 B plates (96-well plates with conical flat-bottom wells) were utilized. To evaluate the effect of buffer components on signal stability, the assay was performed in the original PBS buffer and three other pH 7.5 buffers: 1) 25 mM HEPES-KOH (HEPES), 2) 10 mM K-phosphate (K-Pi), and 3) 25 mM HEPES-KOH, 20 mM β-glycerophosphate (HβG). Each buffer was tested with and without the reducing agent TCEP (Tris(2-carboxyethyl)phosphine; 1.5 mM) and detergent (Tween 20; 0.0075%). Buffers and their parameters are summarized in Table 2. The effect of different buffers on the assay signal window is demonstrated in FIG. 3.

TABLE 2
Starting buffers and their parameters for buffer optimization study

| | PBS | HEPES | HβG | K-Pi |
|---|---|---|---|---|
| Phosphate (mM) | 10 | 0 | 0 | 10 |
| Na⁺ (mM) | 166.7 | 0 | 40 | 0 |
| K⁺ (mM) | 0 | 12.5 | 12.5 | 15 |
| Cl⁻ (mM) | 148 | 0 | 0 | 0 |
| βGP (mM) | 0 | 0 | 20 | 0 |
| HEPES (mM) | 0 | 25 | 25 | 0 |
| Ionic Strength | 175.4 | 12.5 | 72.5 | 27.4 |
| Divalent anions (mM) | 8.7 | 0 | 20 | 8.7 |

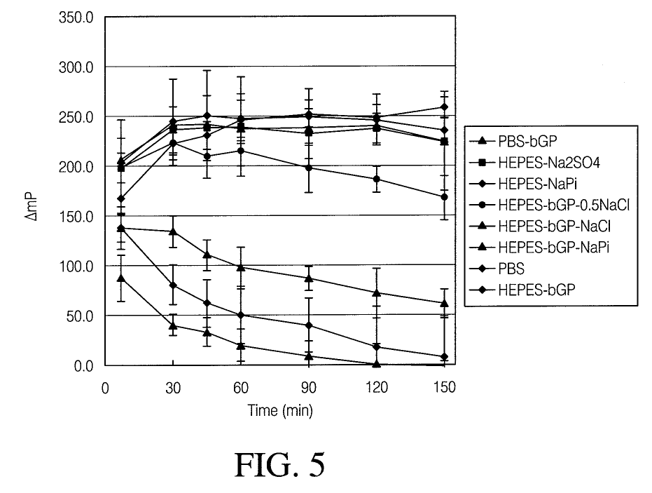

At the 10 min time point, the window of the assay was significant only in PBS and HβG buffers (FIG. 3). The presence of TCEP and Tween 20 improved the signal. When samples were incubated in LJL HE 96 B plates and measured over a period of time, the signal stability was strongly dependent on the buffer used. FIG. 5 depicts the effect of the buffer components and plate material on the fluorescence polarization signal stability for two buffers, PBS and HβG.

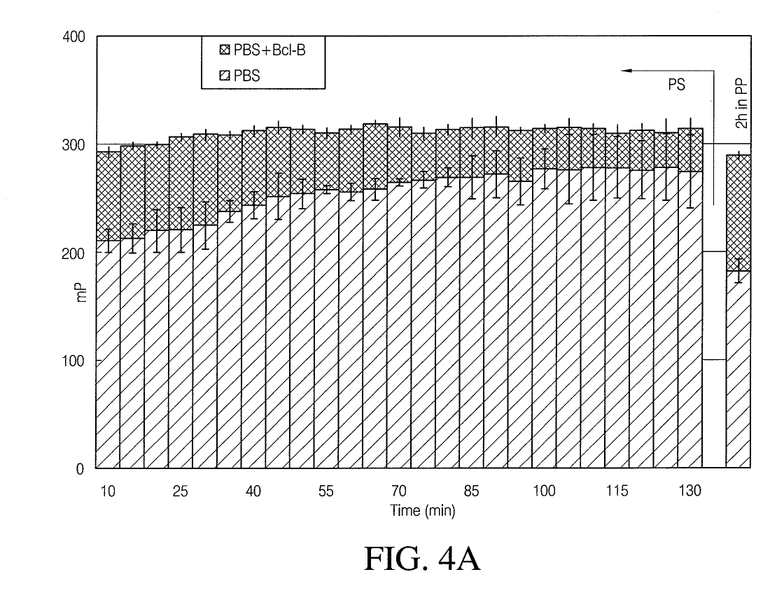

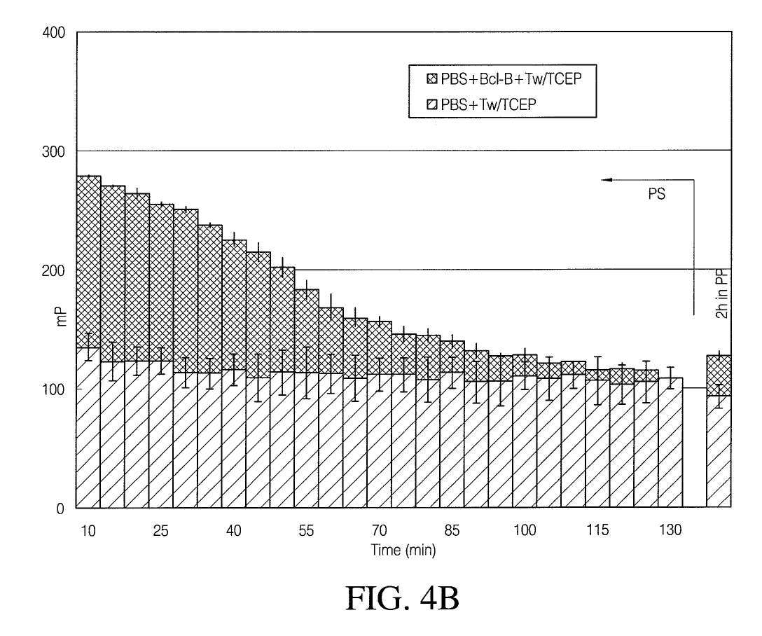

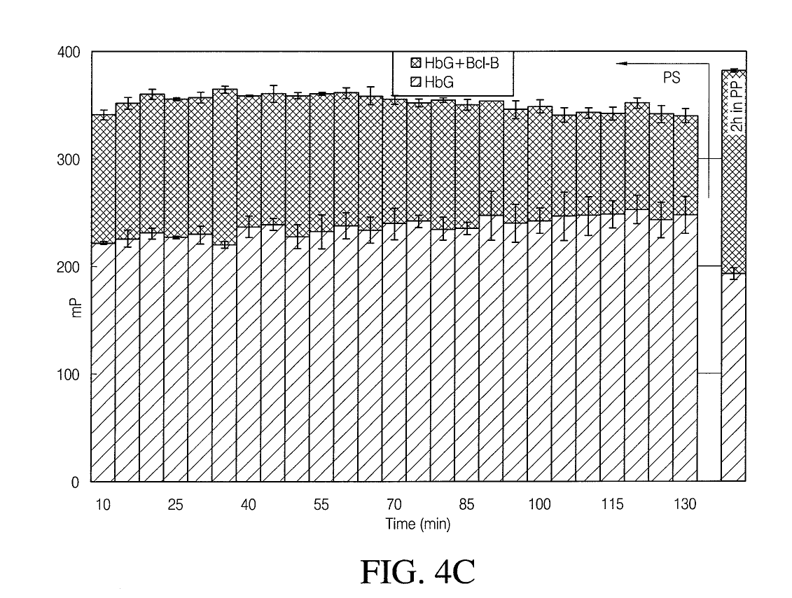

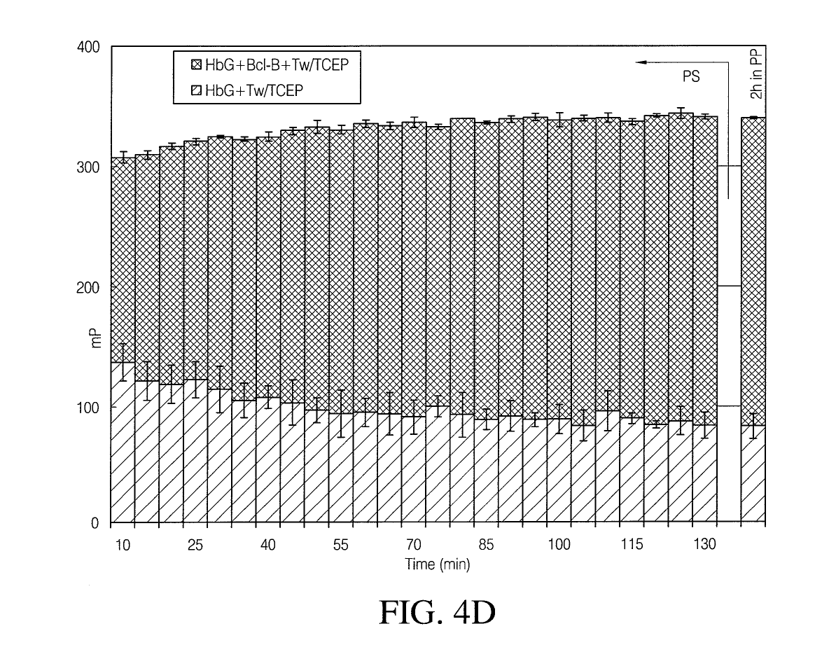

The signal quickly deteriorated in PBS buffer both with and without Tween 20/TCEP. It was more stable in HβG, and developed over time in HβG with Tween 20/TCEP. Some signal enhancement was also observed in HEPES and K-Pi buffers supplemented with Tween 20/TCEP. Polystyrene (PS) is the material utilized in most of the screening plates, whereas polypropylene (PP) is more hydrophilic and is used primarily in the plates for sample preparation and storage. Samples assayed in HOG buffer with Tween 20/TCEP were insensitive to the plate material (see FIG. 4).

Based on the analysis of the above results a new set of buffers (Table 3) was developed and tested. All of these buffers contained TCEP and Tween 20. Stability of the assay signal is shown in FIG. 5.

TABLE 3
Extended buffer panel for Bcl-B FPA buffer optimization.

| | Buffer name | | | | | | | |
|---|---|---|---|---|---|---|---|---|
| | | HEPES- | HEPES- | HEPES-bGP- | HEPES-bGP- | HEPES-bGP- | | |
| | | Na₂SO₄ | NaPi | 0.5NaCl | NaCl | NaPi | | |
| | | 25 mM | 25 mM | 25 mM | 25 mM | 20 mM | | HEPES-bGP |
| | PBS + bGP | HEPES, | HEPES, | HEPES, 20 mM | HEPES, 20 mM | HEPES, 20 mM | | 25 mM |
| | PBS, 20 mM | 20 mM | 25 mM | bGP, 75 mM | bGP, 150 mM | bGP, 10 mM | PBS | HEPES, 20 mM |
| Composition | bGP | Na₂SO₄ | NaPi | NaCl | NaCl | Na-Pi | x1 PBS | bGP |
| Pi | 10 | 0 | 25 | 0 | 0 | 10 | 10 | 0 |
| Na | 206.7 | 40 | 46.75 | 115 | 190 | 58.7 | 166.7 | 40 |
| K | 0 | 12.5 | 12.5 | 12.5 | 12.5 | 12.5 | 0 | 12.5 |
| Cl | 148 | 0 | 0 | 75 | 150 | 0 | 148 | 0 |
| βGP | 20 | 0 | 0 | 20 | 20 | 20 | 0 | 20 |
| HEPES | 0 | 25 | 25 | 25 | 25 | 25 | 0 | 25 |
| Ionic Strength | 235.05 | 72.5 | 81 | 147.5 | 222.5 | 99.9 | 175.4 | 72.5 |
| Divalent anions | 28.7 | 20 | 21.5 | 20 | 20 | 28.7 | 8.7 | 20 |

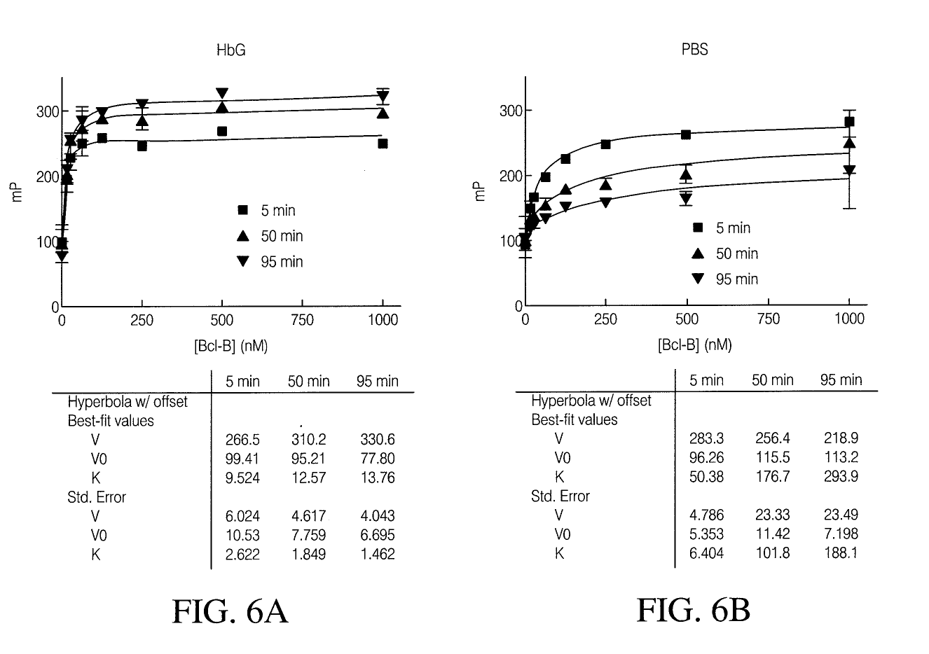

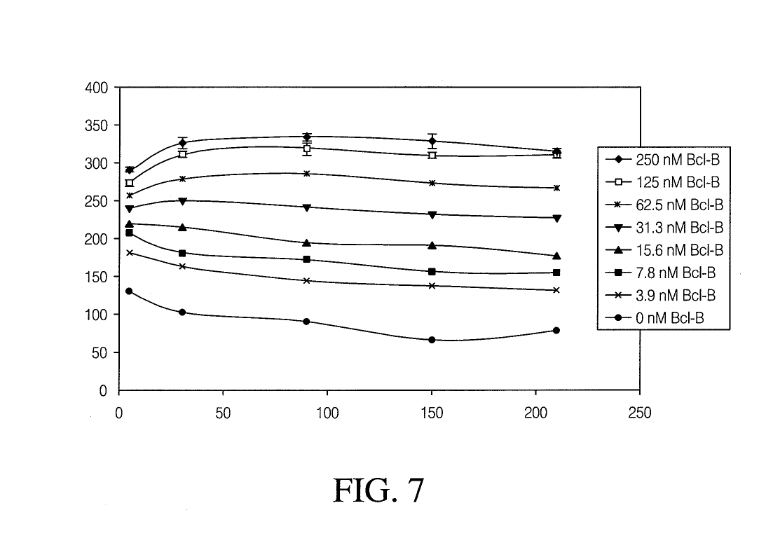

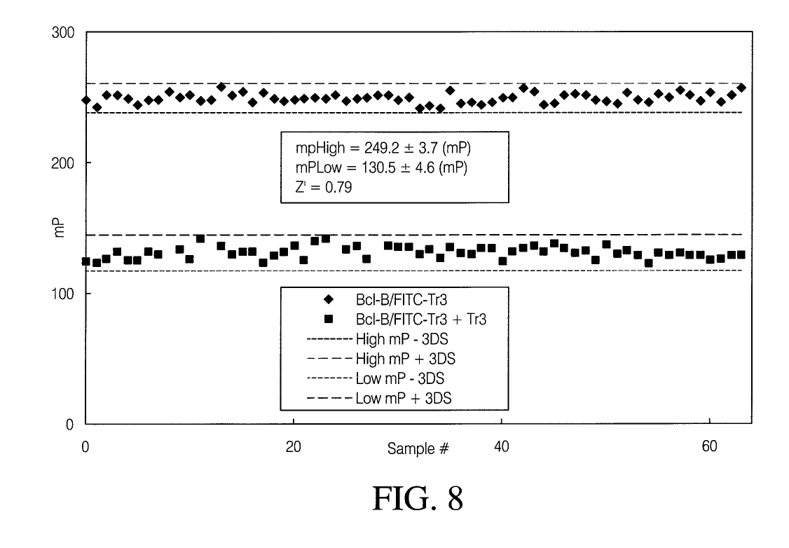

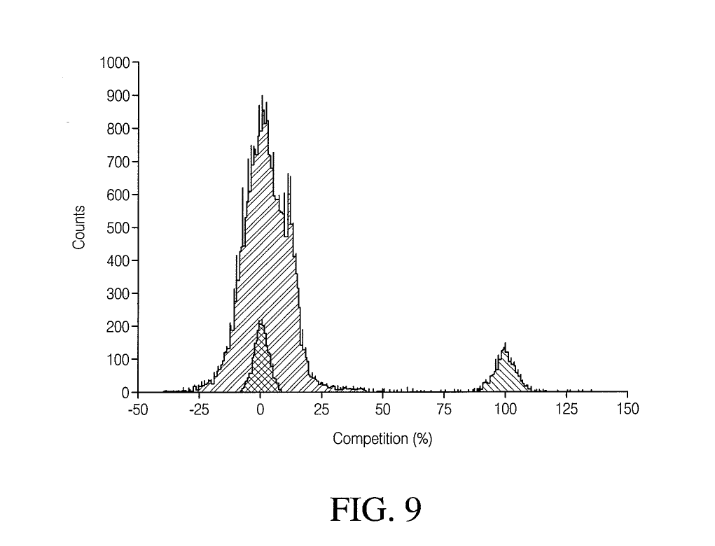

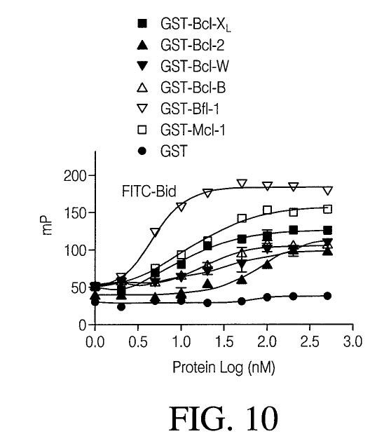

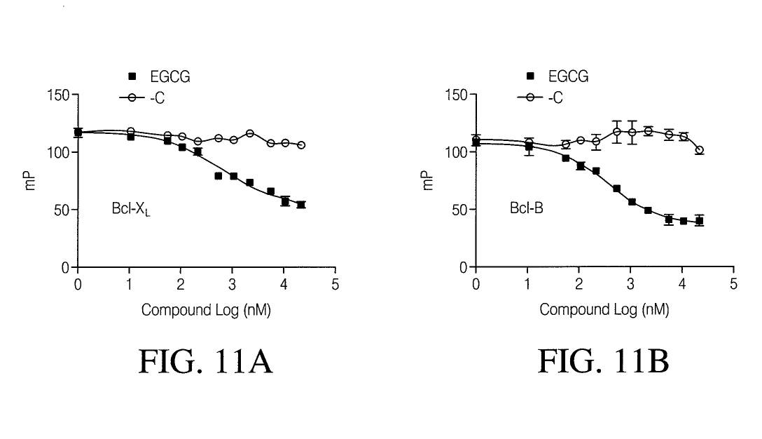

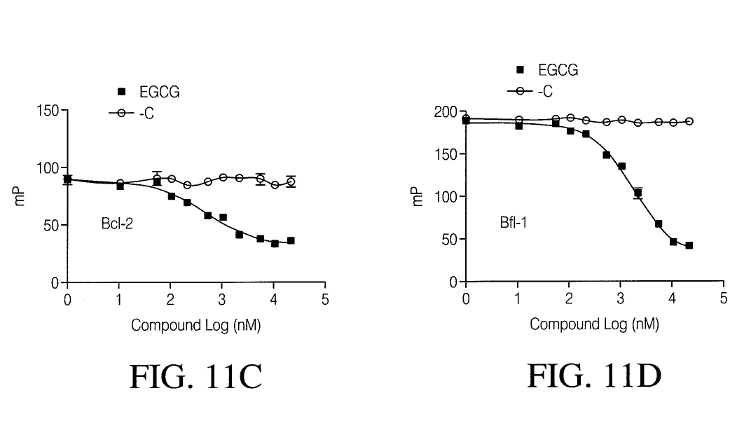

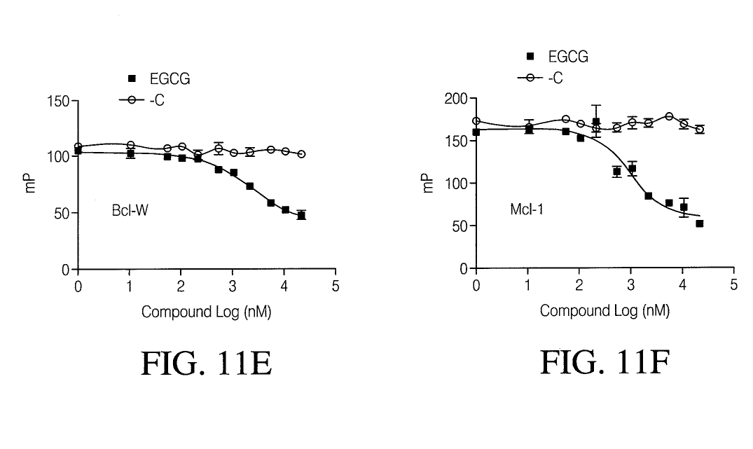

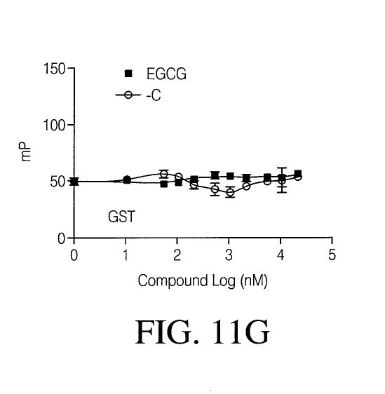

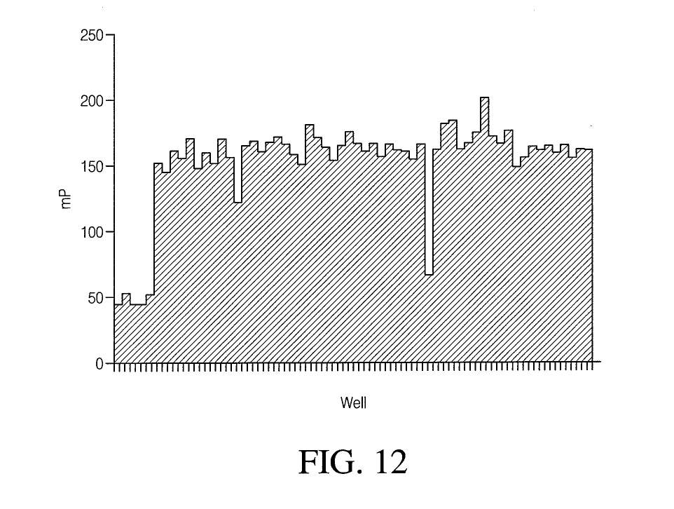

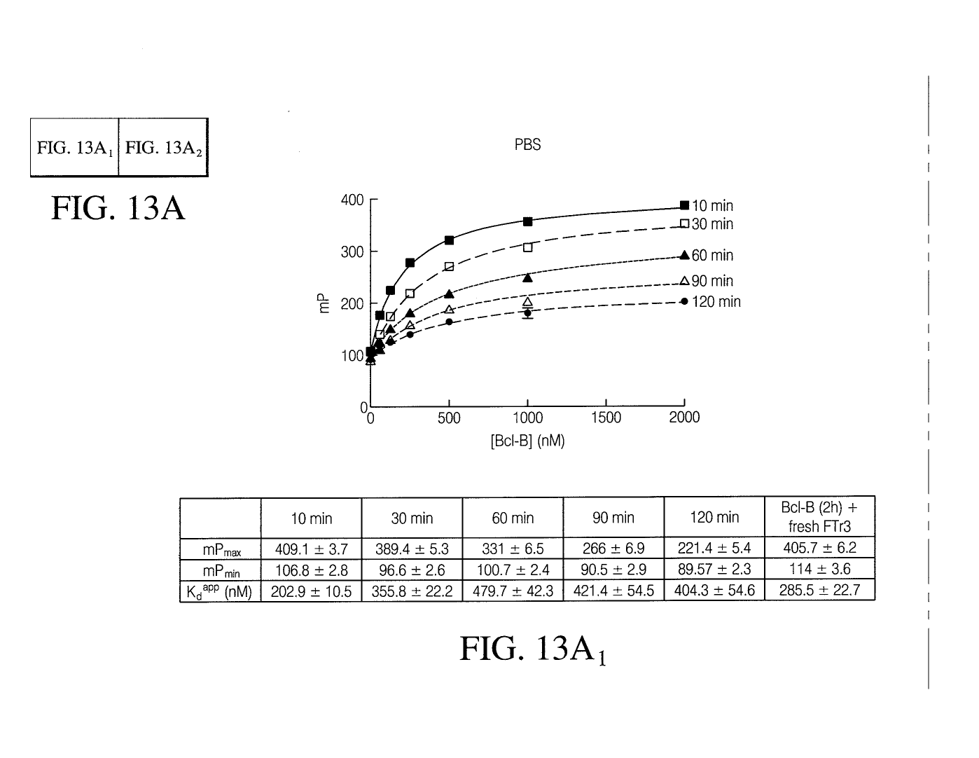

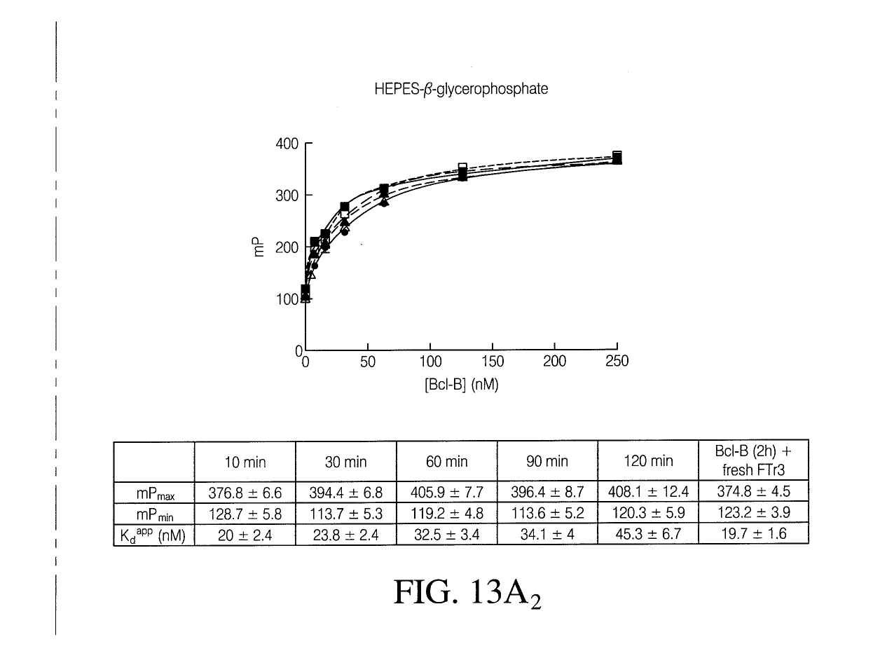

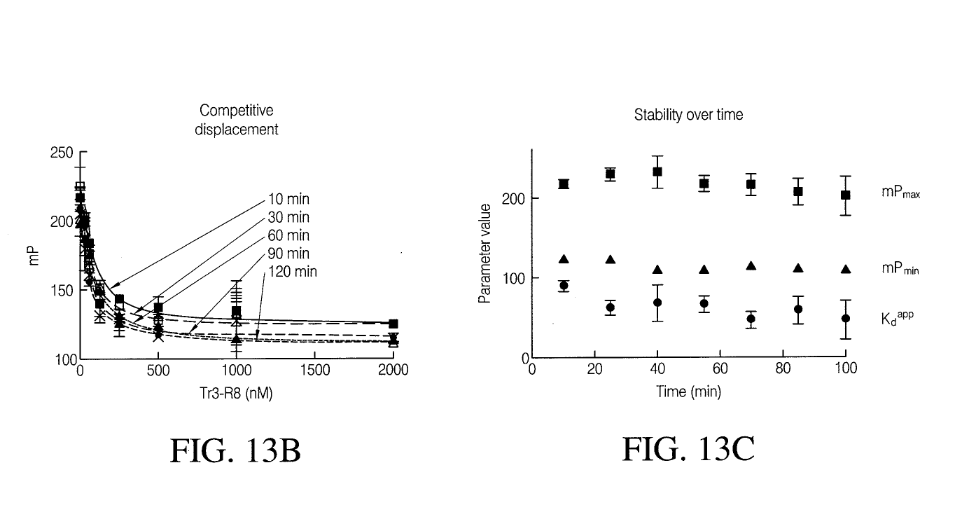

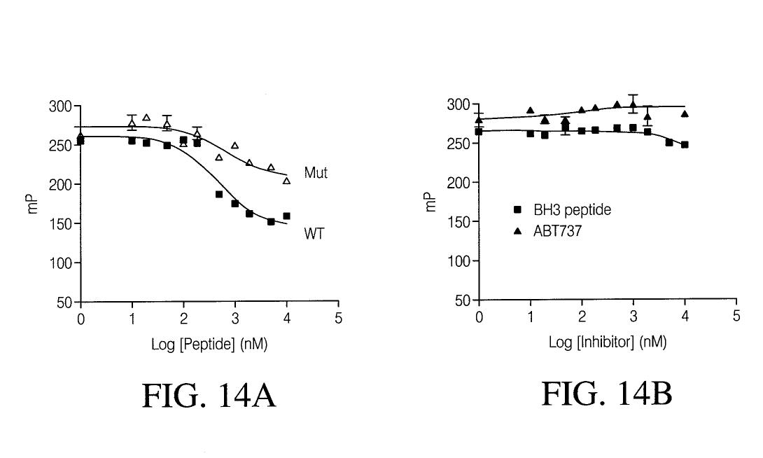

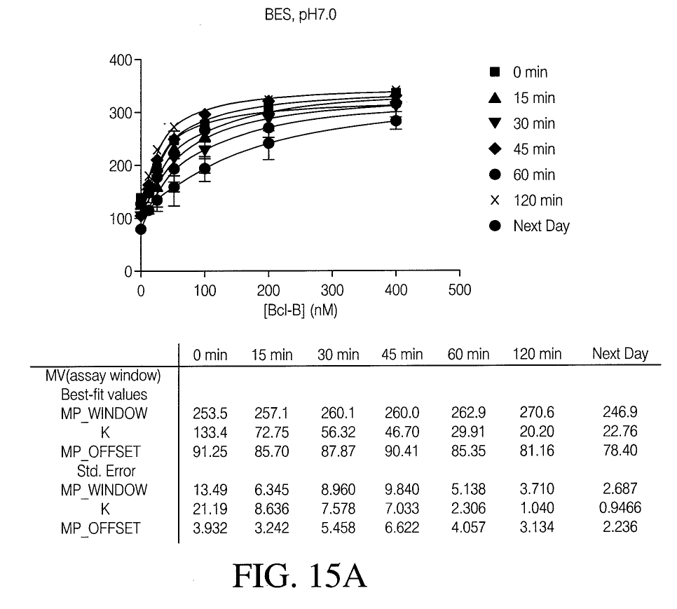

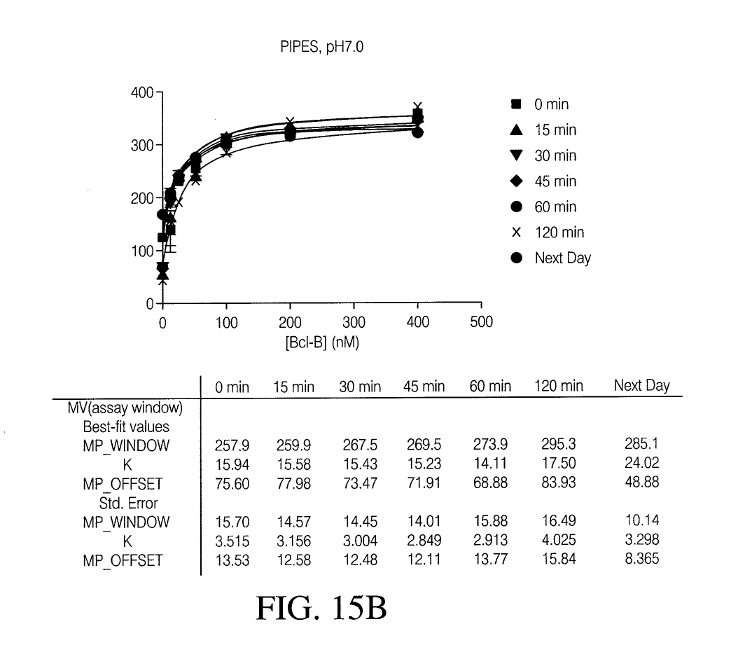

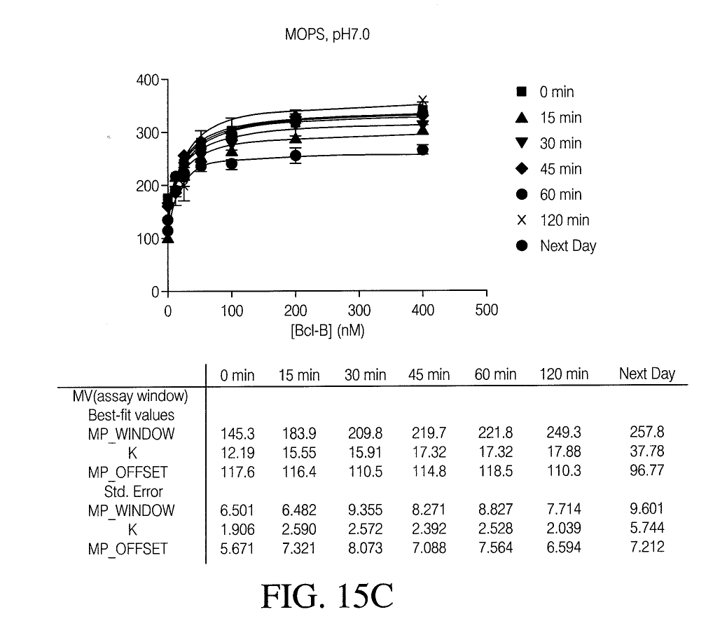

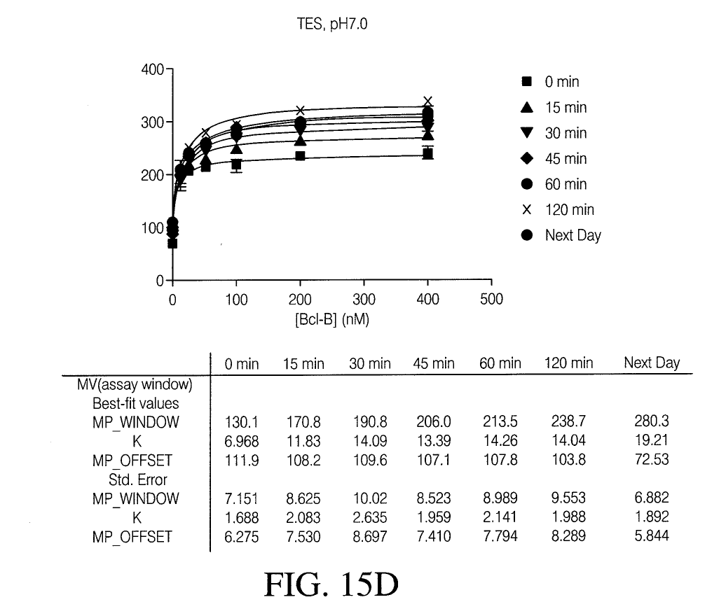

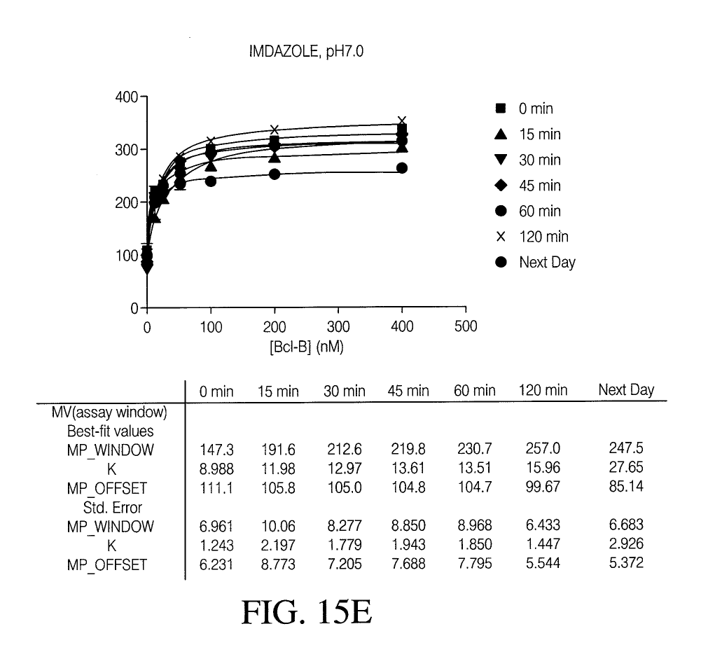

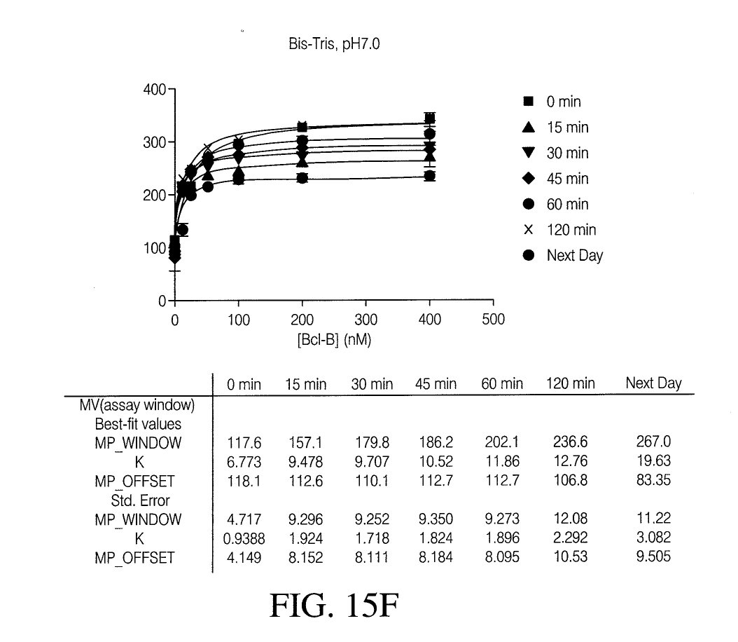

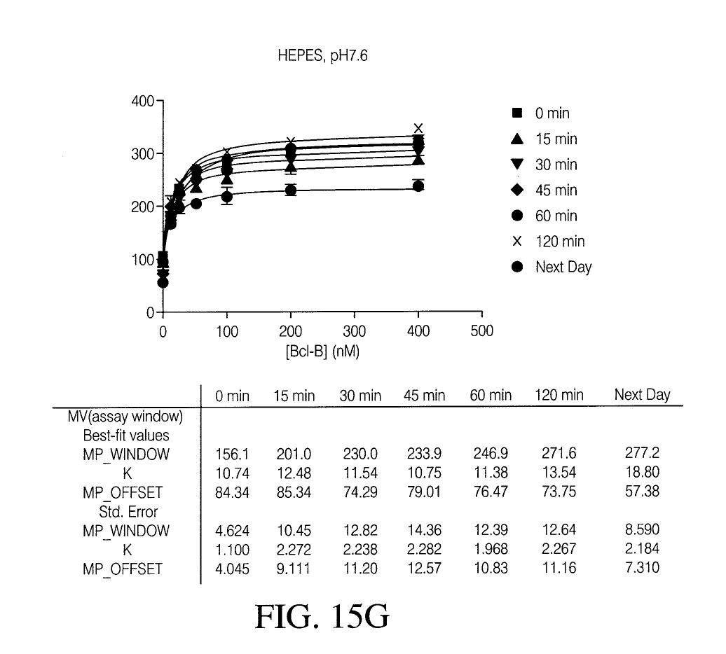

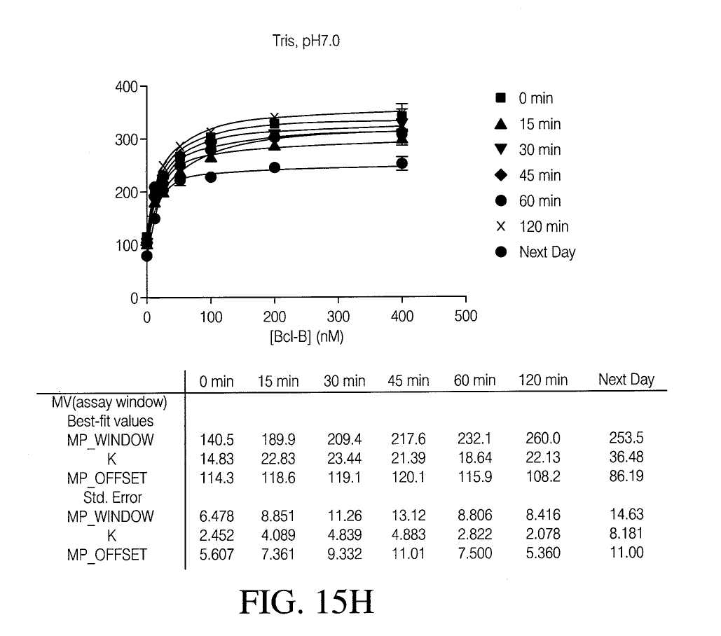

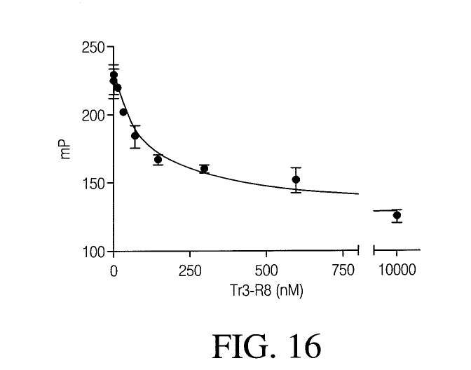

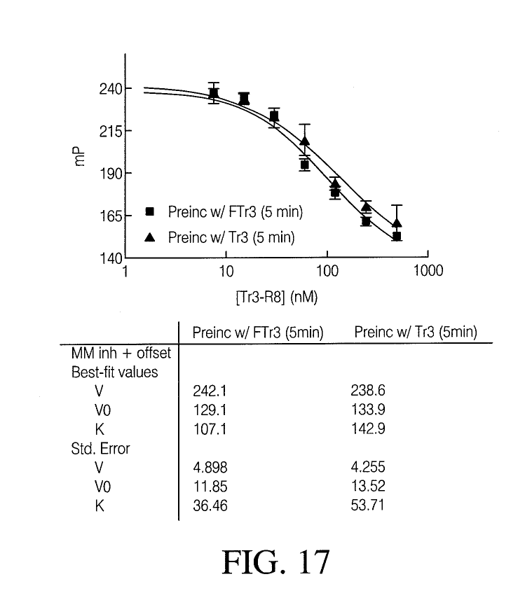

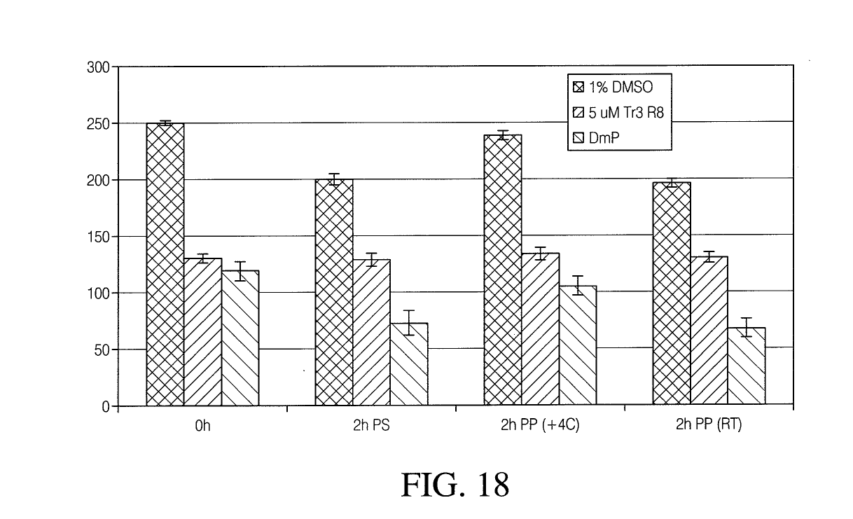

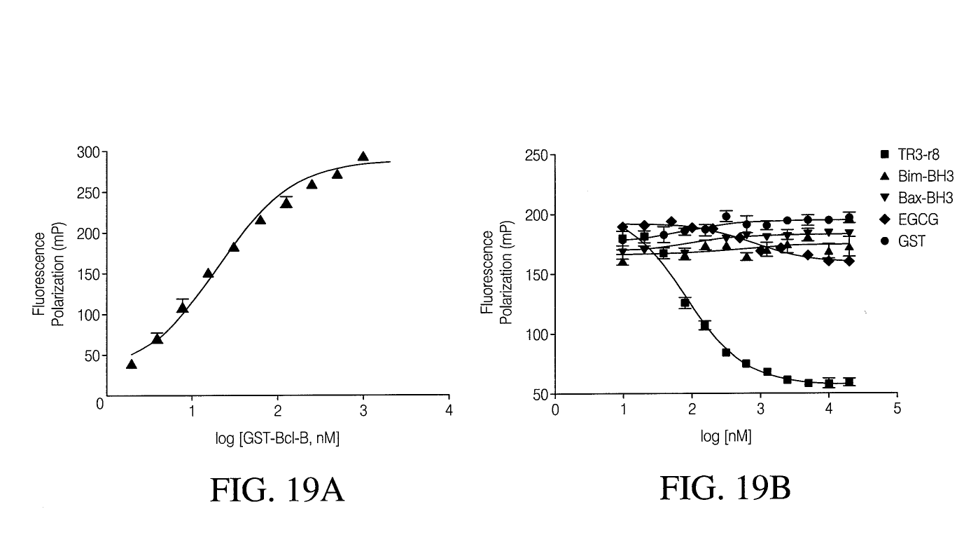

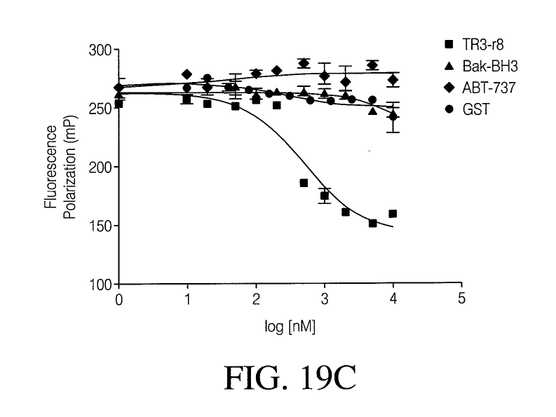

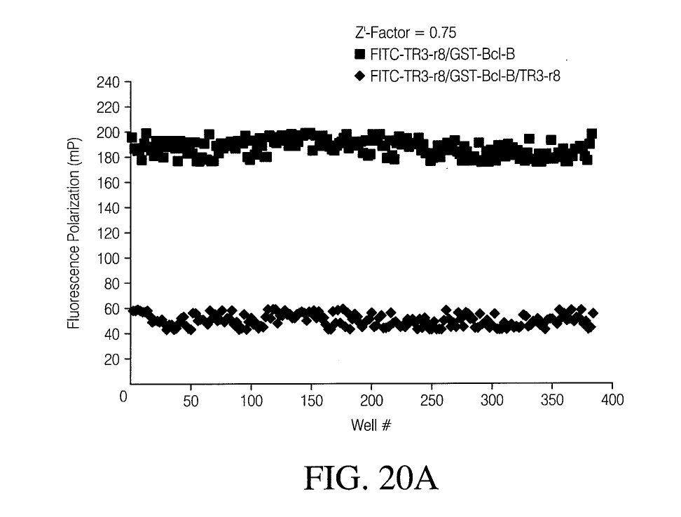

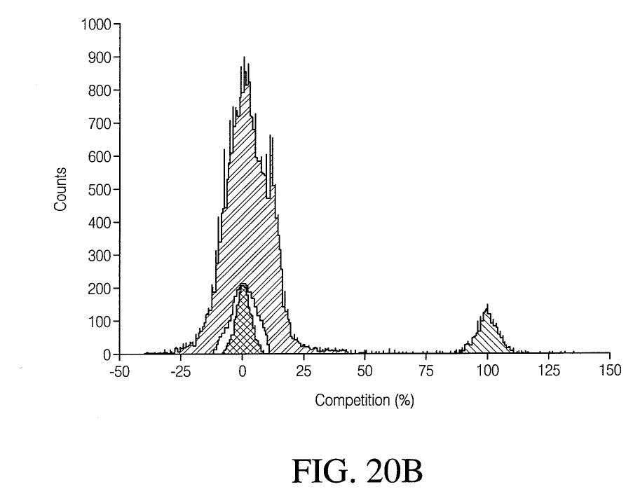

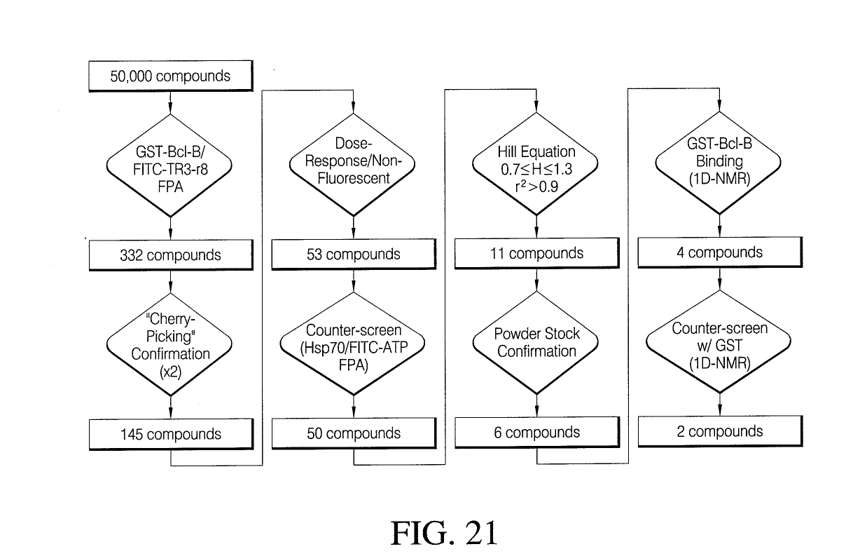

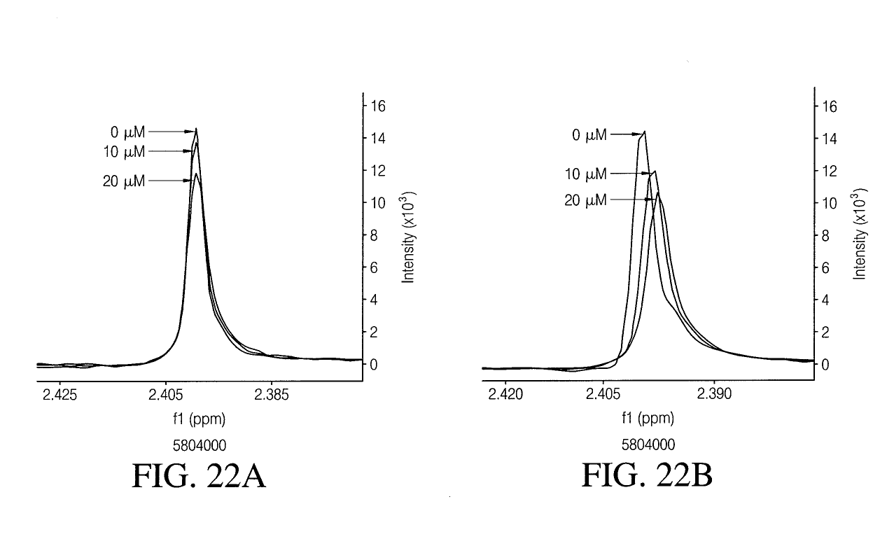

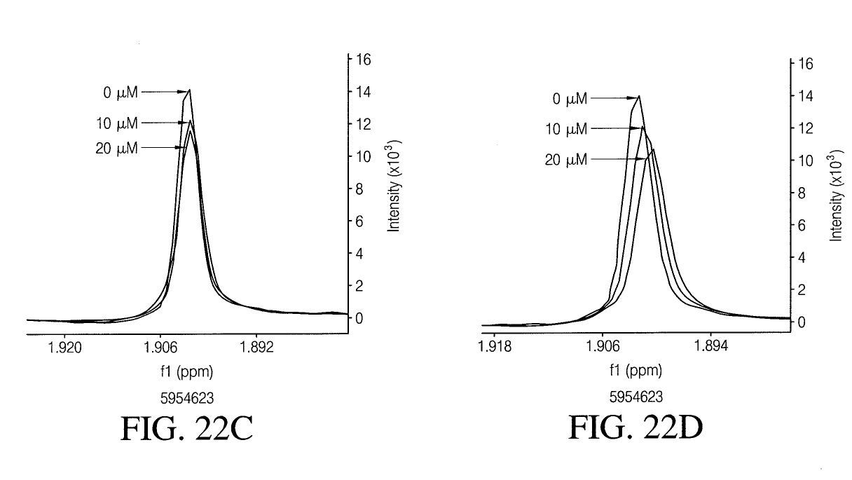

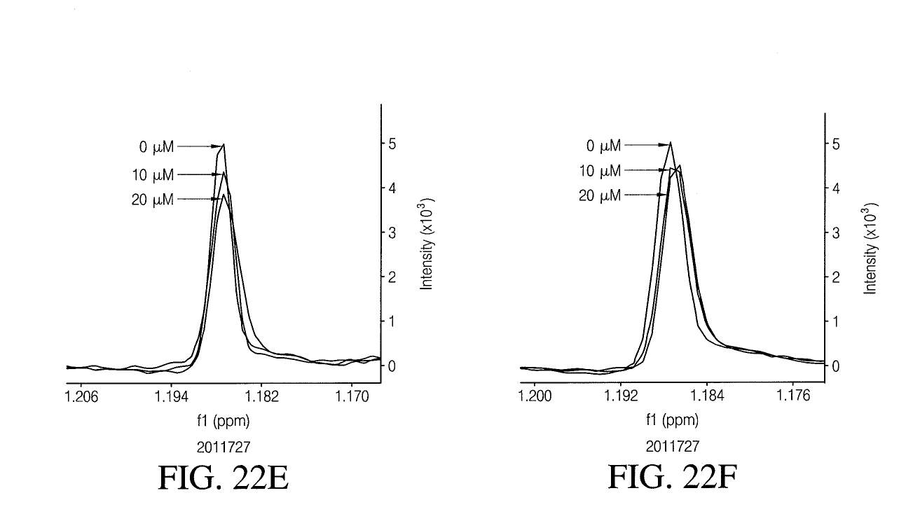

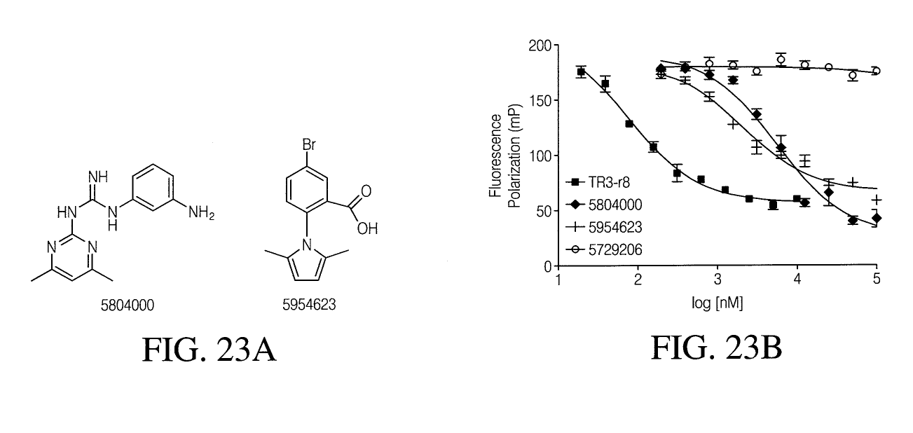

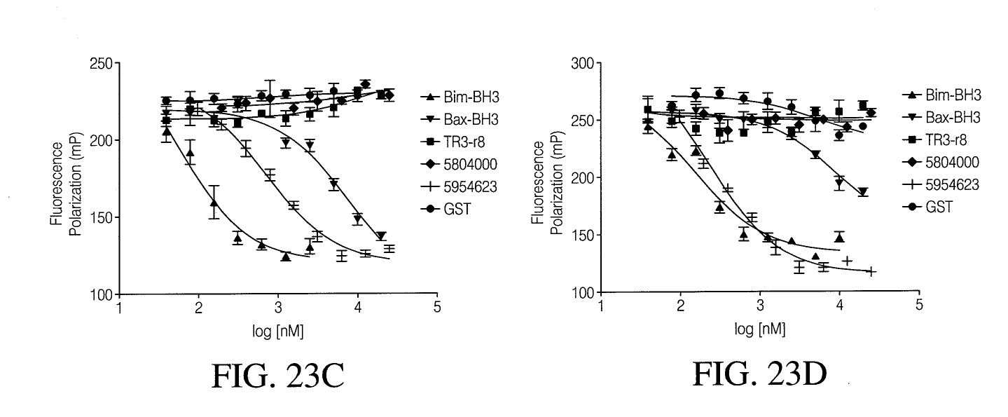

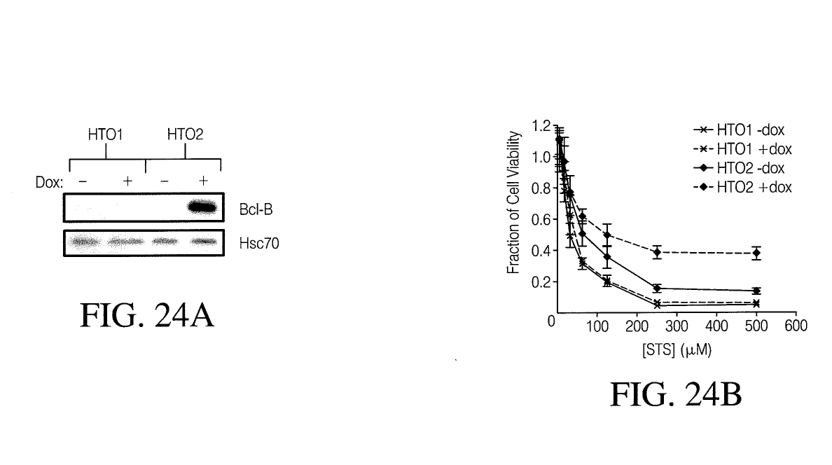

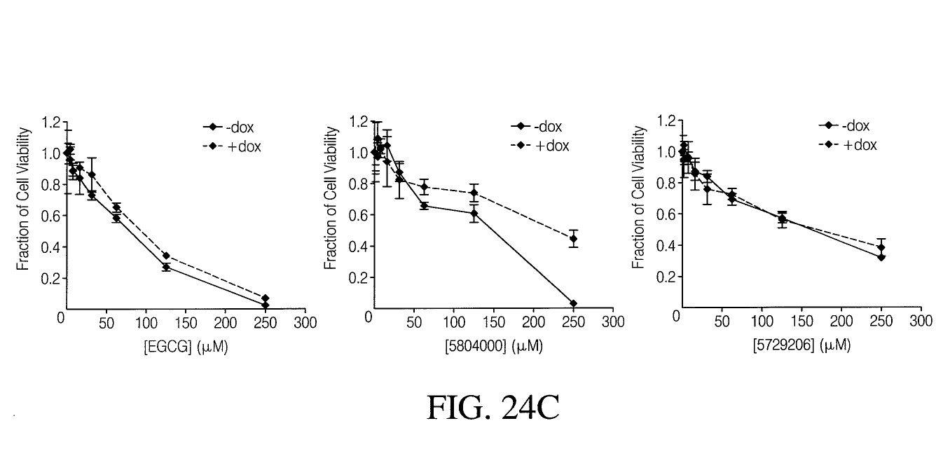

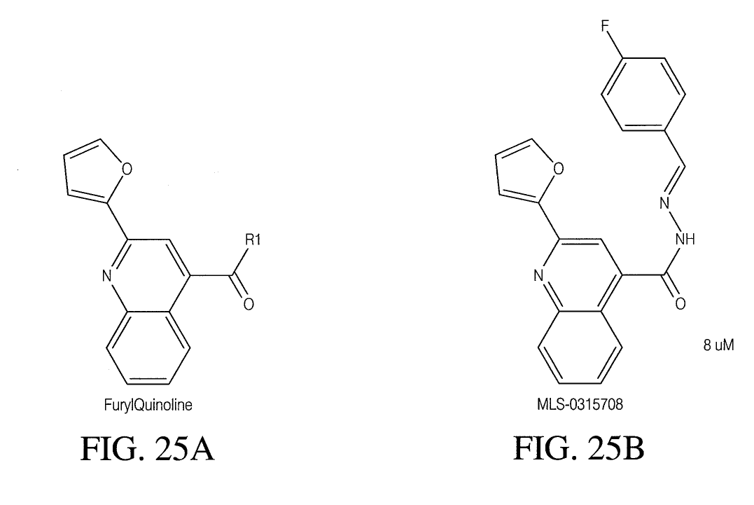

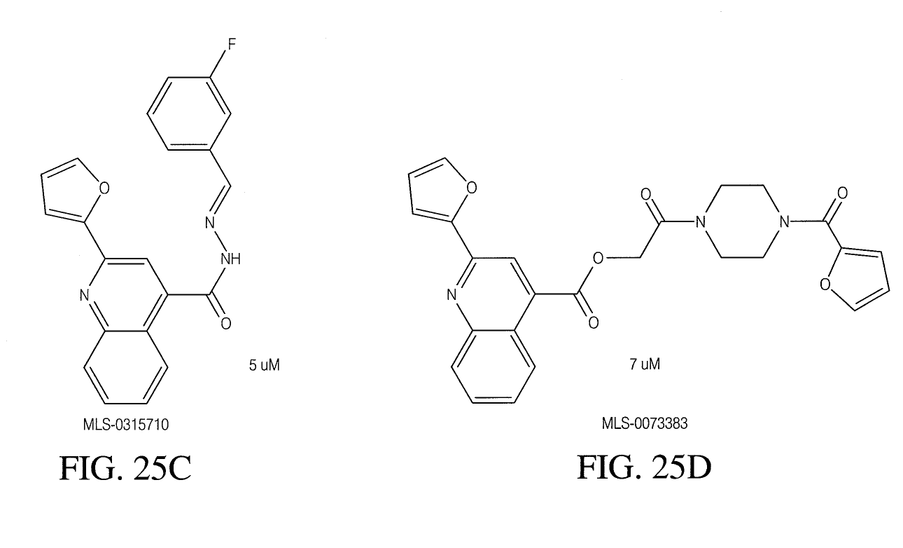

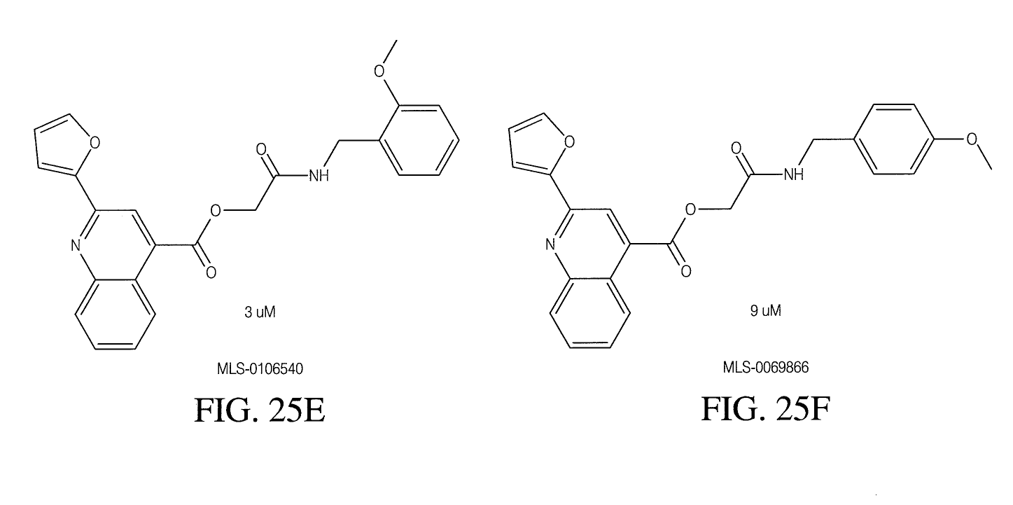

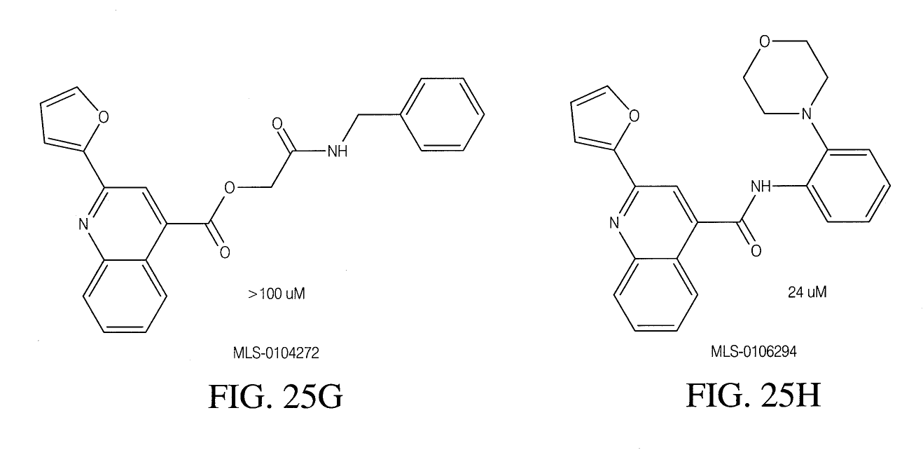

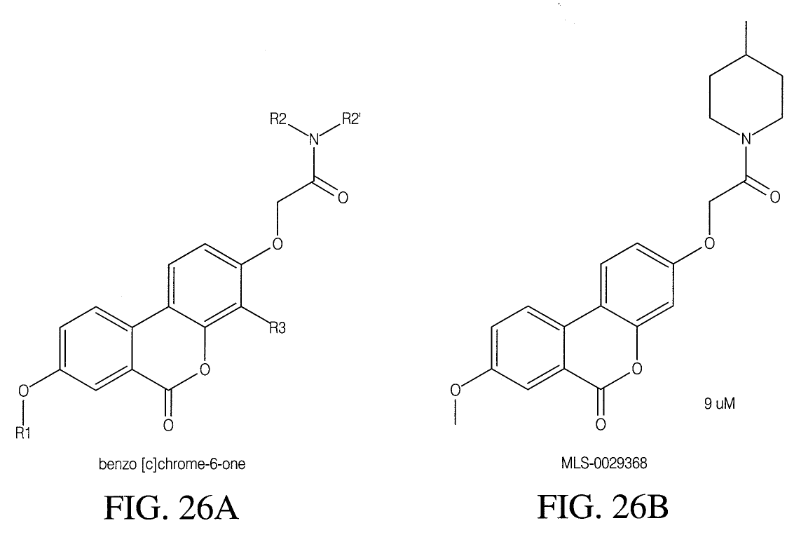

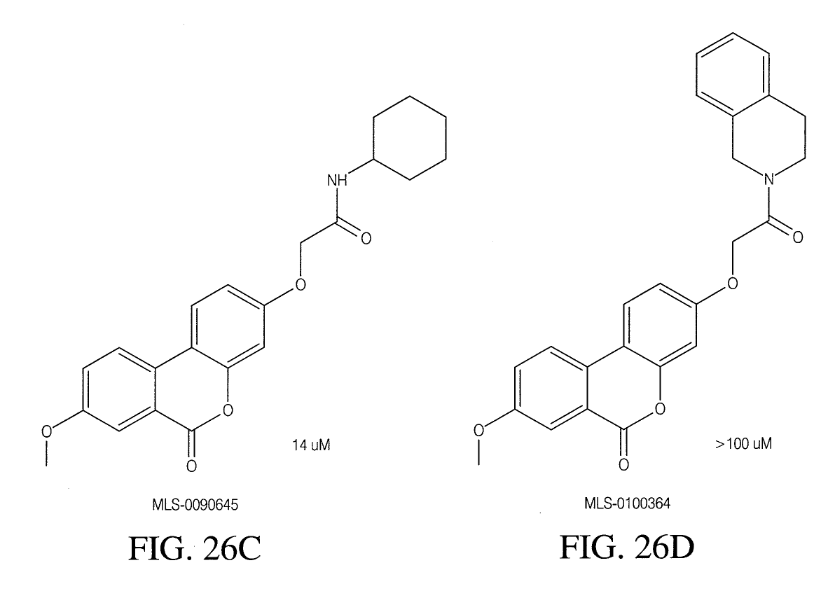

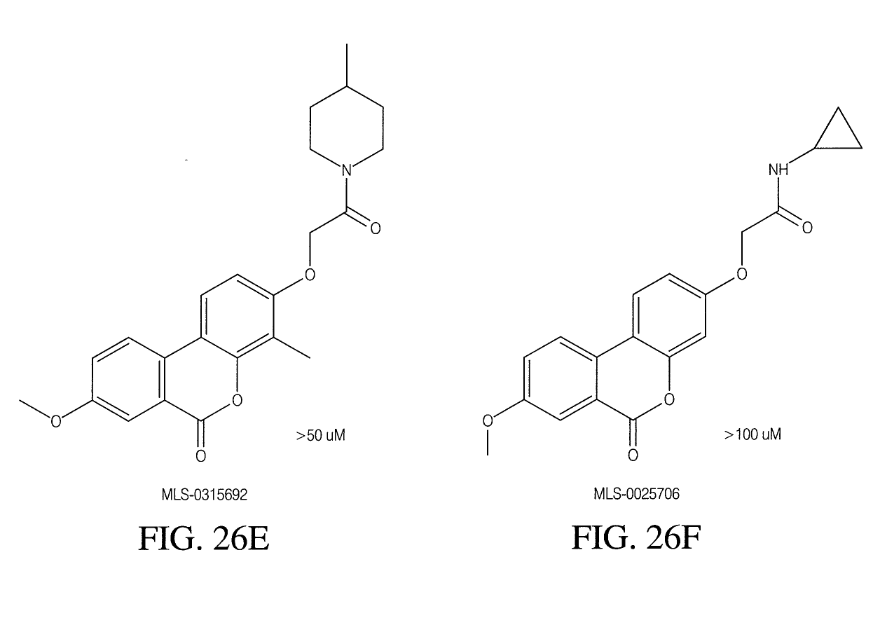

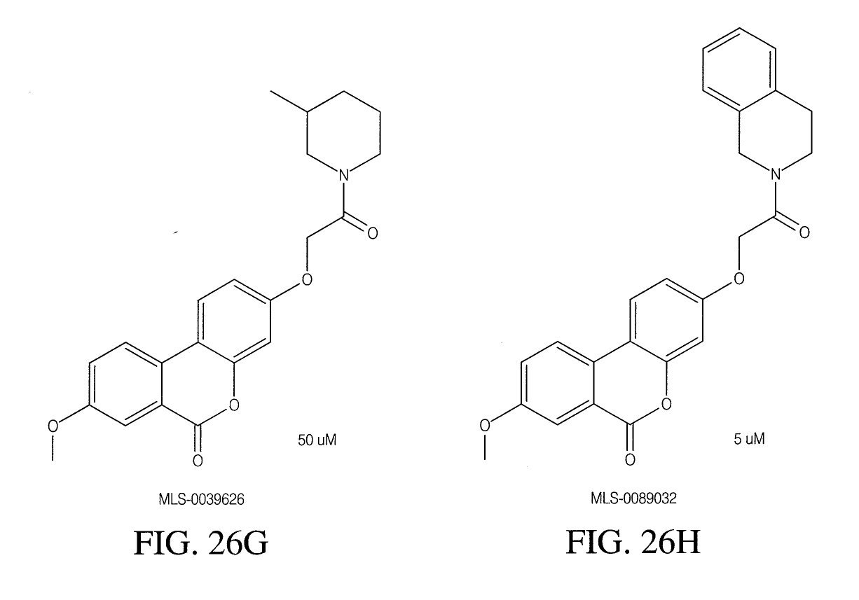

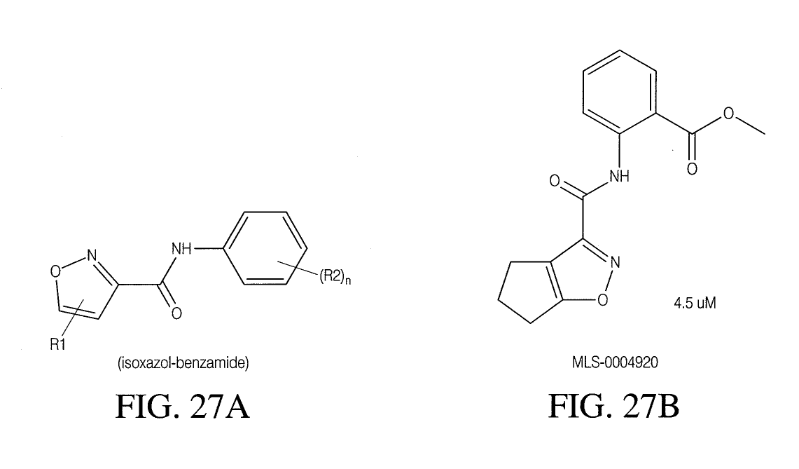

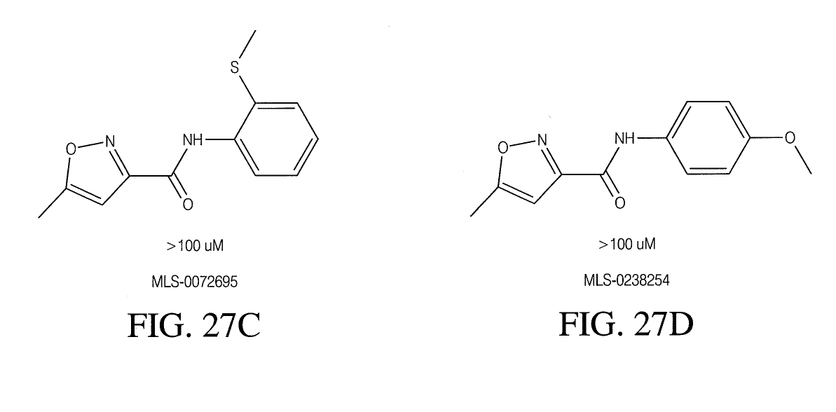

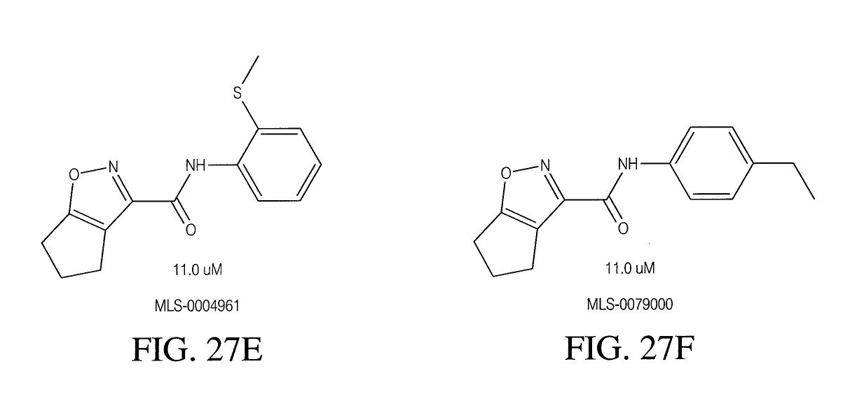

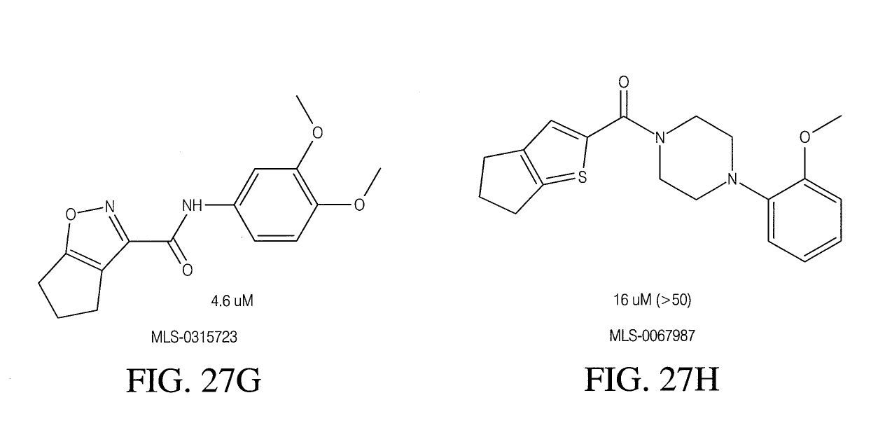

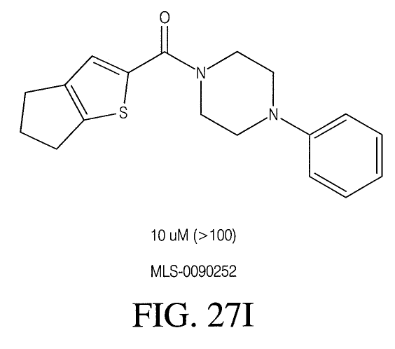

Several buffers resulted in improved signal stability (FIG. 5). Two buffers in particular, HβG and HβG+NaPi, demonstrated even lower signal variability. Since the assay window in these buffers is almost two-fold compared to the one observed in PBS buffer, it is likely that Bcl-B may also have higher affinity to FITC-Tr3. Binding curves for Bcl-B and FITC-Tr3 in HβG buffer are shown in FIG. 6. Signal stability curves at different concentrations of Bcl-B are demonstrated in FIG. 7.

The affinity of FITC-Tr3 binding to Bcl-B increases 5 to 10-fold when measured in HβG buffer compared to PBS buffer. In addition, the affinity in HβG buffer did not deteriorate with time as it does in PBS buffer (FIGS. 6 and 13). Although there was some decrease of fluorescence polarization signal at most of the concentrations of Bcl-B (FIG. 7), the assay window was constant from 1 h to 2.5 h after the assay set-up.

### Example 3

### Fluorescence Polarization Displacement Assay

The ability of unlabeled TR3-r8 (9-mer peptide) to compete with and displace FITC-labeled TR3-r8 for binding to Bcl-B was investigated. A mixture of Bcl-B (25 nM) and FITC-TR3-r8 (20 nM) was added with varied concentrations of unlabeled TR3-r8 in the presence of 1% DMSO. The experiment was performed in HβG buffer supplemented with TCEP and Tween 20. The results of this experiments are demonstrated in FIG. 13B. The parameters of the displacement curves demonstrated stable values over 2 h period. The displacement assay was utilized for screening small-molecule compounds.

### Bcl-B Assay Materials:

1) Bcl-B protein and FITC-TR3-R8 peptide (FITC-Ahx-FSRSLHSLL-GX-R8) were produced at the Burnham Institute for Medical Research, San Diego, Calif., as described in prior publications (Zhai et al., Biochem. J. 15:229-236, 2003; Luciano et al., Blood 109:3849-3855, 2007).

2) Assay buffer: 37.5 mM HEPES-NaOH, pH 7.5, 1.5 mM TCEP, 0.0075% Tween 20.

3) Bcl-B working solution contained 55 nM Bcl-B in assay buffer. Solution was prepared fresh and kept on ice prior to use.

4) Assay buffer with β-GP: 37.5 mM HEPES-NaOH, pH 7.5, 30 mM β-glycerophosphate (β-GP), 1.5 mM TCEP, 0.0075% Tween 20.

5) FITC-TR3 working solution contained 50 nM FITC-TR3-R8 peptide in the assay buffer with β-GP.

### Bcl-B HTS Protocol:

Four microliters of 100 uM compounds in 10% DMSO were dispensed in columns 3-24 of Greiner 384-well black small-volume plates (784076). Columns 1 and 2 contained 4 uL of 10% DMSO. Positive control wells, that contained no Bcl-B, were assigned to column 1. Assay buffer (8 uL) was added to these wells using the WellMate bulk dispenser (Matrix). 8 uL of Bcl-B working solution was added to columns 2-24 using the WellMate bulk dispenser (Matrix). Negative control wells that contained no compounds were assigned to column 2. The plates were briefly centrifuged and 8 uL of freshly prepared FITC-TR3-r8 working solution was added to the whole plate using the WellMate bulk dispenser (Matrix). Final concentrations of the components in the assay were as follows: 25 mM HEPES-NaOH, pH 7.5, 1 mM TCEP, 12 mM 13-glycerophosphate, 0.005% Tween 20, 20 nM FITC-TR3 (columns 1-24), 22 nM Bcl-B (columns 2-24), 2% DMSO (columns 1-24), 20 uM compounds (columns 3-24). The plates were incubated for 15 min at room temperature (protected from direct light). Fluorescence polarization was measured on an Analyst HT plate reader (Molecular Devices, Inc) using fluorescein filters: excitation filter 485 nm, emission filter 530 nm, dichroic mirror 505 nm. The signal acquisition time was 100 ms. Data analysis was performed using CBIS software (ChemInnovations, Inc). Fluorescence intensity of each sample was normalized to the average fluorescence intensity value of the plate negative control wells to calculate F-ratio parameter.

In the FPA, the F-Ratio is the fluorescent intensity of a measurement divided by the average fluorescent intensity of the control. The fluorescent intensity equals the parallel polarization signal+2×G-factor×perpendicular polarization signal, where G-factor is an experimentally determined correction factor to compensate for differences in sensitivity of the parallel and perpendicular PMT measurements For the F-Ratio denominator, the maximum fluorescence intensity signal of the controls is typically used. However, in control wells with the TR3-r8 competitor, the fluorescent intensity values are not the most reliable denominators because the fluorescence intensity increases substantially with very small doses of Tr3-r8 which would skew for low F-ratios even on many fluorescent compounds.

As shown in FIG. 9, unlabeled TR3 was able to competitively inhibit binding of FITC-labeled TR3 to Bcl-B and the mP values obtained in those samples were accepted to represent 100% displacement.

### Example 4

### Screening of Chemical Library Using Competitive FPA

A Chembridge chemical library containing 50,000 compounds was screened using the FPA high throughput protocol described above. The results are shown in FIG. 10. Out of 50,000 compounds screened, 427 exhibited at least 50% competition, and 332 exhibited an F-ratio of less than or equal to 1.25. Other libraries were screened using the same protocol, and the results are summarized in Table 4.

TABLE 4
Bcl-B/TR3 FPA compound screens

| | LOPAC1280 | NCIM | NCIS |
|---|---|---|---|
| Total # | 1280 | 959 | 2442 |
| compounds | | | |
| ≧50% | 21 (1.6%) | 67 (7.0%) | 87 (3.6%) |
| competition | | | |
| #1.25 F-ratio | 16 (1.3%) | 35 (3.6%) | 63 (2.6%) |

Compounds with greater than 50% displacement of FITC-TR3 in the Bcl-B assay at 20 uM concentration, and with an F-ratio parameter less than 1.5 are defined as primary screen actives. Tables 5-8 show the ID numbers, result values and F-ratios of active compounds identified from the Chembridge, LOPAC1280, NCIM, and NCIS libraries, respectively. The “VendorIDs” in Table 5A are from Chembridge (San Diego, Calif.), “PubChemSIDs” in Table 5B are from the Molecular Library Screening Centers Network (MLSCN) (Pubchem; pubchem.ncbi.nlm.nih.gov), VendorIDs in Table 6 are from Sigma-Aldrich (St. Louis, Mo.), Table 7 Vendor IDs are from the NCI Mechanistic library, and Table 8 Vendor IDs are from the NCI Structural library. The NCI mechanistic and structural libraries can be found at http://dtp.nei.nih.gov/does/nsc_all_search.html. For the Chembridge screen, IC50s for each compound with respect to the Bcl-B/FITC-TR3 (In Hepes/β-glycerophophate/tween-20) fluorescence polarization and the Bcl-2/FITC-TR3 (In PBS+tween-20) fluorescence polarization assays were identified. These compounds have low fluorescence interference and have low activity in an HSP/FITC-ATP counter screen.

Compounds identified from the other three libraries have been through the primary assay (with an F-ratio of less than 1.5 and a % competition of 50% or more), and the “Result Value” column shows the % competition. Some compounds appear to have more than 100% competition, which may be due to the compounds being slightly fluorescent, colored, not fully dissolved, or some other reason.

### Example 5

### FPA Using FITC-Bid BH3 Inhibitor Peptide

GST-fusion proteins containing Bcl-XL, Bcl-2, Bcl-B, Bfl-1 and Mcl-1 lacking their C-terminal transmembrane domains (about the last 20 amino acids) (“ΔTM”) were expressed from the pGEX 4T-1 plasmid in XL-1 Blue E. coli cells (Stratagene, La Jolla, Calif.). Briefly, cells were grown in 2 L of LB medium containing 50 μg/ml ampicillin at 37° C. to an OD₆₀₀ₙₘ of 1.0. IPTG (0.5 M) was then added, and the cultures were incubated at 25° C. for 6 h. Cells were then recovered in 20 mM phosphate buffer (pH 7.4), 150 mM NaCl, 1 mM dithiothreitol (DTT), 1 mM EDTA, followed by sonication. Cellular debris was removed by centrifugation at 27,500×g for 20 min, and the resulting supernatants were incubated with 10 ml of glutathione-Sepharose (Pharmacia) at 4° C. for 2 h. The resin was washed 3 times with 20 mM phosphate buffer (pH 7.4), 150 mM NaCl, 1 mM DTT. The resulting GST-fusion proteins were eluted in 50 mM Tris-HCl (pH 8.0) containing 10 mM reduced glutathione. The protein yield for the GST-Bfl-1 protein was about 5 mg per liter of cells with a purity of greater than 95% as determined by Coomassie Blue staining of material analyzed by sodium dodecyl sulfate—polyacrylamide gel electrophoresis (SDS-PAGE). Other Bcl-2 proteins had similar yields and purities.

A FPA was performed to determine the binding affinity of FITC-Bid BH3 peptide to Bcl-2 proteins. Serial concentrations of Bcl-2 proteins were incubated with 5 nM FITC-Bid BH3 peptide (FITC-Ahx-EDIIRNIARHLAQVGDSMDR; SEQ ID NO: 55) in PBS using a 96 well black plate (Greiner bio-one). Fluorescence polarization was measured after 10 min using an Analyst TM AD Assay Detection System (LJL Biosystem, Sunnyvale, Calif.) in PBS (pH 7.4). IC50 determinations were performed using GraphPad Prism software (GraphPad, Inc., San Diego, Calif.). FPA competition assays were performed using the same procedure described above, except that 100 nM of GST-Bfl-1 protein in a volume of 45 μl was incubated with 5 μl (50 μM) test compounds in DMSO per well for 30 min prior to addition of 50 μl (5 nM) FITC-Bid BH3 peptide. The final DMSO concentration was 5% when the reactions brought to fall volume of 100 μl. Fluorescence polarization was measured after 10 min. Compounds that reduced the fluorescence polarization by 50% were considered hits.

Various concentrations of GST-fusion proteins containing ΔTM versions of Bcl-2, Bcl-X_{L}, Bfl-1, Mcl-1, Bcl-W, and Bcl-B were incubated with a fixed concentration of FITC-Bid BH3 peptide and fluorescence polarization (in milli-Polars, mP) was measured. All Bcl-2 family member proteins exhibited fluorescence polarization upon incubation with FITC-Bid BH3 peptide, but to different extents, consistent with their differences in affinities for this peptide (FIG. 10). The best binding (highest fluorescence) was observed for Bfl-1. The control GST protein did not result in fluorescence polarization.

### Example 6

### FPA Competition Analysis of BCl-2 Binding Compounds

The green tea compound epigallecatechin (EGCG) is known to bind both Bcl-2 and Bcl-XL (Leone et al., Cancer Res. 63:8118-8121, 2003). The ability of EGCG to compete with FITC-Bid BH3 peptide was analyzed by FPA using the protocol described above. As illustrated in FIGS. 11A-G, ECGC bound to all six anti-apoptotic members of the Bcl-2 family to different extents. This peptide can be used as a positive control in high throughput library screening protocols.

### Example 7

### Preliminary Screen of Compound Library Using Bfl-1 FPA

The Bfl-1 competitive FPA described above was used to screen a library of 10,000 compounds representing predominantly natural products. The results from one of the plates that contained a “hit” are presented in Table 9 and FIG. 12. From 10,000 compounds, 66 hits were identified. Upon repeat testing, 10 active compounds remained which will be further characterized. Thus, the overall hit rate was 0.1%.

TABLE 9
Example of Bfl-1 FPA competitive screening assay

| | 1 | 2 | 3 | 4 | 5 | 6 | 7 | 8 | 9 | 10 | 11 | 12 |
|---|---|---|---|---|---|---|---|---|---|---|---|---|
| A | 43 | 150 | 154 | 158 | 158 | 160 | 154 | 168 | 166 | 158 | 156 | 148 |
| B | 44 | 145 | 159 | 153 | 167 | 159 | 165 | 156 | 67 | 167 | 176 | 160 |
| C | 52 | 159 | 151 | 148 | 161 | 151 | 175 | 166 | 161 | 175 | 148 | 166 |
| D | 52 | 149 | 163 | 154 | 160 | 180 | 165 | 168 | 163 | 173 | 156 | 158 |
| E | 43 | 155 | 169 | 191 | 168 | 160 | 166 | 163 | 182 | 202 | 166 | 157 |
| F | 44 | 170 | 155 | 168 | 161 | 170 | 161 | 161 | 161 | 173 | 165 | 163 |
| G | 52 | 167 | 121 | 159 | 172 | 163 | 161 | 154 | 183 | 124 | 162 | 155 |
| H | 52 | 148 | 163 | 163 | 165 | 156 | 165 | 162 | 163 | 167 | 166 | 163 |

The first column contains FITC-BH3 peptide without Bfl-1 protein. The last column contains FITC-BH3 and Bfl-1 protein without compounds. Columns 2-11 contain FITC-BH3 peptide, GST-Bfl-1 protein and compounds from the library.

Hits are tested against the other anti-apoptotic members of the Bcl-2 family by FPA to determine the spectrum of activity of the compounds with respect to the competitive binding site that binds BH3 peptides. To exclude compounds that non-specifically interfere with FPAs, compounds are also tested in a FPA for an unrelated protein which involves the BIR3 domain of XIAP binding to rhodamine-conjugated tetrapeptide AVPI, representing the N-terminus of the IAP antagonist SMAC (Liu et al., Nature 408:1004-1008, 2000; Wu et al. Nature 408 1008-1012, 2000).

A cell-based assay was previously generated in which Bcl-XL was co-expressed in HeLa cells with a green fluorescent protein (GFP)-tagged BH3 protein, and compounds were tested for their ability to displace the GFP-tagged BH3 protein from mitochondria-bound Bcl-X_{L} by confocal microscopy, using time-lapsed video microscopy (Becattini et al., Chem. Biol. 11 389-395, 2004; Leone et al., supra.; Oltersdorf et al., Nature 435:677-681, 2005). A similar cell line is engineered using Bfl-1 instead of Bcl-XL, and used as another secondary screen.

A stably transfected human cell line was previously engineered to express Bcl-2 family members using a tetracycline-inducible promoter system. In these cells, turning on expression of the anti-apoptotic Bcl-2 family member Bcl-X_{L} was shown to protect against apoptosis induced by cytotoxic anticancer drugs such as doxorubicin (Wang et al., J. Biol. Chem. 279:48168-48176, 2004). Addition of Bcl-X_{L} neutralizing compounds overcomes this protection. A tetracycline-inducible HeLa cell line is engineered which conditionally expresses Bfl-1, and the ability of Bfl-1 selective compounds to overcome cytoprotection mediated by Bfl-1 with Bcl-X_{L} is compared. Selective compounds will restore apoptosis sensitivity to Bfl-1-expressing, but not Bcl-X_{L}-expressing HeLa cells.

### Example 8

### Bcl-2 Antagonists that Target the TR3 (Nur77) Binding Site

FITC-TR3-9-r8 was tested for binding to Bcl-2 by FPA, demonstrating direct binding to GST-Bcl-2, but not GST, with an apparent K_{d} of <0.1 μM (FIG. 10). This FITC-conjugated 9-mer also bound in a concentration and saturable manner to three of the six anti-apoptotic members of the Bcl-2 family, with apparent K_{d}s of 66 to 239 nM (Bcl-2, Bcl-B, Bfl-1). In contrast, a 9-mer peptide in which the N- and C-terminal residues were converted to alanine did not significantly bind. Assay performance characterization indicates that the FPAs are suitable for high throughput screening with Z′ factors >0.5.

The stability of FITC-TR3-9-r8 binding to Bcl-B was tested in various solutions in 384 well format to identify conditions where the fluorescence polarization signal is stable for several hours. FITC-TR3-9-r8 (20 nM) was incubated with various concentrations of GST-Bcl-B, and fluorescence polarization was measured. FIG. 13A contrasts the results obtained in PBS vs. HEPES with β-glycerol phosphate, showing that binding is stable for several hours in HEPES-β-glycerol phosphate, but not PBS.

Competitive displacement assays were performed using increasing concentrations of unlabeled TR3 peptide to compete with a fixed concentration of FITC-TR3-9-r8 peptide for binding to Bcl-B in β-glycerol phosphate buffer. The maximum polarization attained in the absence of TR3 defines the maximum for the assay. The maximum competition defines the minimum for the assay. The results show that unlabeled TR3 is an effective competitive inhibitor of the FITC-labeled peptide (FIG. 13B). The mP(max), mP(min), and apparent Kd for the FPA were measured at various times from the same plate to assess the stability of the assay when conducted using β-glycerophosphate buffer. Data represent mean±SD for n=3 (FIG. 13C).

### Example 9

### TR3 Peptide Binds a Non-BH3 Site on Bcl-2

To determine whether the TR3 peptide binds the same site on Bcl-2 where BH3 peptides bind, competition assays were performed in which fixed concentrations of FITC-TR3-9-r8 peptide and Bcl-2 protein were incubated in the presence or absence of unlabeled TR3 peptide, mutant TR3 peptide, BH3 peptide, or compound ABT-737, a known inhibitor of Bcl-2 proteins. The results are shown in FIGS. 14A-B. As expected, unlabeled TR3 peptide competed with FITC-TR3-9-r8 for binding to Bcl-2, whereas the mutant TR3 peptide was less active. In contrast, BH3 peptide and ABT-737 failed to block FITC-TR3-9-r8 peptide binding to Bcl-2. Thus, the TR3 peptide binds a different site on Bcl-2 than do BH3 peptides.

### Example 10

### Additional Buffer Optimization Study for BclB/FITC-TR3-r8 FPA

Bcl-B binding curves were obtained in different buffers (25 mM each) supplemented with 1 mM TCEP, 0.005% Tween 20. The FITC-Tr3 concentration was 20 nM. The results (FIG. 15) demonstrated improved signal stability and higher-than-average affinity of Bcl-B to FITC-TR3 in the presence in the presence of PIPES buffer compared to performing the assay in other buffers. Bcl-B (15 nM) in 25 mM PIPES, pH 7.0, containing 1 mM TCEP, 0.005% Tween 20 and 20 nM FITC-TR3 was added with different concentrations of TR3-r8. Fluorescence polarization was measured after 15 min incubation. Non-linear regression analysis was performed using 4-parameter sigmoidal equation (mP=Assay_WINDOW*KD̂H/(KD̂H+[TR3-r8]̂H)+mP_MIN). The results (FIG. 16) show displacement of FITC-TR3-r8 from a complex with Bcl-B and TR3-r8.

### Example 11

### HTS Implementation of Bcl-B/FITC-Tr3 FPA

The effect of different parameters on signal stability of Bcl-B/FITC-Tr3-R8 assay was evaluated. HEPES-β-glycerophosphate buffer (referred to as assay buffer) developed at the assay optimization stage was used. The affinity of FITC-Tr3-R8 binding to Bcl-B does not change with time in this buffer, as previously observed. To confirm that binding and dissociation of FITC-Tr3-R8 peptide is fast, an order of addition experiment was performed. In this experiment, Bcl-B was preincubated for 1 h with FITC-Tr3-R8 peptide or Tr3-R8 peptide, prior to addition Tr3-R9 or FITC-Tr3-R8, respectively (FIG. 17).

As shown in FIG. 17, fluorescence polarization is similar after preincubation with either peptide. These results suggest that binding and dissociation of FITC-Tr3-R8 is fast and that preincubation of Bcl-B with FITC-Tr3-R8 does not alter the binding of its displacer; therefore, the HTS assay can be configured to have Bcl-B and FITC-Tr3-R8 pre-mixed and dispensed in a single-step addition using a liquid bulk dispenser.

To evaluate stability of the assay mixture for bulk dispensing, Bcl-B (25 nM) and FITC-Tr3-R8 (20 nM) were added together in assay buffer. Solution was dispensed using WellMate into wells containing 10% DMSO and Tr3-R8 in 10% DMSO. Final concentrations of Tr3-R8 and DMSO in the assay were 5 uM and 1%, respectively. Fluorescence polarization was measured right after mixing and 2 h after storage in different conditions (FIG. 18). Each storage condition and DMSO/Tr3-R8 combination occupied 4 columns of 384-well plate, e.g. contained 64 data points. Based on data in FIG. 18, one can conclude that assay mixture is more stable at +4° C.; Z′-factor calculated from the data (Table 10) was the same for freshly prepared solution or for the one kept on ice for 2 h. There was also slightly better Z′-factor for mixture kept in polypropylene vs. polystyrene.

TABLE 10
Statistical evaluation of Bcl-B/FITC-Tr3 assay performance

| Conditions | 0 h | 2 h PS | 2 h PP (+4° C.) | 2 h PP (RT) |
|---|---|---|---|---|
| Z′ | 0.79 | 0.55 | 0.77 | 0.63 |

Based on this study, one assay format for HTS is as follows. Compound dispensed to assay plate in 2.5 uL aliquot, 10% DMSO. Control wells contain 2.5-uL aliquots of 10% DMSO or 50 uM Tr3-R8 in 10% DMSO. Bcl-B and FITC-Tr3-R8 premixed in assay buffer at 27.8 nM and 22.2 nM, respectively. The mixture is kept on ice until utilized. The mixture is dispensed using WellMate to add 22.5 uL to each well.

The following sections relate to studies in which the TR3/Bcl-B interaction was used to develop a FPA-based high throughput screen (HTS) for small molecule inhibitors of Bcl-B. A library of about 50,000 compounds was screened, and after several secondary assays, one non-canonical Bcl-B-binding compound was identified.

### Example 12

### Peptide Synthesis

Peptides were synthesized using an Advanced ChemTech (Louisville, Ky.) 396 multiple peptide synthesizer (Luciano et al., Blood 109:3849-3855, 2007). Rink amide p-methylbenzhydrylamine resin with Fmoc synthesis and diisopropylcarbodiimide/1-hydroxybenzotriazole (DIC/HOBt) coupling was used. All peptides were acetylated on their N-termini and amidated on their C-termini. An extended treatment (6 h) was used with standard deprotection solutions to remove Pbf from multiple arginines. The peptides were then purified by high-performance liquid chromatography (HPLC) on C18 columns and confirmed by matrix-assisted laser desorption/ionization (MALDI) mass analyses. The sequences of the peptides were as follows (X represents N-aminocaproic acid, r8 is 8 residues of D-amino acid arginine, and all other amino acid residues are L-amino acids):

| | |
|---|---|
| TR3-r8: | FSRSLHSLL-GX-r8 |
| Bim-BH3: | DMRPEIWIAQELRRIGDFFNAYYAR |
| Bax-BH3: | PQDASTKKLSECLKRIGDELDSNMEL, |
| Bak-BH3: | PSSTMGQVGRQLAIIGDDINRRYDS |
| Puma-BH3: | EEQWAREIGAQLRRMADDLNAQYERR |

Where indicated, peptides were labeled with an N-terminal fluorescein isothiocyanate (FITC) molecule which was coupled to the peptide using an N-aminohexanoic acid linker.

### Example 13

### Protein Purification

Bcl-B and Bcl-2 were expressed in bacteria as glutathione S-transferase (GST)-fusion proteins in which their C-terminal transmembrane domains (˜20 amino acids) were deleted to enhance protein solubility. GST was also expressed in bacteria as a control for the experiments. In all cases, the pGEX-4T-1 vector (GE Healthcare, Piscataway, N.J.) was used and transformed into Escherichia coli BL21 Star (DE3) (Invitrogen, Carlsbad, Calif.). Bacteria were grown at 37° C. in LB media containing 50 μg/mL carbenicillin to a cell density of 0.8 to 1.0 (600 nm). Isopropyl β-D-thiogalactoside (IPTG; 0.4 mM; Invitrogen) was then added, and after 4 h, cells were harvested by centrifugation (4,000×g for 20 min).

For protein purification, cell pellets were resuspended in PBS buffer containing 1 mg/mL lysozyme (Sigma-Aldrich, St. Louis, Mo.), and 1 mM Phenylmethanesulfonyl fluoride (PMSF; Sigma-Aldrich). After 30 min at 4° C., this mixture was sonicated and centrifuged (12,000×g, 30 min). Soluble proteins were purified with Glutathione Sepharose 4B (GE Healthcare) and concentrated.

### Example 14

### Fluorescence Polarization Assays

The fluorescence polarization assays were performed as previously described (Zhai et al., Cell Death Differ. 13:1419-1421, 1006). Briefly, various concentrations of the indicated proteins (e.g. GST-Bcl-B, GST-Bcl-2) were incubated with various concentrations of the indicated peptide (e.g. FITC-TR3-rS, FITC-Puma-BH3, FITC-Bim-BH3) and competitor (e.g. TR3-r8, Bim-BH3, Bax-BH3, compounds) in a 25 mM HEPES-KOH, 20 mM β-glycerophosphate, 0.005% Tween-20, pH 7.5 or a PBS, 0.005% Tween-20 buffer for 10 min. Fluorescence polarization was then measured using an Analyst HT Multi-Mode Plate Reader (LJL Biosystems, Sunnyvale, Calif.). Data were analyzed using GraphPad Prism (GraphPad Software, San Diego, Calif.).

### Example 15

### High Throughput Screening

Approximately 50,000 compounds from the ChemBridge DIVERSet library (ChemBridge, San Diego, Calif.) were plated into black, 384-well, flat bottom plates (Griener Bio-One, Frickenhausen, Germany) using a BioMek FX Laboratory Automation Workstation (Beckman Coulter, Fullerton, Calif.) (37.5 mg/L compounds in 2 μL of 10% DMSO). The ThermoScientific Matrix WellMate bulk dispenser (Thermo Fisher Scientific, Hudson, N.H.) was then used to add GST-Bcl-B (44.4 nM in 9 μL), followed by FITC-TR3-r8 (44.4 nM in 9 μL), both diluted in a 25 mM HEPES-KOH, 20 mM β-glycerophosphate, 0.005% Tween-20, pH 7.5 buffer. Each well thus contained 3.75 mg/L compound, 1% DMSO, 20 nM GST-Bcl-B, and 20 nM FITC-TR3-r8. After 10 min incubation, fluorescence polarization was measured using the Analyst HT Multi-Mode Plate Reader.

### Example 16

### NMR Spectroscopy

NMR experiments were performed at 25° C. on a 500 MHz Bruker Avance spectrometer (Bruker, Madison, Wis.) equipped with a 5 mm TXI probe. Compounds were dissolved in fully deuterated DMSO (d6-methyl sulphoxide; Sigma-Aldrich) to a concentration of 10 mM. ¹H NMR reference spectra were taken for each compound at a final concentration of 1 mM in PBS buffer prepared with 99.9% deuterium oxide. Reference solutions were then used in titration experiments with GST-Bcl-B or GST. All ¹H NMR spectra were obtained with the carrier position set to the water peak signal using WATERGATE. NMR data were processed and analyzed with MestRe Nova (MestReLab Research, Santiago de Compostela, Spain).

### Example 17

### Cell Culture

HeLa Tet-On-Bcl-B cells were generated by stably transfecting the HeLa Tet-On cell line (Clontech, Mountain View, Calif.) with a pTRE2hyg vector (Clontech) containing the Bcl-B gene, using lipofectAMINE PLUS (Invitrogen, Carlsbad, Calif.). Briefly, cells were seeded overnight and transfected at 50% confluency for 3 h. After 24 h, the cells were re-seeded at <10% confluence and cultured in media (DMEM with 10% Tet System Approved FBS (Clontech)) containing G418 (100 μg/mL) and hygromycin B (300 μg/mL) for pTet-On and pTRE2hyg/Bcl-B plasmid maintenance, respectively. Positive foci resistant to both antibiotics were expanded. Colonies were then cultured in the presence or absence of doxycycline (Clontech, 1 μg/mL) for 16 h, and induced Bcl-B expression was confirmed by Western blot analysis. A previously developed polyclonal Bcl-B antibody¹⁷ and an Hsc70 antibody (for protein concentration comparison; Santa Cruz Biotechnology, Santa Cruz, Calif.) were used.

### Example 18

### Cell Viability Studies

Cells were seeded in 96-well plates at 5,000 cells/well in 100 μL of growth medium and allowed to incubate for 24 h. Afterwards, cells were incubated in the presence or absence of doxycycline (1 μg/mL) for another 24 h (to induce Bcl-B expression). Compounds were then added, as indicated, in a volume of 5 μL. The next day, cell viability was measured using the ATPlite Luminescence ATP Detection System (PerkinElmer, Waltham, Mass.) according to the manufacturer's specifications.

### Results

### Fluorescence Polarization Assay Characterization

A fluorescence polarization assay was developed based on the ability of FITC-TR3-r8 to bind Bcl-B. Thr apparent K_{d} for GST-Bcl-B binding to 20 nM FITC-TR3-r8 (the minimal concentration of FITC-TR3-r8 that displayed a 10-fold higher fluorescence intensity over background) was determined (FIG. 19A). The apparent K_{d} for GST-Bcl-B was ˜20 nM. To determine if TR3-r8 could displace FITC-TR3-r8 from GST-Bcl-B, increasing concentrations of the unlabelled TR3-r8 was incubated with a FITC-TR3-r8/GST-Bcl-B solution (FIG. 19B). Indeed, unlabeled TR3-r8 was able to displace FITC-TR3-r8 in a concentration-dependent fashion (EC₅₀=82.8 nM). The FITC-conjugated TR3 (9′mer) peptide displayed higher affinity binding to Bcl-B with the r8 tail, and the unlabeled TR3 (9′mer) peptide demonstrated more complete competitive displacement with the r8 tail, raising the possibility that the r8 tail stabilizes an active conformation of the TR3 peptide.

BH3 peptides derived from Bim (Bim-BH3) and Bax (Bax-BH3), as well as the compound EGCG, can displace FITC-BH3 peptides from GST-Bcl-B (Zhai et al. supra.). However, none of these reagents displaced FITC-TR3-r8 (FIG. 19B), suggesting that FITC-TR3-r8 binds in a non-canonical manner. This experiment was repeated using a FITC-TR3-r8/GST-Bcl-2 fluorescence polarization assay. Bak-BH3 and ABT-737 have been previously shown to displace FITC-BH3 peptides from Bcl-2 (Zhai et al, supra.). However, Bak-BH3 and ABT-737 did not effectively displace FITC-TR3-r8 from Bcl-2, whereas TR3-r8 effectively competed for binding (FIG. 19C).

### High Throughput Screening (HTS)

To determine the quality and reproducibility of the FITC-TR3-r8/GST-Bcl-B fluorescence polarization assay for HTS, GST-Bcl-B was incubated with either FITC-TR3-r8 alone (negative control) or FITC-TR3-r8 and TR3-r8 (positive control) in a 384-well plate. This assay demonstrated robust performance, with a Z′-factor of 0.75 (FIG. 20A).

This HTS assay was then used to screen a ˜50,000 library (ChemBridge DIVERSet library) for compounds that displaced FITC-TR3-r8 from GST-Bcl-B (FIG. 20B). Compounds that induced a=50% decrease in fluorescence polarization and that did not increase fluorescence intensity by=25% (thus eliminating compounds that interfered with the assay due to their fluorescence) were determined to be “hits”. The primary screen yielded 332 hits.

A number of assays were then performed to eliminate non-specific or irreproducible hits (FIG. 21). First, the hit compounds were re-tested at the screening concentration; 145 reproducible hits were observed, representing a confirmed hit rate of 0.29%. Second, compound dose-response curves were generated using the fluorescence polarization assay. Compounds that did not display a sigmoidal dose-response curve, or that increased fluorescence intensity, were eliminated, leaving 53 hits. Third, the remaining compounds were then counter-screened using an unrelated, FITC-based fluorescence polarization assay (FITC-ATP/GST-Hsp70), leaving 50 compounds that appeared to be specific for inhibition of the FITC-TR3-rS/GST-Bcl-B assay. Fourth, further analyses of the previously generated dose-response curves identified 11 compounds with statistically “well-fitted” curves, using the Hill equation (0.7=H=1.3, r²>0.9)²⁰. This stringent criteria eliminates compounds that may act in a cooperative or anti-cooperative manner (Coval, J. Biol. Chem. 245:6335-6336, 1970). Fifth, new powdered-stocks of the active compounds were then dissolved and tested, with 6 compounds confirming. Finally, 1D-NMR was used to test the compounds for binding to GST-Bcl-B versus GST (as a negative control) (FIG. 22). Of the six compounds, two (5804000 and 5954623) bound to GST-Bcl-B in a concentration dependent manner. The chemical structure of these two compounds is shown in Table 5A, pages 7 and 5, respectively. These compounds did not bind to GST, thus indicating specificity. Several compounds did not bind to either GST-Bcl-B or GST, serving as internal negative controls for this experiment (e.g. compound 2011727, FIG. 22).

### Compound Characterization

The two Bcl-B-specific compounds, 5804000 and 5954623, were evaluated using the FITC-TR3-r8/GST-Bcl-B fluorescence polarization assay and yielded EC₅₀ values of 5.6 μM and 2.1 μM, respectively (FIG. 23A, 23B). Unlabeled TR3-r8 peptide (positive control) also displaced FITC-TR3-r8 as expected, while 5729206 (negative control), a HTS-negative compound, did not (FIG. 23B).

The compounds were then tested for their ability to displace BH3 peptides from Bcl-B. FITC-labeled BH3 peptides have been previously shown to bind GST-Bcl-B in a fluorescence polarization assay (Zhai et al, supra.). When unlabeled BH3 peptides derived from Bim or Bax were added, they effectively displaced FITC-Puma-BH3 from GST-Bcl-B (FIG. 23C). Although compound 5954623 displaced FITC-Puma-BH3, 5804000 did not (FIG. 23C). Similarly, in a fluorescence polarization assay using a FITC-labeled peptide derived from the BH3 region of Bim (FITC-Bim-BH3) and GST-Bcl-B, adding unlabeled BH3 peptides or compound 5954623 displaced FITC-Bim-BH3, while compound 5804000 did not (FIG. 23D). These results suggest that 5804000 binds to a region on Bcl-B that differs from the binding site of pro-apoptotic BH3 domains, and thus this compound meets the primary screening objective.

### Compound Biological Activity

To test if 5804000 displays Bcl-B-dependent cellular activity, HeLa cells that expressed Bcl-B under the control of a doxycyline-activated promoter (HeLa Tet-On-Bcl-B; HTO2 cells) were generated (FIG. 24A). As expected, HeLa Tet-On-Bcl-B cells were more resistant to staurosporine-induced cell death in the presence of doxycycline-induced Bcl-B (FIG. 24B). Control HeLa Tet-On cells that did not express Bcl-B in the presence of doxycycline (HTO1 cells) displayed the same level of staurosporine-induced cell death whether or not doxycycline was added, indicating that doxycycline was not confounding the HeLa Tet-On-Bcl-B results (FIG. 24B). In the presence of doxycycline, HeLa Tet-On-Bcl-B cells were more resistant to EGCG (positive control) and 5804000, but not to 5729206 (negative control) (FIG. 24C). For EGCG and 5804000, the concentrations required to reduce HeLa Tet-On-Bcl-B cell viability by 50% increased from 79 μM and ˜148 μM, respectively, in the absence of Bcl-B, to ˜93 μM and ˜226 μM, respectively, in the presence of Bcl-B. In contrast, the expression of Bcl-B did not affect 5729206-induced cell death. Thus, 5804000 acted in a Bcl-B-dependent manner in these genetically engineered cells.

The studies described above provide a novel TR3-derived assay for small molecule Bcl-B inhibitor screening. Using a fluorescence polarization-based strategy, a FITC-TR3-r8/GST-Bcl-B binding assay was optimized for HTS, and approximately 50,000 compounds at 3.75 mg/L were screened, resulting in 332 primary screening hits (non-fluorescent compounds displaying ≧500% FITC-TR3-r8 displacement), of which 145 were reproducible, thus representing a confirmed hit rate of 0.29%. After dose-response analyses and counter-screening with an unrelated fluorescence polarization assay (FITC-ATP/GST-Hsp70), 6 potential compounds remained. Using 1D-NMR, 2 of these compounds were found to bind to GST-Bcl-B, but not to GST, and one of these compounds did not interfere with BH3 peptide binding to Bcl-B, thus fulfilling the primary screening objective.

Anti-apoptotic Bcl-2 family proteins such as Bcl-B contain a hydrophobic cleft to which the BH3 domains of pro-apoptotic Bcl-2 family proteins bind, thereby inducing apoptosis (Reed et al., Nat. Clin. Pract. Oncol. 3:388-398, 2006; Cory et al., Oncogene 22:8590-8607, 2003). A number of effective apoptosis-inducing molecules have been designed or identified based on mimicking BH3 domains (Leone et al., Cancer Res. 63:8118-8121, 2003; Oltersdorf et al, Nature 435:677-681, 2005). This work shows, by several lines of evidence, that Bcl-B-binding compounds may also be identified via a non-BH3 peptide mimicking strategy by using a TR3-derived peptide. First, neither BH3 peptides (derived from Bim and Bax) nor EGCG (previously shown to displace FITC-labeled BH3 peptides from Bcl-B, Zhai et al., supra.) displaced FITC-TR3-r8 from GST-Bcl-B, indicating that TR3-r8 may bind to Bcl-B via an alternate mechanism. Second, neither a Bak-derived BH3 peptide nor ABT-737 (previously shown to displace FITC-labeled BH3 peptides from Bcl-2; Zhai et al, supra.) displaced FITC-TR3-r8 from GST-Bcl-2. Third, one of the compounds identified via the FITC-TR3-r8/GST-Bcl-B screen does not displace FITC-labeled BH3 peptides from GST-Bcl-B. Thus, Bcl-B-binding compounds with novel specificities can be identified using this TR3 peptide-based HTS assay.

With the described assay, the confirmed hit rate was 0.29%, but after compound characterization, only 2 Bcl-B-binding compounds remained. While fluorescence polarization assays are robust, they rely on a fluorescence measurement to derive general molecular rotational correlation time differences induced by binding or displacement (Zhai et al., supra.; Pope et al., Drug Discov. Today, 4:350-362, 1999). Non-specific interactions with the assay components such as with the peptide or protein—are common sources of inaccuracies. Compounds that are fluorescent, fluorescence quenchers, or that precipitate due to solubility problems can produce misleading data, but analyses of the raw fluorescence screening data usually remedies many interference issues. Moreover, utilization of secondary assays provides additional confirmation for the hits. To eliminate compound hits that interfered with the assay, we eliminated compounds that increased fluorescence intensity by=25% in the primary assay, performed dose-response analyses, eliminated compounds that increased fluorescence intensity in a concentration-dependent manner, counter-screened using a biologically unrelated FITC-based fluorescence polarization assay (FITC-ATP/GST-Hsp70), used the Hill equation to eliminate compounds with cooperative or anti-cooperative binding, confirmed compounds using newly dissolved stocks, and used 1D-NMR to distinguish between GST-Bcl-B-binding compounds vs. GST-binding compounds. These stringent criteria reduced fluorescence polarization false positives, although some Bcl-B-specific compounds may also have been eliminated (e.g. fluorescent Bcl-B-binding compounds). Cellular experiments showed Bcl-B-dependent modulation of sensitivity to compound 5804000.

Compounds from the Molecular Library Screening Centers Network (MLSCN) were screened using the Bcl-B/TR³ fluorescence polarization assay described herein, and active inhibitory compounds were identified which comprise three scaffold (backbone structures) that are shown below and in FIGS. 25-27.

Scaffold 1:

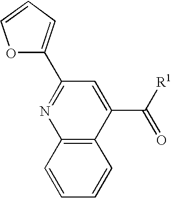

wherein R¹ is selected from the group consisting of —NH=Naryl, —NHaryl, —O[(CH₁)ₚNR¹⁰R¹¹], —O[(CH₂)ₚC(O)NR¹⁰R¹¹], —O[(CH₂)ₚNR¹⁰R¹¹], each optionally substituted with one or more substituents each independently selected from the group consisting of halo, cyano, hydroxy, C₁₋₆ alkyl, C₁₋₆ alkoxy, phenyl, and NR¹⁰R¹¹;
p is 1, 2, or 3; and

R¹⁰ and R¹¹ are each separately selected from hydrogen, C₁₋₆ alkyl, arylC₁₋₆ alkyl; or R¹⁴ and R¹⁵ are taken together with the nitrogen to which they are attached to form indolinyl, pyrrolidinyl, piperidinyl, piperazinyl, or morpholinyl.

Scaffold 2:

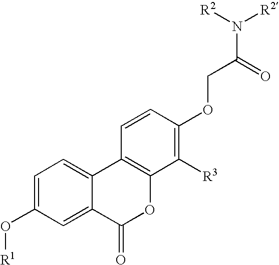

wherein R¹ is selected from the group consisting of hydrogen, aryl, heteroaryl, heterocyclyl, and C₁₋₆ alkyl optionally substituted with up to five fluoro;

R² and R^{2′} are each separately hydrogen or selected from the group consisting of C₁₋₆ alkyl, —(CH₂)_{q}C₃₋₇cycloalkyl, aryl, heteroaryl, and heterocyclyl, each optionally substituted with one or more substituents each independently selected from the group consisting of halo, cyano, hydroxy, —(CH₂)_{q}C₃₋₇cycloalkyl, C₁₋₆ alkyl optionally substituted with up to 5 fluoro, and C₁₋₆ alkoxy optionally substituted with up to 5 fluoro; or R² and R^{2′} are taken together with the nitrogen to which they are attached to form a heterocyclyl;

R³ is hydrogen or selected from the group consisting of C₁₋₆ alkyl, —(CH₂)_{q}C₃₋₇cycloalkyl, and aryl each optionally substituted with one or more substituents each independently selected from the group consisting of halo, cyano, and hydroxy; and

Q is 0, 1, 2, or 3.

Scaffold 3:

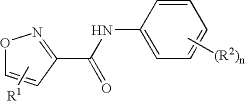

wherein R¹ is hydrogen or selected from the group consisting of C₁₋₆ alkyl, and aryl; or R¹ is a fused C₃₋₇cycloalkyl;

R² is selected from the group consisting of —SC₁₋₆alkyl, C₁₋₆alkoxy, C₁₋₆alkyl, —C(O)OC₁₋₆alkyl, and —C(O)NHC₁₋₆alkyl; and

n is an integer selected from 1, 2, 3, 4, or 5.

As shown in FIGS. 25A-H, all of the compounds tested, with the exception of the structure shown in FIG. 25G, had IC50 values of <25 μM. In contrast, all of these structures had an IC50 of >100 μM against Bfl-1/Bid, thus illustrating the specificity of these compounds in the Bcl-B/TR3 fluorescence polarization assay.

As shown in FIGS. 26A-H, all of the compounds tested, with the exception of the structures shown in FIGS. 26D and F, had IC50 values of =50 μM. In contrast, all of these structures had an IC50 of >50 μM against Bfl-1/Bid, thus illustrating the specificity of these compounds in the Bcl-B/TR3 fluoresce polarization assay. In addition, it appears that a methyl group at position R³ has a negative effect on activity which can be seen by comparing the structures shown in FIGS. 26B and 26E, and FIGS. 26D and 26H. In each on these pairs of compounds, the only difference is the presence or absence of a methyl group at the R3 position, which results in much greater activity when the methyl group is absent. Thus, in one embodiment, R3 is not methyl.

As shown in FIGS. 27A-I, all of the compounds tested, with the exception of the structure shown in FIGS. 27C and D, had IC50 values of <20 μM. In contrast, all of these structures had an IC50 of >100 μM against Bfl-1/Bid, thus illustrating the specificity of these compounds in the Bcl-B/TR3 fluorescence polarization assay.

In other embodiments, any of the generic compounds described above (1, 2 or 3), or the specific compounds shown in FIGS. 25-27, can be used in any of the screening assays described herein. In another embodiment, any of the generic compounds described above (1, 2 or 3), or the specific compounds shown in FIGS. 25-27, can be used to inhibit a Bcl-B protein by contacting the Bcl-B protein with the compound.

In summary, the current fluorescence polarization assay represents a novel screen for identifying potential non-canonical Bcl-2 family inhibitors. A brief screening campaign identified two Bcl-B-binding compounds, one of which appeared to bind via a BH3-independent mode, and thus provides a novel route toward small molecule inhibitors of Bcl-B and other anti-apoptotic Bcl-2 family proteins.

### Example 19

### Treatment of Cancer

A cancer patient is intravenously administered a therapeutically effective amount of one or more of the compounds shown in Tables 5, 6, 7, 8 and FIGS. 25-27, in which the compounds are in a pharmaceutically acceptable excipient or diluent. The compound(s) is administered once daily for 2-3 weeks, at a concentration of 30-80 mg/kg.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

TABLE 5A

| | VendorCompound | Source | Source | | Bcl-B IC50 |
|---|---|---|---|---|---|
| Compound | ID | Plate ID | Well ID | M.W. | (uM) |
| 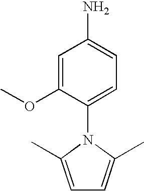 | 5953790 | 10641 | C07 | 216.28 | 32.850934 |
| 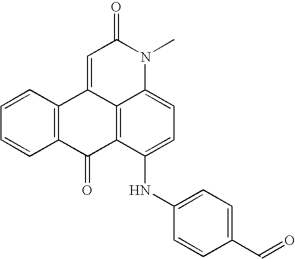 | 5719776 | 10989 | F04 | 380.4 | 8.0389064 |
| 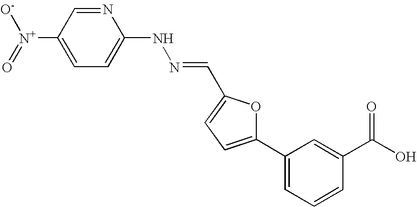 | 5908920 | 11081 | D02 | 352.3 | 9.6395118 |
| 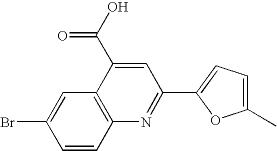 | 6131498 | 11208 | F09 | 332.15 | 16.534698 |
| 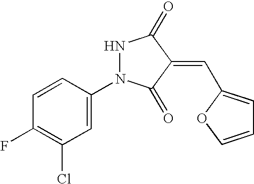 | 5837788 | 11243 | H08 | 306.88 | 33.357245 |

| | | | H 95% | | |
|---|---|---|---|---|---|
| | Bcl-B IC50 | Bcl-B H | Confidence | | Bcl-2 IC50 |
| Compound | (g/L) | Coefficient | Intervals | Bcl-B r2 | (uM) |
| 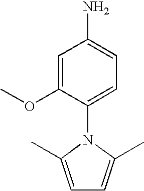 | 0.007105 | 2.719 | 1.956 to 3.481 | 0.9859 | 87.802848 |
| 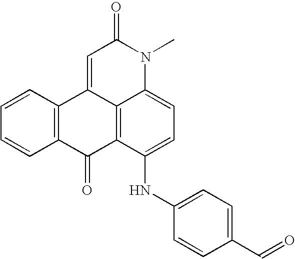 | 0.003058 | 2.489 | 0.2017 to 4.776 | 0.9159 | 20.791272 |
| 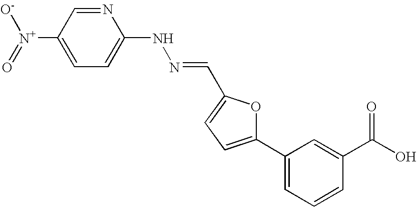 | 0.003396 | 0.6738 | 0.3773 to 0.9703 | 0.8832 | 8.30826 |
| 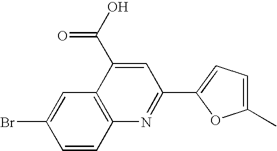 | 0.005492 | 1.896 | 1.197 to 2.596 | 0.967 | 34.863766 |
| 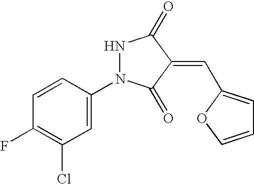 | 0.01023 | 1.772 | 0.7836 to 2.760 | 0.9127 | 8.624625 |

| | Bcl-2 IC50 | | |
|---|---|---|---|
| Compound | (g/L) | Well ID | ID |
| 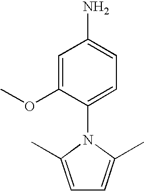 | 0.01899 | F14 | 1286 |
| 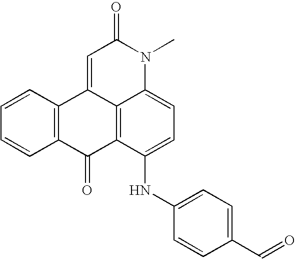 | 0.007909 | L08 | 11792 |
| 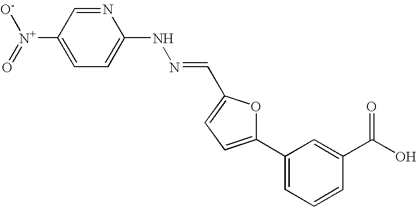 | 0.002927 | H04 | 8236 |
| 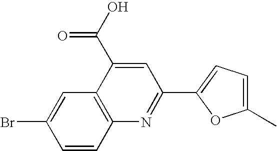 | 0.01158 | L17 | 8345 |
| 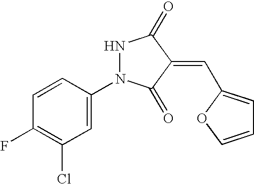 | 0.002645 | O16 | 11872 |

| | VendorCompound | Source | Source | | Bcl-B IC50 |
|---|---|---|---|---|---|
| Compound | ID | Plate | Well | M.W. | (uM) |
| 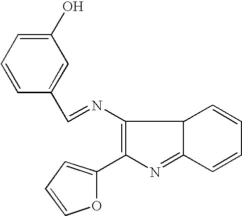 | 5612880 | 10950 | E09 | 303.31 | 9.0699285 |
| 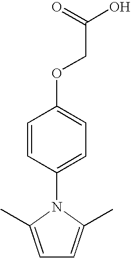 | 5988031 | 10643 | B08 | 245.27 | 8.7454642 |
| 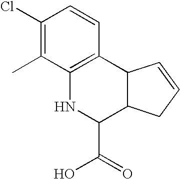 | 5659221 | 10966 | B03 | 263.72 | 5.2062794 |
| 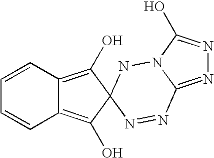 | 5627952 | 10958 | H03 | 288.29 | 23.764265 |
| 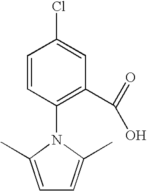 | 5950160 | 11109 | A04 | 249.69 | 0.941968 |
| 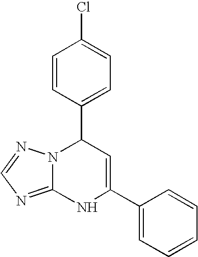 | 5223675 | 10789 | F05 | 308.76 | 14.179298 |
| 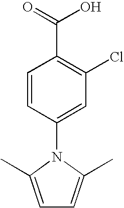 | 5956338 | 11105 | H04 | 249.69 | 9.9563459 |
| 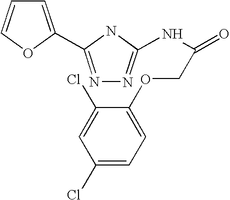 | 5632817 | 10959 | F06 | 370.21 | 17.028173 |
| 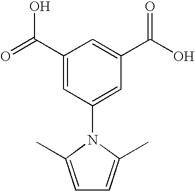 | 5950689 | 11109 | E03 | 259.28 | 0.5415413 |
| 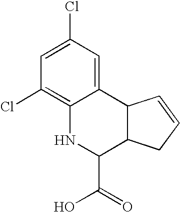 | 5736518 | 10734 | E06 | 284.14 | 1.0280144 |
| 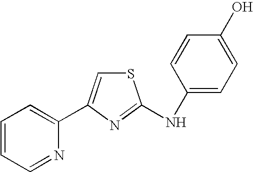 | 5662807 | 10967 | F02 | 269.32 | 3.6907768 |
| 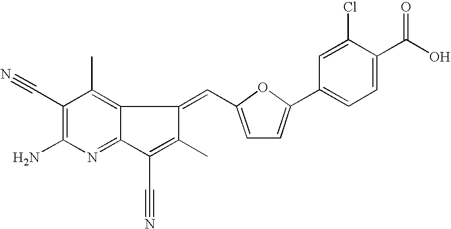 | 6025233 | 11138 | H08 | 442.85 | 1.6486395 |
| 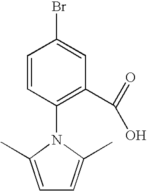 | 5954623 | 10642 | A08 | 280.14 | 16.338259 |
| 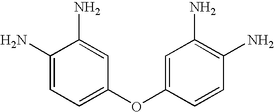 | 5152592 | 10769 | C03 | 230.27 | 5.3719547 |
| 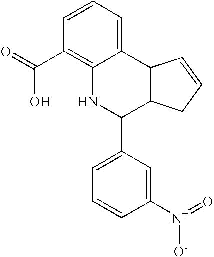 | 5584249 | 10944 | F05 | 336.34 | 2.1032289 |
| 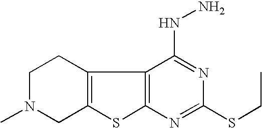 | 5683054 | 10977 | D11 | 295.43 | 7.4264631 |
| 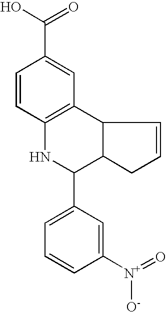 | 5475298 | 10891 | F07 | 336.34 | 1.7523934 |
| 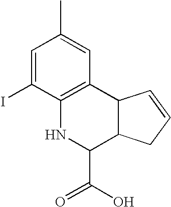 | 6048134 | 11155 | B11 | 355.17 | 20.688684 |
| 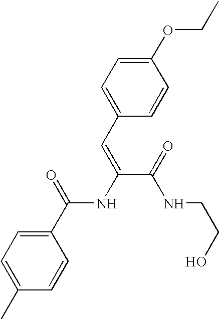 | 5650022 | 10961 | F06 | 368.43 | 13.250821 |
| 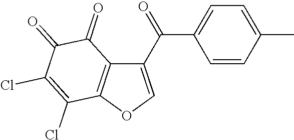 | 5169132 | 11225 | C08 | 335.14 | 6.1675718 |
| 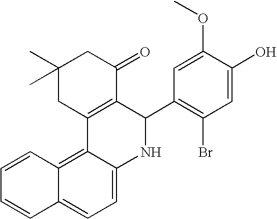 | 5660965 | 10966 | D10 | 478.38 | 13.092103 |
| 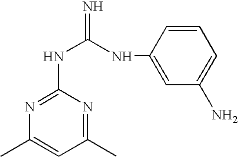 | 5804000 | 11053 | H03 | 256.31 | 11.33003 |
| 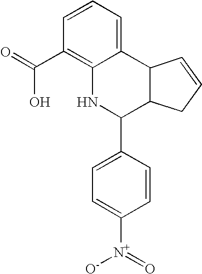 | 5585430 | 10945 | A05 | 336.34 | 5.4082179 |
| 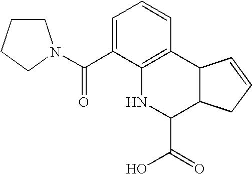 | 5947449 | 11245 | G06 | 312.36 | 4.9494173 |
| 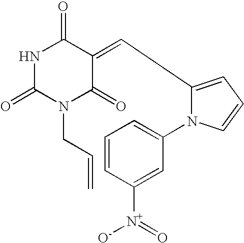 | 5987504 | 11130 | D11 | 382.39 | 5.3557886 |
| 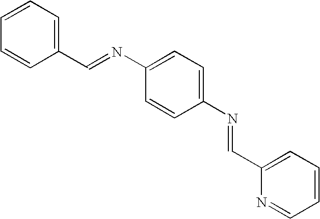 | 5491977 | 10906 | C09 | 286.33 | 4.386547 |
| 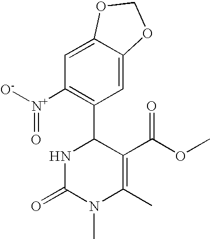 | 5607960 | 10947 | H03 | 349.3 | 17.380475 |
| 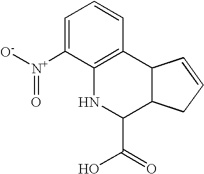 | 5665283 | 10968 | A03 | 260.25 | 1.1996158 |
| 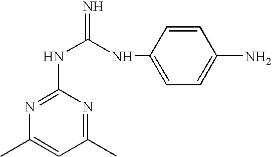 | 5805360 | 11053 | H10 | 256.31 | 5.3802037 |
| 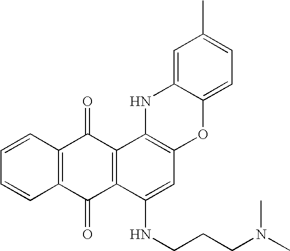 | 5468863 | 10882 | C03 | 427.5 | 0.6729825 |
| 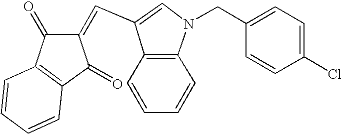 | 5603089 | 10946 | B08 | 397.85 | 14.404926 |
| 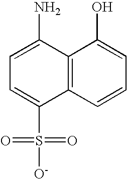 | 5265894 | 10821 | E11 | 238.24 | 1.0535594 |
| 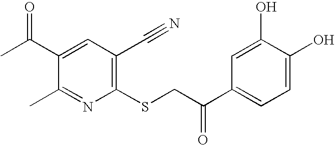 | 6033363 | 11141 | B03 | 342.37 | 10.953062 |
| 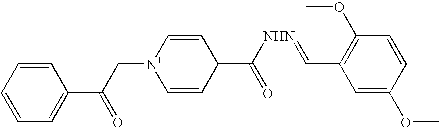 | 5483976 | 10902 | G10 | 404.44 | 1.346306 |
| 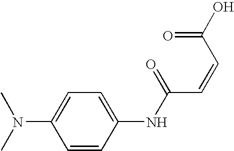 | 5374579 | 10948 | G07 | 234.25 | 20.426894 |
| 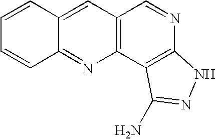 | 6051011 | 10799 | G06 | 235.24 | 3.7404353 |
| 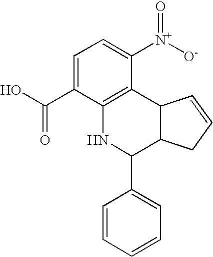 | 5853021 | 11070 | B04 | 336.34 | 3.9186537 |
| 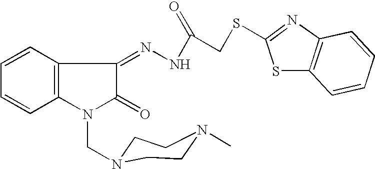 | 6062681 | 11169 | D09 | 480.61 | 12.900273 |
| 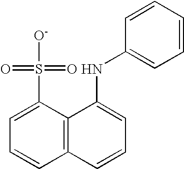 | 5255876 | 10821 | D11 | 298.34 | 2.9452973 |
| 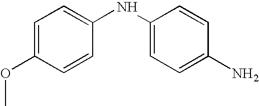 | 5529774 | 10912 | A06 | 214.26 | 7.2155325 |
| 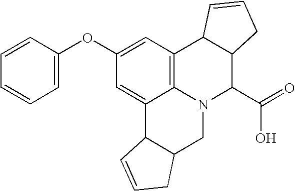 | 5840196 | 11063 | A06 | 385.46 | 4.1275359 |
| 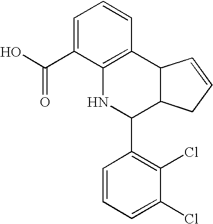 | 6048905 | 11158 | E06 | 360.23 | 2.5702385 |
| 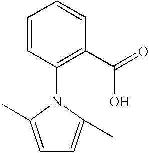 | 5959185 | 11227 | B02 | 215.25 | 0.6206736 |
| 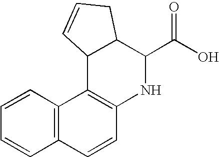 | 5657743 | 10965 | H07 | 265.31 | 7.1463571 |
| 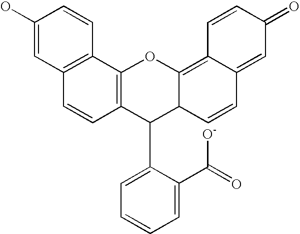 | 5466725 | 10880 | D04 | 432.42 | 1.6842422 |
| 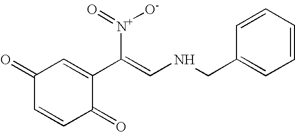 | 5261378 | 11228 | C08 | 284.27 | 5.6988075 |
| 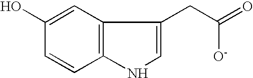 | 5106705 | 10794 | D04 | 190.18 | 7.8767483 |
| 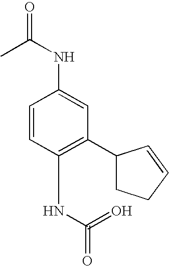 | 6047040 | 11154 | B02 | 272.3 | 4.4399559 |
| 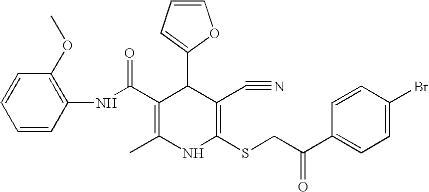 | 5570547 | 10938 | H10 | 564.45 | 6.2379307 |

| | | | H 95% | | |
|---|---|---|---|---|---|
| | Bcl-B IC50 | Bcl-B H | Confidence | | |
| Compound | (g/L) | Coefficient | Intervals | Bcl-B r2 | Bcl-2 IC50 (uM) |
| 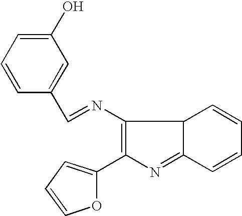 | 0.002751 | 0.5836 | 0.2638 to 0.9035 | 0.818 | 30.20342224 |
| 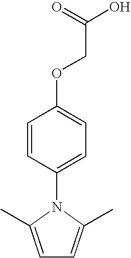 | 0.002145 | 0.6411 | 0.3374 to 0.9448 | 0.8577 | 3.068455172 |
| 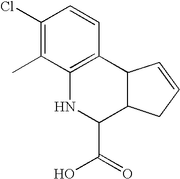 | 0.001373 | 0.6773 | 0.4401 to 0.9145 | 0.9261 | 28.12831791 |
| 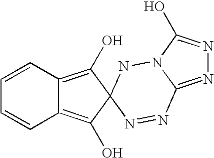 | 0.006851 | 0.9192 | 0.6651 to 1.173 | 0.965 | 23.93423289 |
| 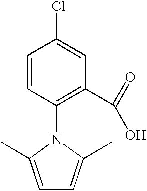 | 0.0002352 | 0.9279 | 0.5433 to 1.313 | 0.9298 | 9.191397333 |
| 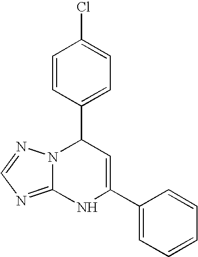 | 0.004378 | 0.939 | 0.7051 to 1.173 | 0.9715 | N/A |
| 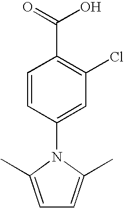 | 0.002486 | 0.9562 | 0.6727 to 1.240 | 0.962 | 41.09095278 |
| 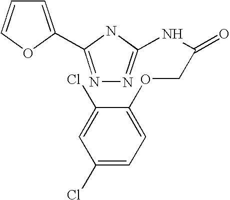 | 0.006304 | 0.9962 | 0.4505 to 1.542 | 0.8597 | 38.51867859 |
| 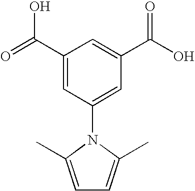 | 0.0001404 | 1.032 | 0.5548 to 1.510 | 0.8973 | 1.134768186 |
| 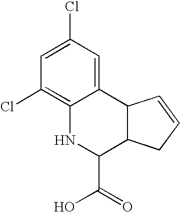 | 0.0002921 | 1.13 | 0.4017 to 1.859 | 0.8997 | 2.140142183 |
| 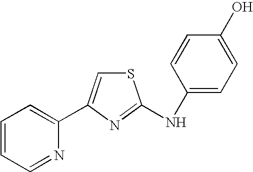 | 0.000994 | 1.182 | 0.8187 to 1.546 | 0.9748 | 13.40412892 |
| 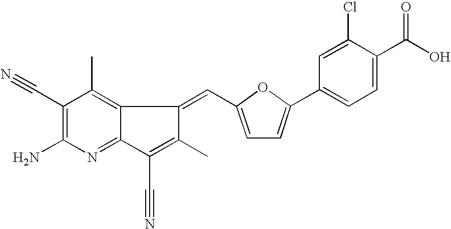 | 0.0007301 | 1.206 | 0.7105 to 1.701 | 0.9583 | 7.381731963 |
| 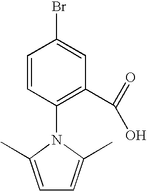 | 0.004577 | 1.233 | 0.6909 to 1.774 | 0.9333 | N/A |
| 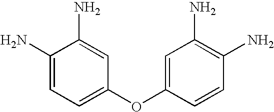 | 0.001237 | 1.303 | 0.8817 to 1.724 | 0.9744 | 13.24532071 |
| 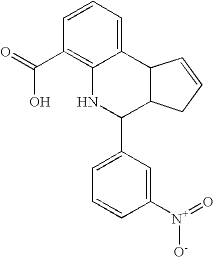 | 0.0007074 | 1.325 | 0.6388 to 2.012 | 0.9435 | 0.971338526 |
| 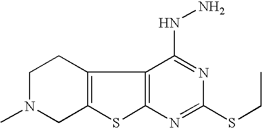 | 0.002194 | 1.332 | 0.9669 to 1.696 | 0.9799 | 40.92339979 |
| 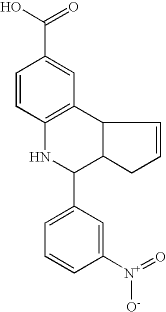 | 0.0005894 | 1.336 | 0.5825 to 2.089 | 0.941 | 5.119819231 |
| 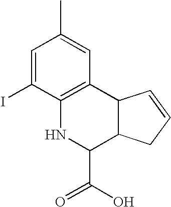 | 0.007348 | 1.406 | 0.9099 to 1.902 | 0.9585 | 29.98564068 |
| 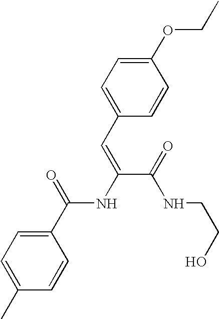 | 0.004882 | 1.428 | 0.9951 to 1.862 | 0.9719 | 29.85641777 |
| 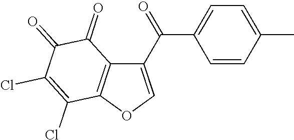 | 0.002067 | 1.432 | 1.077 to 1.788 | 0.9858 | 9.479620457 |
| 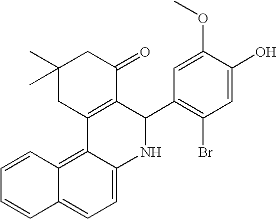 | 0.006263 | 1.507 | 1.138 to 1.875 | 0.9788 | 27.36318408 |
| 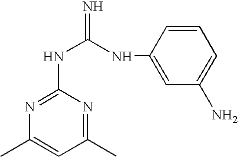 | 0.002904 | 1.599 | 0.9298 to 2.267 | 0.9654 | 19.28524053 |
| 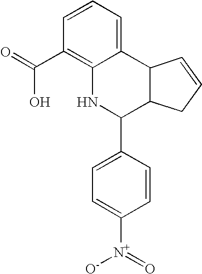 | 0.001819 | 1.735 | 0.1861 to 3.285 | 0.88 | 1.425938039 |
| 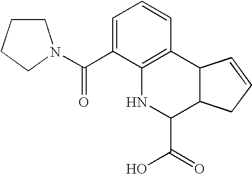 | 0.001546 | 1.761 | 1.248 to 2.273 | 0.9863 | 11.33948009 |
| 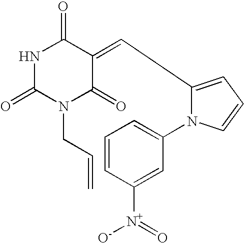 | 0.002048 | 1.773 | 1.157 to 2.389 | 0.9793 | 1.562540861 |
| 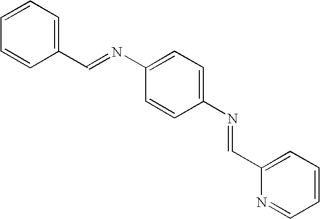 | 0.001256 | 1.819 | 0.0 to 5.672 | 0.7072 | 12.78943876 |
| 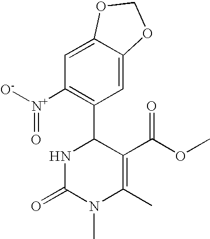 | 0.006071 | 1.821 | 0.7625 to 2.879 | 0.9112 | 22.78557114 |
| 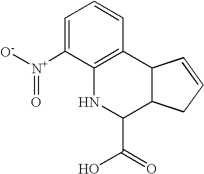 | 0.0003122 | 1.829 | 0.1152 to 3.541 | 0.8943 | 3.519692603 |
| 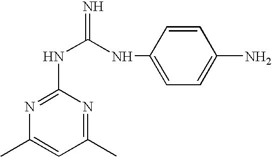 | 0.001379 | 1.878 | 1.057 to 2.698 | 0.9756 | 0.000390543 |
| 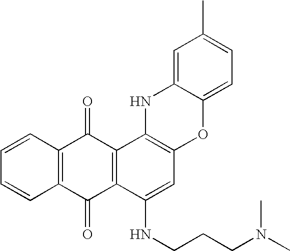 | 0.0002877 | 1.907 | 0.0 to 6.734 | 0.6571 | 3.726315789 |
| 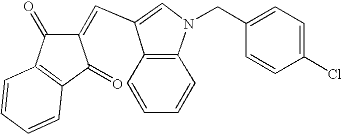 | 0.005731 | 1.917 | 1.261 to 2.574 | 0.9718 | 13.66092749 |
| 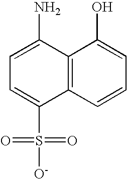 | 0.000251 | 2.075 | 0.3037 to 3.846 | 0.8556 | 1.996306246 |
| 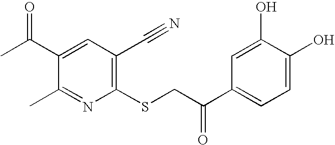 | 0.00375 | 2.08 | 0.6285 to 3.532 | 0.9242 | 3.443642843 |
| 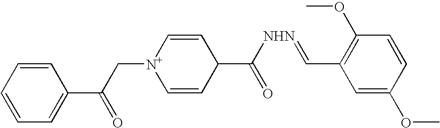 | 0.0005445 | 2.127 | 0.7803 to 3.474 | 0.9584 | 3.904163782 |
| 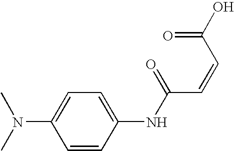 | 0.004785 | 2.167 | 0.9766 to 3.357 | 0.9464 | 28.27321238 |
| 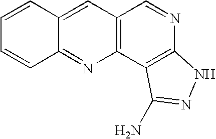 | 0.0006799 | 2.36 | 0.8778 to 3.843 | 0.9675 | 28.71960551 |
| 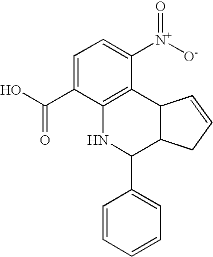 | 0.001318 | 2.481 | 0.04727 to 4.915 | 0.9275 | 6.139620622 |
| 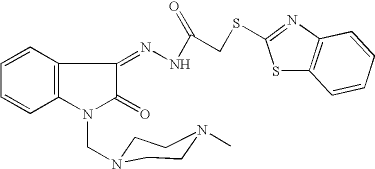 | 0.0062 | 2.531 | 1.589 to 3.472 | 0.977 | 32.95811573 |
| 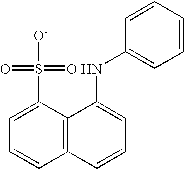 | 0.0008787 | 2.628 | 0.0 to 11.91 | 0.6396 | 12.6064222 |
| 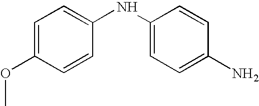 | 0.001546 | 2.718 | 0.0 to 6.324 | 0.8899 | 6.328759451 |
| 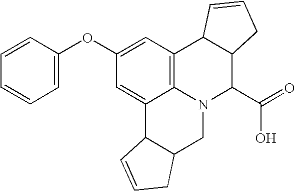 | 0.001591 | 2.774 | 0.0 to 8.225 | 0.8093 | 9.370622114 |
| 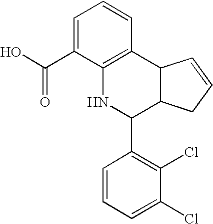 | 0.0009619 | 2.809 | 0.0 to 11.25 | 0.7205 | 3.13133276 |
| 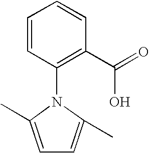 | 0.0001336 | 2.868 | 0.0 to 6.163 | 0.8842 | 1.596747987 |
| 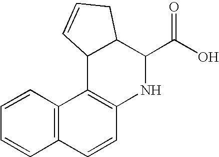 | 0.001896 | 3.005 | 0.6158 to 5.394 | 0.9574 | 11.7334439 |
| 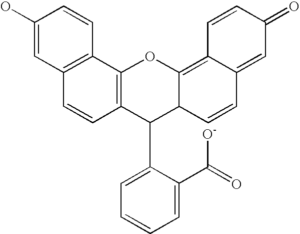 | 0.0007283 | 3.03 | 0.0 to 13.09 | 0.674 | 1.611396328 |
| 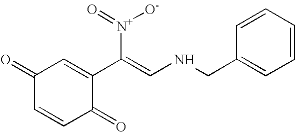 | 0.00162 | 3.214 | 0.0 to 7.490 | 0.9046 | 3.53185352 |
| 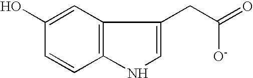 | 0.001498 | 3.385 | 0.0 to 8.943 | 0.9003 | 9.827531812 |
| 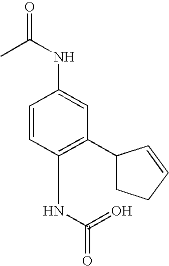 | 0.001209 | 3.664 | 0.0 to 7.858 | 0.9088 | 7.05471906 |
| 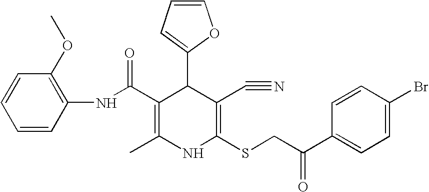 | 0.003521 | 18.57 | 0.0 to 21459 | 0.981 | 11.2215431 |

| | Bcl-2 IC50 | | | Bcl-2 H | H 95% Confidence |
|---|---|---|---|---|---|
| Compound | (g/L) | Well ID | ID | Coefficient | Intervals |
| 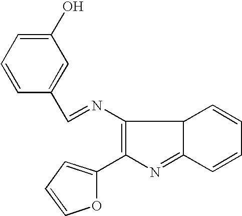 | 0.009161 | I17 | 8273 | | |
| 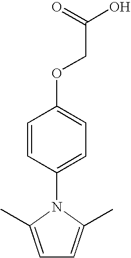 | 0.0007526 | C18 | 1600 | | |
| 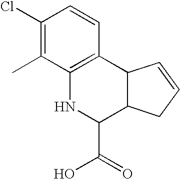 | 0.007418 | C05 | 9653 | | |
| 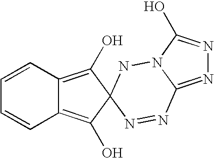 | 0.0069 | O05 | 9173 | 1.575 | 0.5994 to 2.551 |
| 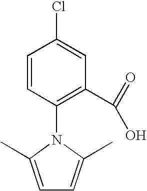 | 0.002295 | B08 | 10784 | 1.457 | 0.4701 to 2.445 |
| 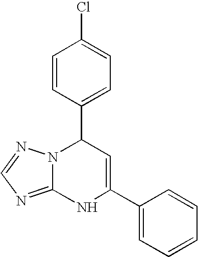 | XXX | L10 | 4114 | x | x |
| 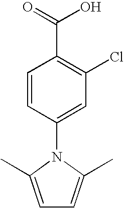 | 0.01026 | P08 | 10736 | 1.653 | 0.3319 to 2.975 |
| 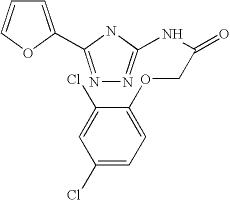 | 0.01426 | K12 | 9084 | 1.078 | 0.5507 to 1.605 |
| 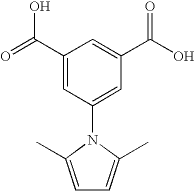 | 0.0002942 | J06 | 0.6186 | 0.3491 to 0.8880 | |
| 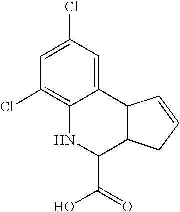 | 0.0006081 | I11 | 2.956 | 1.610 to 4.302 | |
| 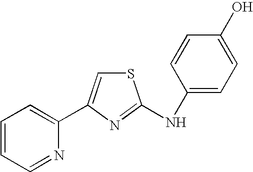 | 0.00361 | K04 | 9844 | 1.241 | 0.7725 to 1.709 |
| 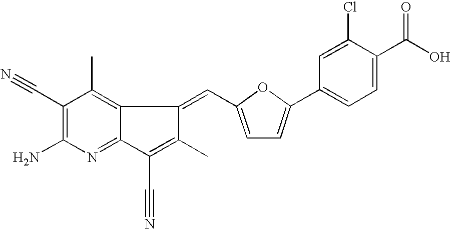 | 0.003269 | O15 | 1887 | 3.081 | 0.6160 to 5.547 |
| 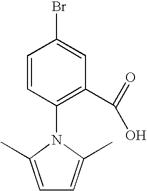 | | A15 | 1551 | | |
| 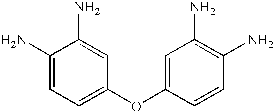 | 0.00305 | F06 | 2046 | 1.428 | 1.032 to 1.825 |
| 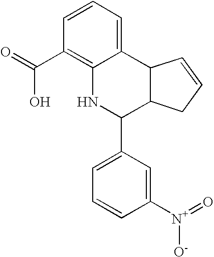 | 0.0003267 | L09 | 7569 | | |
| 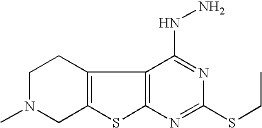 | 0.01209 | H22 | 10558 | | |
| 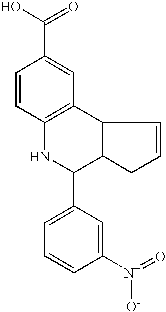 | 0.001722 | K14 | 2558 | | |
| 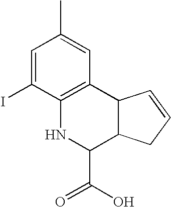 | 0.01055 | C22 | 3142 | | |
| 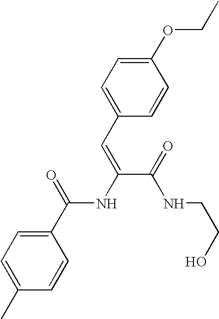 | 0.011 | L12 | 9108 | | |
| 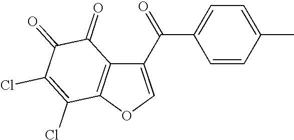 | 0.003177 | F16 | 9736 | | |
| 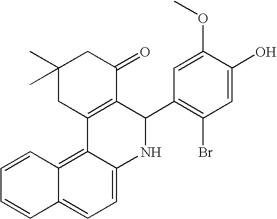 | 0.01309 | G19 | 9763 | | |
| 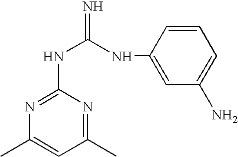 | 0.004943 | P06 | 5742 | | |
| 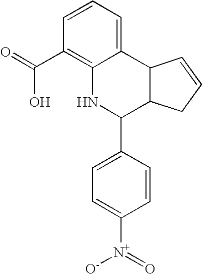 | 0.0004796 | B10 | 7330 | | |
| 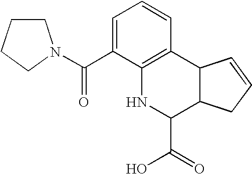 | 0.003542 | N18 | 11850 | | |
| 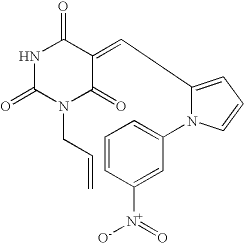 | 0.0005975 | G21 | 933 | | |
| 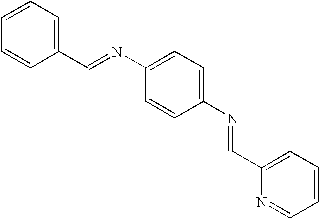 | 0.003662 | E17 | 3953 | | |
| 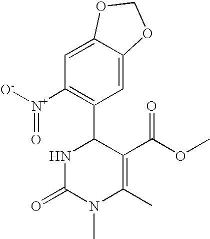 | 0.007959 | O06 | 8022 | | |
| 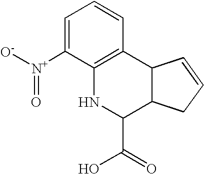 | 0.000916 | B05 | 9629 | | |
| 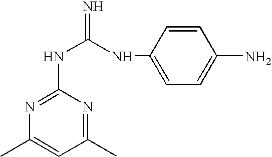 | 1.00E-07 | P20 | 5756 | | |
| 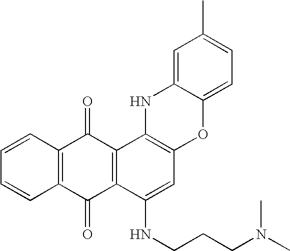 | 0.001593 | E05 | 1637 | | |
| 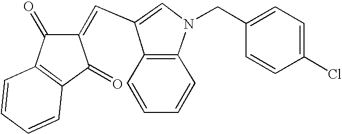 | 0.005436 | C15 | 7743 | | |
| 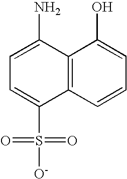 | 0.0004755 | J22 | 7150 | | |
| 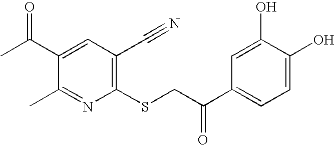 | 0.001179 | D06 | 1614 | | |
| 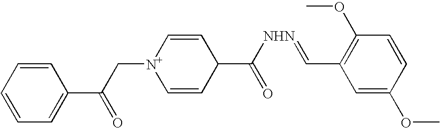 | 0.001579 | M19 | 3763 | | |
| 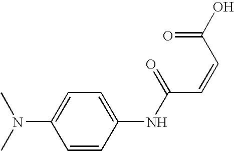 | 0.006623 | N13 | 8005 | | |
| 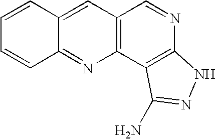 | 0.006756 | M12 | 5292 | | |
| 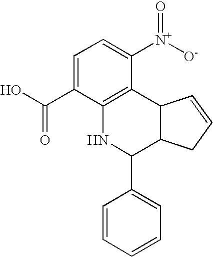 | 0.002065 | C07 | 7351 | | |
| 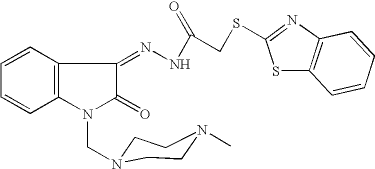 | 0.01584 | H18 | 4410 | | |
| 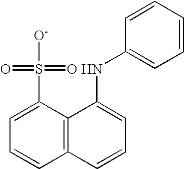 | 0.003761 | H22 | 7102 | | |
| 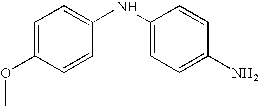 | 0.001356 | B11 | 4259 | | |
| 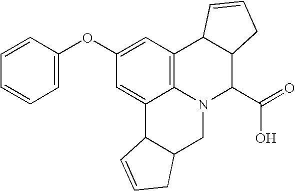 | 0.003612 | A12 | 6540 | | |
| 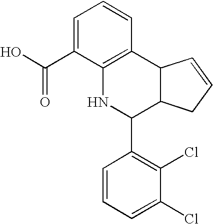 | 0.00128 | I11 | 3659 | | |
| 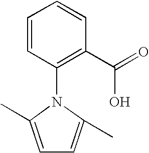 | 0.0003437 | C04 | 10036 | | |
| 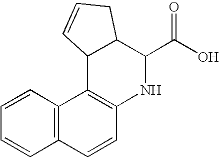 | 0.003113 | P14 | 9590 | | |
| 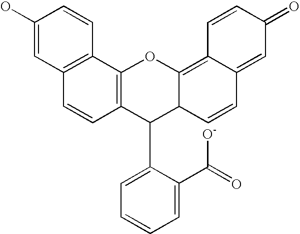 | 0.0006968 | H07 | 1327 | | |
| 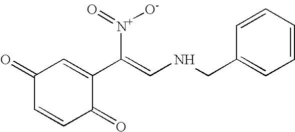 | 0.001004 | F16 | 10119 | | |
| 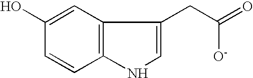 | 0.001869 | G07 | 4759 | | |
| 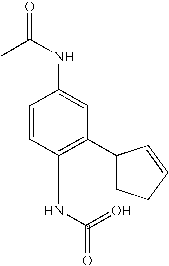 | 0.001921 | C03 | 3123 | | |
| 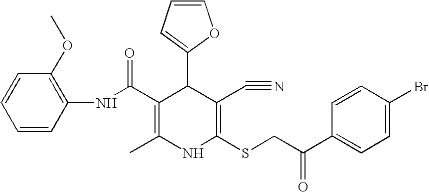 | 0.006834 | O19 | 7267 | | |

| | | HSP70 |
|---|---|---|
| Compound | Bcl-2 r2 | IC50 (Hill) |
| 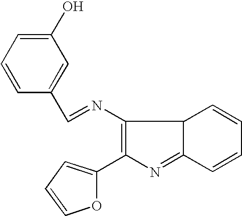 | | |
| 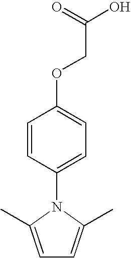 | | |
| 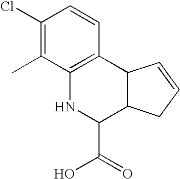 | | |
| 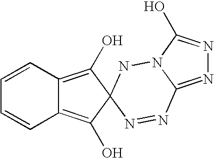 | 0.8803 | X |
| 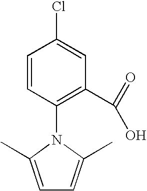 | 0.9127 | high |
| 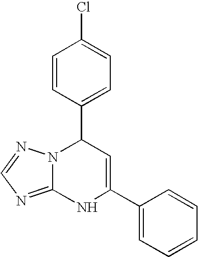 | x | high |
| 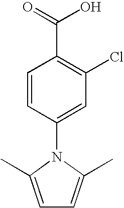 | 0.8386 | X |
| 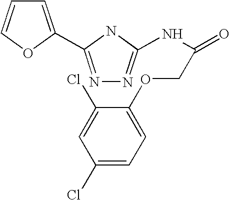 | 0.9003 | high (0.03272 roughly?) |
| 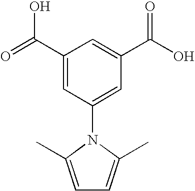 | 0.8688 | x |
| 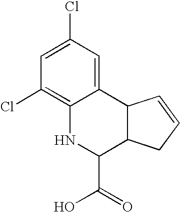 | 0.9828 | 0.0105 |
| 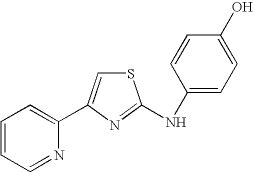 | 0.9554 | x |
| 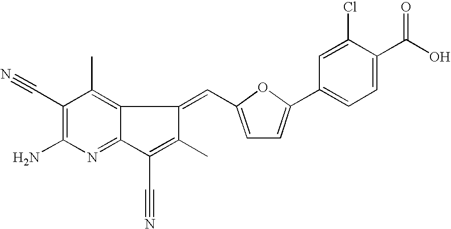 | 0.9551 | high |
| 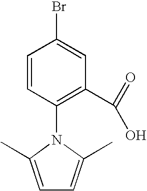 | | |
| 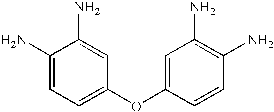 | 0.9812 | High |

TABLE 5B

| | | Percent | | |
|---|---|---|---|---|
| STRUCTURE | PUBCHEM_SID | Efficacy | % Activity | FRatio |
| 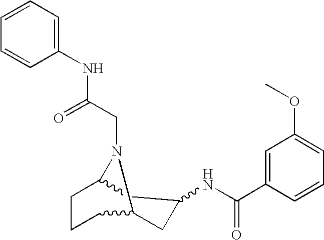 #NAME? | 847772 | 90 | 90 | 0.72 |
| 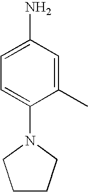 #NAME? | 14744983 | 86.5 | 86.5 | 0.71 |
| 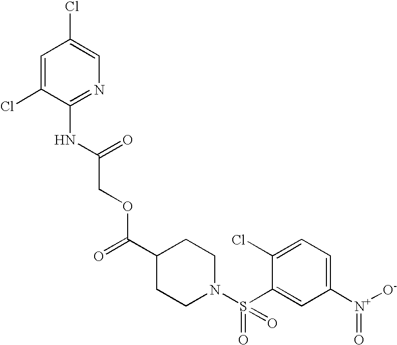 #NAME? | 14744641 | 75.9 | 75.9 | 0.93 |
| 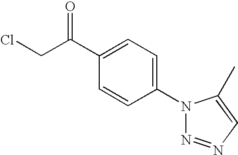 #NAME? | 14744853 | 52.9 | 52.9 | 0.82 |
| 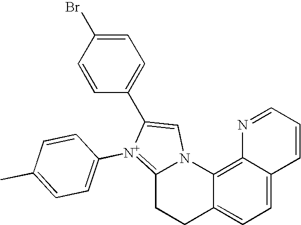 #NAME? | 14745910 | 56.2 | 56.2 | 0.81 |
| 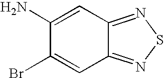 #NAME? | 17407909 | 58.5 | 58.5 | 1.42 |
| 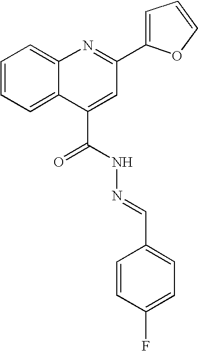 #NAME? | 17408998 | 84.8 | 84.8 | 0.87 |
| 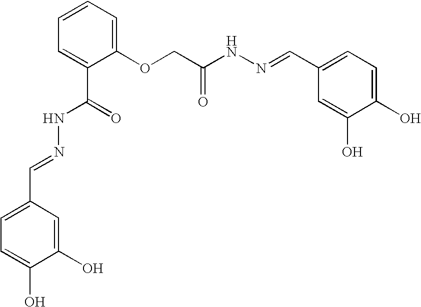 #NAME? | 17409262 | 70.9 | 70.9 | 0.77 |
| 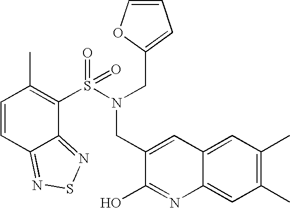 #NAME? | 14720830 | 56 | 56 | 1.29 |
| 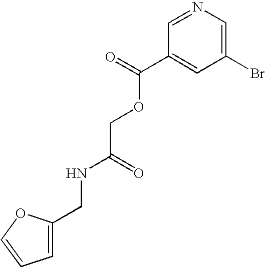 #NAME? | 3715512 | 55.4 | 55.4 | 0.95 |
| 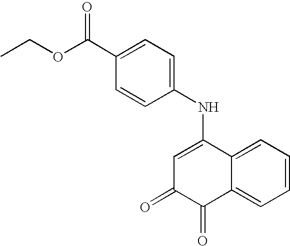 #NAME? | 14737410 | 89.5 | 89.5 | 1.15 |
| 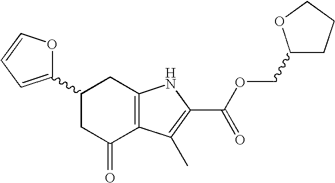 #NAME? | 4244684 | 54.3 | 54.3 | 0.79 |
| 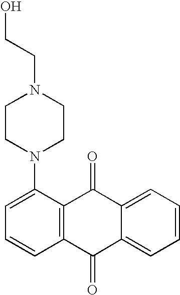 #NAME? | 17403435 | 56.9 | 56.9 | 1.3 |
| 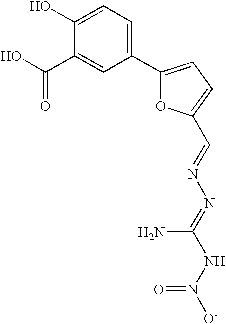 #NAME? | 17416093 | 84.4 | 84.4 | 0.7 |
| 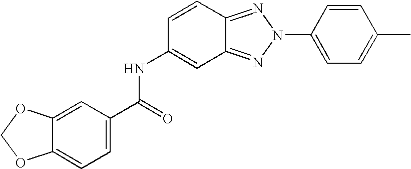 #NAME? | 14732083 | 69.5 | 69.5 | 0.83 |
| 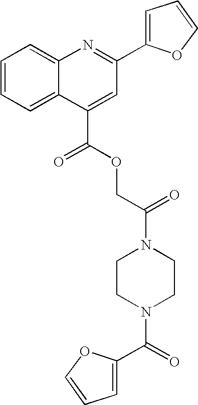 #NAME? | 14745657 | 73.4 | 73.4 | 0.97 |
| 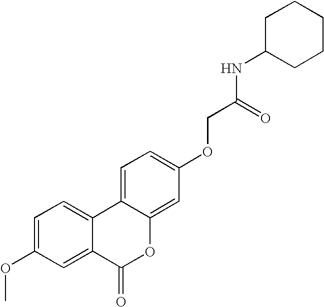 #NAME? | 14730237 | 78.9 | 78.9 | 0.91 |
| 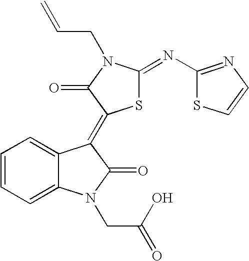 #NAME? | 17401584 | 131.8 | 131.8 | 0.83 |
| 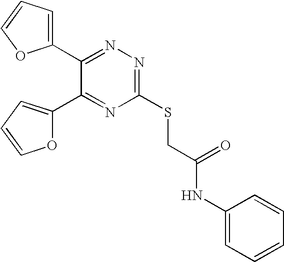 #NAME? | 860234 | 71.5 | 71.5 | 1.17 |
| 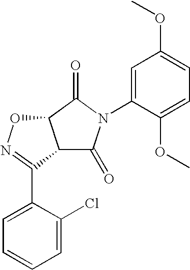 #NAME? | 864036 | 68.5 | 68.5 | 0.89 |
| 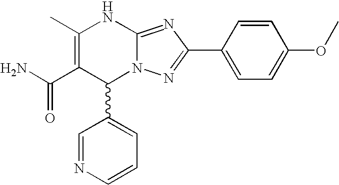 #NAME? | 4250160 | 88.5 | 88.5 | 0.81 |
| 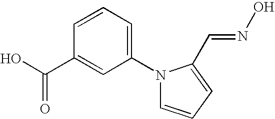 #NAME? | 4243236 | 70 | 70 | 0.86 |
| 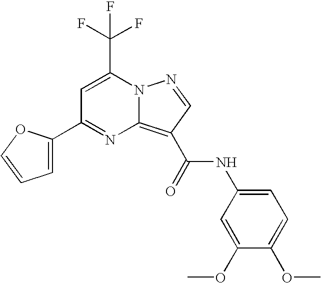 #NAME? | 7974080 | 52 | 52 | 0.9 |
| 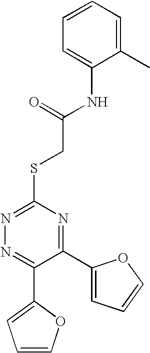 #NAME? | 14742395 | 56.3 | 56.3 | 1.09 |
| 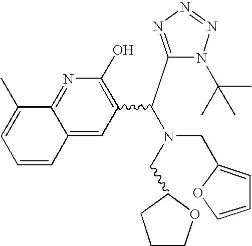 #NAME? | 14720806 | 64.5 | 64.5 | 0.95 |
| 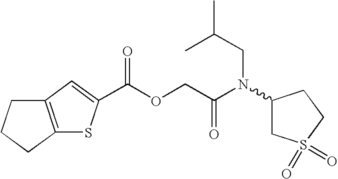 #NAME? | 3714271 | 53.1 | 53.1 | 0.67 |
| 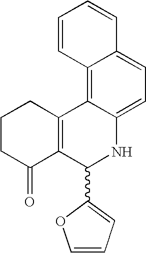 #NAME? | 7975176 | 52.2 | 52.2 | 0.73 |
| 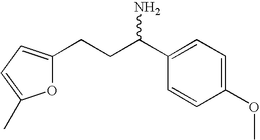 #NAME? | 4244526 | 67.6 | 67.6 | 0.84 |
| 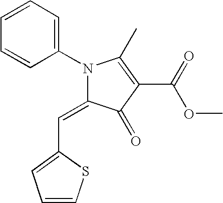 #NAME? | 17415043 | 65.7 | 65.7 | 0.53 |
| 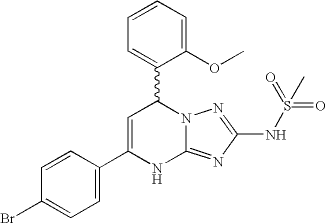 #NAME? | 8611447 | 50.7 | 50.7 | 1.24 |
| 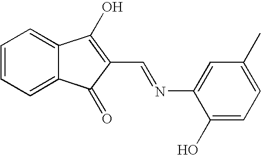 #NAME? | 7974116 | 56 | 56 | 0.92 |
| 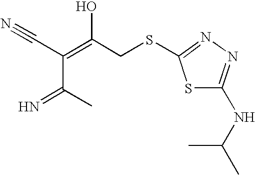 #NAME? | 3717601 | 84.1 | 84.1 | 0.47 |
| 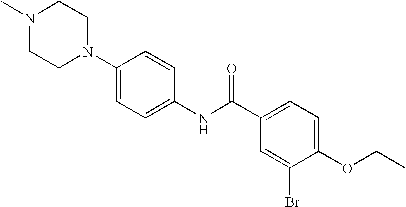 #NAME? | 4256635 | 70 | 70 | 0.53 |
| 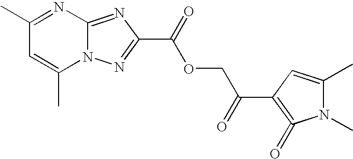 #NAME? | 3712105 | 69.9 | 69.9 | 0.7 |
| 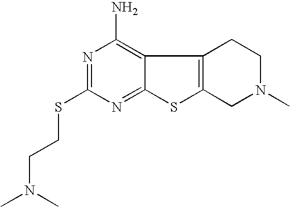 #NAME? | 862887 | 72.8 | 72.8 | 0.98 |
| 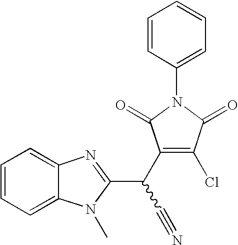 #NAME? | 3716185 | 89.8 | 89.8 | 0.68 |
| 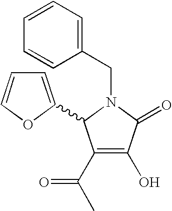 #NAME? | 851207 | 52.6 | 52.6 | 0.52 |
| 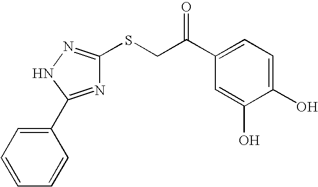 #NAME? | 851690 | 75.1 | 75.1 | 0.43 |
| 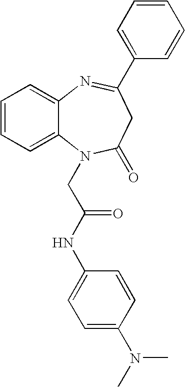 #NAME? | 853473 | 62.6 | 62.6 | 0.6 |
| 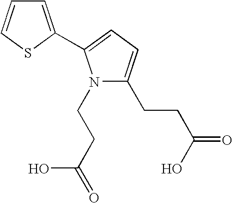 #NAME? | 848328 | 74.6 | 74.6 | 0.59 |
| 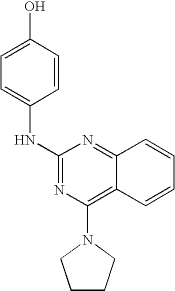 #NAME? | 852615 | 96.1 | 96.1 | 0.46 |
| 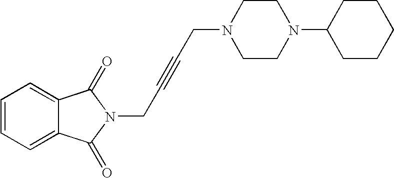 #NAME? | 856035 | 56.3 | 56.3 | 0.7 |
| 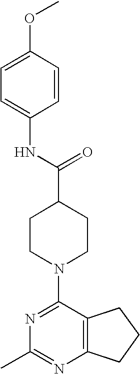 #NAME? | 852694 | 66.2 | 66.2 | 1.37 |
| 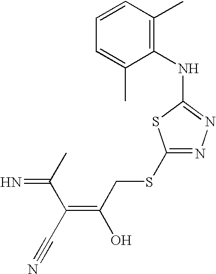 #NAME? | 3715042 | 63.1 | 63.1 | 0.63 |
| 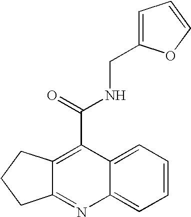 #NAME? | 3715091 | 62.4 | 62.4 | 0.86 |
| 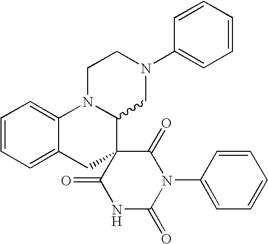 #NAME? | 865821 | 53 | 53.1 | 1.21 |
| 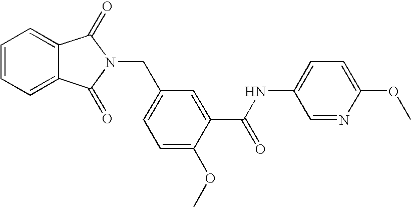 #NAME? | 3713992 | 66.9 | 66.9 | 1.47 |
| 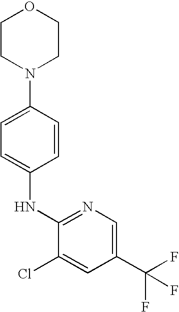 #NAME? | 3713921 | 51.4 | 51.4 | 0.51 |
| 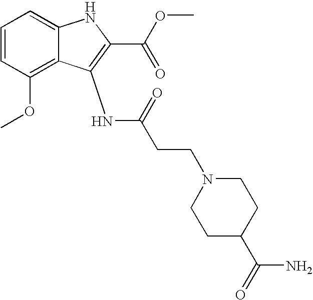 #NAME? | 863493 | 71.7 | 71.7 | 0.76 |
| 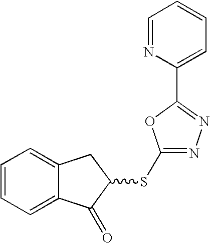 #NAME? | 842657 | 65.7 | 65.7 | 1.17 |
| 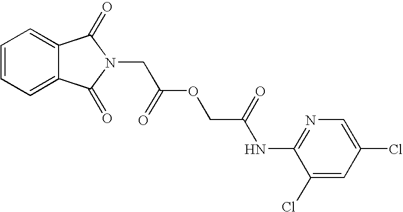 #NAME? | 3711621 | 58.7 | 58.7 | 1.36 |
| 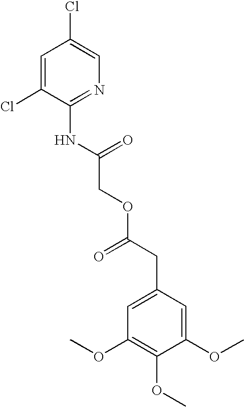 #NAME? | 3714968 | 54 | 54 | 1.42 |
| 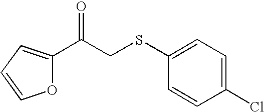 #NAME? | 3711335 | 75.2 | 75.2 | 0.83 |
| 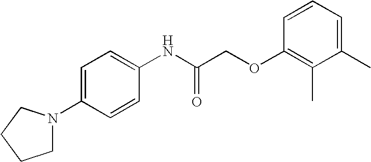 #NAME? | 7975075 | 76 | 76.1 | 0.72 |
| 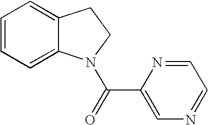 #NAME? | 3713383 | 80.1 | 80.1 | 0.72 |
| 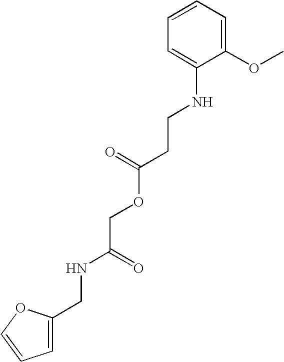 #NAME? | 3712399 | 85.2 | 85.2 | 0.95 |
| 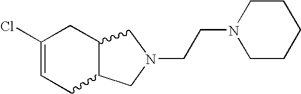 #NAME? | 860752 | 67.7 | 67.7 | 0.79 |
| 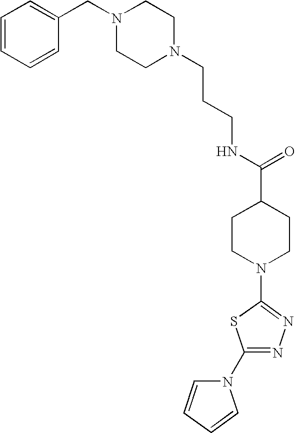 #NAME? | 4250401 | 50.8 | 50.8 | 0.86 |
| 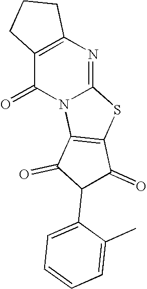 #NAME? | 7977096 | 50.8 | 50.8 | 0.88 |
| 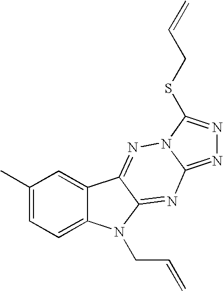 #NAME? | 7973953 | 69.4 | 69.4 | 0.82 |
| 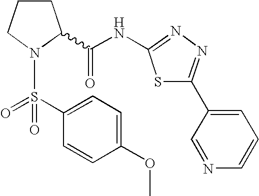 #NAME? | 3713012 | 50.9 | 50.9 | 0.9 |
| 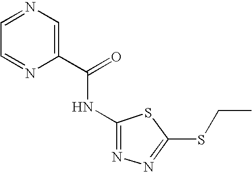 #NAME? | 7977261 | 58.3 | 58.3 | 1.19 |
| 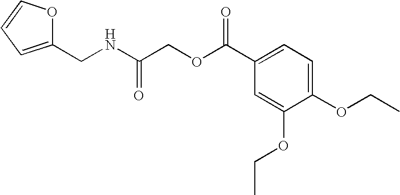 #NAME? | 3714498 | 71.4 | 71.4 | 1 |
| 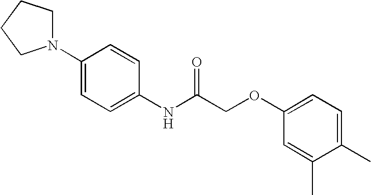 #NAME? | 7974807 | 90.6 | 90.6 | 0.76 |
| 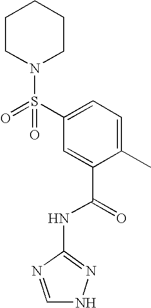 #NAME? | 3716232 | 50.5 | 50.5 | 0.86 |
| 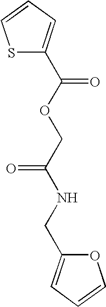 #NAME? | 3712480 | 79.7 | 79.7 | 0.96 |
| 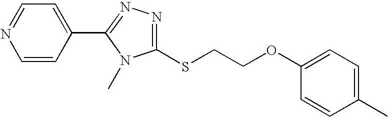 #NAME? | 3712590 | 68.9 | 68.9 | 0.91 |
| 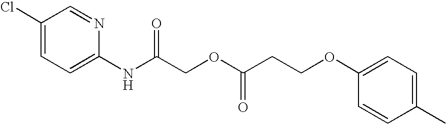 #NAME? | 3713376 | 54.8 | 54.8 | 1.43 |
| 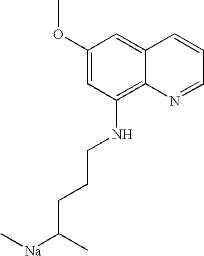 #NAME? | 850375 | 98.5 | 98.5 | 0.87 |
| 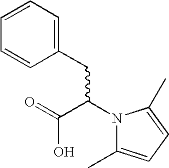 #NAME? | 852148 | 82.9 | 82.9 | 1.37 |
| 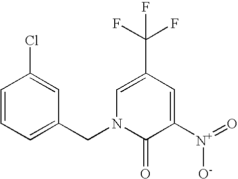 #NAME? | 17402915 | 89.8 | 89.8 | 0.61 |
| 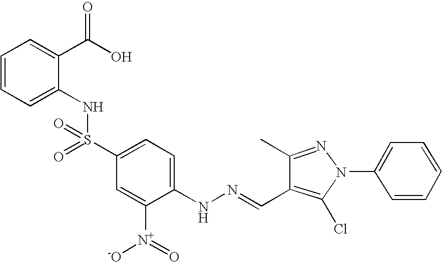 #NAME? | 14745518 | 82.4 | 82.4 | 0.71 |
| 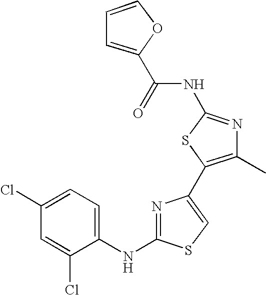 #NAME? | 17410012 | 54.4 | 54.4 | 0.7 |
| 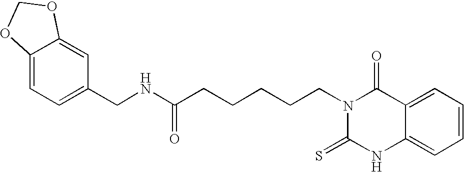 #NAME? | 17402018 | 52.9 | 52.9 | 1.37 |
| 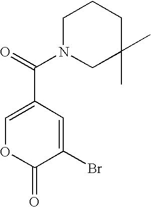 #NAME? | 862800 | 80.8 | 80.8 | 0.65 |
| 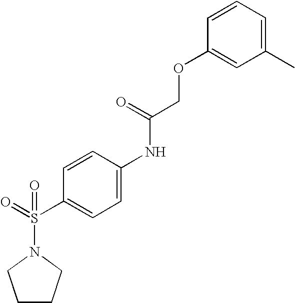 #NAME? | 7973703 | 79.2 | 79.2 | 0.82 |
| 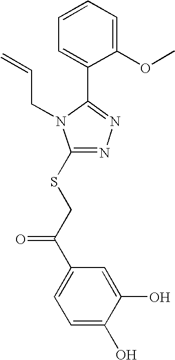 #NAME? | 863840 | 54.2 | 54.2 | 0.88 |
| 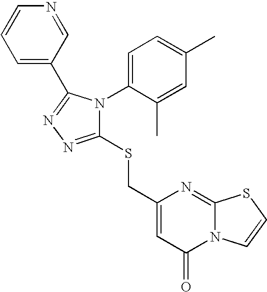 #NAME? | 3712302 | 80.3 | 80.3 | 0.96 |
| 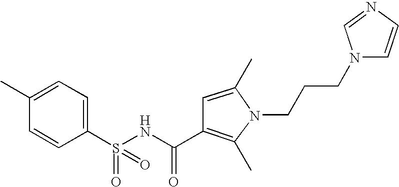 #NAME? | 3173759 | 61.5 | 61.5 | 0.99 |
| 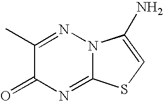 #NAME? | 846443 | 56.4 | 56.4 | 1.35 |
| 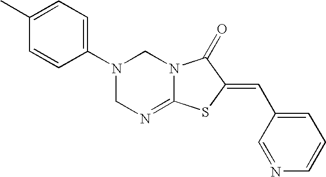 #NAME? | 858221 | 78.6 | 78.6 | 0.88 |
| 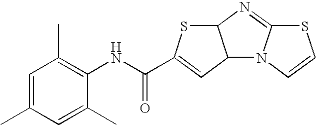 #NAME? | 3712588 | 66.1 | 66.1 | 1.05 |
| 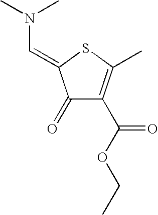 #NAME? | 7972859 | 68.4 | 68.4 | 0.91 |
| 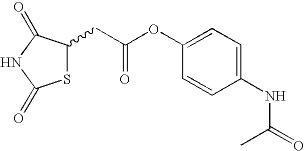 #NAME? | 860139 | 68.2 | 68.2 | 1.06 |
| 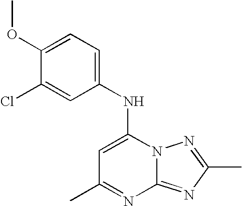 #NAME? | 7967129 | 82.3 | 82.3 | 1.11 |
| 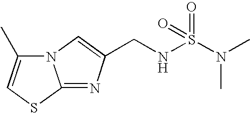 #NAME? | 7970333 | 53.8 | 53.8 | 0.96 |
| 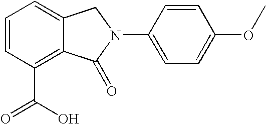 #NAME? | 7969281 | 113.7 | 113.7 | 0.86 |
| 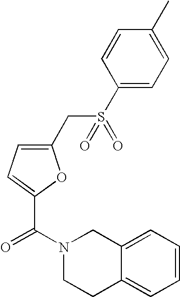 #NAME? | 14733336 | 60.8 | 60.8 | 0.93 |
| 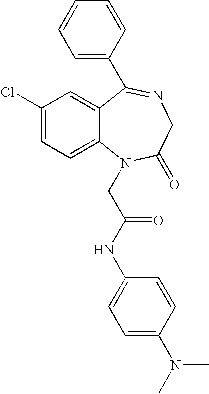 #NAME? | 14738707 | 66.4 | 66.4 | 0.84 |
| 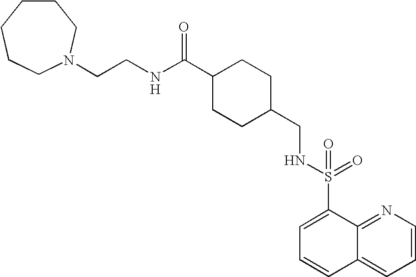 #NAME? | 7969436 | 51.8 | 51.8 | 0.97 |
| 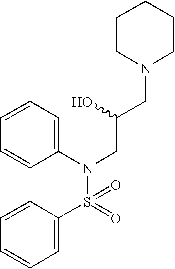 #NAME? | 859970 | 53.9 | 53.9 | 0.98 |
| 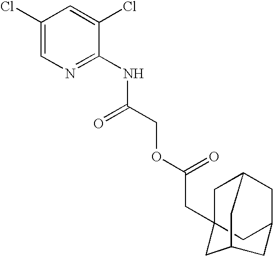 #NAME? | 14744137 | 52 | 52 | 1.16 |
| 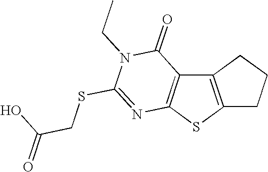 #NAME? | 856717 | 83 | 83 | 0.85 |
| 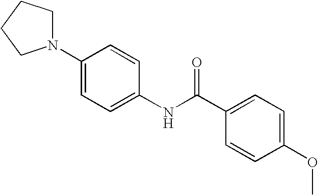 #NAME? | 14739290 | 78.8 | 78.8 | 0.85 |
| 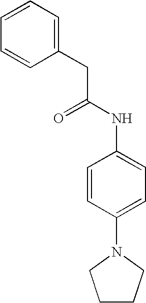 #NAME? | 14739360 | 59.1 | 59.1 | 0.93 |
| 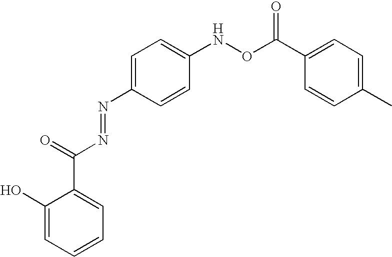 #NAME? | 14732345 | 108.7 | 108.7 | 0.83 |
| 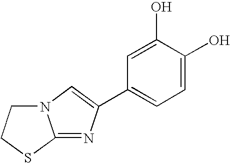 #NAME? | 14746201 | 77.7 | 77.7 | 0.91 |
| 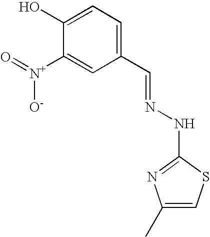 #NAME? | 14746238 | 57.1 | 57.1 | 0.84 |
| 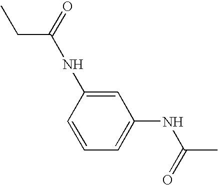 #NAME? | 14746435 | 92.4 | 92.4 | 0.85 |
| 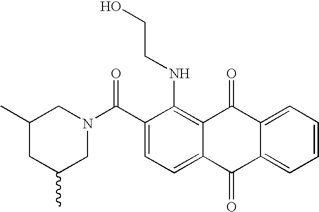 #NAME? | 14734023 | 83.7 | 83.7 | 0.97 |
| 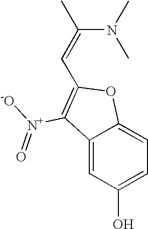 #NAME? | 14746248 | 61.2 | 61.2 | 0.61 |
| 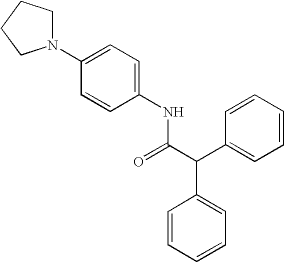 #NAME? | 14730995 | 64.1 | 64.1 | 0.9 |
| 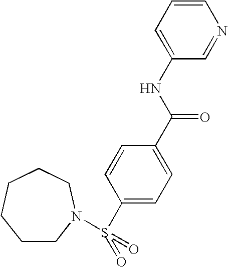 #NAME? | 14744015 | 88.4 | 88.4 | 0.84 |
| 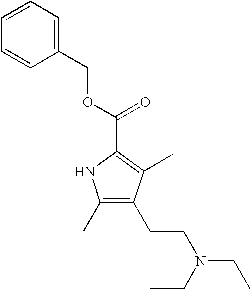 #NAME? | 853427 | 70.8 | 70.8 | 1 |
| 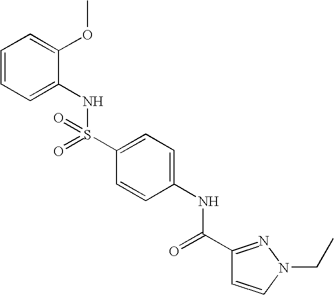 #NAME? | 4241115 | 82 | 82 | 0.87 |
| 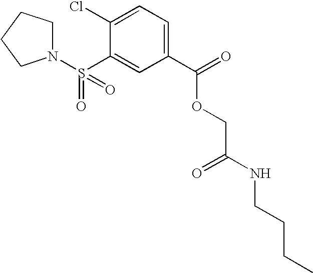 #NAME? | 16952426 | 54.8 | 54.8 | 0.92 |
| 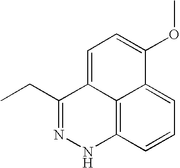 #NAME? | 14740154 | 83.6 | 83.6 | 1 |
| 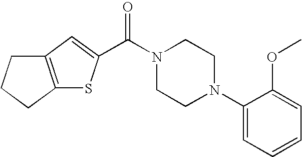 #NAME? | 14746697 | 74.1 | 74.1 | 0.9 |
| 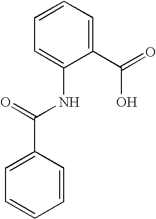 #NAME? | 14723278 | 85.4 | 85.4 | 0.87 |
| 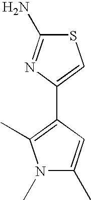 #NAME? | 14746541 | 69.5 | 69.5 | 0.94 |
| 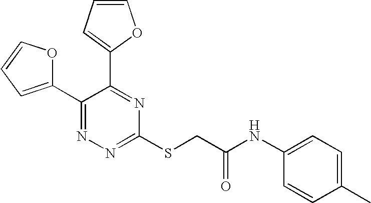 #NAME? | 860242 | 56.9 | 56.9 | 1.34 |
| 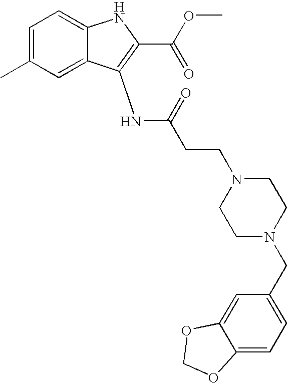 #NAME? | 862218 | 69.7 | 69.7 | 0.81 |
| 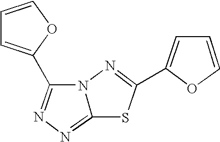 #NAME? | 853208 | 60.4 | 60.4 | 0.83 |
| 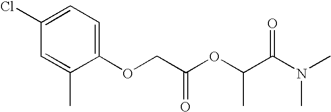 #NAME? | 867049 | 53.3 | 53.3 | 0.78 |
| 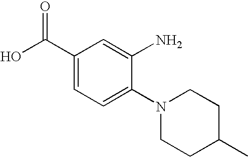 #NAME? | 852908 | 73.3 | 73.3 | 0.75 |
| 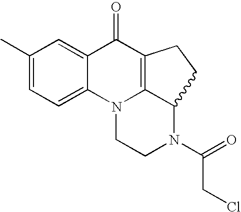 #NAME? | 14742276 | 72.4 | 72.4 | 0.88 |
| 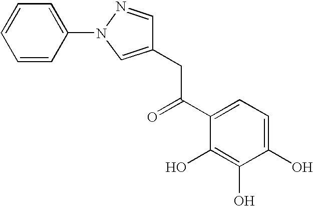 #NAME? | 14732615 | 57.8 | 57.8 | 0.74 |
| 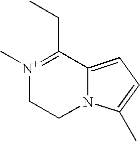 #NAME? | 14734567 | 57.1 | 57.1 | 1 |
| 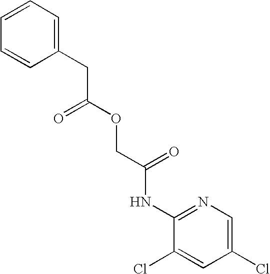 #NAME? | 14744122 | 58.8 | 58.8 | 1.03 |
| 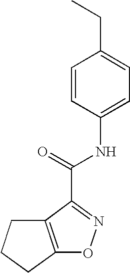 #NAME? | 14733222 | 68 | 68.1 | 0.73 |
| 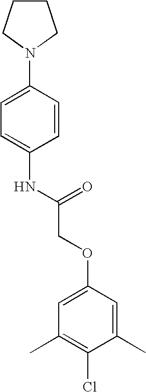 #NAME? | 14734365 | 59.3 | 59.3 | 0.71 |
| 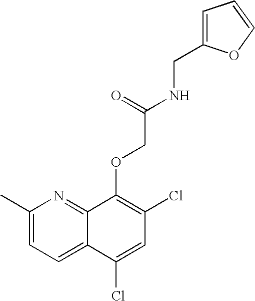 #NAME? | 14747102 | 56.5 | 56.5 | 0.8 |
| 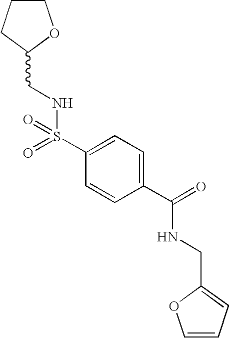 #NAME? | 843210 | 104.5 | 104.5 | 0.63 |
| 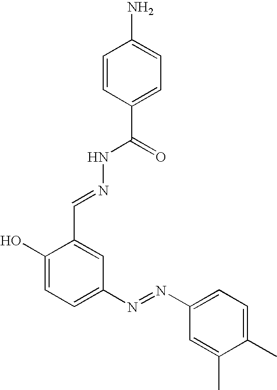 #NAME? | 16952937 | 91.7 | 91.7 | 0.71 |
| 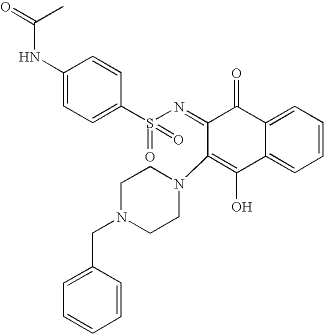 #NAME? | 14742166 | 50.6 | 50.6 | 0.73 |
| 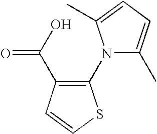 #NAME? | 14729511 | 80 | 80 | 0.77 |
| 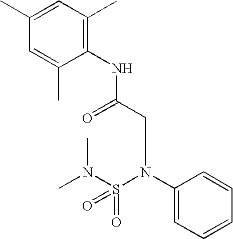 #NAME? | 14746364 | 90.5 | 90.5 | 0.65 |
| 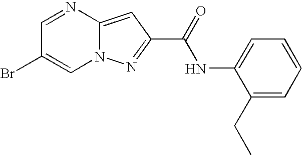 #NAME? | 14733824 | 74.2 | 74.2 | 0.79 |
| 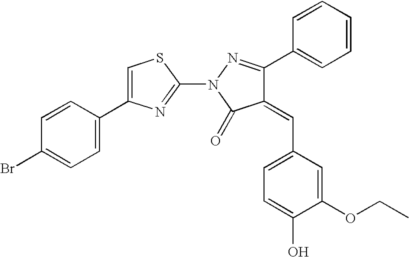 #NAME? | 14733887 | 68.2 | 68.2 | 0.76 |
| 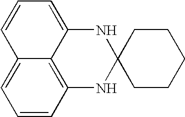 #NAME? | 14724643 | 52.7 | 52.7 | 0.82 |
| 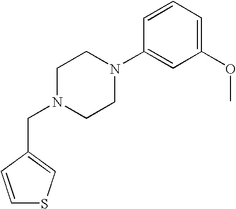 #NAME? | 14746122 | 56.1 | 56.1 | 0.85 |
| 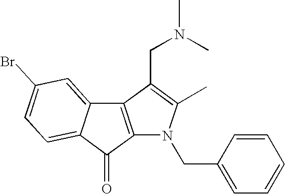 #NAME? | 14742663 | 78.5 | 78.5 | 0.61 |
| 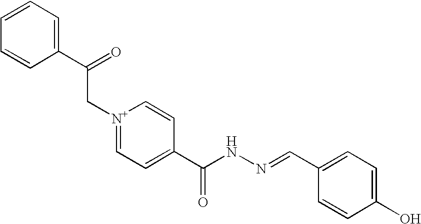 #NAME? | 14746260 | 57.9 | 57.9 | 0.72 |
| 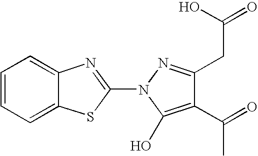 #NAME? | 14746319 | 69 | 69 | 0.7 |
| 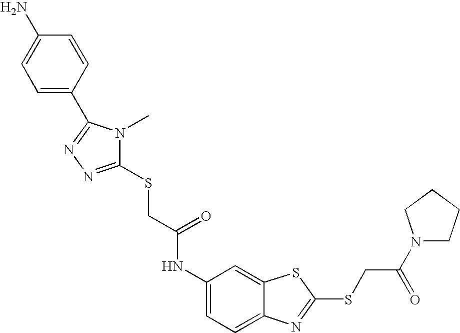 #NAME? | 17386645 | 53.9 | 53.9 | 0.79 |
| 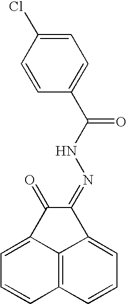 #NAME? | 16953557 | 68.6 | 68.6 | 0.84 |
| 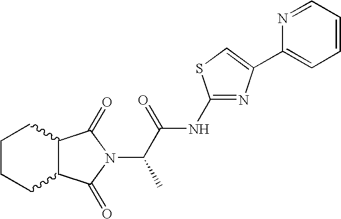 #NAME? | 16952892 | 51.1 | 51.1 | 1.04 |
| 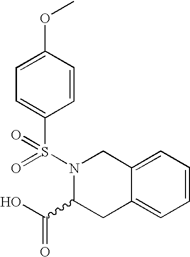 #NAME? | 16953199 | 53 | 53 | 0.85 |
| 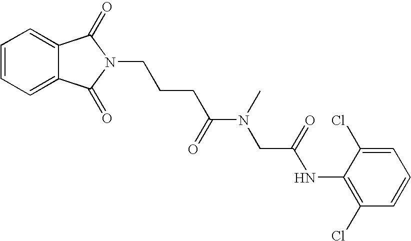 #NAME? | 17388283 | 57.1 | 57.1 | 0.97 |
| 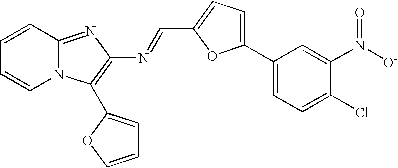 #NAME? | 17388349 | 60.5 | 60.5 | 0.99 |
| 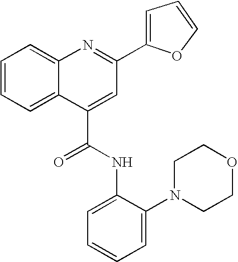 #NAME? | 14744733 | 61.4 | 61.4 | 0.83 |
| 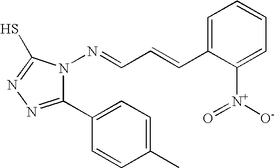 #NAME? | 14729394 | 55.5 | 55.5 | 0.81 |
| 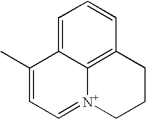 #NAME? | 14748512 | 76.6 | 76.6 | 0.76 |
| 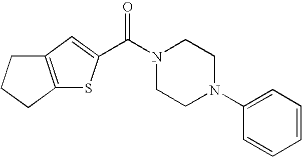 #NAME? | 14745443 | 71.6 | 71.6 | 0.73 |
| 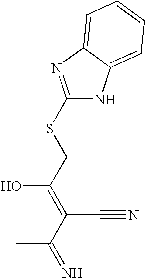 #NAME? | 17385704 | 105.8 | 105.8 | 0.67 |
| 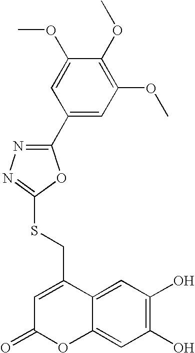 #NAME? | 17385711 | 68.3 | 68.3 | 0.7 |
| 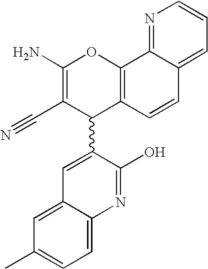 #NAME? | 14720783 | 55 | 55 | 0.84 |
| 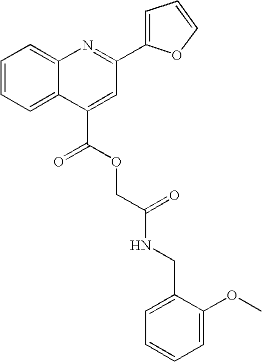 #NAME? | 17385737 | 70.8 | 70.8 | 0.81 |
| 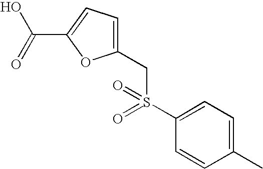 #NAME? | 14737647 | 66.5 | 66.5 | 0.86 |
| 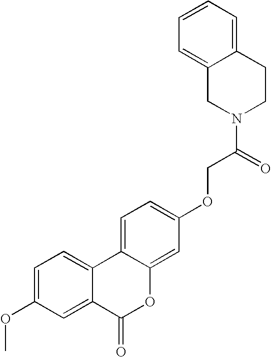 #NAME? | 14730322 | 59.7 | 59.7 | 0.75 |
| 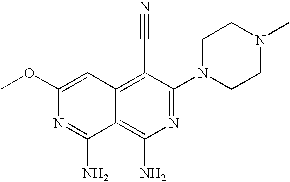 #NAME? | 14746488 | 51.4 | 51.4 | 0.92 |
| 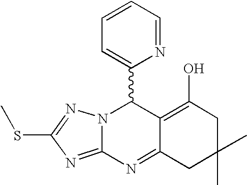 #NAME? | 14742716 | 61.8 | 61.8 | 0.84 |
| 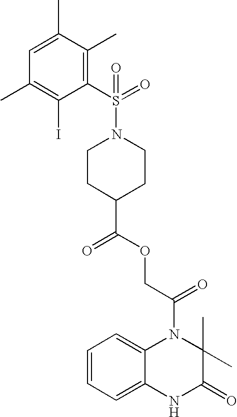 #NAME? | 16952989 | 72.9 | 72.9 | 0.93 |
| 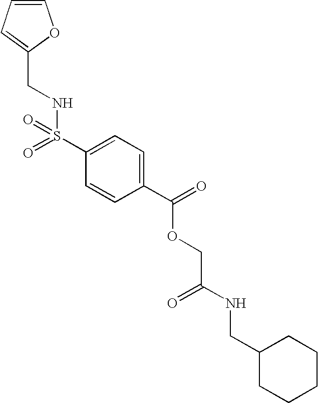 #NAME? | 16953469 | 60.3 | 60.3 | 1.01 |
| 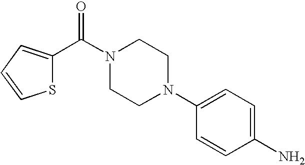 #NAME? | 14726238 | 57.8 | 57.8 | 0.87 |
| 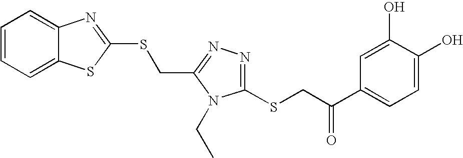 #NAME? | 17388455 | 74.1 | 74.1 | 0.8 |
| 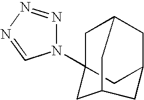 #NAME? | 14725293 | 60.7 | 60.7 | 0.98 |
| 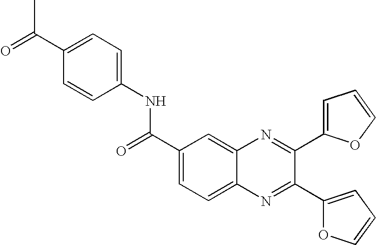 #NAME? | 14740115 | 59.5 | 59.5 | 0.97 |
| 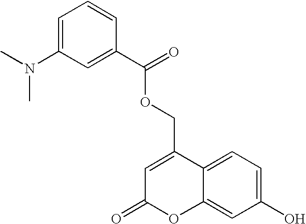 #NAME? | 17388495 | 71.7 | 71.7 | 1 |
| 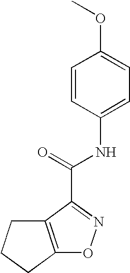 #NAME? | 14733727 | 102.1 | 102.1 | 0.93 |
| 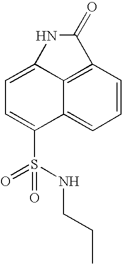 #NAME? | 14723132 | 58.3 | 58.3 | 1.25 |
| 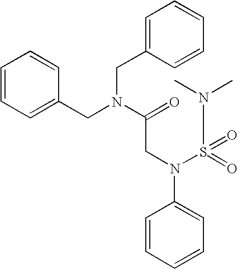 #NAME? | 14736293 | 90.4 | 90.4 | 0.73 |
| 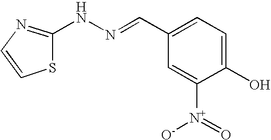 #NAME? | 14746394 | 107 | 107 | 0.75 |
| 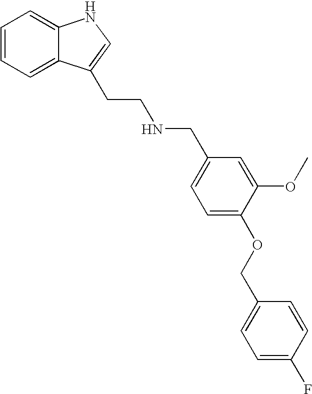 #NAME? | 17386700 | 76.5 | 76.5 | 0.84 |
| 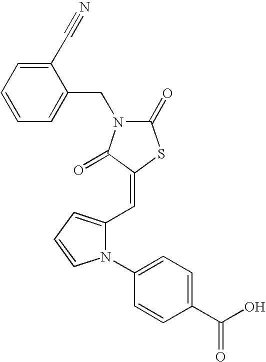 #NAME? | 17385953 | 63.5 | 63.5 | 0.75 |
| 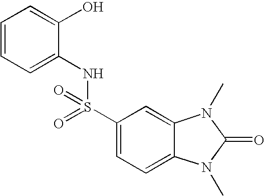 #NAME? | 14723355 | 52.9 | 52.9 | 0.98 |
| 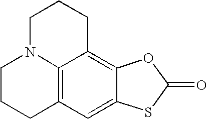 #NAME? | 7978324 | 66.9 | 66.9 | 1.02 |
| 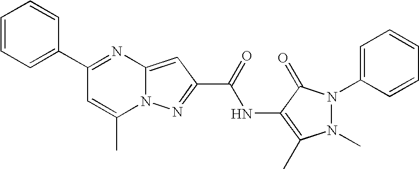 #NAME? | 844334 | 63.5 | 63.5 | 0.88 |
| 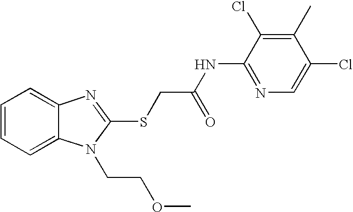 #NAME? | 3717339 | 64.7 | 64.7 | 0.94 |
| 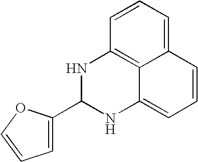 #NAME? | 844697 | 78.1 | 78.1 | 1.08 |
| 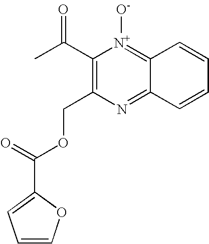 #NAME? | 863818 | 68.3 | 68.3 | 0.88 |
| 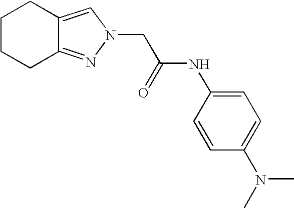 #NAME? | 7968138 | 57.6 | 57.6 | 0.56 |
| 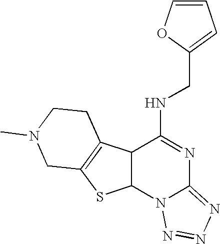 #NAME? | 864825 | 96.4 | 96.4 | 1.26 |
| 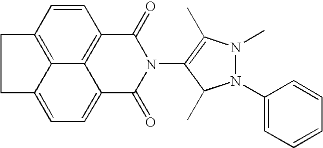 #NAME? | 7978392 | 111.5 | 111.5 | 0.39 |
| 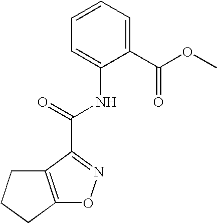 #NAME? | 4245926 | 72.8 | 72.8 | 0.46 |
| 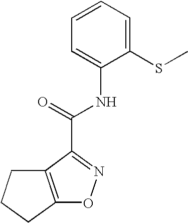 #NAME? | 4244309 | 75.7 | 75.7 | 0.44 |
| 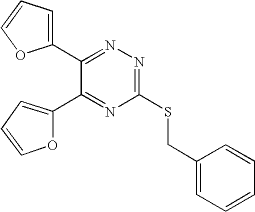 #NAME? | 4260465 | 93.8 | 93.8 | 0.72 |
| 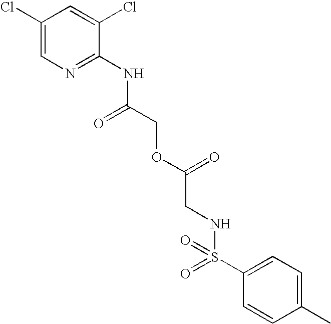 #NAME? | 3711819 | 63.4 | 63.4 | 0.8 |
| 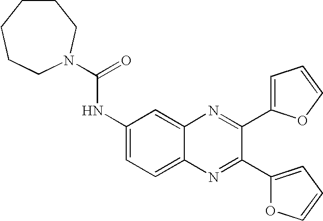 #NAME? | 4258795 | 83.2 | 83.2 | 0.96 |
| 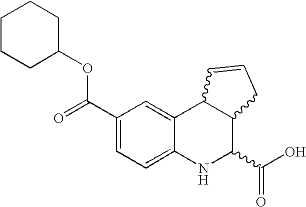 #NAME? | 4249621 | 78.5 | 78.5 | 0.38 |
| 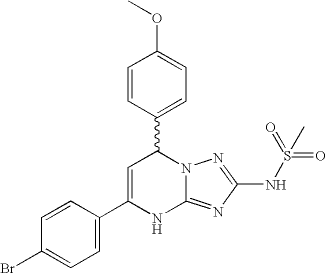 #NAME? | 17401655 | 54.6 | 54.6 | 1.46 |
| 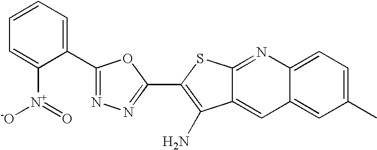 #NAME? | 17410550 | 52.7 | 52.7 | 0.7 |
| 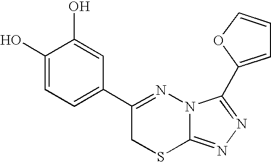 #NAME? | 17401638 | 56.6 | 56.6 | 0.82 |
| 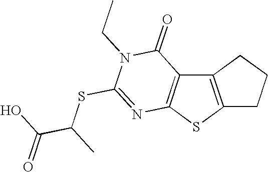 #NAME? | 856662 | 95.1 | 95.1 | 0.59 |
| 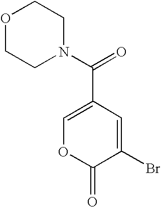 #NAME? | 860621 | 84.9 | 84.9 | 0.55 |
| 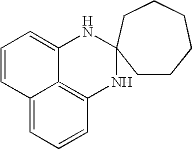 #NAME? | 4263637 | 64.3 | 64.3 | 0.58 |
| 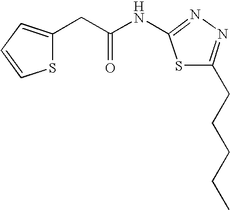 #NAME? | 4259217 | 69.2 | 69.2 | 0.51 |
| 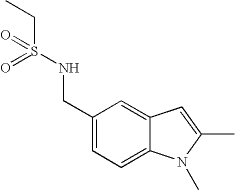 #NAME? | 14733498 | 51.8 | 51.8 | 1 |
| 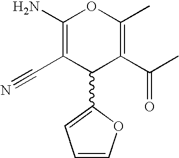 #NAME? | 857779 | 65.4 | 65.4 | 1.13 |
| 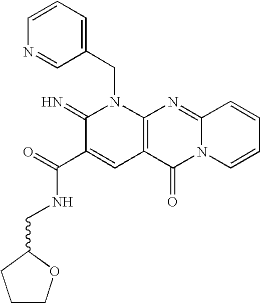 #NAME? | 844196 | 63.4 | 63.4 | 0.93 |
| 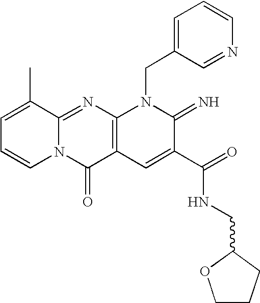 #NAME? | 849581 | 60.6 | 60.6 | 0.89 |
| 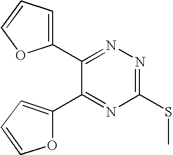 #NAME? | 859770 | 107 107 | 0.53 | |
| 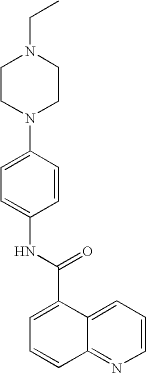 #NAME? | 842582 | 76.3 | 76.3 | 0.21 |
| 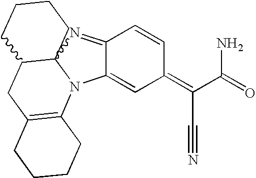 #NAME? | 856266 | 74.3 | 74.3 | 0.62 |
| 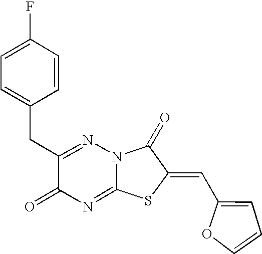 #NAME? | 863170 | 58.4 | 58.4 | 0.32 |
| 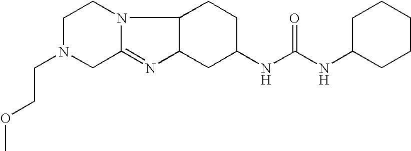 #NAME? | 865452 | 79.7 | 79.7 | 0.73 |
| 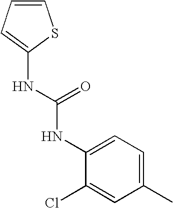 #NAME? | 14734202 | 66.3 | 66.3 | 1.39 |
| 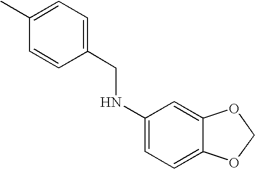 #NAME? | 4257119 | 50.6 | 50.6 | 1.2 |
| 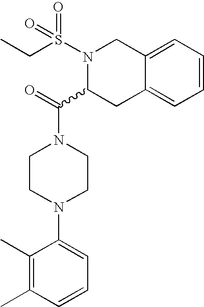 #NAME? | 3712504 | 54.6 | 54.7 | 0.25 |
| 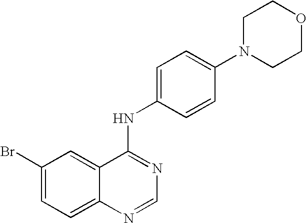 #NAME? | 7975247 | 86.8 | 86.8 | 0.87 |
| 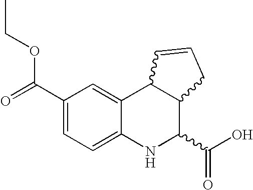 #NAME? | 4263567 | 111.5 | 111.5 | 0.4 |
| 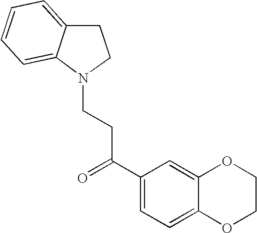 #NAME? | 857910 | 79.4 | 79.4 | 0.24 |
| 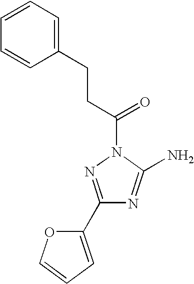 #NAME? | 4254649 | 63.1 | 63.2 | 0.83 |
| 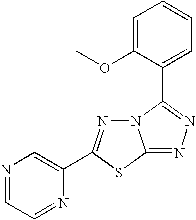 #NAME? | 4265558 | 75.3 | 75.3 | 0.59 |
| 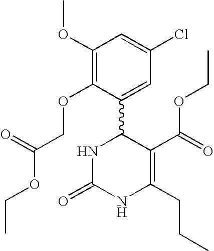 #NAME? | 4262430 | 50.9 | 50.9 | 1.19 |
| 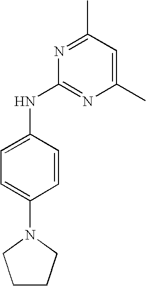 #NAME? | 4256031 | 108.1 | 108.1 | 0.2 |
| 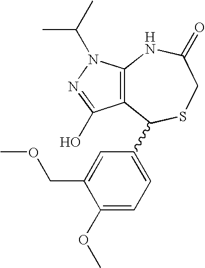 #NAME? | 4258907 | 67.1 | 67.1 | 1.2 |
| 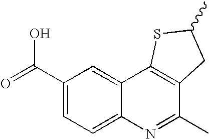 #NAME? | 4259932 | 54.7 | 54.7 | 0.6 |
| 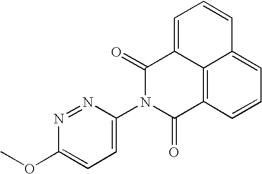 #NAME? | 4265126 | 58.8 | 58.8 | 0.88 |
| 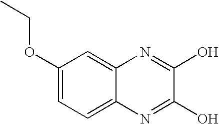 #NAME? | 4261310 | 69.6 | 69.6 | 1.46 |
| 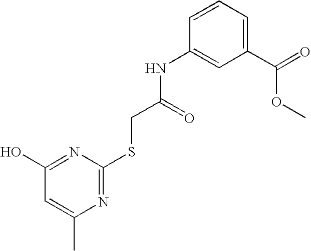 #NAME? | 4262437 | 59.9 | 59.9 | 0.64 |
| 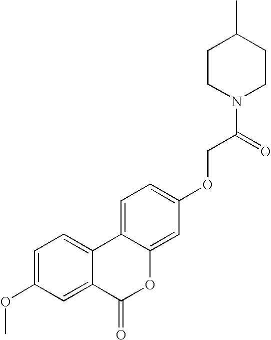 #NAME? | 4257486 | 104.8 | 104.8 | 0.49 |
| 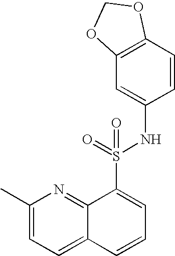 #NAME? | 4262919 | 80.6 | 80.6 | 0.67 |
| 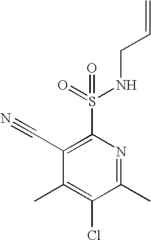 #NAME? | 4251530 | 69.9 | 69.9 | 0.59 |
| 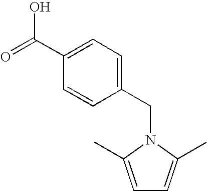 #NAME? | 858431 | 76.8 | 76.8 | 0.63 |
| 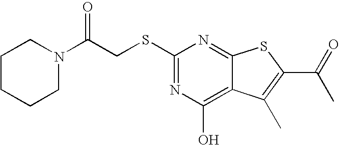 #NAME? | 4259313 | 57 | 57 | 0.82 |
| 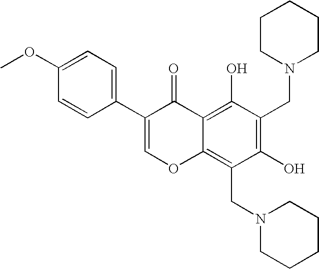 #NAME? | 4244723 | 82.5 | 82.5 | 0.51 |
| 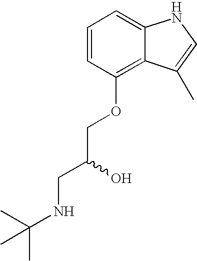 #NAME? | 856249 | 52.2 | 52.2 | 0.65 |
| 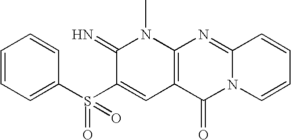 #NAME? | 859963 | 50.4 | 50.4 | 1.49 |
| 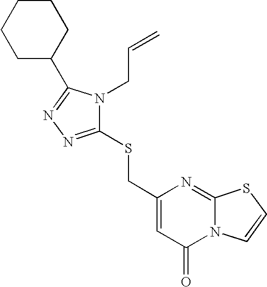 #NAME? | 3716212 | 54.8 | 54.8 | 0.6 |
| 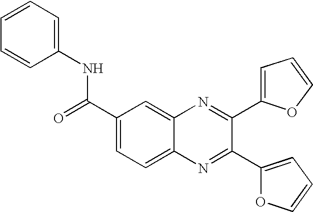 #NAME? | 4258186 | 52.2 | 52.2 | 0.88 |
| 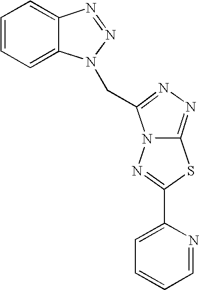 #NAME? | 865899 | 82.6 | 82.6 | 1.32 |
| 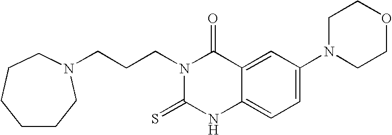 #NAME? | 4251175 | 62.3 | 62.3 | 0.65 |
| 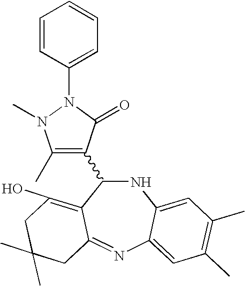 #NAME? | 4265815 | 58.3 | 58.3 | 1.46 |
| 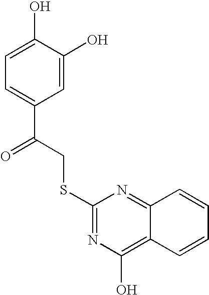 #NAME? | 4242102 | 64.2 | 64.2 | 0.57 |
| 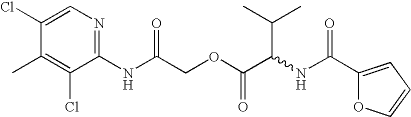 #NAME? | 3711777 | 108.1 | 108.1 | 0.66 |
| 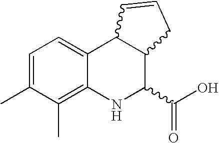 #NAME? | 844096 | 98.3 | 98.3 | 0.53 |
| 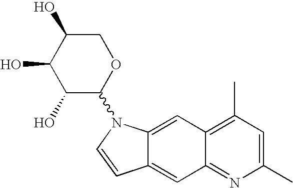 #NAME? | 4247900 | 52.1 | 52.1 | 1.1 |
| 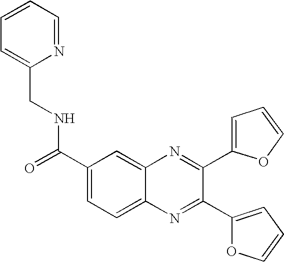 #NAME? | 4245216 | 107.1 | 107.1 | 1.18 |
| 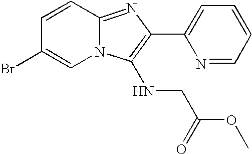 #NAME? | 4251133 | 51.2 | 51.2 | 1.16 |
| 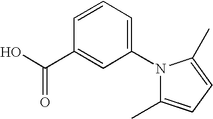 #NAME? | 4257870 | 95.8 | 95.8 | 0.65 |
| 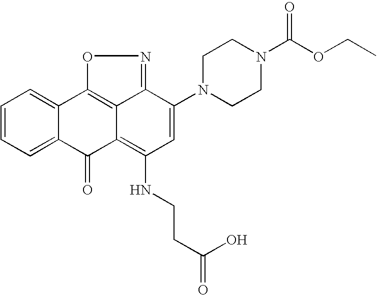 #NAME? | 4252106 | 78.1 | 78.1 | 0.86 |
| 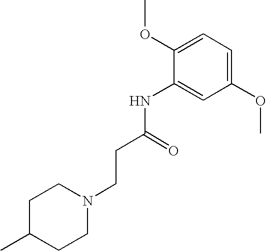 #NAME? | 846609 | 73 | 73 | 0.65 |
| 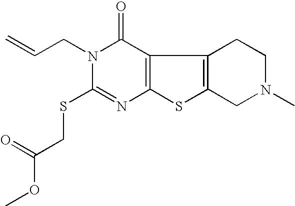 #NAME? | 856802 | 53.2 | 53.2 | 1.23 |
| 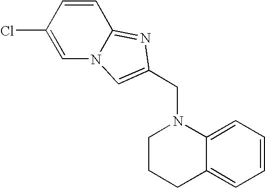 #NAME? | 3711377 | 61.3 | 61.3 | 0.75 |

| | | | | SourcePlate |
|---|---|---|---|---|
| STRUCTURE | VendorCompoundID | PlateID | WellID | ID |
| 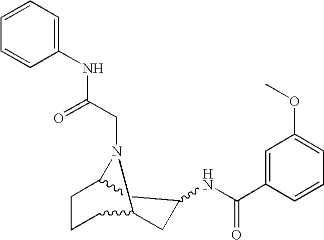 #NAME? | ASN 06417932 | PN-001309.S01 | N24 | ML0000007195 |
| 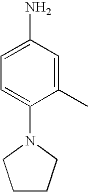 #NAME? | T5247192 | PN-001310.S01 | J22 | ML0000007197 |
| 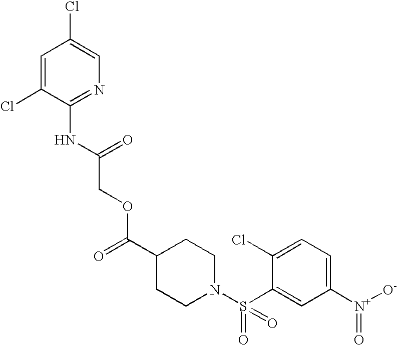 #NAME? | T0510-9152 | PN-001312.S01 | A03 | ML0000007201 |
| 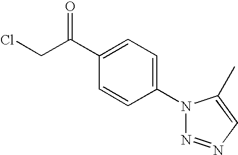 #NAME? | T0500-7689 | PN-001312.S01 | E11 | ML0000007201 |
| 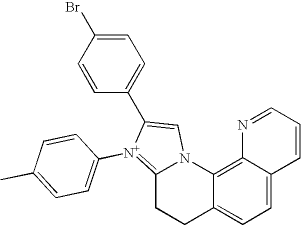 #NAME? | T0503-5937 | PN-001312.S01 | M14 | ML0000007201 |
| 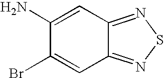 #NAME? | BAS 00253421 | PN-001315.S01 | D11 | ML0000007207 |
| 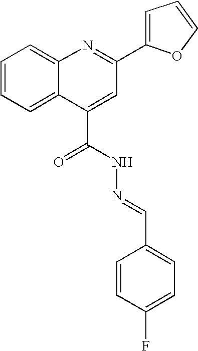 #NAME? | AK-968/41025763 | PN-001317.S01 | B06 | ML0000007211 |
| 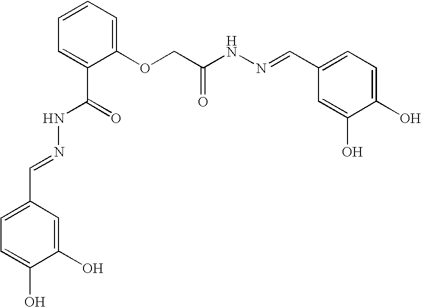 #NAME? | AG-205/09891056 | PN-001317.S01 | L21 | ML0000007211 |
| 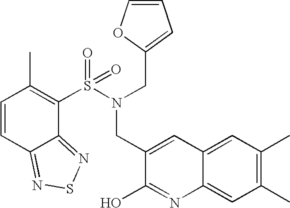 #NAME? | ASN 04194299 | PN-001318.S01 | P16 | ML0000007213 |
| 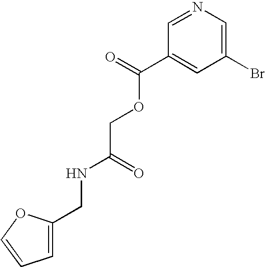 #NAME? | T0508-6314 | PN-001319.S01 | A10 | ML0000007215 |
| 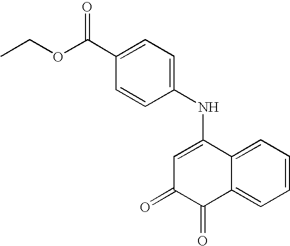 #NAME? | 8006-7595 | PN-001324.S01 | I19 | ML0000007225 |
| 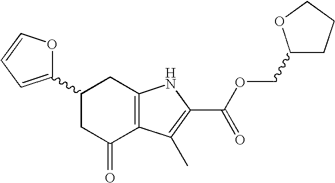 #NAME? | C276-1398 | PN-001326.S01 | O22 | ML0000007229 |
| 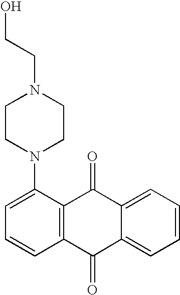 #NAME? | BAS 02282285 | PN-001330.S01 | I12 | ML0000007238 |
| 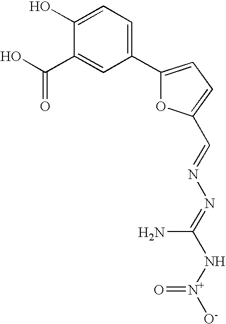 #NAME? | 5377079 | PN-001330.S01 | H10 | ML0000007238 |
| 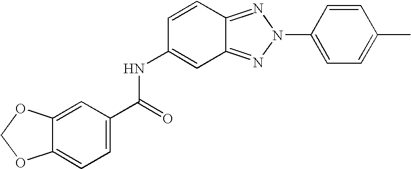 #NAME? | 7793433 | PN-001332.S01 | K05 | ML0000007242 |
| 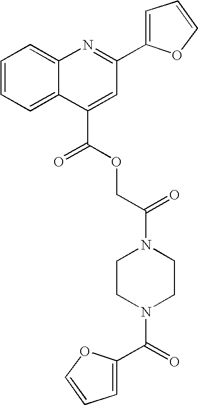 #NAME? | T0516-3143 | PN-001335.S01 | N06 | ML0000007248 |
| 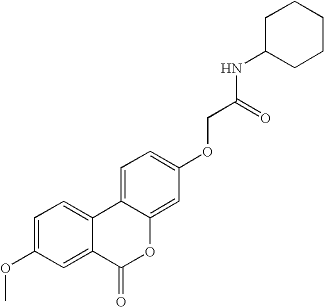 #NAME? | 7683031 | PN-001338.S01 | J21 | ML0000007254 |
| 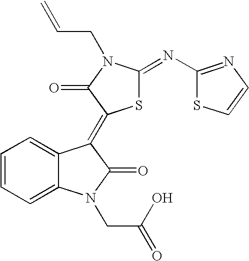 #NAME? | STOCK5S-47864 | PN-001340.S01 | C20 | ML0000007259 |
| 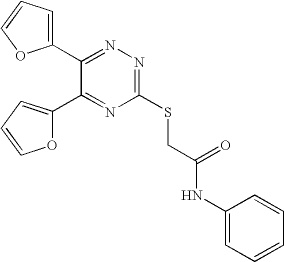 #NAME? | STOCK5S-55671 | PN-001343.S01 | A10 | ML0000007265 |
| 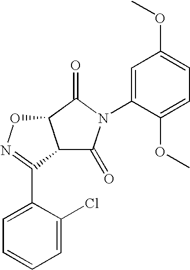 #NAME? | STOCK5S-93840 | PN-001343.S01 | D18 | ML0000007265 |
| 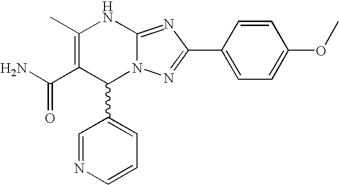 #NAME? | 5225-0199 | PN-001350.S01 | I10 | ML0000007279 |
| 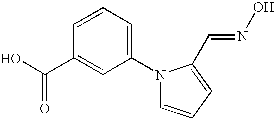 #NAME? | 5842-0941 | PN-001354.S01 | J20 | ML0000007290 |
| 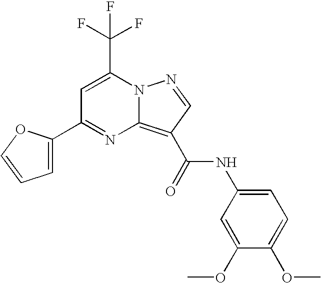 #NAME? | 5839134 | PN-001356.S01 | J10 | ML0000007294 |
| 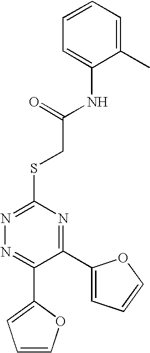 #NAME? | STOCK5S-66791 | PN-001358.S01 | A22 | ML0000007299 |
| 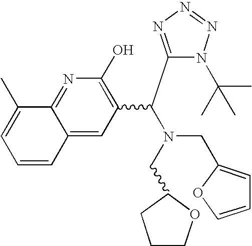 #NAME? | BAS 04419702 | PN-001358.S01 | M12 | ML0000007299 |
| 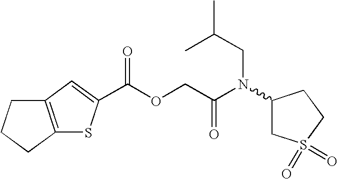 #NAME? | T5339763 | PN-001359.S01 | J15 | ML0000007301 |
| 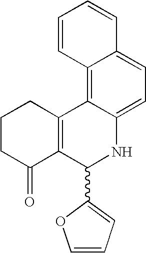 #NAME? | 6486491 | PN-001360.S01 | F03 | ML0000007303 |
| 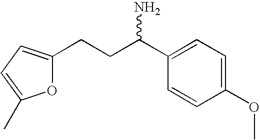 #NAME? | R162-1740 | PN-001361.S01 | E18 | ML0000007305 |
| 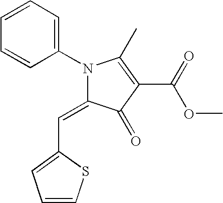 #NAME? | 5919628 | PN-001362.S01 | C23 | ML0000007307 |
| 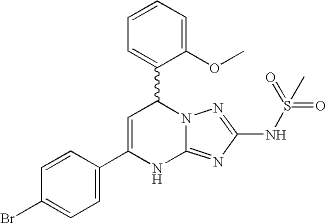 #NAME? | STOCK4S-11694 | PN-001365.S01 | C11 | ML0000007321 |
| 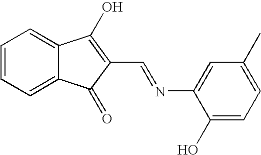 #NAME? | 5248703 | PN-001369.S01 | E12 | ML0000007329 |
| 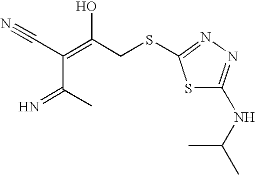 #NAME? | ZU-4312746 | PN-001369.S01 | L04 | ML0000007329 |
| 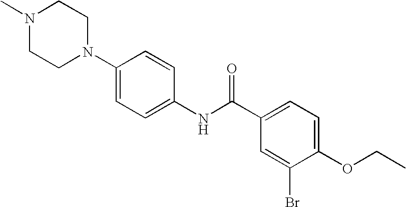 #NAME? | 7665342 | PN-001369.S01 | O15 | ML0000007329 |
| 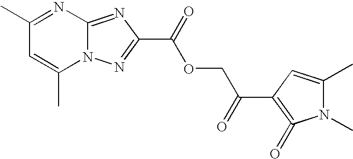 #NAME? | T5274202 | PN-001370.S01 | O10 | ML0000007331 |
| 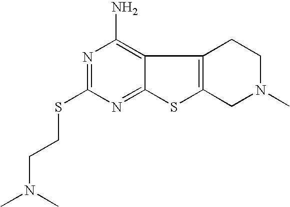 #NAME? | STOCK4S-59419 | PN-001370.S01 | O16 | ML0000007331 |
| 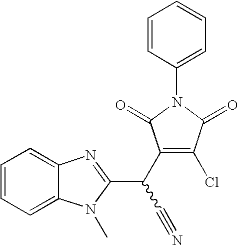 #NAME? | T0502-0502 | PN-001371.S01 | A21 | ML0000007333 |
| 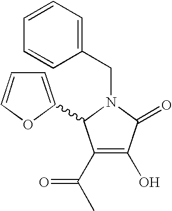 #NAME? | BAS 05211164 | PN-001371.S01 | D23 | ML0000007333 |
| 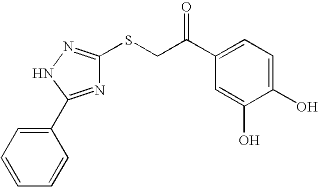 #NAME? | BAS 03014739 | PN-001372.S01 | E20 | ML0000007335 |
| 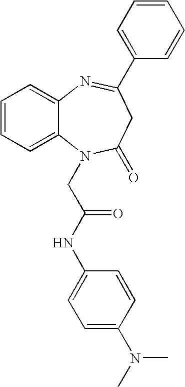 #NAME? | ASN 09400529 | PN-001372.S01 | F15 | ML0000007335 |
| 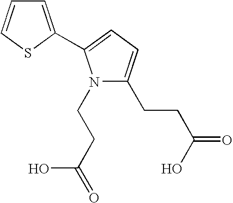 #NAME? | BAS 06347781 | PN-001373.S01 | A12 | ML0000007337 |
| 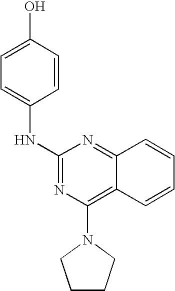 #NAME? | BAS 04913588 | PN-001373.S01 | C16 | ML0000007337 |
| 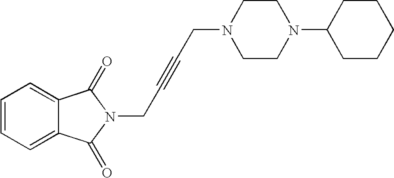 #NAME? | STOCK1S-03427 | PN-001375.S01 | G09 | ML0000007341 |
| 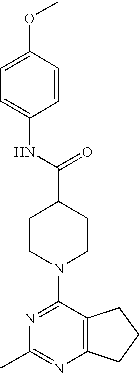 #NAME? | ASN 09857998 | PN-001375.S01 | N21 | ML0000007341 |
| 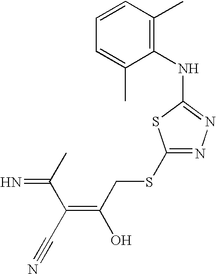 #NAME? | T5228553 | PN-001376.S01 | H04 | ML0000007343 |
| 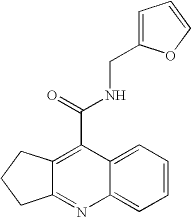 #NAME? | T5275921 | PN-001376.S01 | H06 | ML0000007343 |
| 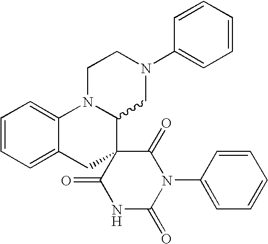 #NAME? | STOCK5S-55833 | PN-001376.S01 | K06 | ML0000007343 |
| 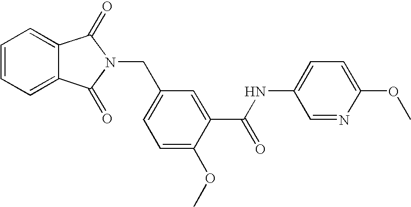 #NAME? | T5275464 | PN-001377-S01 | H03 | ML0000007345 |
| 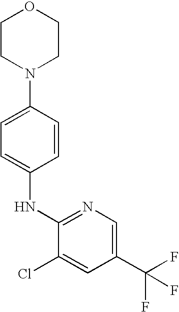 #NAME? | T5337184 | PN-001377.S01 | O12 | ML0000007345 |
| 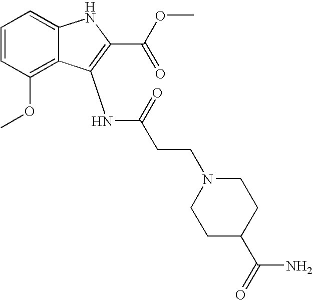 #NAME? | STOCK4S-77697 | PN-001379.S01 | P04 | ML0000007349 |
| 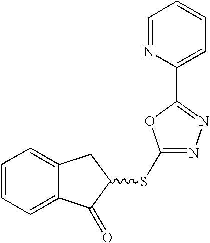 #NAME? | ASN 06347021 | PN-001380.S01 | H11 | ML0000007351 |
| 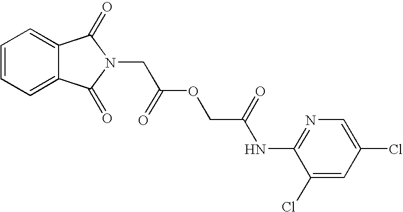 #NAME? | T0513-0062 | PN-001380.S01 | J06 | ML0000007351 |
| 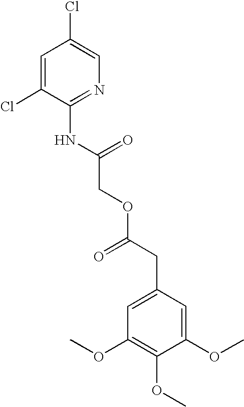 #NAME? | T0512-9804 | PN-001380.S01 | L16 | ML0000007351 |
| 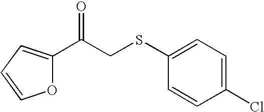 #NAME? | T0506-1917 | PN-001382.S01 | P06 | ML0000007355 |
| 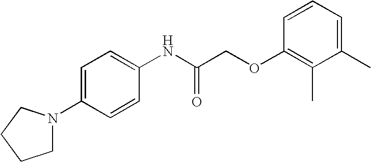 #NAME? | 6713361 | PN-001383.S01 | M19 | ML0000007357 |
| 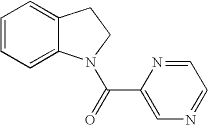 #NAME? | T5304647 | PN-001386.S01 | A11 | ML0000007363 |
| 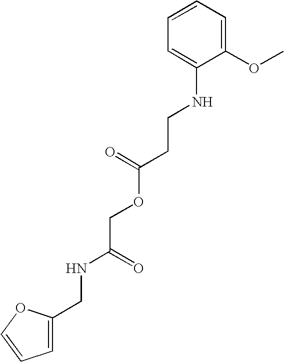 #NAME? | T0509-6338 | PN-001386.S01 | A20 | ML0000007363 |
| 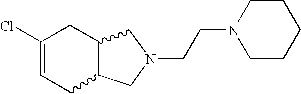 #NAME? | STOCK3S-91670 | PN-001386.S01 | L13 | ML0000007363 |
| 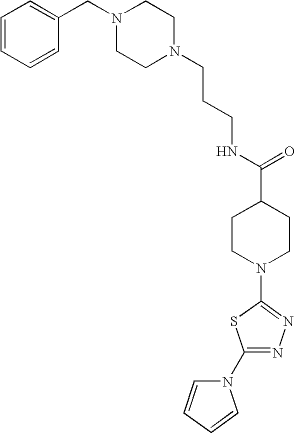 #NAME? | C857-0187 | PN-001388.S01 | F09 | ML0000007367 |
| 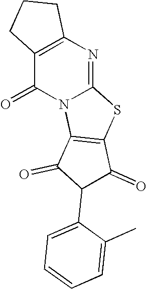 #NAME? | 6649044 | PN-001388.S01 | K14 | ML0000007367 |
| 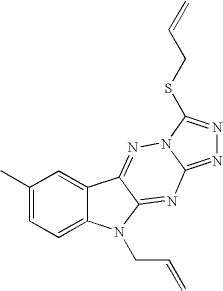 #NAME? | 6377727 | PN-001388.S01 | O14 | ML0000007367 |
| 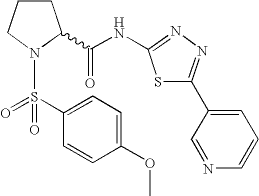 #NAME? | T0503-7007 | PN-001391.S01 | A03 | ML0000007373 |
| 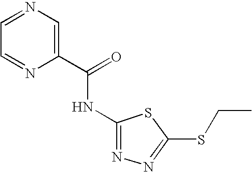 #NAME? | 5895750 | PN-001391.S01 | D09 | ML0000007373 |
| 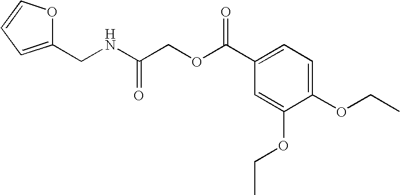 #NAME? | T0508-6267 | PN-001393.S01 | O12 | ML0000007377 |
| 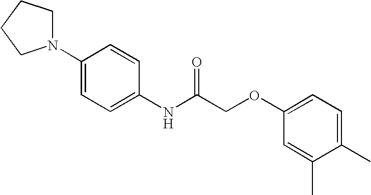 #NAME? | 6732698 | PN-001395.S01 | A04 | ML0000007381 |
| 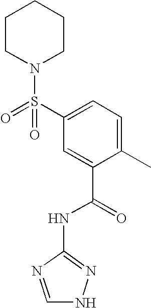 #NAME? | T0506-1245 | PN-001395.S01 | H04 | ML0000007381 |
| 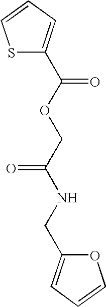 #NAME? | T0506-8050 | PN-001396.S01 | G07 | ML0000007383 |
| 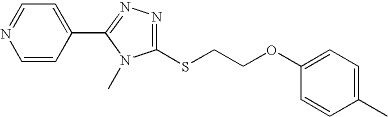 #NAME? | T5272611 | PN-001398.S01 | L07 | ML0000007383 |
| 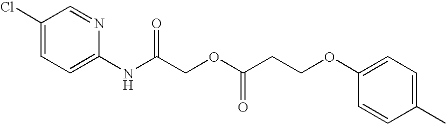 #NAME? | T5238252 | PN-001396.S01 | O06 | ML0000007383 |
| 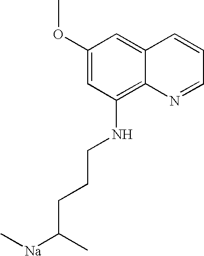 #NAME? | BAS 00347621 | PN-001397.S01 | A13 | ML0000007385 |
| 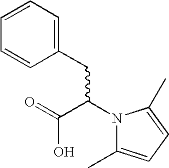 #NAME? | BAS 01191302 | PN-001290.S01 | G14 | ML0000007142 |
| 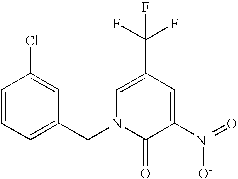 #NAME? | 8E-503 | PN-001295.S01 | J24 | ML0000007154 |
| 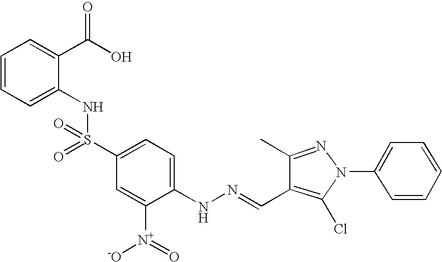 #NAME? | T0516-5863 | PN-001296.S01 | O16 | ML0000007156 |
| 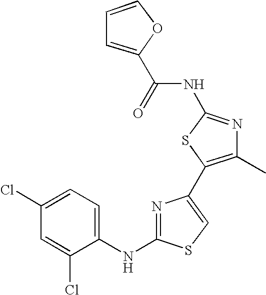 #NAME? | CLY_Corr_E05b | PN-001298.S01 | H14 | ML0000007166 |
| 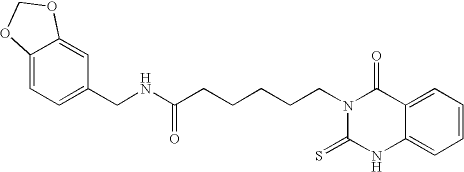 #NAME? | K284-1423 | PN-001200.S01 | H12 | ML0000007172 |
| 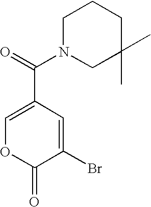 #NAME? | STOCK4S-53737 | PN-001305.S01 | C10 | ML0000007187 |
| 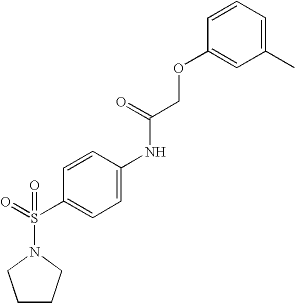 #NAME? | 6449957 | PN-001408.S01 | N19 | ML0000007407 |
| 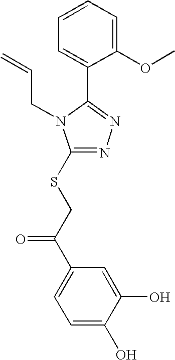 #NAME? | STOCK4S-90308 | PN-001409.S01 | D19 | ML0000007409 |
| 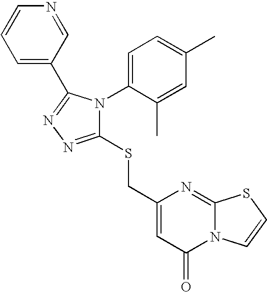 #NAME? | ZT-4757552 | PN-001409.S01 | F11 | ML0000007409 |
| 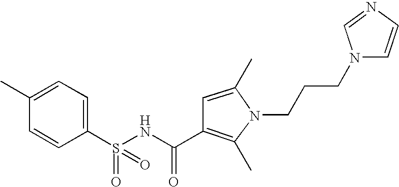 #NAME? | T5234796 | PN-001409.S01 | H10 | ML0000007409 |
| 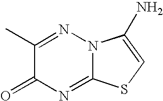 #NAME? | BAS 04280313 | PN-001410.S01 | E11 | ML0000007411 |
| 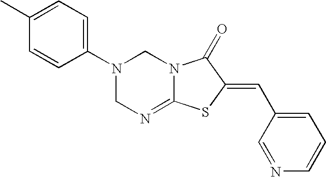 #NAME? | STOCK2S-30514 | PN-001410.S01 | E24 | ML0000007411 |
| 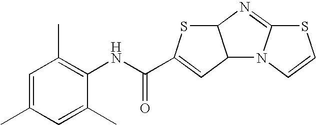 #NAME? | T5229528 | PN-001411.S01 | L20 | ML0000007413 |
| 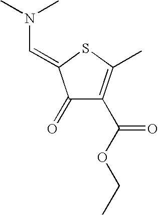 #NAME? | 6050623 | PN-001413.S01 | B21 | ML0000007417 |
| 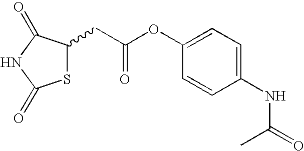 #NAME? | STOCK3S-51792 | PN-001416.S01 | C12 | ML0000007423 |
| 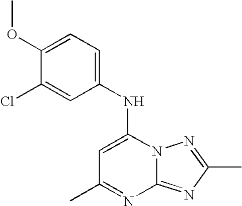 #NAME? | C301-0787 | PN-001420.S01 | B03 | ML0000007431 |
| 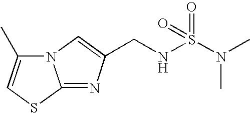 #NAME? | F0690-0118 | PN-001420.S01 | I11 | ML0000007431 |
| 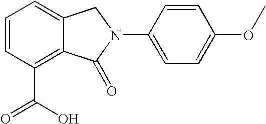 #NAME? | C429-0410 | PN-001420.S01 | P16 | ML0000007431 |
| 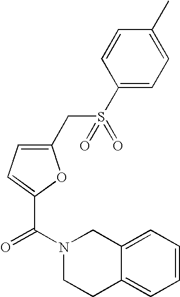 #NAME? | C521-0121 | PN-001421.S01 | I10 | ML0000007433 |
| 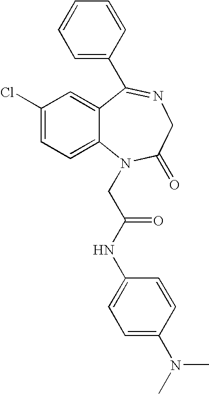 #NAME? | K788-4835 | PN-001423.S01 | D07 | ML0000007437 |
| 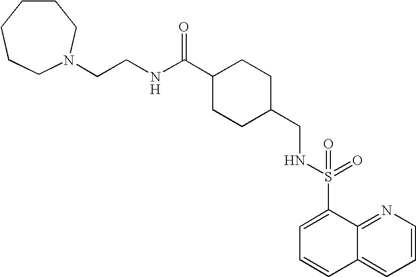 #NAME? | C737-1720 | PN-001425.S01 | G16 | ML0000007441 |
| 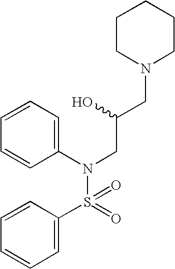 #NAME? | STOCK3S-45702 | PN-001427.S01 | B24 | ML0000007445 |
| 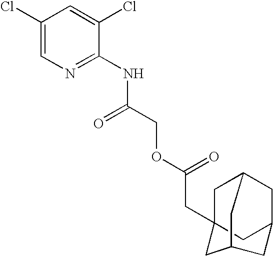 #NAME? | T0513-0723 | PN-001427.S01 | G07 | ML0000007445 |
| 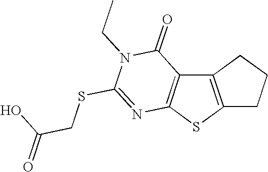 #NAME? | STOCK1S-29467 | PN-001428.S01 | I13 | ML0000007447 |
| 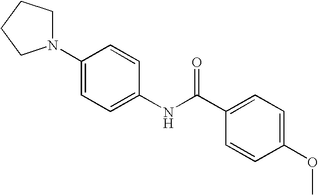 #NAME? | 6763181 | PN-001429.S01 | D07 | ML0000007449 |
| 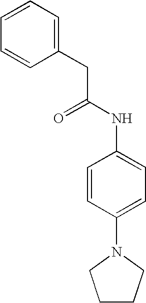 #NAME? | 6776639 | PN-001429.S01 | D23 | ML0000007449 |
| 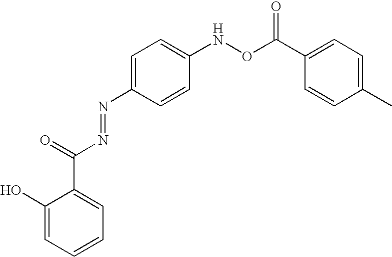 #NAME? | 4234-0342 | PN-001432.S01 | A11 | ML0000007455 |
| 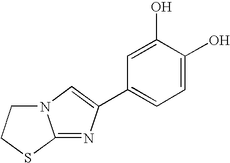 #NAME? | 5981208 | PN-001432.S01 | D04 | ML0000007455 |
| 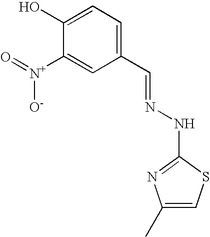 #NAME? | 6159280 | PN-001432.S01 | P07 | ML0000007455 |
| 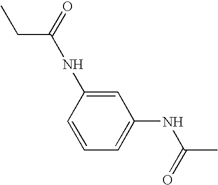 #NAME? | 5836613 | PN-001434.S01 | F12 | ML0000007459 |
| 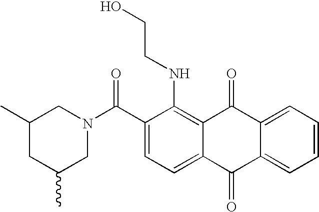 #NAME? | C092-0016 | PN-001434.S01 | I05 | ML0000007459 |
| 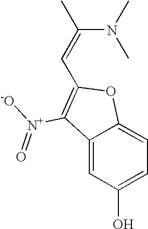 #NAME? | 5266966 | PN-001434.S01 | N18 | ML0000007459 |
| 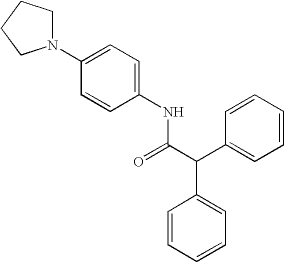 #NAME? | STOCK3S-20539 | PN-001435.S01 | H14 | ML0000007461 |
| 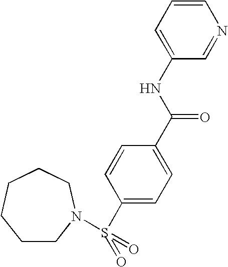 #NAME? | T5322635 | PN-001436.S01 | J07 | ML0000007463 |
| 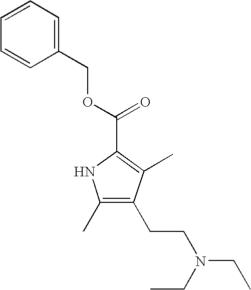 #NAME? | BAS 02787969 | PN-001436.S01 | K18 | ML0000007463 |
| 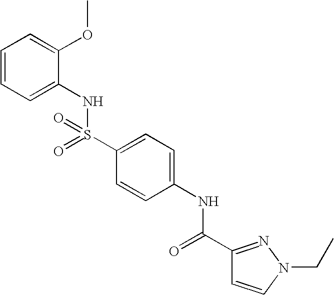 #NAME? | 6049-2386 | PN-001436.S01 | O24 | ML0000007463 |
| 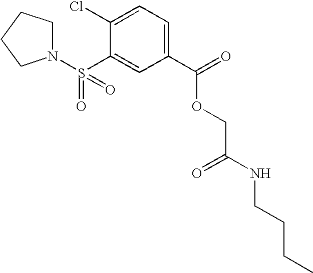 #NAME? | T0516-3159 | PN-001436.S01 | P04 | ML0000007463 |
| 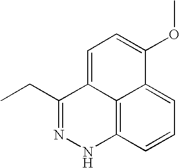 #NAME? | 5921611 | PN-001463.S01 | P19 | ML0000007463 |
| 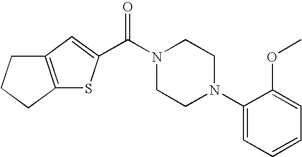 #NAME? | T5302686 | PN-001440.S01 | A18 | ML0000007473 |
| 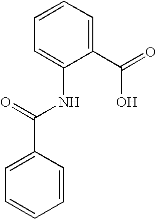 #NAME? | BAS 00077860 | PN-001441.S01 | F13 | ML0000007475 |
| 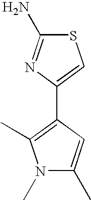 #NAME? | T5302811 | PN-001442.S01 | O08 | ML0000007477 |
| 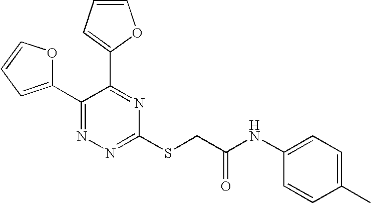 #NAME? | STOCK3S-58114 | PN-001400.S01 | C13 | ML0000007391 |
| 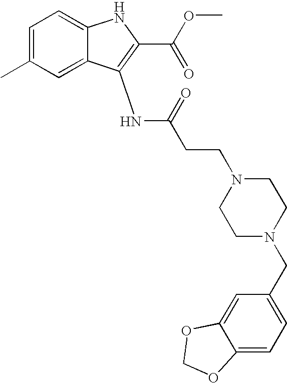 #NAME? | STOCK4S-35398 | PN-001400.S01 | I16 | ML0000007391 |
| 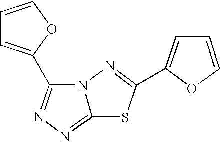 #NAME? | BAS 09636908 | PN-001402.S01 | B20 | ML0000007395 |
| 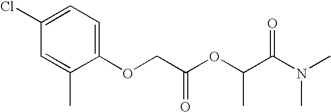 #NAME? | STOCK1S-50800 | PN-001404.S01 | C10 | ML0000007399 |
| 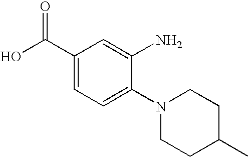 #NAME? | BAS 05307648 | PN-001404.S01 | N06 | ML0000007399 |
| 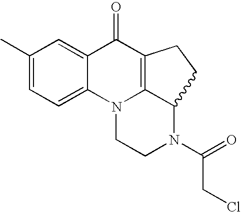 #NAME? | STOCK2S-06744 | PN-001445.S01 | A06 | ML0000007483 |
| 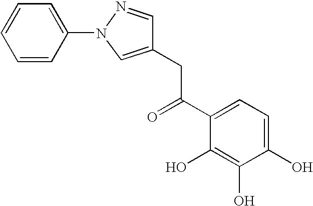 #NAME? | 6680720 | PN-001445.S01 | K15 | ML0000007483 |
| 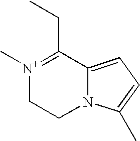 #NAME? | 5101962 | PN-001447.S01 | J20 | ML0000007487 |
| 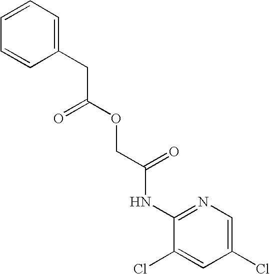 #NAME? | T0510-5451 | PN-001447.S01 | K12 | ML0000007487 |
| 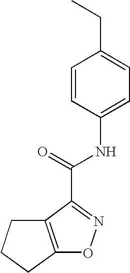 #NAME? | C226-3252 | PN-001447.S01 | L13 | ML0000007487 |
| 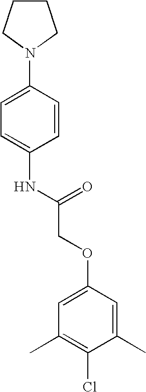 #NAME? | 6746256 | PN-001448.S01 | G16 | ML0000007489 |
| 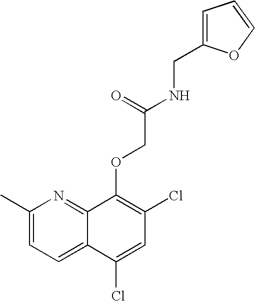 #NAME? | T0510-5554 | PN-001449.S01 | F23 | ML0000007491 |
| 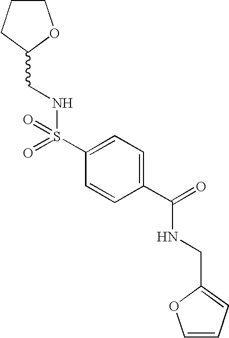 #NAME? | ASN 05061912 | PN-001449.S01 | O17 | ML0000007491 |
| 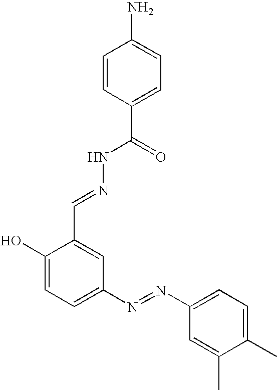 #NAME? | BAS 01231848 | PN-001450.S01 | J19 | ML0000007493 |
| 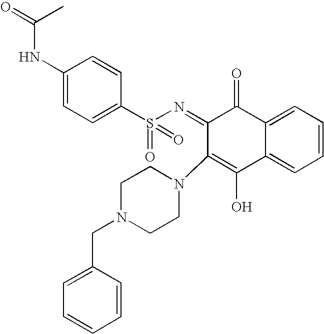 #NAME? | STOCK2S-81354 | PN-001450.S01 | O14 | ML0000007493 |
| 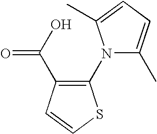 #NAME? | 7R-0044 | PN-001451.S01 | J19 | ML0000007495 |
| 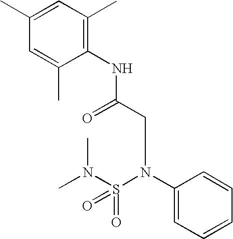 #NAME? | 7965886 | PN-001452.S01 | F18 | ML0000007497 |
| 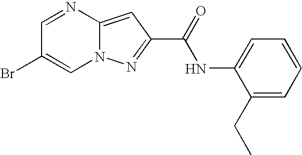 #NAME? | 7979933 | PN-001452.S01 | G12 | ML0000007497 |
| 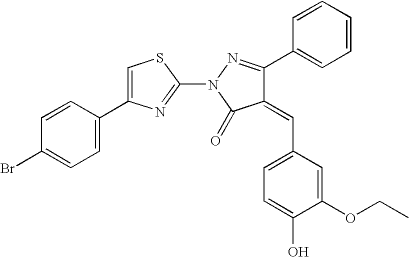 #NAME? | 5800688 | PN-001452.S01 | I23 | ML0000007497 |
| 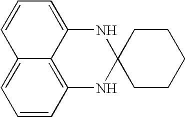 #NAME? | BAS 00631059 | PN-001452.S01 | L05 | ML0000007497 |
| 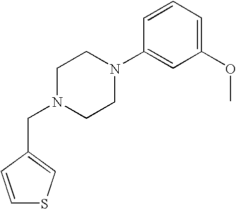 #NAME? | 5419872 | PN-001452.S01 | L06 | ML0000007497 |
| 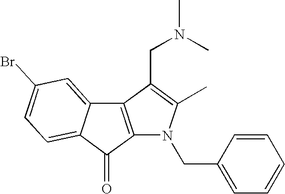 #NAME? | STOCK4S-50075 | PN-001452.S01 | O13 | ML0000007497 |
| 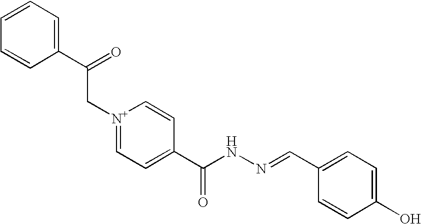 #NAME? | 5481530 | PN-001452.S01 | P06 | ML0000007497 |
| 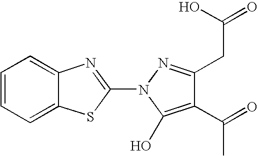 #NAME? | 7525499 | PN-001452.S01 | P24 | ML0000007497 |
| 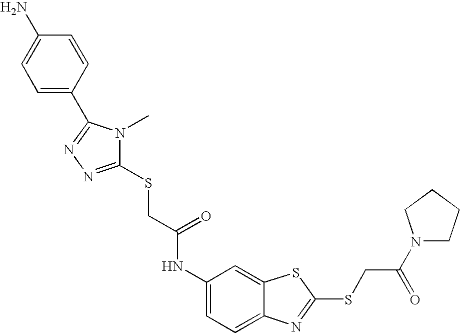 #NAME? | AN-988/15131278 | PN-001453.S01 | C17 | ML0000007499 |
| 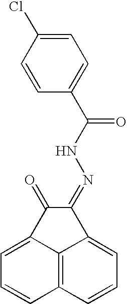 #NAME? | T0519-5270 | PN-001453.S01 | L23 | ML0000007499 |
| 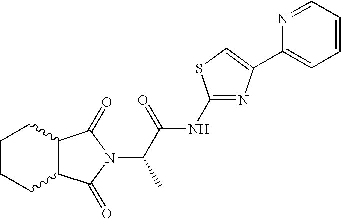 #NAME? | T5292641 | PN-001455.S01 | C23 | ML0000007503 |
| 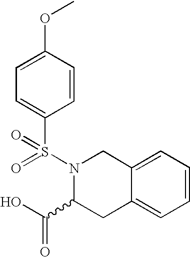 #NAME? | T0514-5204 | PN-001458.S01 | O19 | ML0000007509 |
| 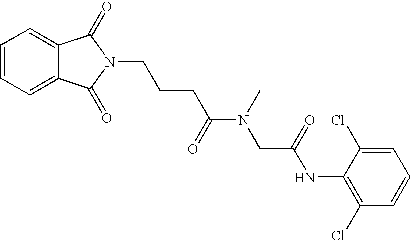 #NAME? | T5297657 | PN-001480.S01 | A23 | ML0000007513 |
| 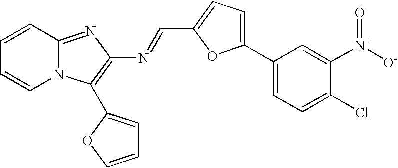 #NAME? | AG-690/36158020 | PN-001460.S01 | C11 | ML0000007513 |
| 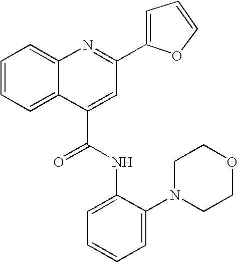 #NAME? | T0506-7588 | PN-001460.S01 | G12 | ML0000007513 |
| 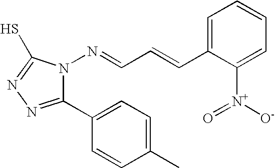 #NAME? | STOCK3S-19552 | PN-001460.S01 | M12 | ML0000007513 |
| 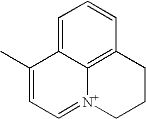 #NAME? | T0400-2347 | PN-001462.S01 | A08 | ML0000007517 |
| 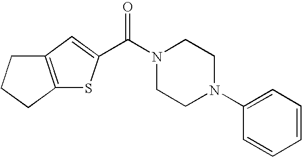 #NAME? | T5327864 | PN-001462.S01 | A10 | ML0000007517 |
| 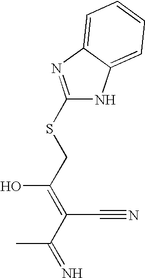 #NAME? | T5213479 | PN-001462.S01 | B17 | ML0000007517 |
| 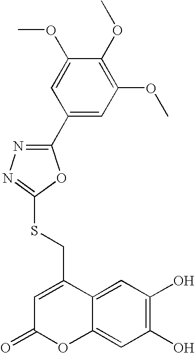 #NAME? | T5216715 | PN-001462.S01 | B19 | ML0000007517 |
| 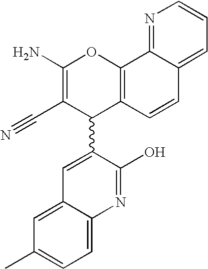 #NAME? | ASN 02750734 | PN-001462.S01 | D16 | ML0000007517 |
| 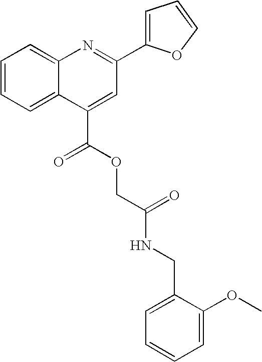 #NAME? | T5275583 | PN-001452.S01 | F17 | ML0000007517 |
| 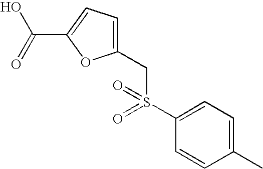 #NAME? | K780-0463 | PN-001463.S01 | H23 | ML0000007519 |
| 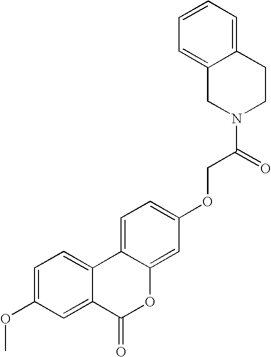 #NAME? | 7692727 | PN-001463.S01 | M11 | ML0000007519 |
| 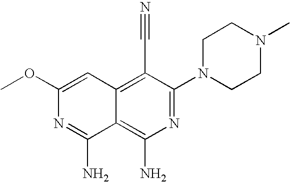 #NAME? | T0508-2714 | PN-001464.S01 | I19 | ML0000007521 |
| 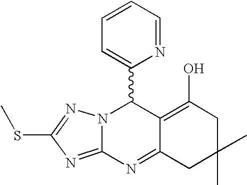 #NAME? | STOCK5S-53253 | PN-001465.S01 | P05 | ML0000007523 |
| 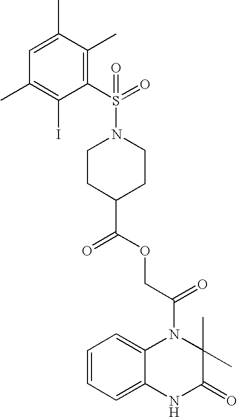 #NAME? | T5297301 | PN-001467.S01 | D24 | ML0000007527 |
| 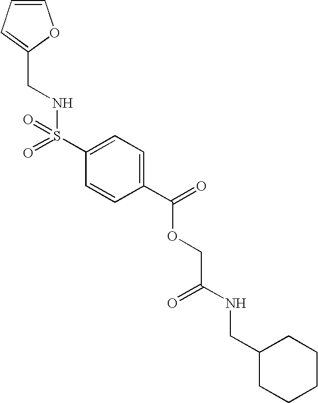 #NAME? | T5244807 | PN-001467.S01 | J06 | ML0000007527 |
| 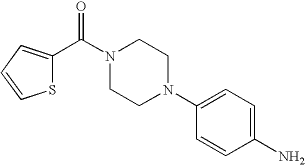 #NAME? | BAS 03236849 | PN-001467.S01 | K09 | ML0000007527 |
| 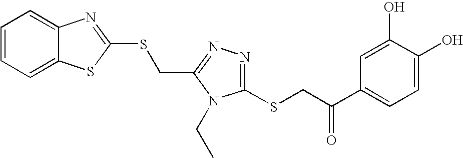 #NAME? | T5235502 | PN-001467.S01 | M14 | ML0000007527 |
| 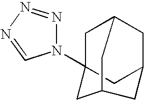 #NAME? | BAS 02889328 | PN-001469.S01 | B20 | ML0000007531 |
| 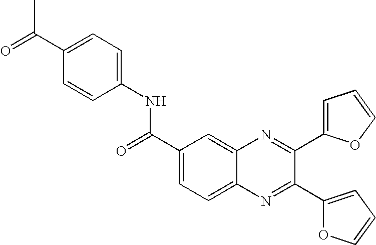 #NAME? | 7097922 | PN-001469.S01 | K05 | ML0000007531 |
| 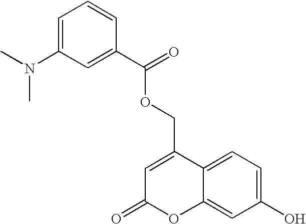 #NAME? | T5237824 | PN-001470.S01 | F21 | ML0000007533 |
| 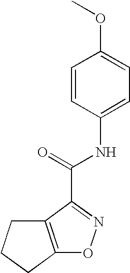 #NAME? | C226-3282 | PN-001470.S01 | L16 | ML0000007533 |
| 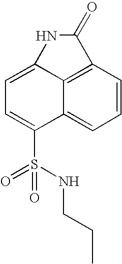 #NAME? | BAS 00460194 | PN-001472.S01 | I17 | ML0000007537 |
| 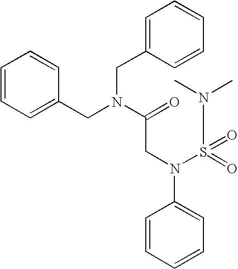 #NAME? | 7938889 | PN-001474.S01 | D22 | ML0000007541 |
| 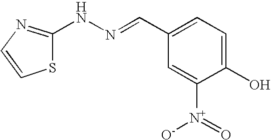 #NAME? | 6359947 | PN-001475.S01 | J07 | ML0000007543 |
| 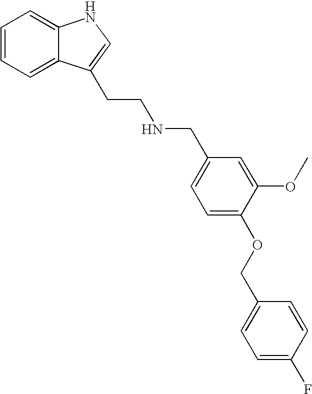 #NAME? | AP-970/42897565 | PN-001475.S01 | E10 | ML0000007545 |
| 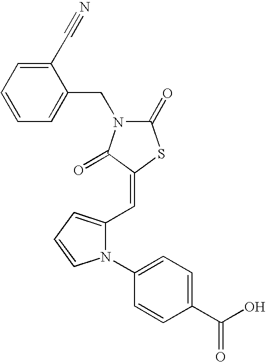 #NAME? | AH-487/42143412 | PN-001478.S01 | E11 | ML0000007545 |
| 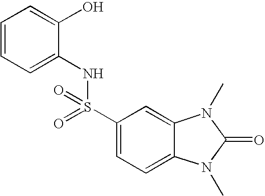 #NAME? | BAS 09106162 | PN-001476.S01 | L21 | ML0000007545 |
| 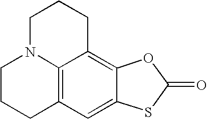 #NAME? | 6231943 | PN-001477.S01 | L17 | ML0000007546 |
| 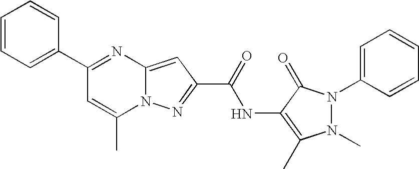 #NAME? | BAS 00481591 | PN-001478.S01 | D23 | ML0000007548 |
| 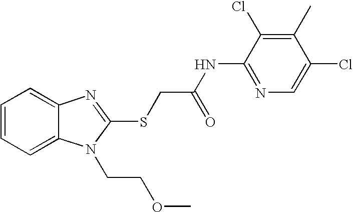 #NAME? | T5226074 | PN-001478.S01 | G13 | ML0000007548 |
| 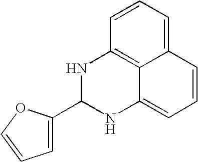 #NAME? | BAS 00922281 | PN-001478.S01 | J04 | ML0000007548 |
| 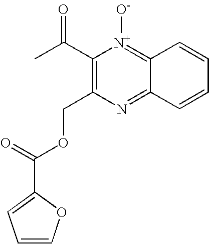 #NAME? | STOCK4S-86442 | PN-001480.S01 | P09 | ML0000007552 |
| 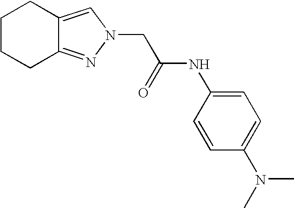 #NAME? | C881-0493 | PN-001536.S01 | C18 | ML0000007669 |
| 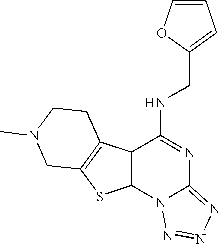 #NAME? | STOCK5S-30189 | PN-001538.S01 | P19 | ML0000007872 |
| 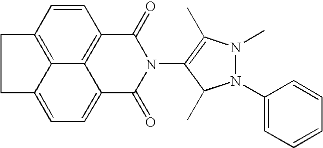 #NAME? | 5924692 | PN-001540.S01 | O20 | ML0000007675 |
| 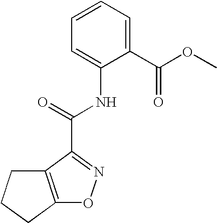 #NAME? | C226-3251 | PN-001542.S01 | H03 | ML0000007879 |
| 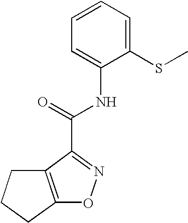 #NAME? | C228-3265 | PN-001452.S01 | P05 | ML0000007579 |
| 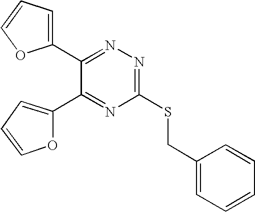 #NAME? | 7491376 | PN-001543.S01 | A16 | ML0000007681 |
| 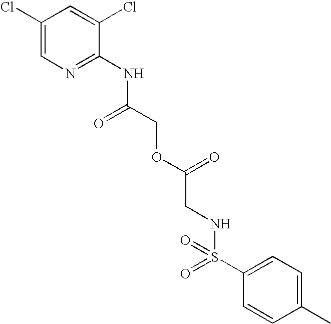 #NAME? | T0513-0552 | PN-001545.S01 | D11 | ML0000007685 |
| 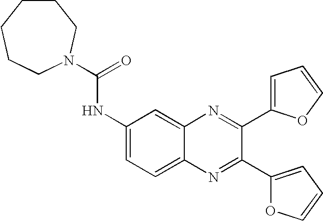 #NAME? | 7864945 | PN-001546.S01 | B20 | ML0000007687 |
| 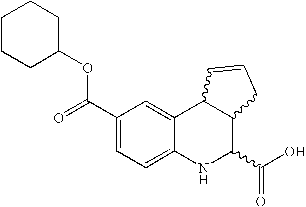 #NAME? | 4696-0791 | PN-001546.S01 | I08 | ML0000007687 |
| 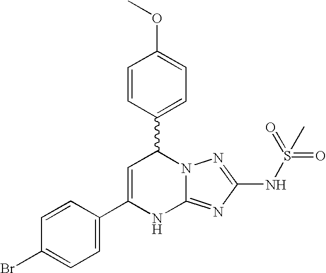 #NAME? | STOCK4S-04497 | PN-001548.S01 | D09 | ML0000010804 |
| 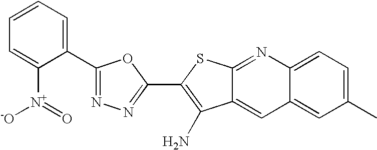 #NAME? | BAS 02240467 | PN-001548.S01 | N05 | ML0000010804 |
| 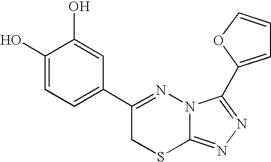 #NAME? | STOCK4S-27331 | PN-001548.S01 | P22 | ML0000010804 |
| 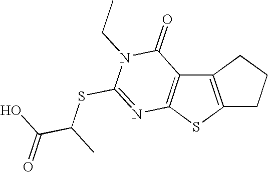 #NAME? | STOCK1S-22805 | PN-001552.S01 | F06 | ML0000010812 |
| 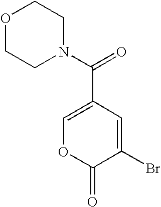 #NAME? | STOCK3S-79419 | PN-001552.S01 | G08 | ML0000010812 |
| 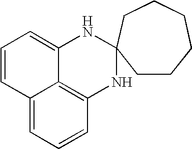 #NAME? | 6947630 | PN-001554.S01 | N13 | ML0000010816 |
| 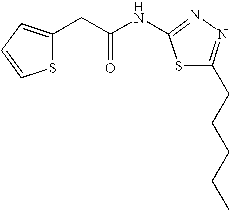 #NAME? | 7796680 | PN-001556.S01 | M08 | ML0000010820 |
| 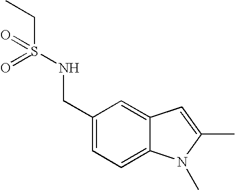 #NAME? | F0651-0388 | PN-001558.S01 | G05 | ML0000010824 |
| 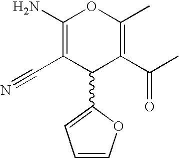 #NAME? | STOCK1S-88261 | PN-001483.S01 | F16 | ML0000007558 |
| 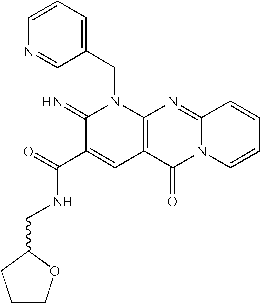 #NAME? | BAS 04995534 | PN-001483.S01 | G23 | ML0000007558 |
| 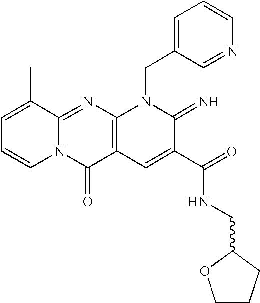 #NAME? | BAS 04995535 | PN-001483.S01 | I17 | ML0000007558 |
| 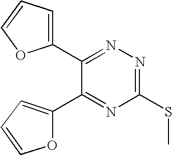 #NAME? | STOCK3S-33394 | PN-001483.S01 | N05 | ML0000007558 |
| 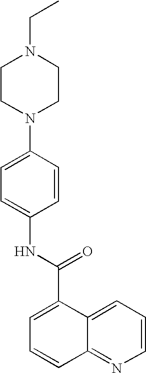 #NAME? | ASN 06896699 | PN-001483.S01 | N16 | ML0000007558 |
| 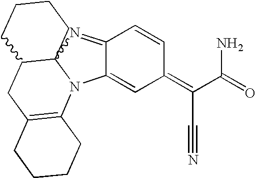 #NAME? | STOCK1S-09987 | PN-001483.S01 | O13 | ML0000007558 |
| 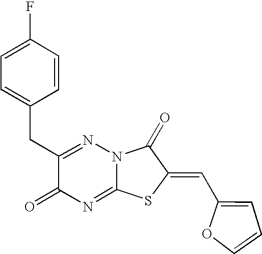 #NAME? | STOCK4S-62602 | PN-001485.S01 | K07 | ML0000007562 |
| 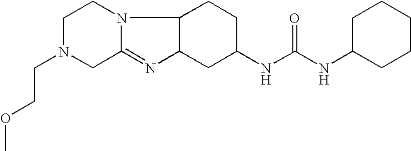 #NAME? | STOCK5S-42893 | PN-001487.S01 | L20 | ML0000007556 |
| 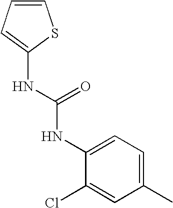 #NAME? | C795-0384 | PN-001490.S01 | N21 | ML0000007575 |
| 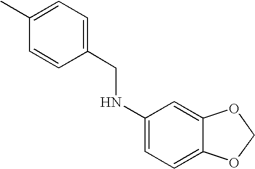 #NAME? | 5457980 | PN-001491.S01 | H24 | ML0000007579 |
| 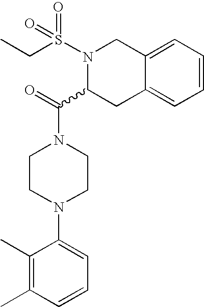 #NAME? | T5270883 | PN-001483.S01 | D20 | ML0000007583 |
| 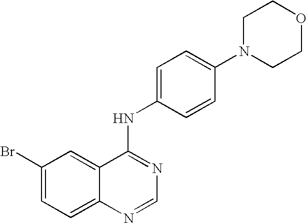 #NAME? | 6988256 | PN-001493.S01 | I11 | ML0000007583 |
| 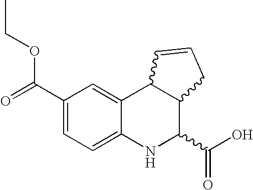 #NAME? | 5667569 | PN-001495.S01 | E21 | ML0000007587 |
| 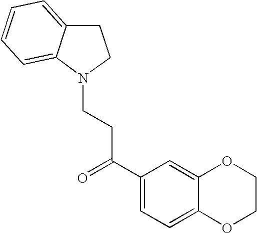 #NAME? | STOCK2S-13275 | PN-001495.S01 | K04 | ML0000007587 |
| 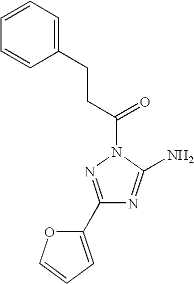 #NAME? | 7675581 | PN-001496.S01 | N09 | ML0000007589 |
| 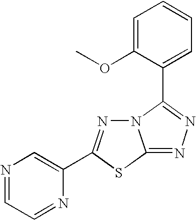 #NAME? | 7961197 | PN-001496.S01 | O12 | ML0000007589 |
| 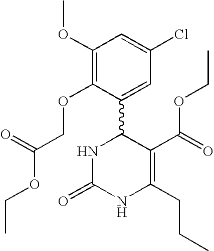 #NAME? | 7975174 | PN-001498.S01 | E07 | ML0000007593 |
| 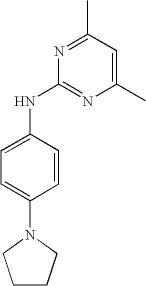 #NAME? | 7941479 | PN-001500.S01 | C22 | ML0000007597 |
| 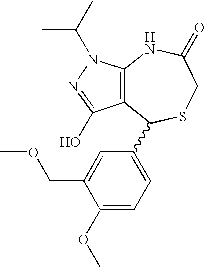 #NAME? | 7933061 | PN-001501.S01 | O09 | ML0000007599 |
| 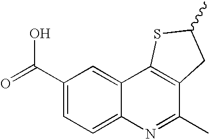 #NAME? | 5684977 | PN-001505.S01 | D22 | ML0000007607 |
| 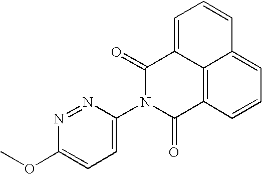 #NAME? | 7825307 | PN-001505.S01 | F20 | ML0000007607 |
| 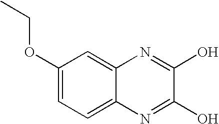 #NAME? | 7861435 | PN-001505.S01 | K23 | ML0000007607 |
| 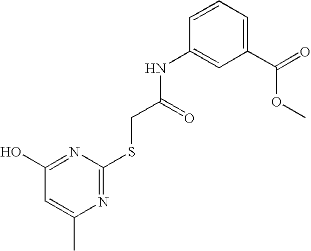 #NAME? | 7408041 | PN-001509.S01 | A03 | ML0000007615 |
| 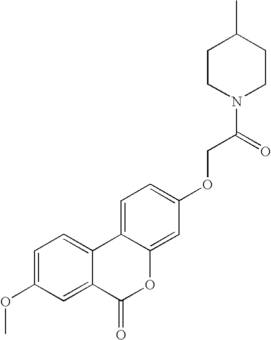 #NAME? | 7385666 | PN-001509.S01 | A13 | ML0000007615 |
| 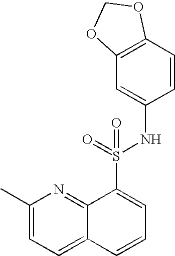 #NAME? | 7847947 | PN-001509.S01 | J06 | ML0000007615 |
| 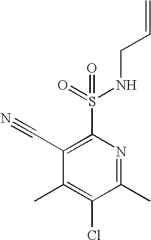 #NAME? | 8008-0703 | PN-001510.S01 | E23 | ML0000007617 |
| 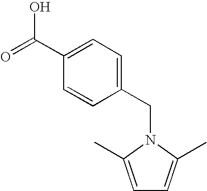 #NAME? | STOCK2S-44418 | PN-001510.S01 | O10 | ML0000007617 |
| 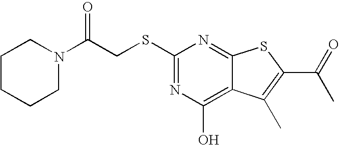 #NAME? | 7929184 | PN-001512.S02 | L13 | ML0000007621 |
| 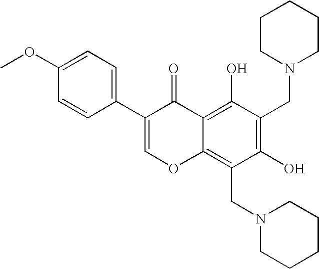 #NAME? | 6737-1234 | PN-001516.S01 | P14 | ML0000007829 |
| 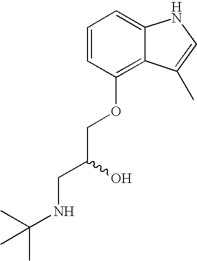 #NAME? | STOCK1S-09484 | PN-001518.S01 | C24 | ML0000007633 |
| 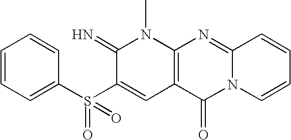 #NAME? | STOCK3S-38690 | PN-001518.S01 | G10 | ML0000007633 |
| 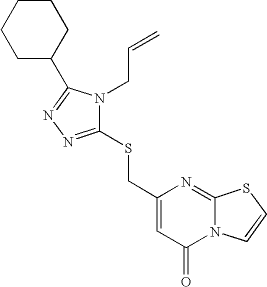 #NAME? | ZT-4771590 | PN-001518.S01 | K20 | ML0000007633 |
| 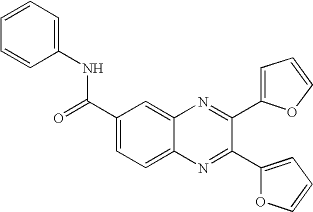 #NAME? | 7846194 | PN-1518.S01 | M24 | ML0000007633 |
| 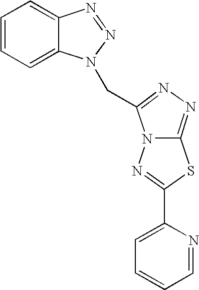 #NAME? | STOCK5S-55046 | PN-001518.S01 | O22 | ML0000007833 |
| 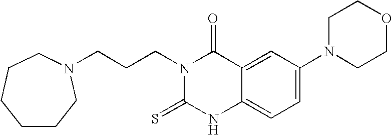 #NAME? | K284-6261 | PN-001520.S01 | D06 | ML0000007637 |
| 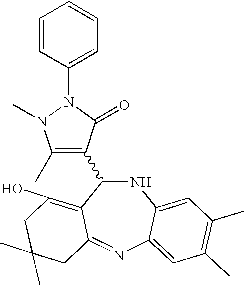 #NAME? | 7745596 | PN-001521.S01 | E12 | ML0000007639 |
| 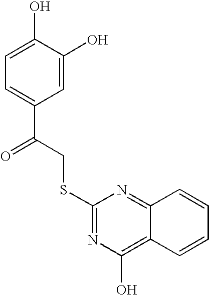 #NAME? | 4226-3191 | PN-001522.S01 | C21 | ML0000007641 |
| 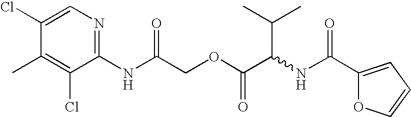 #NAME? | T5229618 | PN-001522.S01 | N12 | ML0000007641 |
| 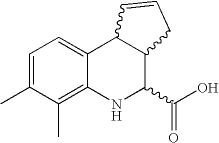 #NAME? | BAS 02818514 | PN-001523.S01 | B22 | ML0000007643 |
| 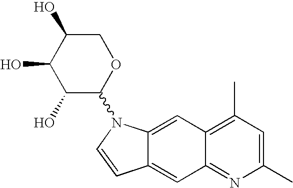 #NAME? | 7093-1605 | PN-001523.S01 | H18 | ML0000007643 |
| 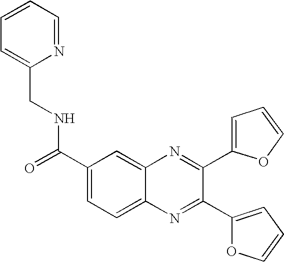 #NAME? | K216-1625 | PN-001524.S01 | O05 | ML0000007645 |
| 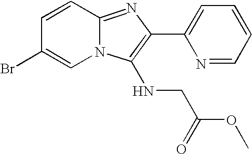 #NAME? | C177-1541 | PN-001525.S01 | H12 | ML0000007647 |
| 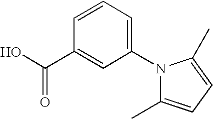 #NAME? | 5128773 | PN-001528.S01 | J23 | ML0000007653 |
| 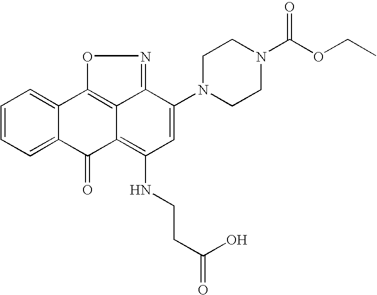 #NAME? | C090-0557 | PN-001528.S01 | P19 | ML0000007653 |
| 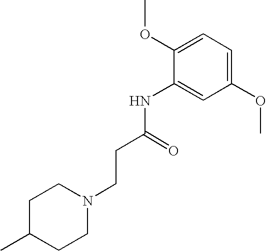 #NAME? | BAS 03086473 | PN-001529.S01 | E22 | ML0000007655 |
| 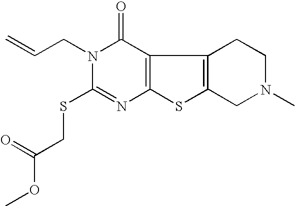 #NAME? | STOCK1S-28981 | PN-001531.S01 | D14 | ML0000007659 |
| 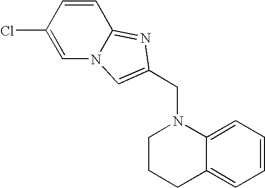 #NAME? | T0502-2716 | PN-001531.S01 | H10 | ML0000007659 |

| | ProjectPlate | Project |
|---|---|---|
| STRUCTURE | ID | Well ID |
| 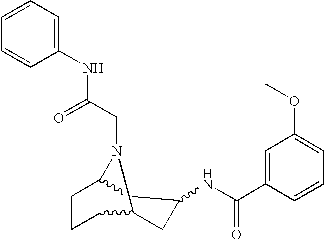 #NAME? | PA-004360 | N24 |
| 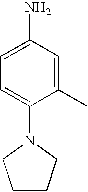 #NAME? | PA-004361 | J22 |
| 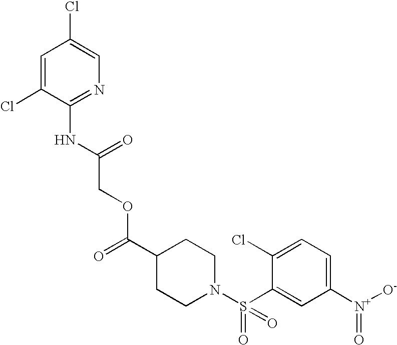 #NAME? | PA-004363 | A03 |
| 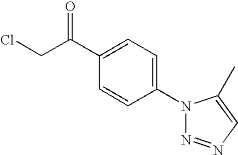 #NAME? | PA-004383 | E11 |
| 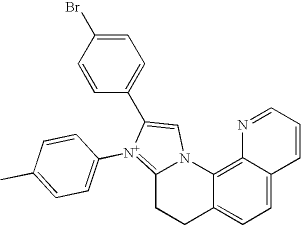 #NAME? | PA-004363 | M14 |
| 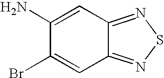 #NAME? | PA-004366 | D11 |
| 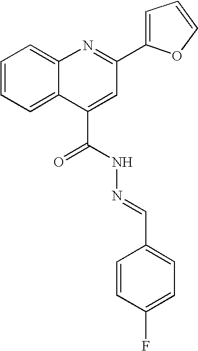 #NAME? | PA-004366 | B06 |
| 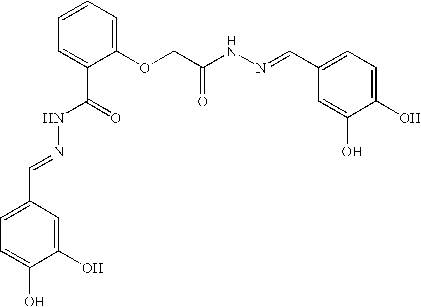 #NAME? | PA-004366 | L21 |
| 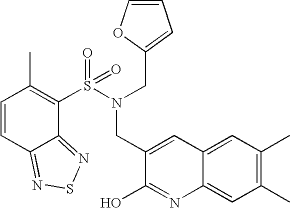 #NAME? | PA-004369 | P16 |
| 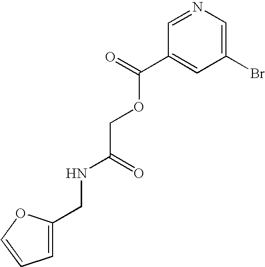 #NAME? | PA-004370 | A10 |
| 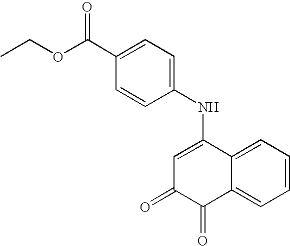 #NAME? | PA-004375 | I19 |
| 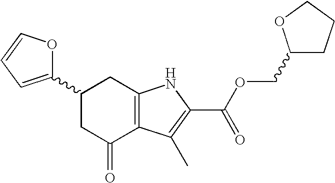 #NAME? | PA-004377 | O22 |
| 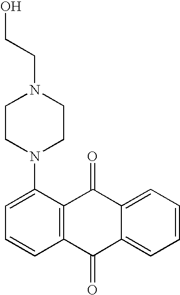 #NAME? | PA-004381 | I12 |
| 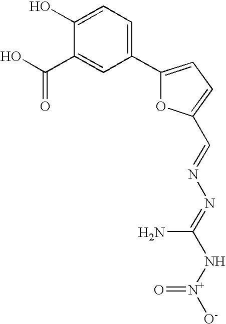 #NAME? | PA-004381 | J10 |
| 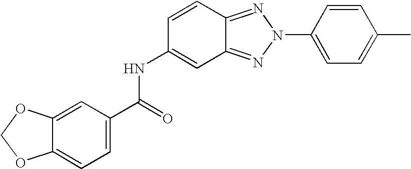 #NAME? | PA-004383 | K05 |
| 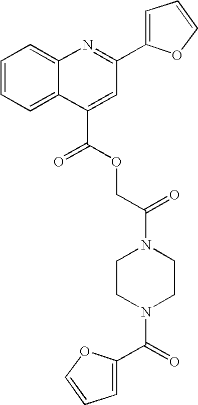 #NAME? | PA-004386 | N06 |
| 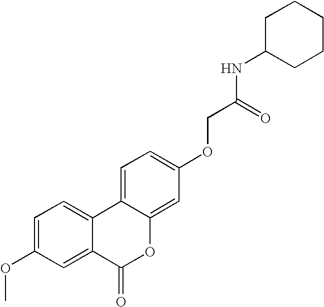 #NAME? | PA-004389 | J21 |
| 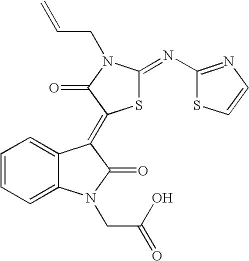 #NAME? | PA-004391 | C20 |
| 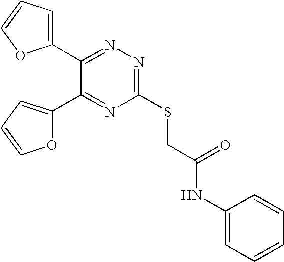 #NAME? | PA-004394 | A10 |
| 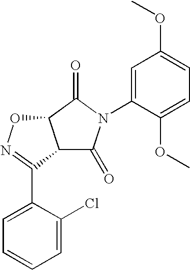 #NAME? | PA-004394 | D18 |
| 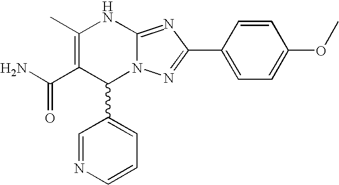 #NAME? | PA-004401 | I10 |
| 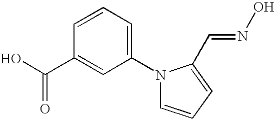 #NAME? | PA-004405 | J20 |
| 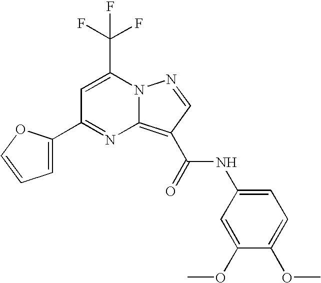 #NAME? | PA-004407 | J10 |
| 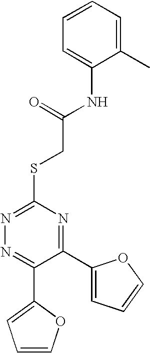 #NAME? | PA-004409 | A22 |
| 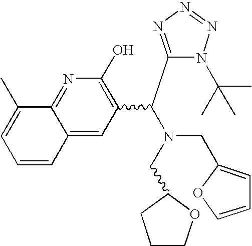 #NAME? | PA-004409 | M12 |
| 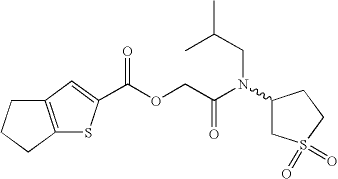 #NAME? | PA-004490 | J15 |
| 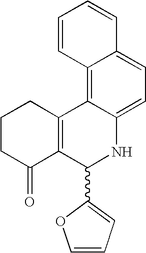 #NAME? | PA-004491 | F03 |
| 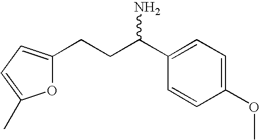 #NAME? | PA-004492 | E18 |
| 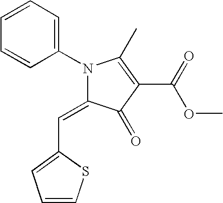 #NAME? | PA-004493 | C23 |
| 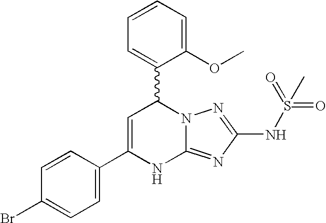 #NAME? | PA-004496 | C11 |
| 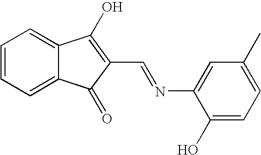 #NAME? | PA-004500 | E12 |
| 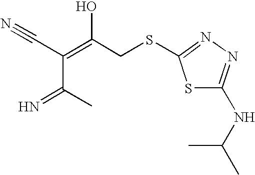 #NAME? | PA-004500 | L04 |
| 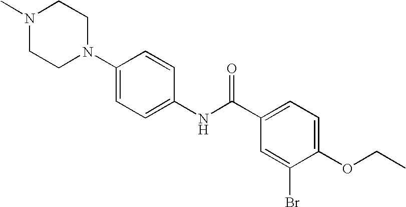 #NAME? | PA-004500 | O15 |
| 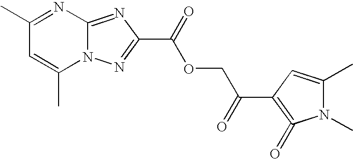 #NAME? | PA-004501 | O10 |
| 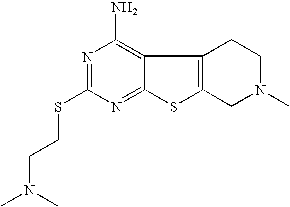 #NAME? | PA-004501 | O16 |
| 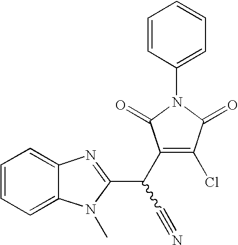 #NAME? | PA-004502 | A21 |
| 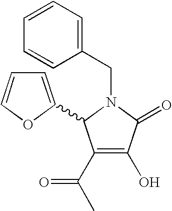 #NAME? | PA-004502 | D23 |
| 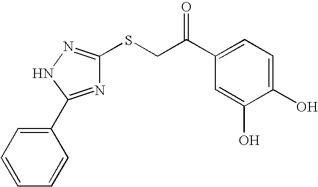 #NAME? | PA-004503 | E20 |
| 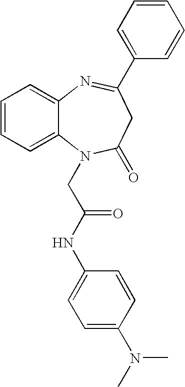 #NAME? | PA-004503 | F15 |
| 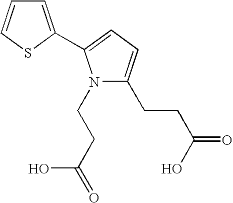 #NAME? | PA-004504 | A12 |
| 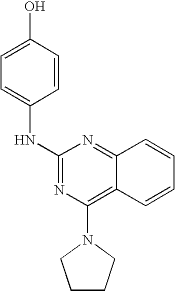 #NAME? | PA-004504 | C16 |
| 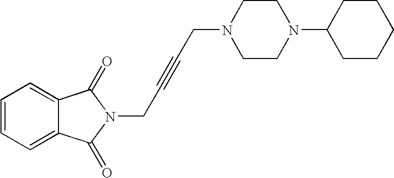 #NAME? | PA-004506 | G09 |
| 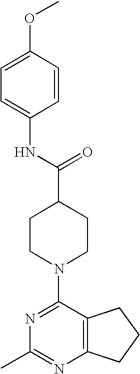 #NAME? | PA-004506 | N21 |
| 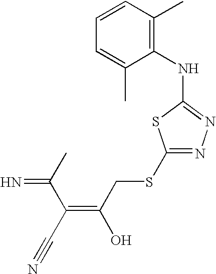 #NAME? | PA-004507 | H04 |
| 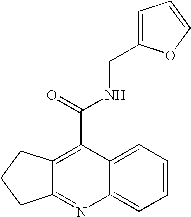 #NAME? | PA-004507 | H06 |
| 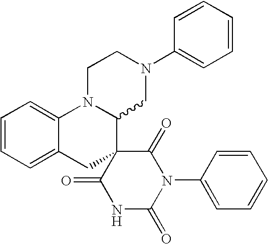 #NAME? | PA-004507 | K06 |
| 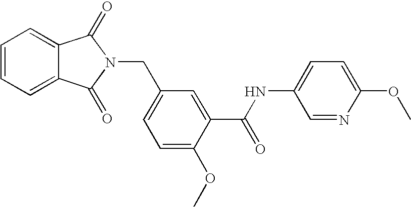 #NAME? | PA-004508 | H03 |
| 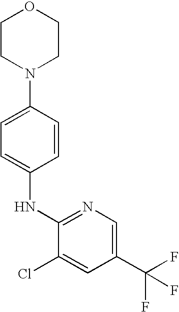 #NAME? | PA-004508 | O12 |
| 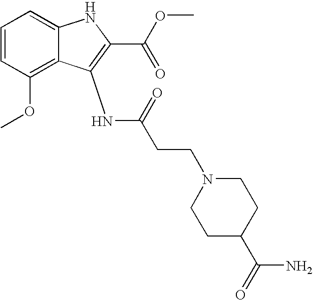 #NAME? | PA-004510 | P04 |
| 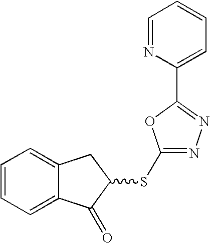 #NAME? | PA-004511 | H11 |
| 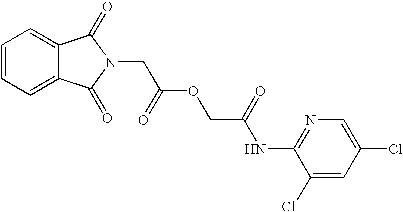 #NAME? | PA-004511 | J06 |
| 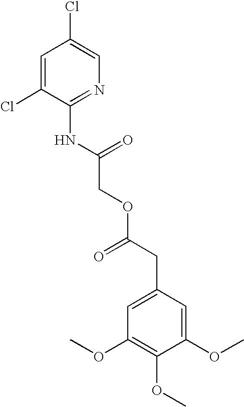 #NAME? | PA-004511 | L16 |
| 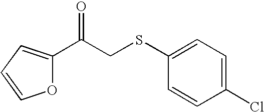 #NAME? | PA-004513 | P06 |
| 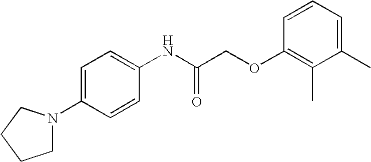 #NAME? | PA-004514 | M19 |
| 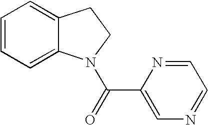 #NAME? | PA-004517 | A11 |
| 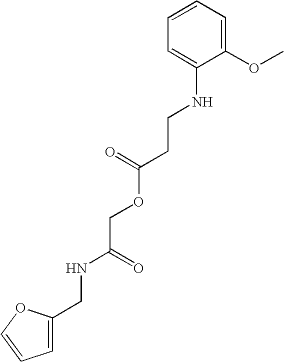 #NAME? | PA-004517 | A20 |
| 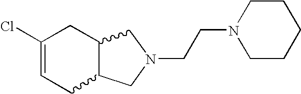 #NAME? | PA-004517 | L13 |
| 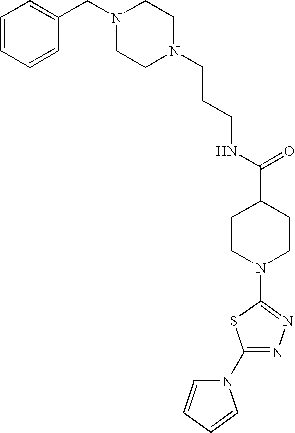 #NAME? | PA-004519 | F09 |
| 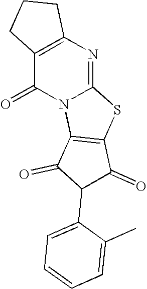 #NAME? | PA-004519 | K14 |
| 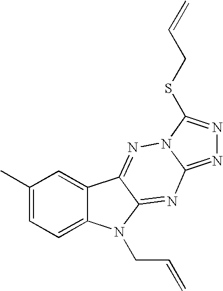 #NAME? | PA-004519 | O14 |
| 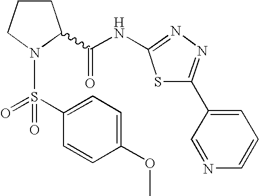 #NAME? | PA-004522 | A03 |
| 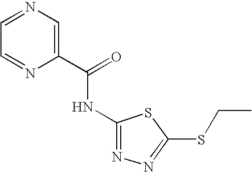 #NAME? | PA-004522 | D09 |
| 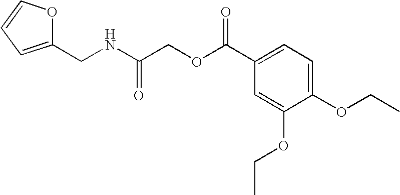 #NAME? | PA-004524 | O12 |
| 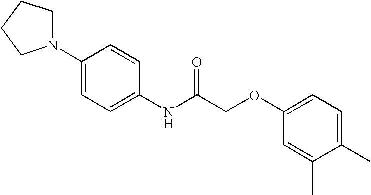 #NAME? | PA-004526 | A04 |
| 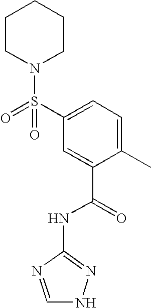 #NAME? | PA-004526 | H04 |
| 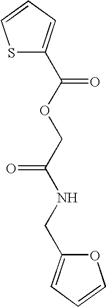 #NAME? | PA-004527 | G07 |
| 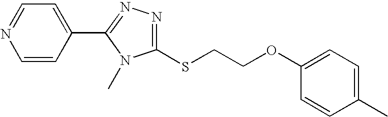 #NAME? | PA-004527 | L07 |
| 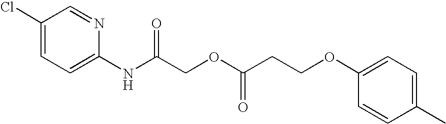 #NAME? | PA-004527 | O06 |
| 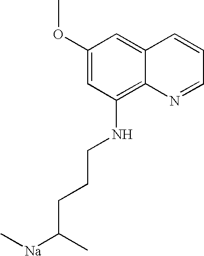 #NAME? | PA-004528 | A13 |
| 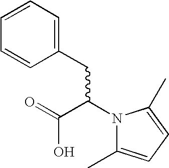 #NAME? | PA-004611 | G14 |
| 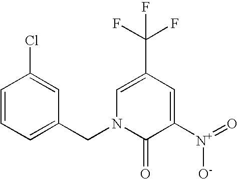 #NAME? | PA-004616 | J24 |
| 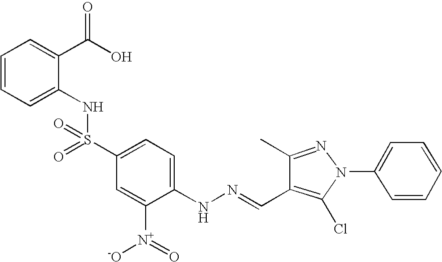 #NAME? | PA-004617 | O16 |
| 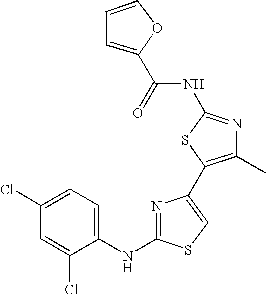 #NAME? | PA-004619 | H14 |
| 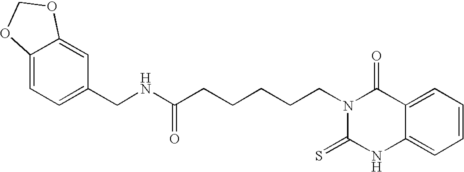 #NAME? | PA-004621 | H12 |
| 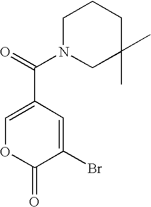 #NAME? | PA-004626 | C10 |
| 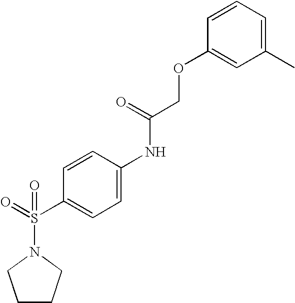 #NAME? | PA-004633 | N19 |
| 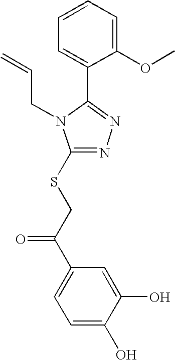 #NAME? | PA-004634 | D19 |
| 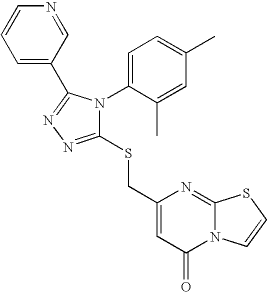 #NAME? | PA-004634 | F11 |
| 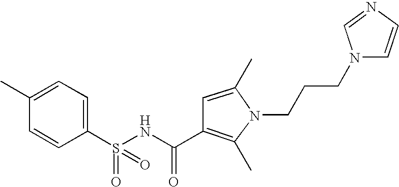 #NAME? | PA-004634 | H10 |
| 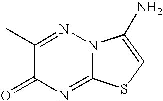 #NAME? | PA-004635 | E11 |
| 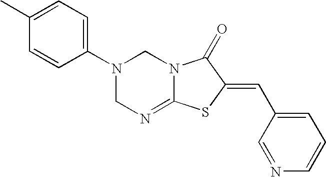 #NAME? | PA-004635 | E24 |
| 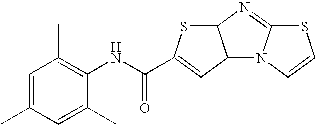 #NAME? | PA-004636 | L20 |
| 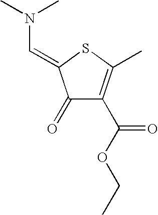 #NAME? | PA-004636 | B21 |
| 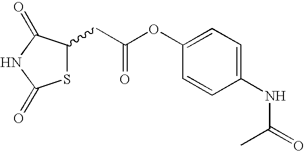 #NAME? | PA-004641 | C12 |
| 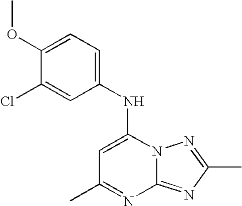 #NAME? | PA-004645 | B03 |
| 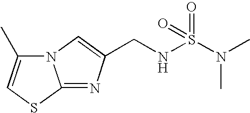 #NAME? | PA-004645 | I11 |
| 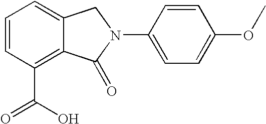 #NAME? | PA-004645 | P16 |
| 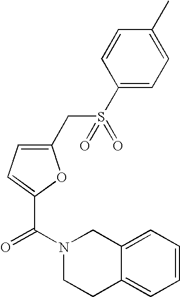 #NAME? | PA-004646 | I10 |
| 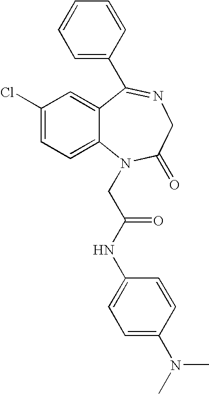 #NAME? | PA-004646 | D07 |
| 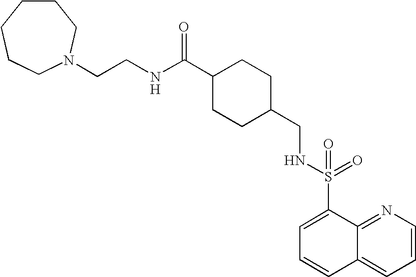 #NAME? | PA-004650 | G18 |
| 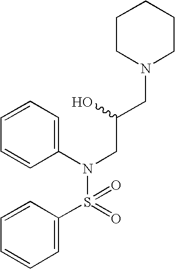 #NAME? | PA-004652 | B24 |
| 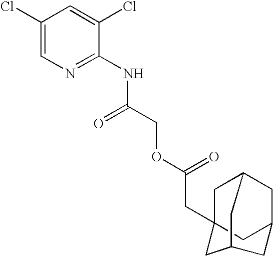 #NAME? | PA-004652 | G07 |
| 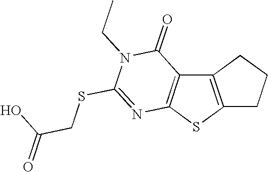 #NAME? | PA-004653 | I13 |
| 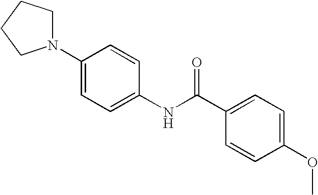 #NAME? | PA-004654 | D07 |
| 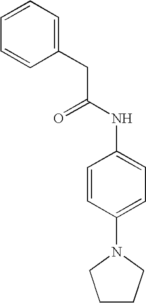 #NAME? | PA-004654 | D23 |
| 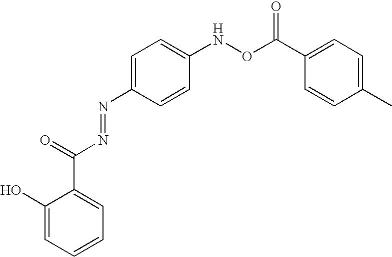 #NAME? | PA-004657 | A11 |
| 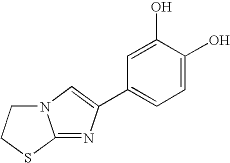 #NAME? | PA-004657 | D04 |
| 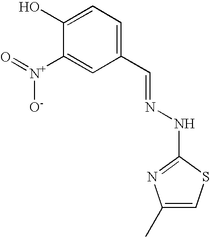 #NAME? | PA-004657 | P07 |
| 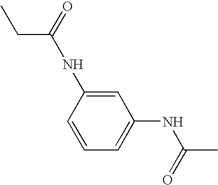 #NAME? | PA-004659 | F12 |
| 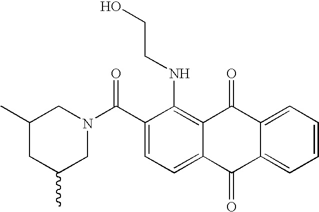 #NAME? | PA-004659 | I05 |
| 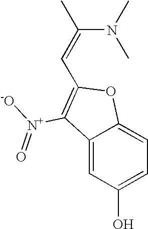 #NAME? | PA-004659 | N18 |
| 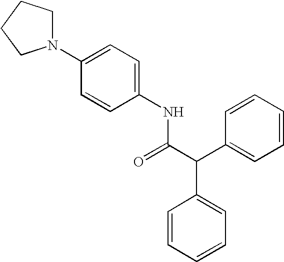 #NAME? | PA-004660 | H14 |
| 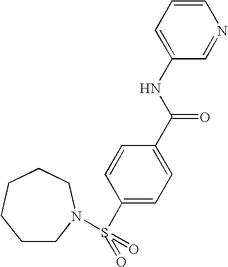 #NAME? | PA-004661 | J07 |
| 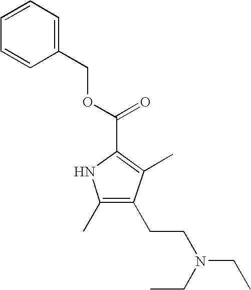 #NAME? | PA-004461 | K18 |
| 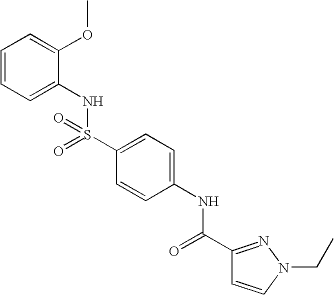 #NAME? | PA-004661 | O24 |
| 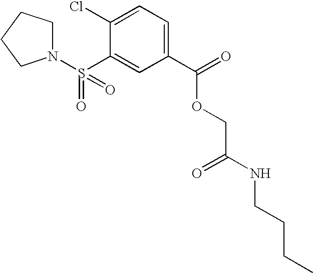 #NAME? | PA-004661 | P04 |
| 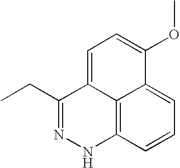 #NAME? | PA-004661 | P19 |
| 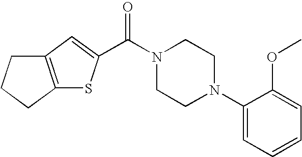 #NAME? | PA-004665 | A18 |
| 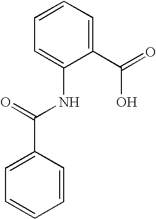 #NAME? | PA-004666 | F13 |
| 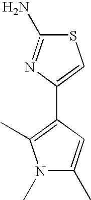 #NAME? | PA-004667 | O08 |
| 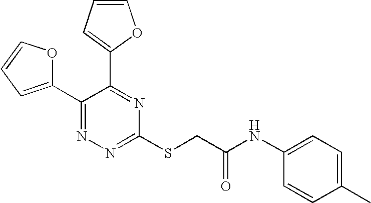 #NAME? | PA-004715 | C13 |
| 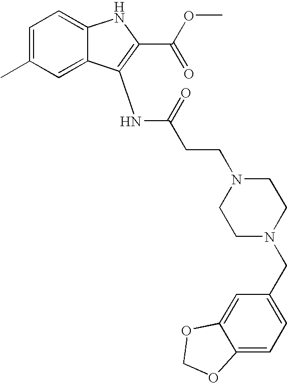 #NAME? | PA-004715 | I16 |
| 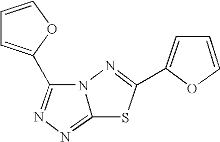 #NAME? | PA-004717 | B20 |
| 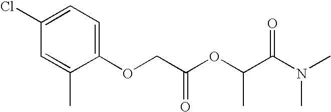 #NAME? | PA-004719 | C10 |
| 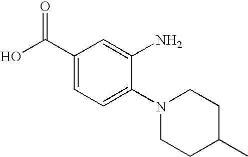 #NAME? | PA-004719 | N06 |
| 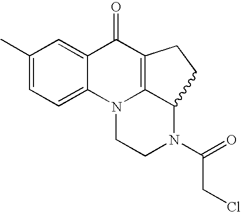 #NAME? | PA-004720 | A06 |
| 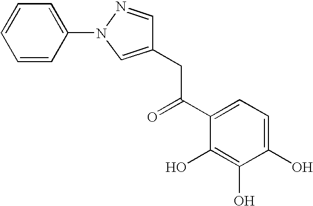 #NAME? | PA-004720 | K15 |
| 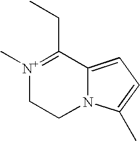 #NAME? | PA-004722 | J20 |
| 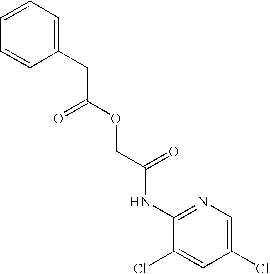 #NAME? | PA-004722 | K12 |
| 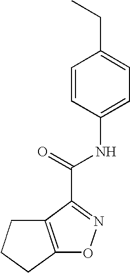 #NAME? | PA-004722 | L13 |
| 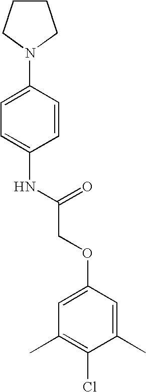 #NAME? | PA-004723 | G16 |
| 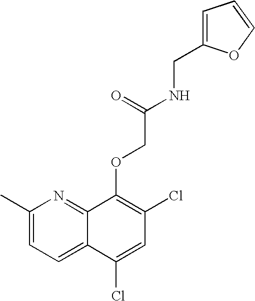 #NAME? | PA-004724 | F23 |
| 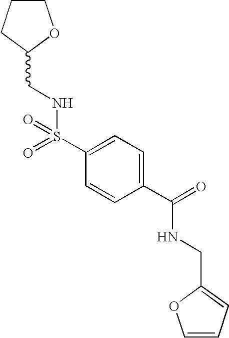 #NAME? | PA-004724 | O17 |
| 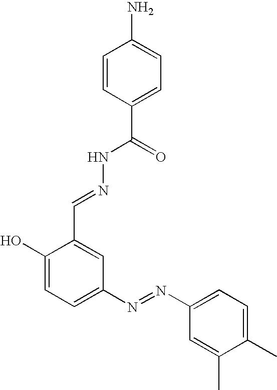 #NAME? | PA-004725 | J19 |
| 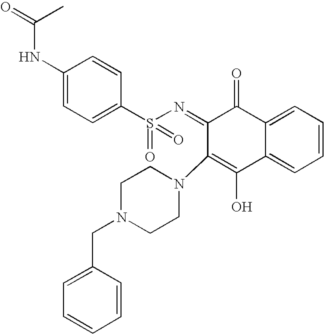 #NAME? | PA-004725 | O14 |
| 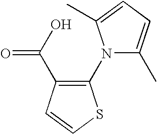 #NAME? | PA-004726 | J19 |
| 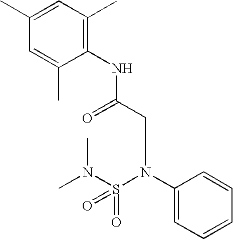 #NAME? | PA-004727 | F18 |
| 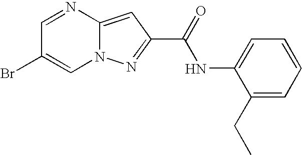 #NAME? | PA-004727 | G12 |
| 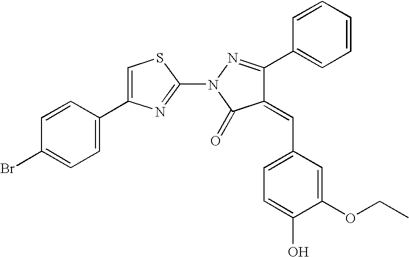 #NAME? | PA-004727 | I23 |
| 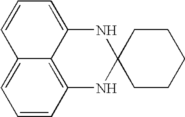 #NAME? | PA-004727 | L05 |
| 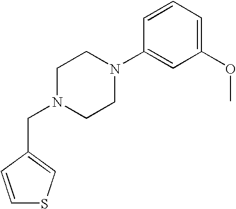 #NAME? | PA-004727 | L06 |
| 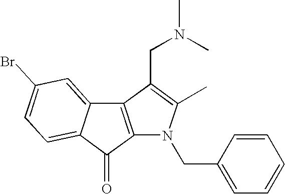 #NAME? | PA-004727 | O13 |
| 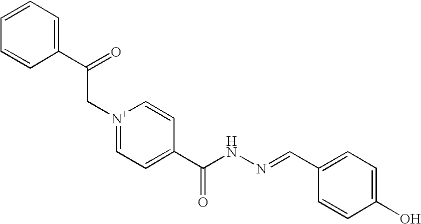 #NAME? | PA-004727 | P06 |
| 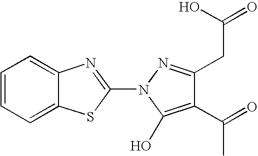 #NAME? | PA-004727 | P24 |
| 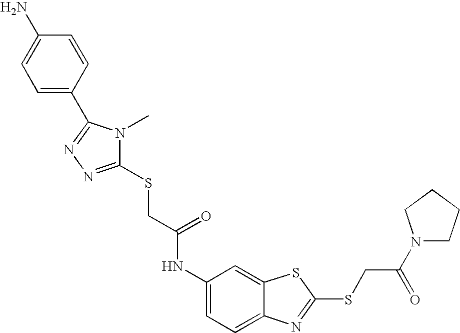 #NAME? | PA-004728 | C17 |
| 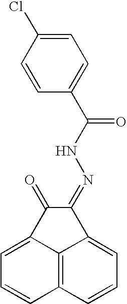 #NAME? | PA-004728 | L23 |
| 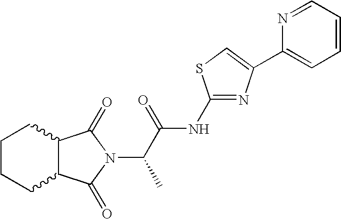 #NAME? | PA-004730 | C23 |
| 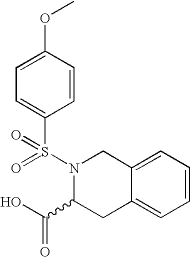 #NAME? | PA-004733 | O19 |
| 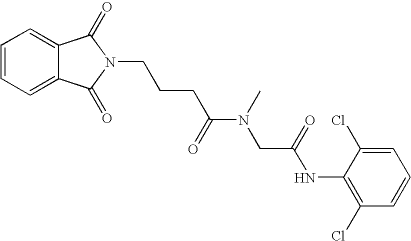 #NAME? | PA-004735 | A23 |
| 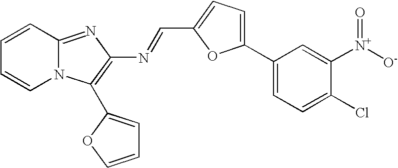 #NAME? | PA-004735 | C11 |
| 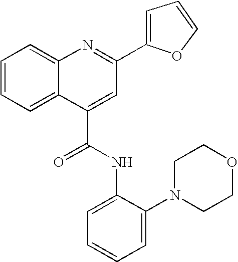 #NAME? | PA-004735 | G12 |
| 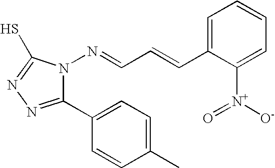 #NAME? | PA-004735 | M12 |
| 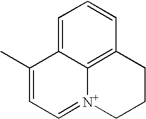 #NAME? | PA-004737 | A08 |
| 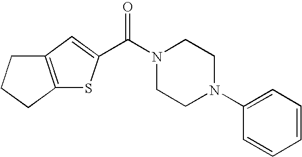 #NAME? | PA-004737 | A10 |
| 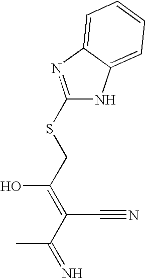 #NAME? | PA-004737 | B17 |
| 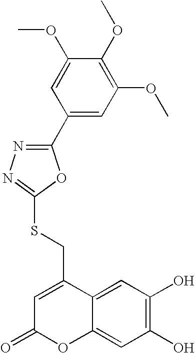 #NAME? | PA-004737 | B19 |
| 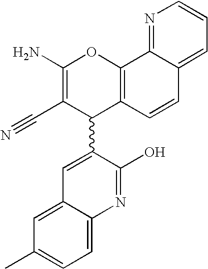 #NAME? | PA-004737 | D16 |
| 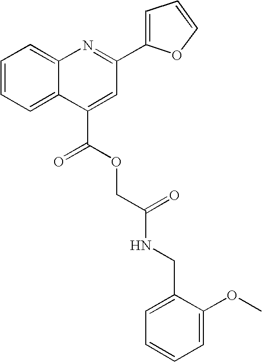 #NAME? | PA-004737 | F17 |
| 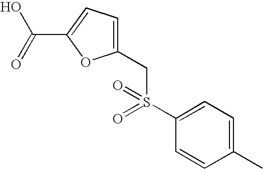 #NAME? | PA-004738 | H23 |
| 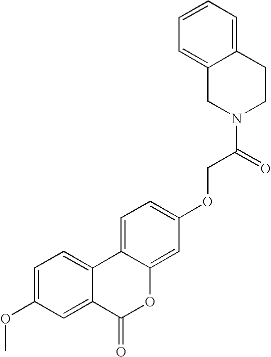 #NAME? | PA-004738 | M11 |
| 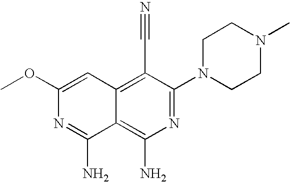 #NAME? | PA-004739 | I19 |
| 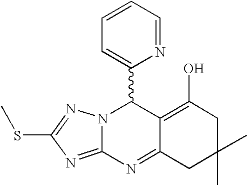 #NAME? | PA-004740 | P05 |
| 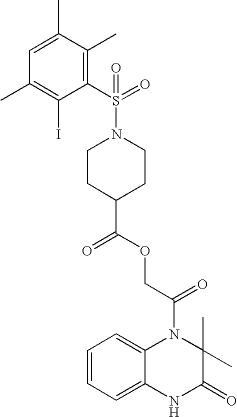 #NAME? | PA-004742 | D24 |
| 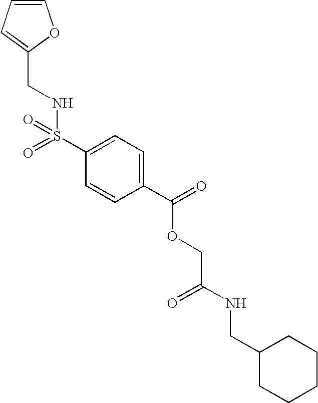 #NAME? | PA-004742 | J06 |
| 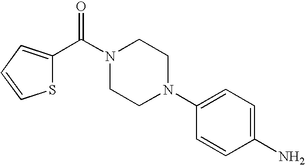 #NAME? | PA-004742 | K09 |
| 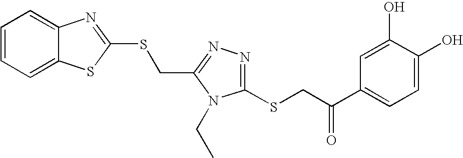 #NAME? | PA-004742 | M14 |
| 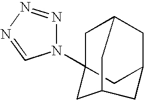 #NAME? | PA-004744 | B20 |
| 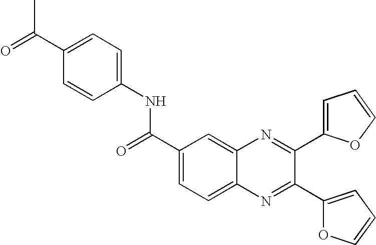 #NAME? | PA-004744 | K05 |
| 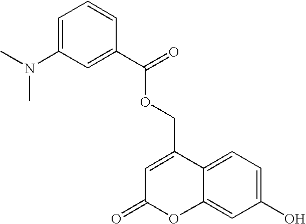 #NAME? | PA-004745 | F21 |
| 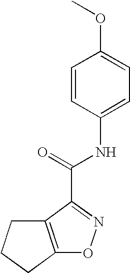 #NAME? | PA-004745 | L16 |
| 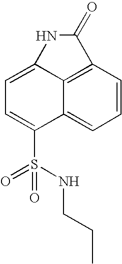 #NAME? | PA-004747 | I17 |
| 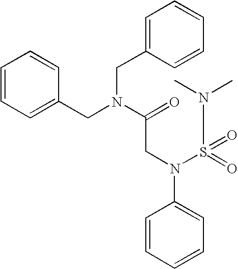 #NAME? | PA-004749 | D22 |
| 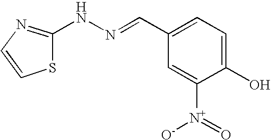 #NAME? | PA-004750 | J07 |
| 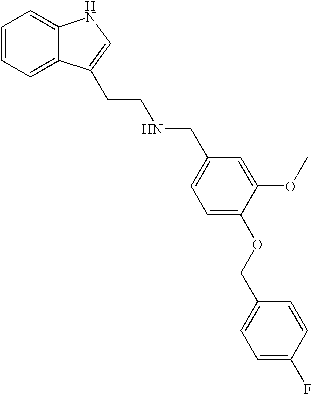 #NAME? | PA-004751 | E10 |
| 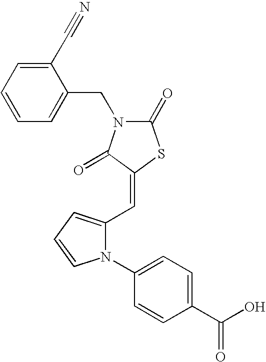 #NAME? | PA-004751 | E11 |
| 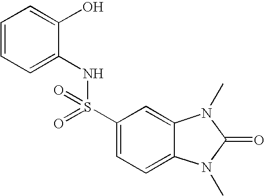 #NAME? | PA-004751 | L21 |
| 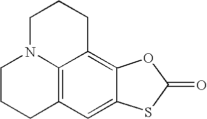 #NAME? | PA-004752 | L17 |
| 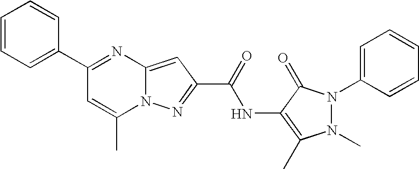 #NAME? | PA-004753 | D23 |
| 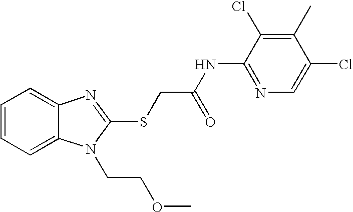 #NAME? | PA-004753 | G13 |
| 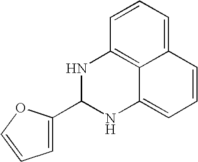 #NAME? | PA-004753 | J04 |
| 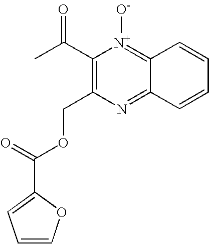 #NAME? | PA-004755 | P09 |
| 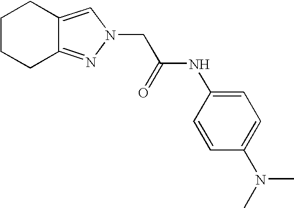 #NAME? | PA-004791 | C18 |
| 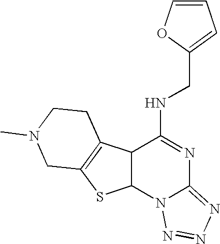 #NAME? | PA-004793 | P19 |
| 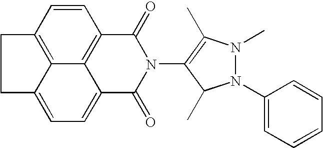 #NAME? | PA-004795 | O20 |
| 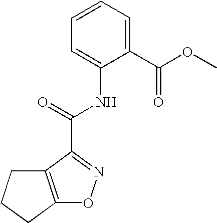 #NAME? | PA-004797 | H03 |
| 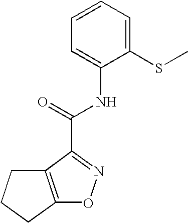 #NAME? | PA-004797 | P05 |
| 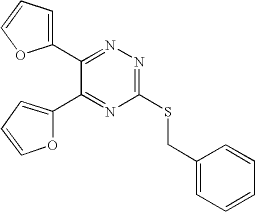 #NAME? | PA-004798 | A16 |
| 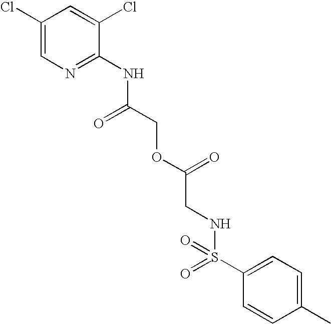 #NAME? | PA-004800 | D11 |
| 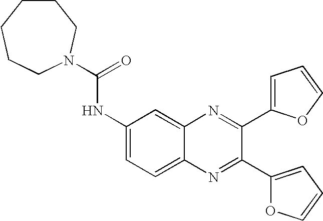 #NAME? | PA-004801 | B20 |
| 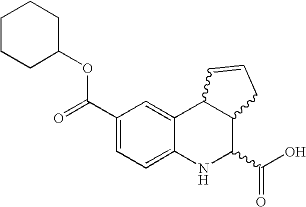 #NAME? | PA-004801 | I08 |
| 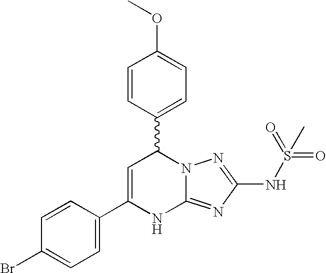 #NAME? | PA-004803 | D09 |
| 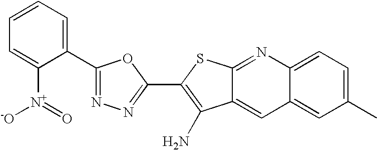 #NAME? | PA-004803 | N05 |
| 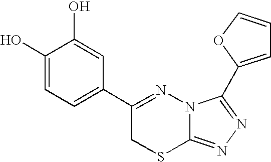 #NAME? | PA-004803 | P22 |
| 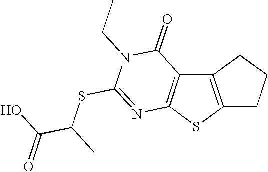 #NAME? | PA-004807 | F06 |
| 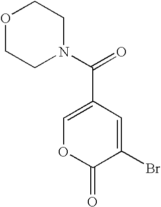 #NAME? | PA-004807 | G08 |
| 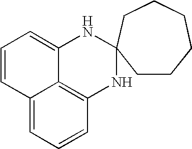 #NAME? | PA-004808 | N13 |
| 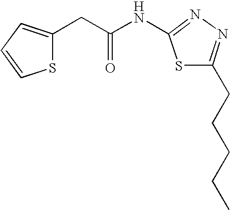 #NAME? | PA-004811 | N08 |
| 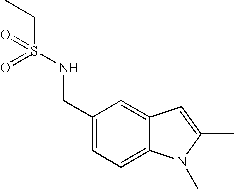 #NAME? | PA-004813 | G05 |
| 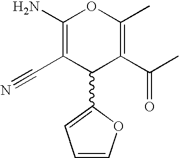 #NAME? | PA-004818 | F16 |
| 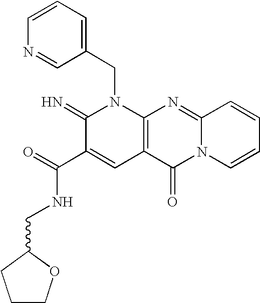 #NAME? | PA-004818 | G23 |
| 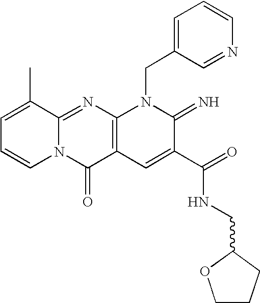 #NAME? | PA-004818 | I17 |
| 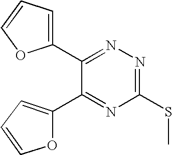 #NAME? | PA-004818 | N05 |
| 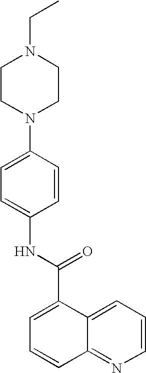 #NAME? | PA-004818 | N16 |
| 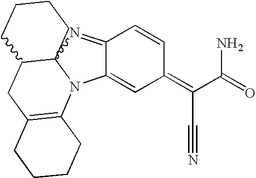 #NAME? | PA-004818 | O13 |
| 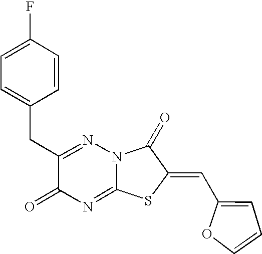 #NAME? | PA-004820 | K07 |
| 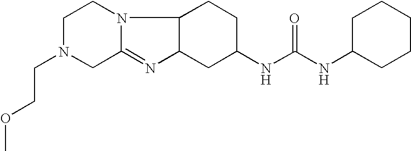 #NAME? | PA-004822 | L20 |
| 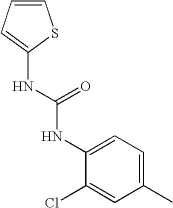 #NAME? | PA-004825 | N21 |
| 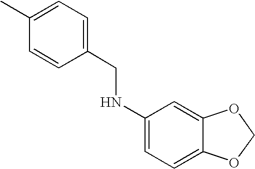 #NAME? | PA-004826 | H24 |
| 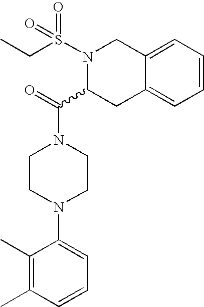 #NAME? | PA-004828 | D20 |
| 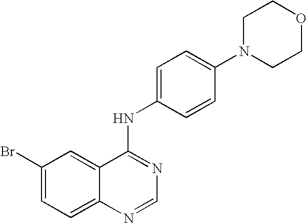 #NAME? | PA-004828 | I11 |
| 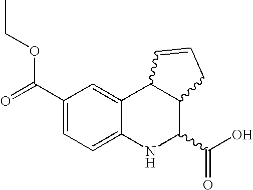 #NAME? | PA-004830 | E21 |
| 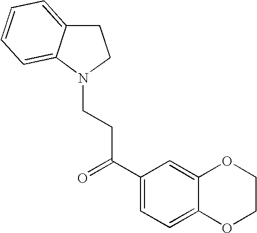 #NAME? | PA-004830 | K04 |
| 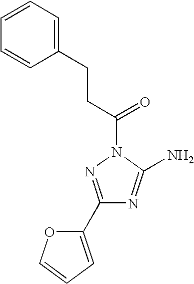 #NAME? | PA-004831 | N09 |
| 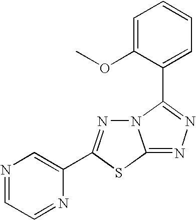 #NAME? | PA-004831 | O12 |
| 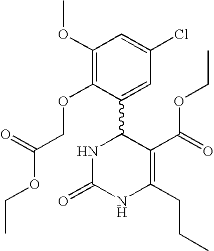 #NAME? | PA-004833 | E07 |
| 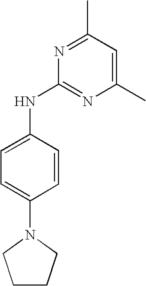 #NAME? | PA-004835 | C22 |
| 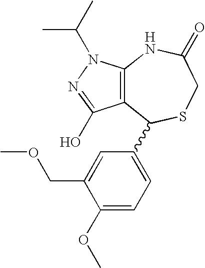 #NAME? | PA-004836 | O09 |
| 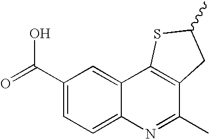 #NAME? | PA-004840 | D22 |
| 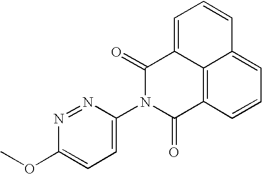 #NAME? | PA-004840 | F20 |
| 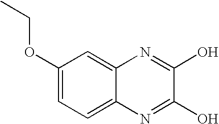 #NAME? | PA-004840 | K23 |
| 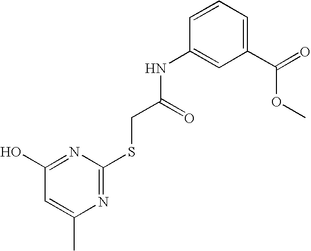 #NAME? | PA-004844 | A03 |
| 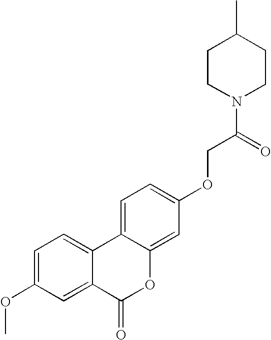 #NAME? | PA-004844 | A13 |
| 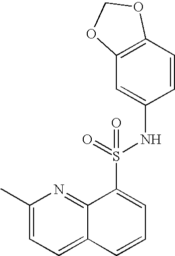 #NAME? | PA-004844 | J06 |
| 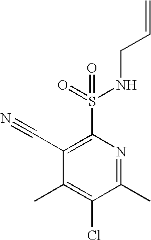 #NAME? | PA-004845 | E23 |
| 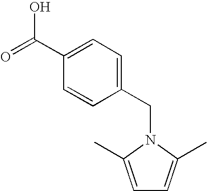 #NAME? | PA-004845 | O10 |
| 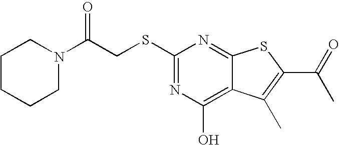 #NAME? | PA-004847 | L13 |
| 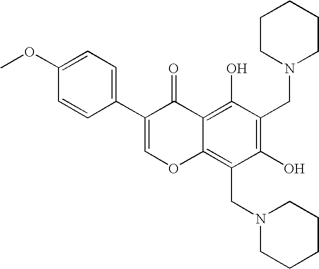 #NAME? | PA-004851 | P14 |
| 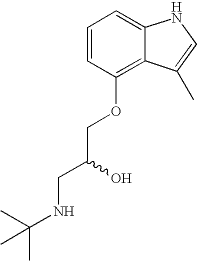 #NAME? | PA-004853 | C24 |
| 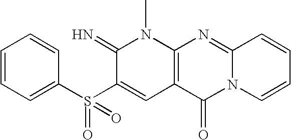 #NAME? | PA-004853 | G10 |
| 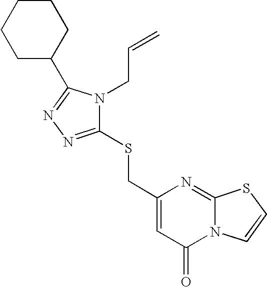 #NAME? | PA-004853 | K20 |
| 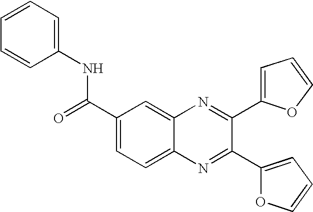 #NAME? | PA-004853 | M24 |
| 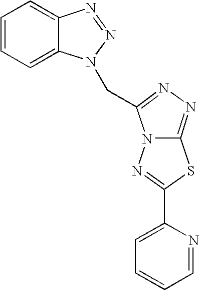 #NAME? | PA-004853 | O22 |
| 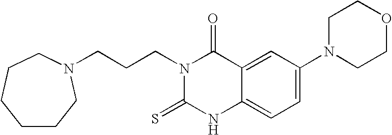 #NAME? | PA-004855 | D06 |
| 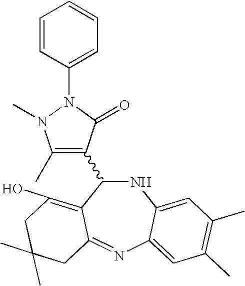 #NAME? | PA-004856 | E12 |
| 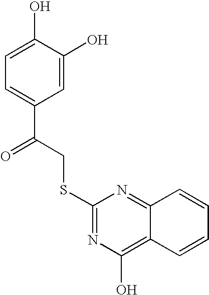 #NAME? | PA-004857 | C21 |
| 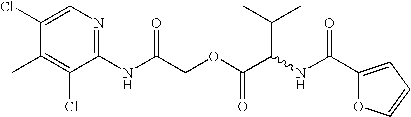 #NAME? | PA-004857 | N12 |
| 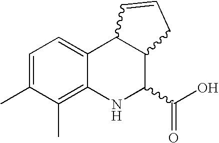 #NAME? | PA-004858 | B22 |
| 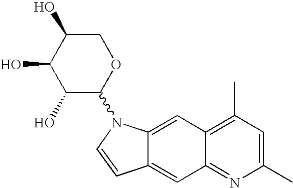 #NAME? | PA-004858 | H18 |
| 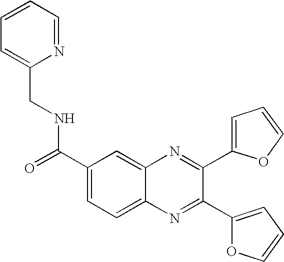 #NAME? | PA-004859 | O05 |
| 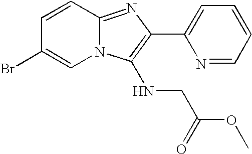 #NAME? | PA-004860 | H12 |
| 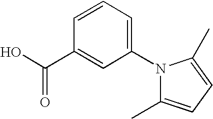 #NAME? | PA-004863 | J23 |
| 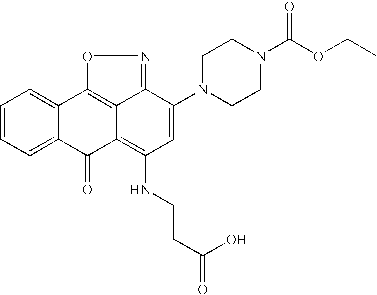 #NAME? | PA-004863 | P19 |
| 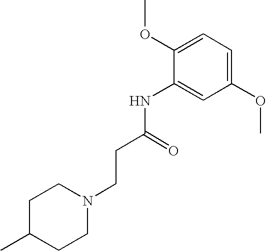 #NAME? | PA-004864 | E22 |
| 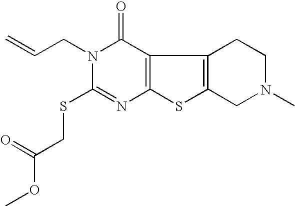 #NAME? | PA-004866 | D14 |
| 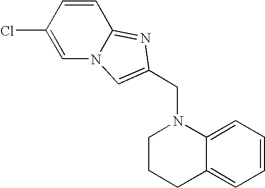 #NAME? | PA-004866 | H10 |

TABLE 6

| | | | | Concen- | | |
|---|---|---|---|---|---|---|
| | | Result | Concen- | tration | Assay | Assay |
| Structure | Assay Target | Value | tration | Unit | Name | Format |
| 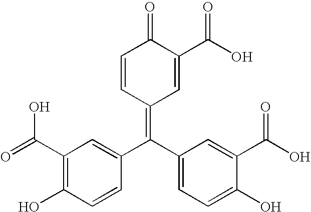 | BCL-b/TR3 | 107.34 | 0.02 | uM | Binding Assay | FP |
| 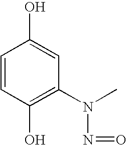 | BCL-b/TR3 | 95.333 | 0.02 | uM | Binding Assay | FP |
| 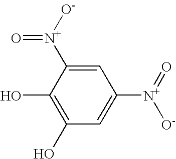 | BCL-b/TR3 | 68.095 | 0.02 | uM | Binding Assay | FP |
| 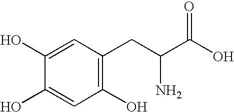 | BCL-b/TR3 | 135.72 | 0.02 | uM | Binding Assay | FP |
| 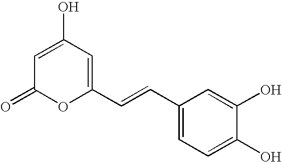 | BCL-b,TR3 | 77.018 | 0.02 | uM | Binding Assay | FP |
| 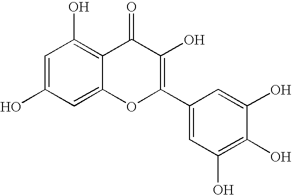 | BCL-b/TR3 | 60.993 | 0.02 | uM | Binding Assay | FP |
| 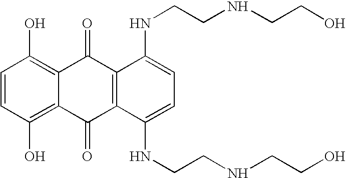 | BCL-b/TR3 | 148.07 | 0.02 | uM | Binding Assay | FP |
| 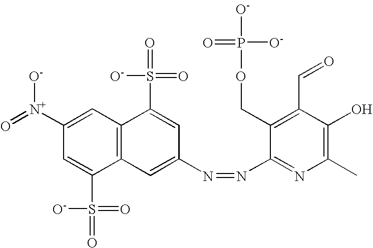 | BCL-b/TR3 | 66.551 | 0.02 | uM | Binding Assay | FP |
| 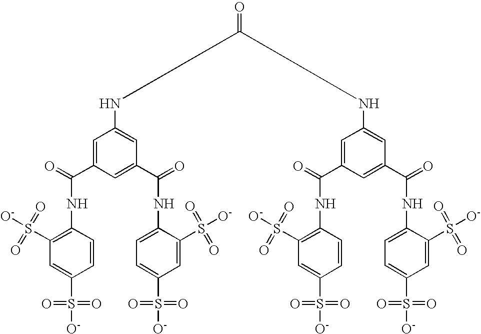 | BCL-b/TR3 | 64.698 | 0.02 | uM | Binding Assay | FP |
| 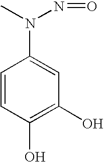 | BCL-b/TR3 | 121.2 | 0.02 | uM | Binding Assay | FP |
| 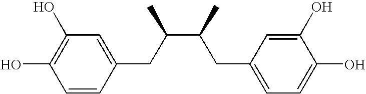 | BCL-b/TR3 | 67.477 | 0.02 | uM | Binding Assay | FP |
| 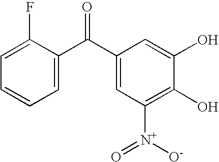 | BCL-b/TR3 | 69.641 | 0.02 | uM | Binding Assay | FP |
| 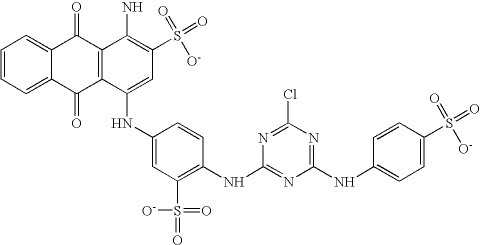 | BCL-b/TR3 | 80.802 | 0.02 | uM | Binding Assay | FP |
| 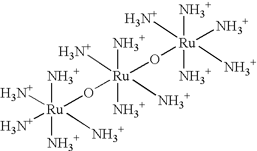 | BCL-b/TR3 | 144.05 | 0.02 | uM | Binding Assay | FP |
| 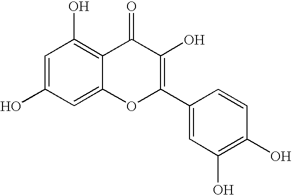 | BCL-b/TR3 | 51.969 | 0.02 | uM | Binding Assay | FP |
| 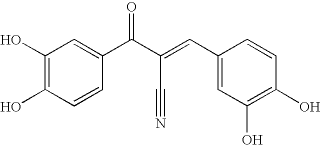 | BCL-b/TR3 | 61.735 | 0.02 | uM | Binding Assay | FP |

| | | | | Vendor | |
|---|---|---|---|---|---|
| Structure | Result Type | Is Hit | F Ratio | Compound ID | ID |
| 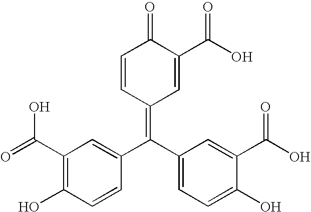 | % Inhibition | Yes | 0.07629 | A 1895 | 546233 |
| 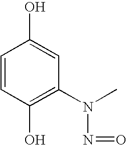 | % Inhibition | Yes | 0.11515 | D 8065 | 546352 |
| 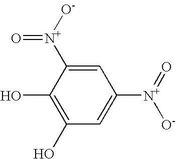 | % Inhibition | Yes | 0.14638 | D-131 | 546637 |
| 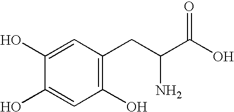 | % Inhibition | Yes | 0.06315 | H 2380 | 546649 |
| 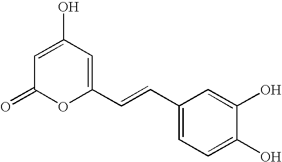 | % Inhibition | Yes | 0.0971 | H 5257 | 546651 |
| 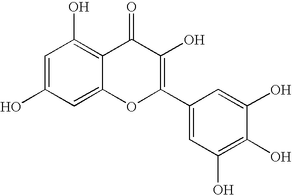 | % Inhibition | Yes | 0.12473 | M 6750 | 546723 |
| 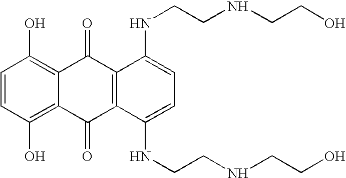 | % Inhibition | Yes | 1.1021 | M 6545 | 545881 |
| 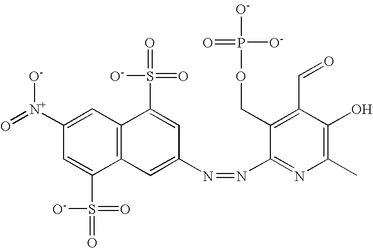 | % Inhibition | Yes | 0.26356 | P 2738 | 546897 |
| 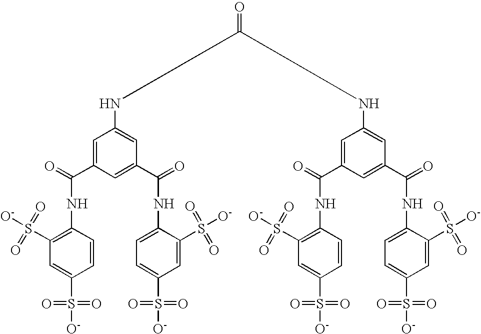 | % Inhibition | Yes | 1.0398 | N 4784 | 546936 |
| 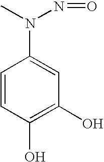 | % Inhibition | Yes | 0.08483 | M 9440 | 646964 |
| 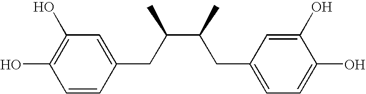 | % Inhibition | Yes | 0.09564 | N 5023 | 546976 |
| 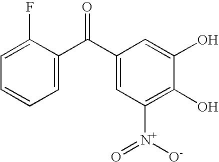 | % Inhibition | Yes | 0.1164 | R-108 | 546990 |
| 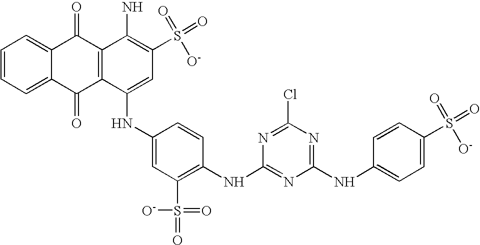 | % Inhibition | Yes | 0.16524 | R-115 | 547030 |
| 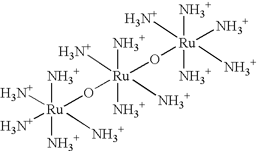 | % Inhibition | Yes | 1.1267 | R 2751 | 547104 |
| 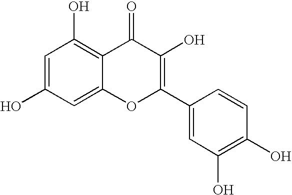 | % Inhibition | Yes | 0.14243 | Q 0125 | 547119 |
| 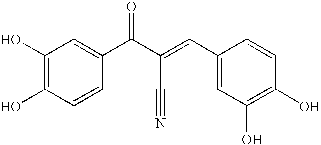 | % Inhibition | Yes | 0.10363 | T 7822 | 547242 |

TABLE 7

| Structure | Assay Target | Result Value |
|---|---|---|
| 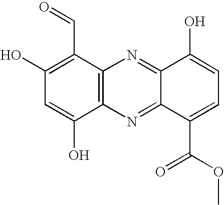 | BCL-b/TR3 | 98.94 |
| 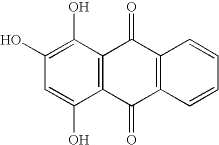 | BCL-b/TR3 | 146.42 |
| 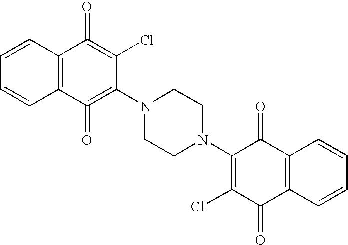 | BCL-b/TR3 | 54.176 |
| 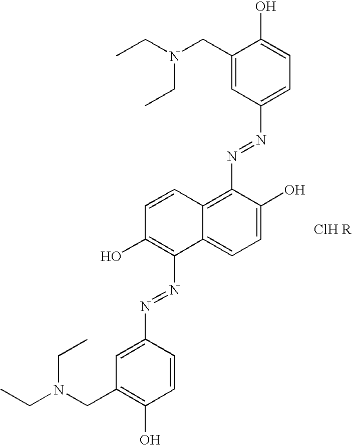 | BCL-b/TR3 | 94.871 |
| 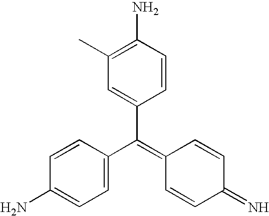 | BCL-b/TR3 | 78.593 |
| 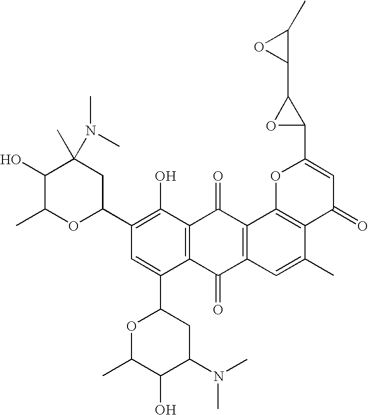 | BCL-b/TR3 | 104.82 |
| 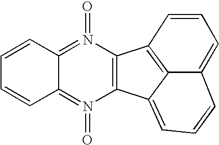 | BCL-b/TR3 | 72.262 |
| 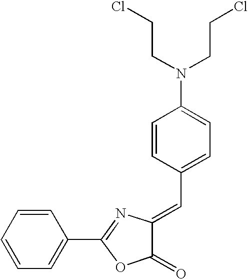 | BCL-b/TR3 | 62.767 |
| 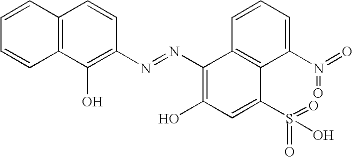 | BCL-b/TR3 | 140.54 |
| 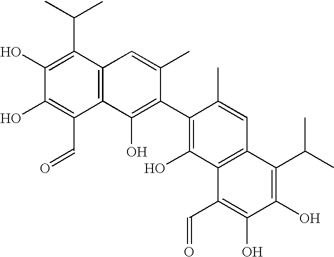 | BCL-b/TR3 | 57.341 |
| 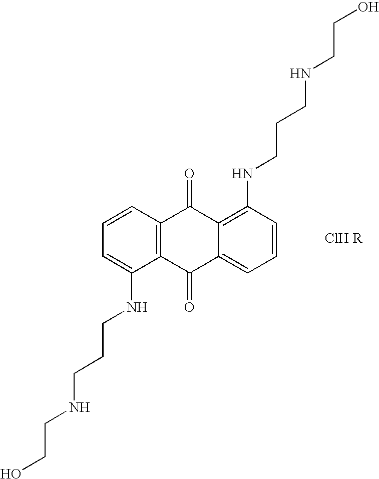 | BCL-b/TR3 | 114.77 |
| | BCL-b/TR3 | 89.897 |
| 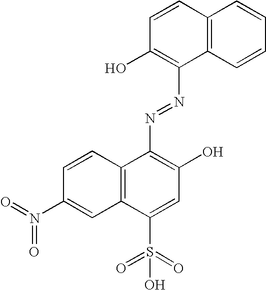 | BCL-b/TR3 | 124.71 |
| 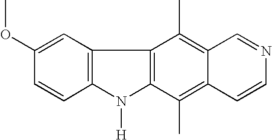 | BCL-b/TR3 | 96.679 |
| 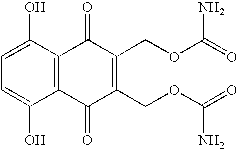 | BCL-b/TR3 | 144.51 |
| 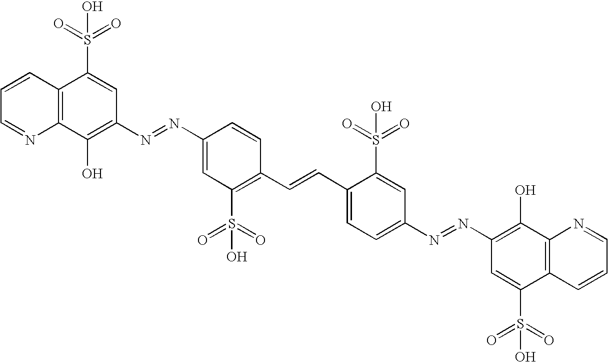 | BCL-b/TR3 | 93.966 |
| 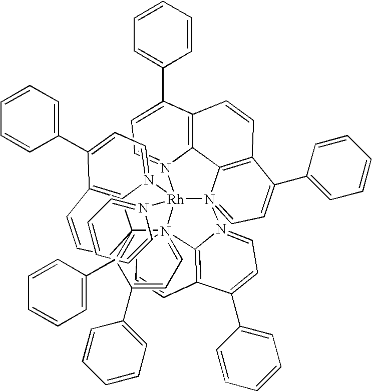 | BCL-b/TR3 | 104.9 |
| 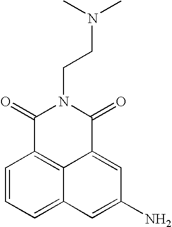 | BCL-b/TR3 | 87.591 |
| 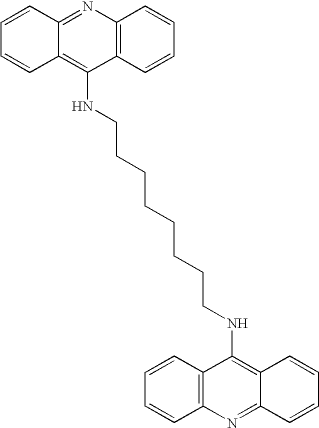 | BCL-b/TR3 | 53.437 |
| 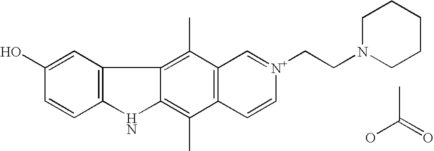 | BCL-b/TR3 | 119.92 |
| 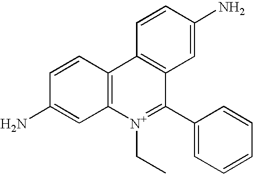 | BCL-b/TR3 | 149.07 |
| 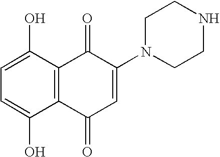 | BCL-b/TR3 | 68.464 |
| 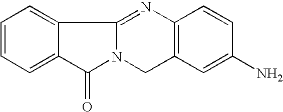 | BCL-b/TR3 | 60.723 |
| 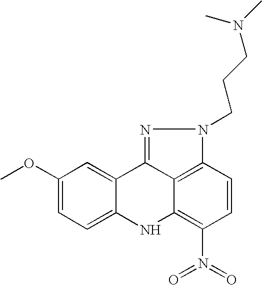 | BCL-b/TR3 | 64.386 |
| 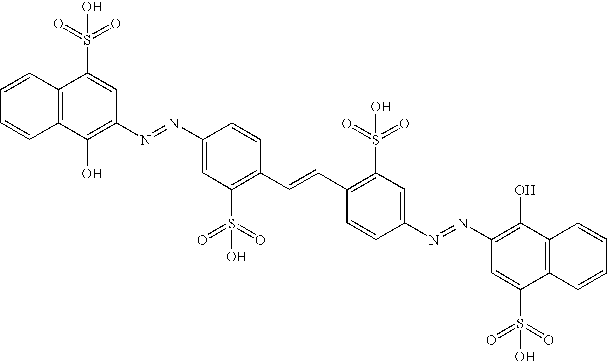 | BCL-b/TR3 | 80.305 |
| | BCL-b/TR3 | 83.948 |
| 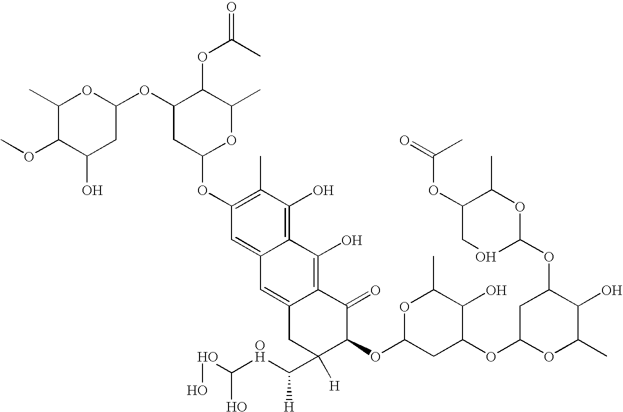 | BCL-b/TR3 | 83.037 |
| 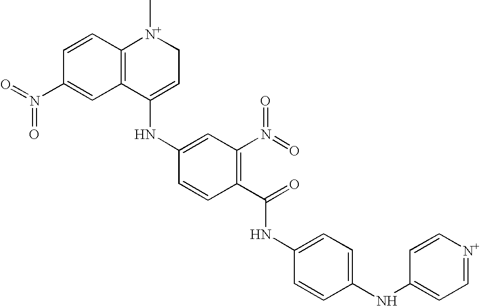 | BCL-b/TR3 | 105.56 |
| | BCL-b/TR3 | 114.73 |
| 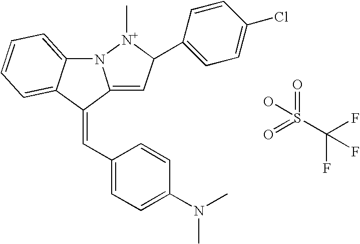 | BCL-b/TR3 | 60.625 |
| | BCL-b/TR3 | 120.23 |
| 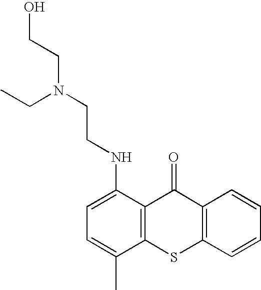 | BCL-b/TR3 | 74.38 |
| 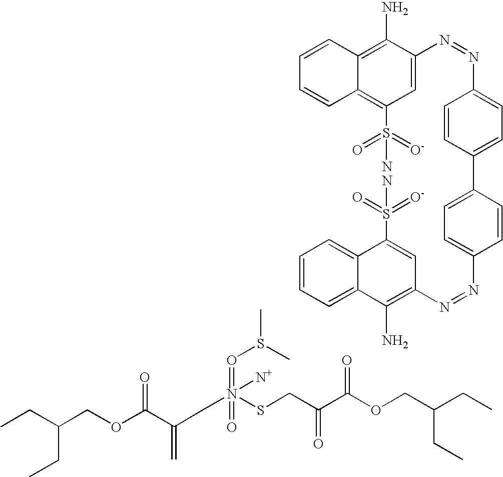 | BCL-b/TR3 | 76.673 |
| 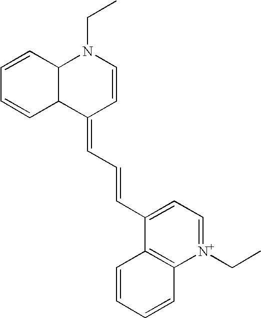 | BCL-b/TR3 | 103.73 |
| 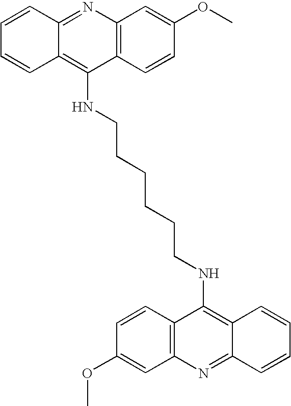 | BCL-b/TR3 | 53.288 |
| 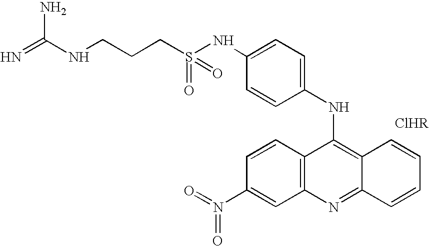 | BCL-b/TR3 | 51.913 |
| 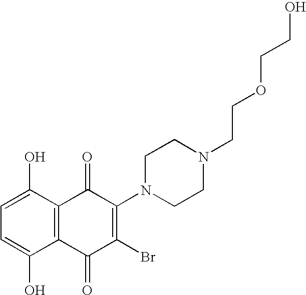 | BCL-b/TR3 | 134.45 |
| 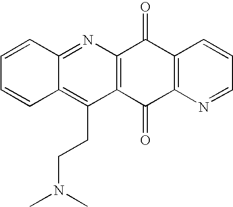 | BCL-b/TR3 | 82.634 |
| 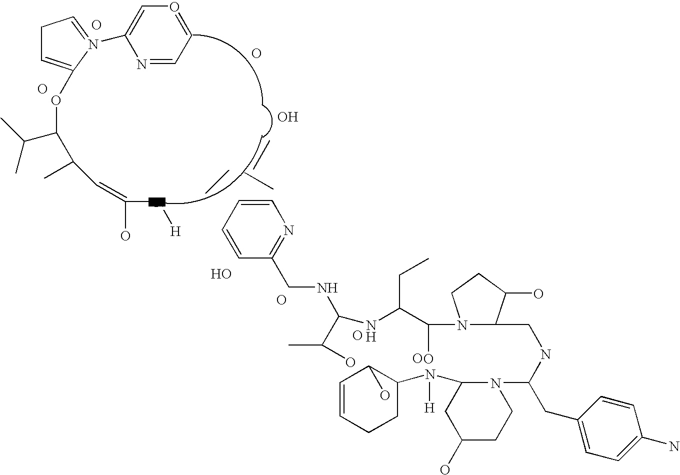 | BCL-b/TR3 | 50.079 |

| | Concentration | | | Result | | | Vendor | |
|---|---|---|---|---|---|---|---|---|
| Concentration | Unit | Assay Name | Assay Format | Type | Is Hit | F Ratio | Compound ID | ID |
| 0.02 | uM | Binding Assay | FP | % Inhibition | Yes | 0.080271 | 106995 | 549286 |
| 0.02 | uM | Binding Assay | FP | % Inhibition | Yes | 0.55319 | 10447 | 549312 |
| 0.02 | uM | Binding Assay | FP | % Inhibition | Yes | 0.41706 | 40341 | 549362 |
| 0.02 | uM | Binding Assay | FP | % Inhibition | Yes | 0.087735 | 359463 | 549433 |
| 0.02 | uM | Binding Assay | FP | % Inhibition | Yes | 0.10802 | 93739 | 549452 |
| 0.02 | uM | Binding Assay | FP | % Inhibition | Yes | 0.1099 | 70929 | 549458 |
| 0.02 | uM | Binding Assay | FP | % Inhibition | Yes | 1.4371 | 60309 | 549460 |
| 0.02 | uM | Binding Assay | FP | % Inhibition | Yes | 1.2733 | 104117 | 549492 |
| 0.02 | uM | Binding Assay | FP | % Inhibition | Yes | 0.064891 | 85561 | 549524 |
| 0.02 | uM | Binding Assay | FP | % Inhibition | Yes | 0.14725 | 56817 | 549547 |
| 0.02 | uM | Binding Assay | FP | % Inhibition | Yes | 0.22129 | 363998 | 549553 |
| 0.02 | uM | Binding Assay | FP | % Inhibition | Yes | 0.13752 | 85700 | 549564 |
| 0.02 | uM | Binding Assay | FP | % Inhibition | Yes | 0.059471 | 73413 | 549576 |
| 0.02 | uM | Binding Assay | FP | % Inhibition | Yes | 0.14038 | 69157 | 549584 |
| 0.02 | uM | Binding Assay | FP | % Inhibition | Yes | 1.1456 | 224124 | 549598 |
| 0.02 | uM | Binding Assay | FP | % Inhibition | Yes | 0.04471 | 635352 | 549624 |
| 0.02 | uM | Binding Assay | FP | % Inhibition | Yes | 0.056247 | 619179 | 649633 |
| 0.02 | uM | Binding Assay | FP | % Inhibition | Yes | 0.63451 | 308847 | 549659 |
| 0.02 | uM | Binding Assay | FP | % Inhibition | Yes | 0.20077 | 219734 | 549678 |
| 0.02 | uM | Binding Assay | FP | % Inhibition | Yes | 0.27501 | 311153 | 549798 |
| 0.02 | uM | Binding Assay | FP | % Inhibition | Yes | 0.38491 | 268986 | 549805 |
| 0.02 | uM | Binding Assay | FP | % Inhibition | Yes | 0.20934 | 658144 | 549822 |
| 0.02 | uM | Binding Assay | FP | % Inhibition | Yes | 1.4904 | 320846 | 549524 |
| 0.02 | uM | Binding Assay | FP | % Inhibition | Yes | 0.18073 | 627168 | 549848 |
| 0.02 | uM | Binding Assay | FP | % Inhibition | Yes | 0.050897 | 34931 | 549662 |
| 0.02 | uM | Binding Assay | FP | % Inhibition | Yes | 0.067104 | 76027 | 549902 |
| 0.02 | uM | Binding Assay | FP | % Inhibition | Yes | 1.3993 | 58514 | 549912 |
| 0.02 | uM | Binding Assay | FP | % Inhibition | Yes | 0.074846 | 218439 | 549940 |
| 0.02 | uM | Binding Assay | FP | % Inhibition | Yes | 0.066683 | 622116 | 549975 |
| 0.02 | uM | Binding Assay | FP | % Inhibition | Yes | 0.17928 | 699479 | 550004 |
| 0.02 | uM | Binding Assay | FP | % inhibition | Yes | 0.055844 | 622124 | 550005 |
| 0.02 | uM | Binding Assay | FR | % Inhibition | Yes | 0.64115 | 20534 | 550026 |
| 0.02 | uM | Binding Assay | FP | % Inhibition | Yes | 0.50571 | 615593 | 550028 |
| 0.02 | uM | Binding Assay | FP | % Inhibition | Yes | 0.24862 | 34391 | 550036 |
| 0.02 | uM | Binding Assay | FP | % Inhibition | Yes | 0.269 | 260610 | 550057 |
| 0.02 | uM | Binding Assay | FP | % Inhibition | Yes | 0.43903 | 177365 | 550089 |
| 0.02 | uM | Binding Assay | FP | % Inhibition | Yes | 0.27223 | 659999 | 550109 |
| 0.02 | uM | Binding Assay | FP | % Inhibition | Yes | 0.14326 | 680734 | 550116 |
| 0.02 | uM | Binding Assay | FP | % Inhibition | Yes | 0.35785 | 125176 | 550124 |

TABLE 8

| Structure | Assay Target | Result Value |
|---|---|---|
| 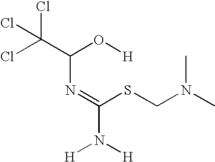 | BCL-b/TR3 | 59.915 |
| 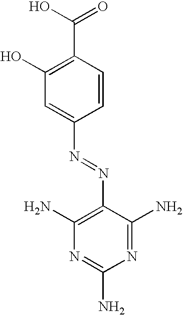 | BCL-b/TR3 | 59.855 |
| 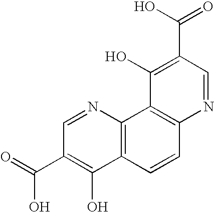 | BCL-b/TR3 | 55.304 |
| 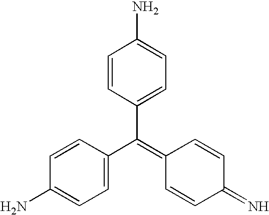 | BCL-b/TR3 | 81.698 |
| 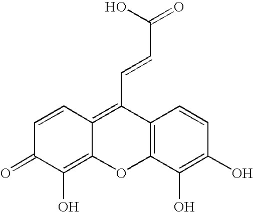 | BCL-b/TR3 | 122.7 |
| 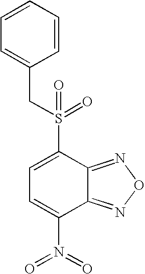 | BCL-b/TR3 | 73.396 |
| 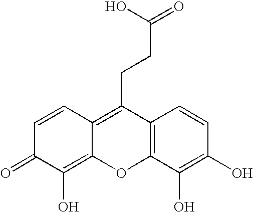 | BCL-b/TR3 | 157.53 |
| 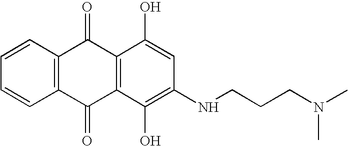 | BCL-b/TR3 | 110.49 |
| 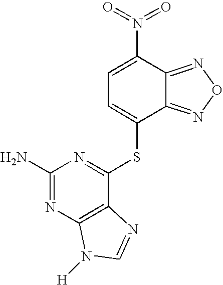 | BCL-b/TR3 | 68.572 |
| 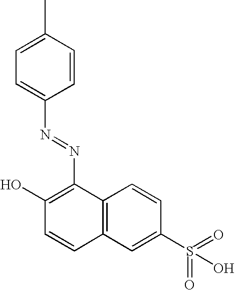 | BCL-b/TR3 | 54.252 |
| 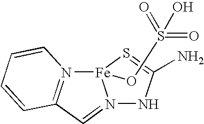 | BCL-b/TR3 | 89.228 |
| 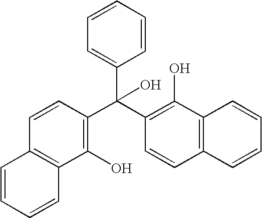 | BCL-b/TR3 | 86.966 |
| 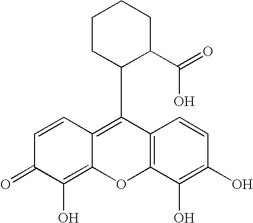 | BCL-b/TR3 | 118.52 |
| 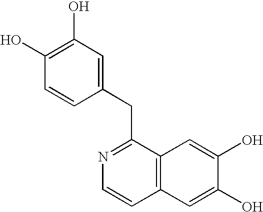 | BCL-b/TR3 | 77.414 |
| 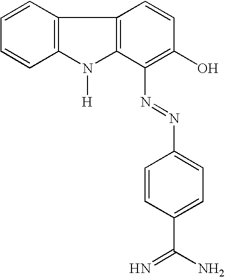 | BCL-b/TR3 | 119.9 |
| 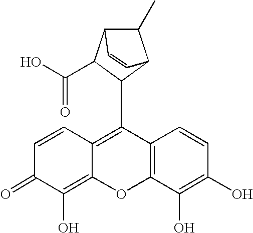 | BCL-b/TR3 | 130.52 |
| 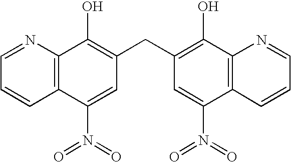 | BCL-b/TR3 | 115.28 |
| 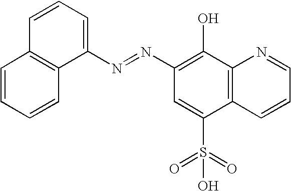 | BCL-b/TR3 | 102.35 |
| 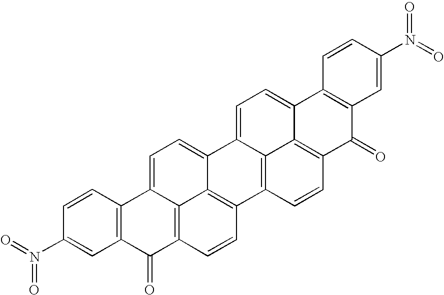 | BCL-b/TR3 | 145.9 |
| 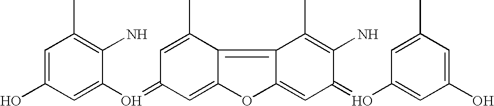 | BCL-b/TR3 | 138.93 |
| 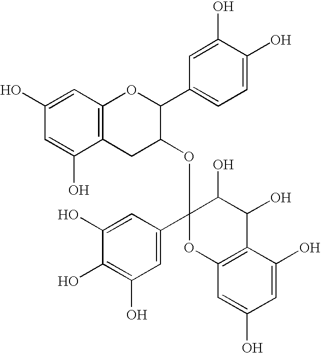 | BCL-b/TR3 | 82.768 |
| 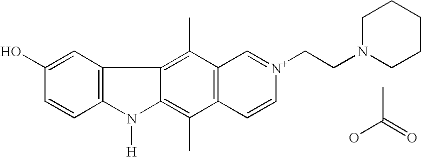 | BCL-b/TR3 | 127.79 |
| 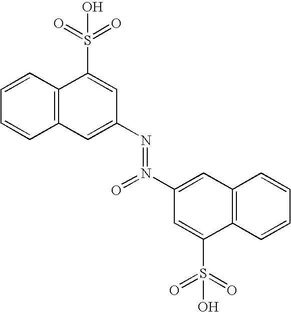 | BCL-b/TR3 | 84.624 |
| 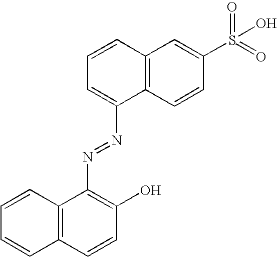 | BCL-b/TR3 | 118.51 |
| 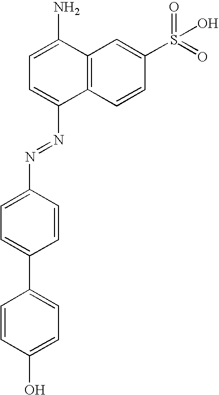 | BCL-b/TR3 | 71.628 |
| 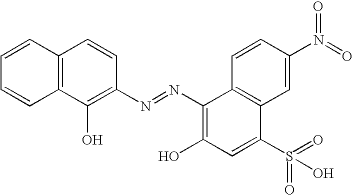 | BCL-b/TR3 | 139.4 |
| 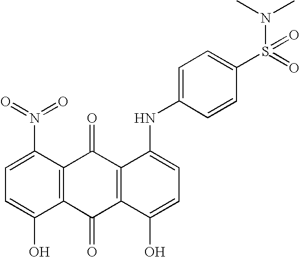 | BCL-b/TR3 | 50.276 |
| 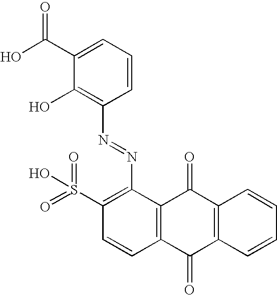 | BCL-b/TR3 | 123.61 |
| 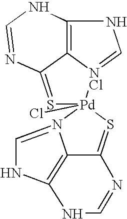 | BCL-b/TR3 | 52.596 |
| 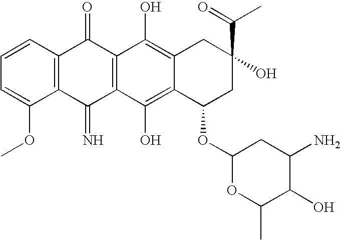 | BCL-b/TR3 | 138 |
| 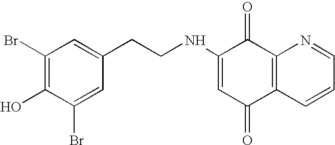 | BCL-b/TR3 | 81.839 |
| 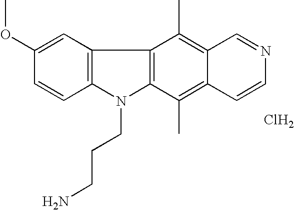 | BCL-b/TR3 | 106.9 |
| 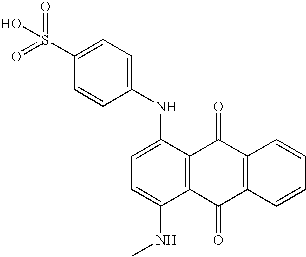 | BCL-b/TR3 | 165.85 |
| 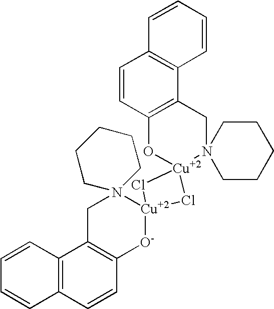 | BCL-b/TR3 | 99.942 |
| 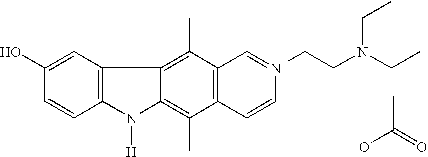 | BCL-b/TR3 | 127.79 |
| 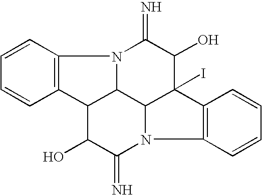 | BCL-b/TR3 | 62.344 |
| 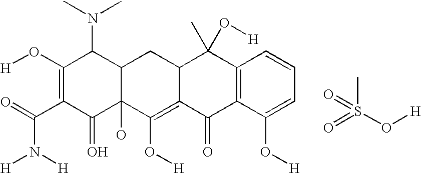 | BCL-b/TR3 | 93.444 |
| 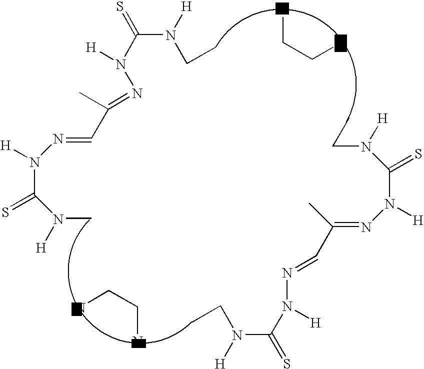 | BCL-b/TR3 | 96.693 |
| 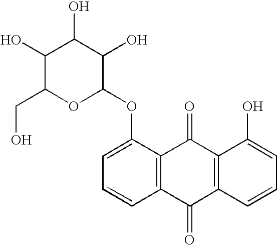 | BCL-b/TR3 | 72.556 |
| 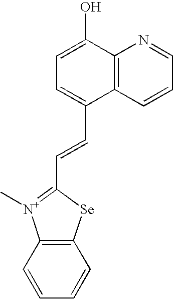 | BCL-b/TR3 | 61.416 |
| 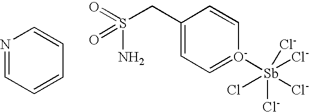 | BCL-b/TR3 | 69.771 |
| 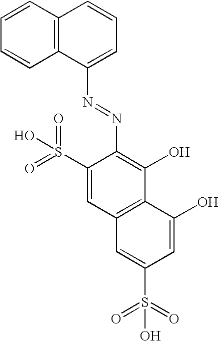 | BCL-b/TR3 | 66.057 |
| 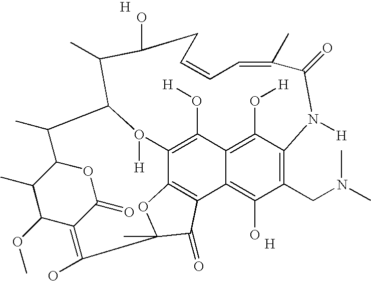 | BCL-b/TR3 | 53.989 |
| 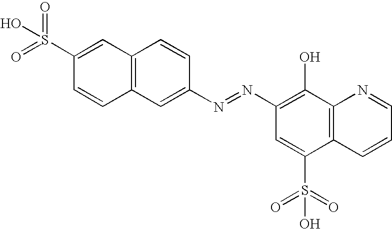 | BCL-b/TR3 | 66.057 |
| 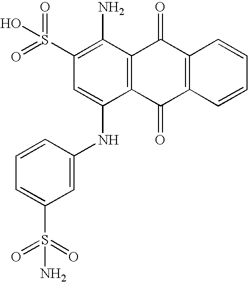 | BCL-b/TR3 | 163.07 |
| 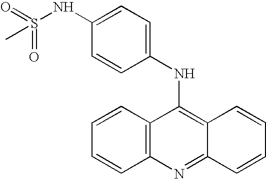 | BCL-b/TR3 | 58.631 |
| 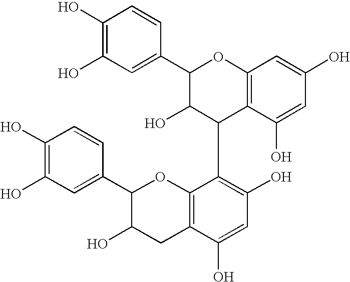 | BCL-b/TR3 | 84.624 |
| 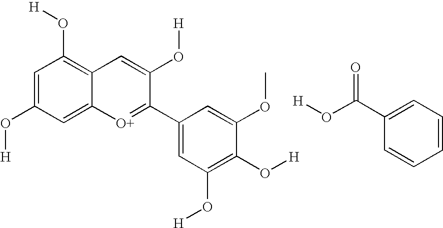 | BCL-b/TR3 | 108.3 |
| 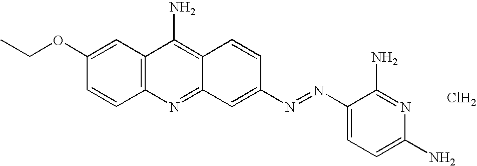 | BCL-b/TR3 | 150.07 |
| 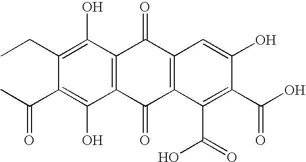 | BCL-b/TR3 | 123.15 |
| 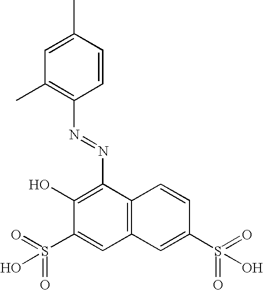 | BCL-b/TR3 | 50.74 |
| 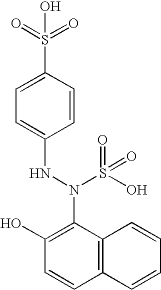 | BCL-b/TR3 | 50.74 |
| 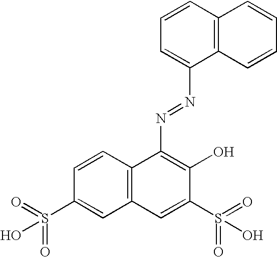 | BCL-b/TR3 | 84.624 |
| 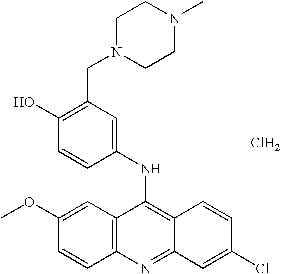 | BCL-b/TR3 | 132.9 |
| 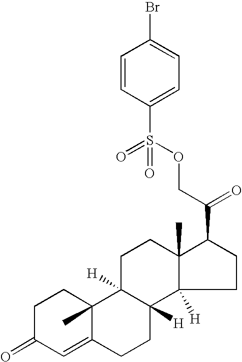 | BCL-b/TR3 | 61.416 |
| 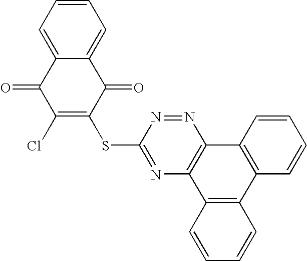 | BCL-b/TR3 | 56.31 |
| 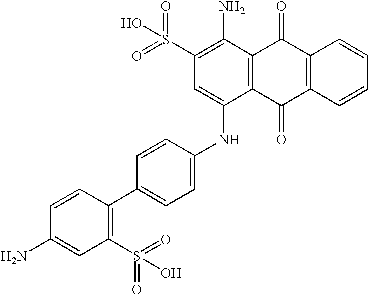 | BCL-b/TR3 | 123.15 |
| 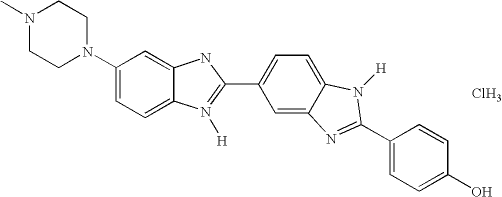 | BCL-b/TR3 | 71.626 |
| 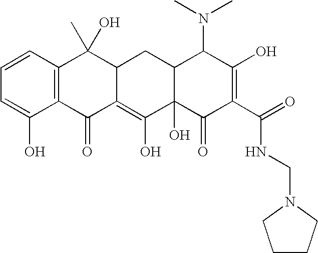 | BCL-b/TR3 | 122.22 |
| 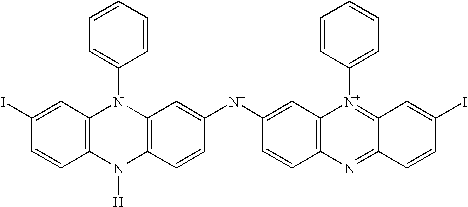 | BCL-b/TR3 | 61.822 |
| 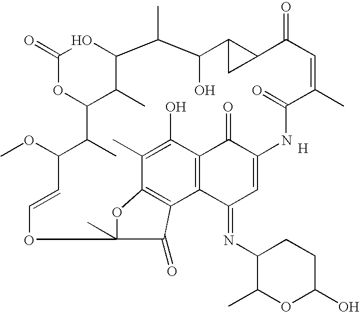 | BCL-b/TR3 | 54.847 |
| 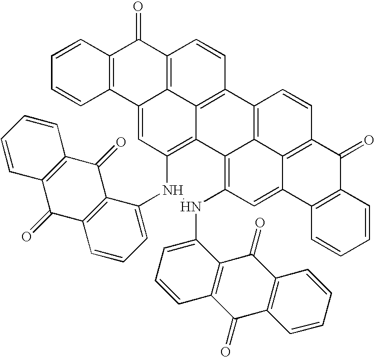 | BCL-b/TR3 | 105.07 |
| 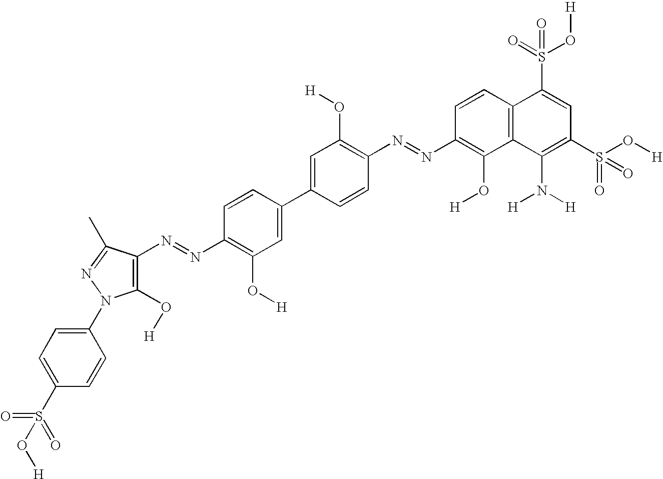 | BCL-b/TR3 | 154.37 |
| 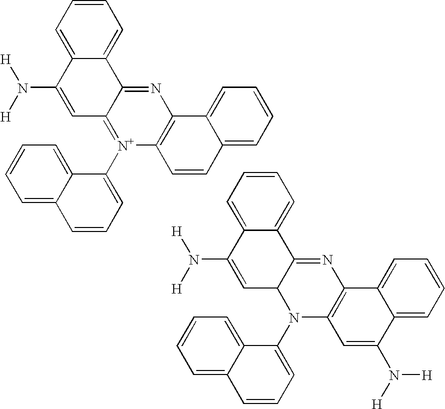 | BCL-b/TR3 | 167.39 |

| | Concentration | | | Result | | | Vendor | |
|---|---|---|---|---|---|---|---|---|
| Concentration | Unit | Assay Name | Assay Format | Type | Is Hit | F Ratio | Compound ID | ID |
| 0.02 | uM | Binding Assay | FP | % Inhibition | Yes | 0.67671 | 14555 | 547889 |
| 0.02 | uM | Binding Assay | FP | % Inhibition | Yes | 0.29542 | 117285 | 546109 |
| 0.02 | uM | Binding Assay | FP | % Inhibition | Yes | 0.20501 | 128884 | 548180 |
| 0.02 | uM | Binding Assay | FP | % Inhibition | Yes | 0.13781 | 10460 | 548249 |
| 0.02 | uM | Binding Assay | FP | % Inhibition | Yes | 0.079916 | 119911 | 548323 |
| 0.02 | uM | Binding Assay | FP | % Inhibition | Yes | 1.1364 | 228148 | 548350 |
| 0.02 | uM | Binding Assay | FP | % Inhibition | Yes | 0.53174 | 119915 | 548363 |
| 0.02 | uM | Binding Assay | FP | % Inhibition | Yes | 0.14158 | 339585 | 548415 |
| 0.02 | uM | Binding Assay | FP | % Inhibition | Yes | 1.4788 | 348401 | 548535 |
| 0.02 | uM | Binding Assay | FP | % Inhibition | Yes | 0.27999 | 7814 | 548566 |
| 0.02 | uM | Binding Assay | FP | % Inhibition | Yes | 1.0155 | 299130 | 548567 |
| 0.02 | uM | Binding Assay | FP | % Inhibition | Yes | 0.77041 | 13984 | 548576 |
| 0.02 | uM | Binding Assay | FP | % Inhibition | Yes | 0.15819 | 119910 | 548623 |
| 0.02 | uM | Binding Assay | FP | % Inhibition | Yes | 0.093694 | 99799 | 548647 |
| 0.02 | uM | Binding Assay | FP | % Inhibition | Yes | 0.069887 | 357777 | 548653 |
| 0.02 | uM | Binding Assay | FP | % Inhibition | Yes | 0.097195 | 119913 | 548823 |
| 0.02 | uM | Binding Assay | FP | % Inhibition | Yes | 0.19686 | 130798 | 546527 |
| 0.02 | uM | Binding Assay | FP | % Inhibition | Yes | 0.070446 | 148354 | 548831 |
| 0.02 | uM | Binding Assay | FP | % Inhibition | Yes | 0.4823 | 51535 | 546919 |
| 0.02 | uM | Binding Assay | FP | % Inhibition | Yes | 0.83759 | 610930 | 548930 |
| 0.02 | uM | Binding Assay | FP | % Inhibition | Yes | 0.18686 | 143101 | 548932 |
| 0.02 | uM | Binding Assay | FP | % Inhibition | Yes | 0.39733 | 311153 | 548940 |
| 0.02 | uM | Binding Assay | FP | % Inhibition | Yes | 0.40219 | 37031 | 548945 |
| 0.02 | uM | Binding Assay | FP | % Inhibition | Yes | 0.059448 | 45576 | 548947 |
| 0.02 | uM | Binding Assay | FP | % Inhibition | Yes | 0.16093 | 65528 | 548949 |
| 0.02 | uM | Binding Assay | FP | % Inhibition | Yes | 0.057123 | 7223 | 548951 |
| 0.02 | uM | Binding Assay | FP | % Inhibition | Yes | 0.93505 | 117949 | 548956 |
| 0.02 | uM | Binding Assay | FP | % Inhibition | Yes | 0.22149 | 125908 | 548958 |
| 0.02 | uM | Binding Assay | FP | % Inhibition | Yes | 0.14274 | 51857 | 548962 |
| 0.02 | uM | Binding Assay | FP | % Inhibition | Yes | 0.52323 | 254681 | 548969 |
| 0.02 | uM | Binding Assay | FP | % Inhibition | Yes | 0.17765 | 668394 | 548971 |
| 0.02 | uM | Binding Assay | FP | % Inhibition | Yes | 1.0005 | 176327 | 548986 |
| 0.02 | uM | Binding Assay | FP | % inhibition | Yes | 0.99285 | 128437 | 548994 |
| 0.02 | uM | Binding Assay | FP | % Inhibition | Yes | 0.12931 | 109268 | 549016 |
| 0.02 | uM | Binding Assay | FP | % Inhibition | Yes | 0.77112 | 311152 | 549029 |
| 0.02 | uM | Binding Assay | FP | % Inhibition | Yes | 0.51279 | 118176 | 549036 |
| 0.02 | uM | Binding Assay | FP | % Inhibition | Yes | 0.37457 | 69343 | 549039 |
| 0.02 | uM | Binding Assay | FP | % Inhibition | Yes | 0.2064 | 91767 | 549049 |
| 0.02 | uM | Binding Assay | FP | % Inhibition | Yes | 0.51423 | 255980 | 549067 |
| 0.02 | uM | Binding Assay | FP | % Inhibition | Yes | 0.094436 | 86374 | 549074 |
| 0.02 | uM | Binding Assay | FP | % Inhibition | Yes | 0.18821 | 13950 | 549081 |
| 0.02 | uM | Binding Assay | FP | % Inhibition | Yes | 0.12193 | 45208 | 549083 |
| 0.02 | uM | Binding Assay | FP | % Inhibition | Yes | 0.96093 | 145612 | 549089 |
| 0.02 | uM | Binding Assay | FP | % Inhibition | Yes | 0.38221 | 87677 | 549105 |
| 0.02 | uM | Binding Assay | FP | % Inhibition | Yes | 0.4053 | 117079 | 549118 |
| 0.02 | uM | Binding Assay | FP | % Inhibition | Yes | 0.12077 | 156305 | 549130 |
| 0.02 | uM | Binding Assay | FP | % Inhibition | Yes | 0.16059 | 143099 | 549132 |
| 0.02 | uM | Binding Assay | FP | % Inhibition | Yes | 0.082485 | 114885 | 549145 |
| 0.02 | uM | Binding Assay | FP | % Inhibition | Yes | 1.1279 | 354961 | 549147 |
| 0.02 | uM | Binding Assay | FP | % Inhibition | Yes | 0.15521 | 170005 | 549149 |
| 0.02 | uM | Binding Assay | FP | % Inhibition | Yes | 0.44058 | 10455 | 549151 |
| 0.02 | uM | Binding Assay | FP | % Inhibition | Yes | 0.43615 | 9600 | 549157 |
| 0.02 | uM | Binding Assay | FP | % Inhibition | Yes | 0.10694 | 45583 | 549163 |
| 0.02 | uM | Binding Assay | FP | % Inhibition | Yes | 0.076963 | 130813 | 549175 |
| 0.02 | uM | Binding Assay | FP | % Inhibition | Yes | 0.26475 | 88915 | 549199 |
| 0.02 | uM | Binding Assay | FP | % Inhibition | Yes | 0.23056 | 327705 | 549204 |
| 0.02 | uM | Binding Assay | FP | % Inhibition | Yes | 0.2439 | 125910 | 549212 |
| 0.02 | uM | Binding Assay | FP | % Inhibition | Yes | 0.50554 | 322921 | 549213 |
| 0.02 | uM | Binding Assay | FP | % Inhibition | Yes | 0.21534 | 50352 | 549214 |
| 0.02 | uM | Binding Assay | FP | % Inhibition | Yes | 0.62609 | 402959 | 549224 |
| 0.02 | uM | Binding Assay | FP | % Inhibition | Yes | 0.8795 | 177383 | 549233 |
| 0.02 | uM | Binding Assay | FP | % Inhibition | Yes | 0.17217 | 23128 | 549243 |
| 0.02 | uM | Binding Assay | FP | % Inhibition | Yes | 0.089665 | 306711 | 549260 |
| 0.02 | uM | Binding Assay | FP | % Inhibition | Yes | 0.90109 | 11241 | 549272 |

What is claimed is:

1. A method of screening for compounds capable of converting a Bcl-B protein from an antiapoptotic form to a proapoptotic form, comprising:
providing a Bcl-B protein;
providing a fluorescently labeled compound known to bind to and convert said Bcl-B protein to a proapoptotic form; and
contacting said Bcl-B protein and said binding compound in the presence or absence of a test compound or library of test compounds; and
determining fluorescence of said Bcl-B protein, wherein a decrease in fluorescence indicates that said test compound inhibits binding of said binding compound to said Bcl-B protein.

2. The method of claim 1, wherein said test compound is a natural product or natural product derivative.

3. The method of claim 1, wherein said fluorescent label is selected from the group consisting of Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

4. The method of claim 1, wherein said compound known to bind to and convert said Bcl-B protein to an apoptotic form is selected from the group consisting of a peptide, peptide analog and small molecule.

5. The method of claim 4, wherein said peptide is TR3-9-r8 peptide.

6. The method of claim 1, further comprising at least one secondary screen to confirm that said test compound converts said Bcl-B protein from an antiapoptotic to a proapoptotic form.

7. The method of claim 6, wherein said secondary screen is an apoptosis assay.

8. The method of claim 1, wherein said screening method is in high throughput format.

9. The method of claim 1, wherein said fluorescence is measured by fluorescence polarization.

10. The method of claim 1, wherein said fluorescence is measured by time-resolved fluorescence resonance energy transfer (TR-FRET), solid phase amplification (SPA) or an ELISA-like assay.

11. The method of claim 1, wherein said decrease in fluorescence is at least 20%.

12. The method of claim 1, wherein said decrease in fluorescence is at least 30%.

13. The method of claim 1, wherein said decrease in fluorescence is at least 40%.

14. The method of claim 1, wherein said decrease in fluorescence is at least 50%.

15. A method of converting a Bcl-B protein from an antiapoptotic form to a proapoptotic form, comprising contacting said Bcl-B protein with a small molecule.

16. The method of claim 15, wherein said small molecule is selected from the group of molecules shown in Tables 5, 6, 7 and 8, or an analog thereof.

17. A method of screening for compounds capable of inhibiting a Bcl-B protein, comprising:
providing a Bcl-B protein;
providing a fluorescently labeled compound known to bind to said Bcl-B protein;
contacting said Bcl-B protein and said fluorescently labeled binding compound in the presence or absence of a test compound or library of test compounds; and
determining fluorescence of said Bcl-B protein, wherein a decrease in fluorescence indicates that said test compound inhibits binding of said fluorescently labeled binding compound to said Bcl-B protein.

18. The method of claim 17, wherein said test compound is a natural product or natural product derivative.

19. The method of claim 17, wherein said fluorescent label is selected from the group consisting of Alexa 350, Alexa 430, AMCA, BODIPY 630/650, BODIPY 650/665, BODIPY-FL, BODIPY—R6G, BODIPY-TMR, BODIPY-TRX, Cascade Blue, Cy2, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

20. The method of claim 17, wherein said compound known to bind to said Bcl-2 protein is selected from the group consisting of a peptide, peptide analog and small molecule.

21. The method of claim 20, wherein said peptide is TR3-9-r8 peptide.

22. The method of claim 17, further comprising at least one secondary screen to confirm that said test compound inhibits said Bcl-2 family protein.

23. The method of claim 22, wherein said secondary screen is an apoptosis assay.

24. The method of claim 17, wherein said screening method is in high throughput format.

25. The method of claim 17, wherein said fluorescence is measured by fluorescence polarization.

26. The method of claim 17, wherein said fluorescence is measured by time-resolved fluorescence resonance energy transfer (TR-FRET), solid phase amplification (SPA) or an ELISA-like assays.

27. The method of claim 17, wherein said decrease in fluorescence is at least 20%.

28. The method of claim 17, wherein said decrease in fluorescence is at least 30%.

29. The method of claim 17, wherein said decrease in fluorescence is at least 40%.

30. The method of claim 17, wherein said decrease in fluorescence is at least 50%.

31. A method of inhibiting a Bcl-B protein, comprising contacting said Bcl-B protein with a small molecule.

32. The method of claim 31, wherein said small molecule is selected from the group of molecules shown in Tables 5, 6, 7 and 8, or an analog thereof.

33. The method of claim 31, wherein said small molecule has the structure 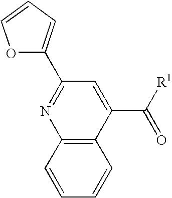
wherein R¹ is selected from the group consisting of —NH═Naryl, —NHaryl, —O[(CH₂)ₚNR¹⁰R¹¹], —O[(CH₂)ₚC(O)NR¹⁰R¹¹], —O[(CH₂)ₚNR¹⁰R¹¹], each optionally substituted with one or more substituents each independently selected from the group consisting of halo, cyano, hydroxy, C₁₋₆ alkyl, C₁₋₆ alkoxy, phenyl, and NR¹⁰R¹¹;
p is 1, 2, or 3; and
R¹⁰ and R¹¹ are each separately selected from hydrogen, C₁₋₆ alkyl, aryl C₁₋₆ alkyl; or R¹⁴ and R¹⁵ are taken together with the nitrogen to which they are attached to form indolinyl, pyrrolidinyl, piperidinyl, piperazinyl, or morpholinyl.

34. The method of claim 31, wherein said small molecule has the structure 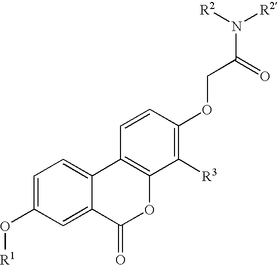
wherein R¹ is selected from the group consisting of hydrogen, aryl, heteroaryl, heterocyclyl, and C₁₋₆ alkyl optionally substituted with up to five fluoro;
R² and R^{2′} are each separately hydrogen or selected from the group consisting of C₁₋₆ alkyl, —(CH₂)_{q}C₃₋₇cycloalkyl, aryl, heteroaryl, and heterocyclyl, each optionally substituted with one or more substituents each independently selected from the group consisting of halo, cyano, hydroxy, —(CH₂)_{q}C₃₋₇cycloalkyl, C₁₋₆ alkyl optionally substituted with up to 5 fluoro, and C₁₋₆ alkoxy optionally substituted with up to 5 fluoro; or R² and R^{2′} are taken together with the nitrogen to which they are attached to form a heterocyclyl;
R³ is hydrogen or selected from the group consisting of C₁₋₆ alkyl, —(CH₂)_{q}C₃₋₇cycloalkyl, and aryl each optionally substituted with one or more substituents each independently selected from the group consisting of halo, cyano, and hydroxy; and
Q is 0, 1, 2, or 3.

35. The method of claim 31, wherein said small molecule has the structure 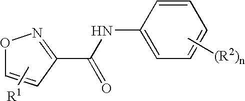
wherein R¹ is hydrogen or selected from the group consisting of C₁₋₆ alkyl, and aryl; or R¹ is a fused C₃₋₇cycloalkyl;
R² is selected from the group consisting of —SC₁₋₆alkyl, C₁₋₆alkoxy, C₁₋₆alkyl, C(O)OC₁₋₆alkyl, and —C(O)NHC₁₋₆alkyl; and
n is an integer selected from 1, 2, 3, 4, or 5.

36. The method of claim 33, wherein said small molecule in one selected from the group consisting of the structures shown in FIG. 25.

37. The method of claim 34, wherein said small molecule in one selected from the group consisting of the structures shown in FIG. 26.

38. The method of claim 35, wherein said small molecule in one selected from the group consisting of the structures shown in FIG. 27.

39. A method of optimizing a target compound, comprising:
providing a Bcl-B protein;
providing a fluorescently labeled compound known to bind to said Bcl-B protein;
contacting said Bcl-B protein and said fluorescently labeled binding compound in the presence or absence of a test compound or library of test compounds;
determining fluorescence of said Bcl-B protein, wherein a decrease in fluorescence indicates that said test compound inhibits binding of said fluorescently labeled binding compound to said Bcl-B protein;
reacting said test compound with a library of chemical fragments in the presence of Bcl-B protein to identify one or more chemical fragments that bind to a site adjacent said test compound; and
linking said chemical fragment to said test compound if the chemical fragment binds adjacent said test compound.
